SEQUENCE LISTING (934 sequences)

SEQ ID NO: 1 - DNA, 5491 bp, Artificial Sequence
  Composed of CCC1p-FTL-CCC1t-CCC1p-FTH-CCC1p- Pcbp1-Adh3t. Thissequence includes the genes for ferritin light chain protein (FTL), ferritin heavy chain protein (FTH) and poly(RC) binding protein (Pcbp1) (HOMO sapiens).
aacccctcagcgtcaggaagacgccacggatccaacaacatcatcgacgaatgaattatctgccgctgagccaacaatggtcacttcgacacatgccactaagacaatacaggctcaaacacaagatcctcccacgaagcacaagaagtccagtttcttcactaaactgttcaagaaaaaaagcagccgttagcagttgtttactcgaattttgcaatcaaccctaatttttgaagcctggttttagatttattctcttctttttcttcttgtgaacttcaattactaatgtaacttaatttttaatataacttttacagtttaataatattgatttttttcggtctggaccaatcgcgccgcatttctcactaatattactaacataccctcttctcattggctcggtacccctttcgtgacccgcattttttgttttctttgttagcccgaatgtctcacaatgaagatgtaaaattaagattatatatgaaaaattgatacaaaacaaaatagttcaaataatcaagttaaagcgacatcactctcaatttttgctttttttaggtttatagaatagaaatataacagaacgaagcctttagactctttttttttattggtatattgaacaaagaaactttttttttgtcctcccatatctcgtgcacacaaatattatgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgcgctctcttccaggacatcaagaagccagctgaagatgagtggggtaaaaccccagacgccatgaaagctgccatggccctggagaaaaagctgaaccaggcccttttggatcttcatgccctgggttctgcccgcacggacccccatctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaagtgtaaagtaacaacactatacatatttattgtaaagaaatttgggattgagaagctttgctatatacttggaactgggctggattttctcgacatacatttcttttacgttttaatttgtttctatattctcctctttaaatttatttatattaatgaatttcaaactagtttctttttctattgccaagaggcccatcaggtgatccatgataacctttattcgcattgaatcgtttttcattggatctaattcgtcatttggtcgccgcctttctttgctccttctgcttttttctttttctctttgactatcaggtcataagatcttctcctccattatgcccaagtgtttttcttttttttgcctttatataaattacaaaatacatacatatacttctcgtgcagtatactggaatcagctttataccatatgaagcacaacttggatcaggaccgctcttcattaaaaatcgaagaattatctaaataattaaaacccctcagcgtcaggaagacgccacggatccaacaacatcatcgacgaatgaattatctgccgctgagccaacaatggtcacttcgacacatgccactaagacaatacaggctcaaacacaagatcctcccacgaagcacaagaagtccagtttcttcactaaactgttcaagaaaaaaagcagccgttagcagttgtttactcgaattttgcaatcaaccctaatttttgaagcctggttttagatttattctcttctttttcttcttgtgaacttcaattactaatgtaacttaatttttaatataacttttacagtttaataatattgatttttttcggtctggaccaatcgcgccgcatttctcactaatattactaacataccctcttctcattggctcggtacccctttcgtgacccgcattttttgttttctttgttagcccgaatgtctcacaatgaagatgtaaaattaagattatatatgaaaaattgatacaaaacaaaatagttcaaataatcaagttaaagcgacatcactctcaatttttgctttttttaggtttatagaatagaaatataacagaacgaagcctttagactctttttttttattggtatattgaacaaagaaactttttttttgtcctcccatatctcgtgcacacaaatattatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagtgtaaagtaacaacactatacatatttattgtaaagaaatttgggattgagaagctttgctatatacttggaactgggctggattttctcgacatacatttcttttacgttttaatttgtttctatattctcctctttaaatttatttatattaatgaatttcaaactagtttctttttctattgccaagaggcccatcaggtgatccatgataacctttattcgcattgaatcgtttttcattggatctaattcgtcatttggtcgccgcctttctttgctccttctgcttttttctttttctctttgactatcaggtcataagatcttctcctccattatgcccaagtgtttttcttttttttgcctttatataaattacaaaatacatacatatacttctcgtgcagtatactggaatcagctttataccatatgaagcacaacttggatcaggaccgctcttcattaaaaatcgaagaattatctaaataattaaaacccctcagcgtcaggaagacgccacggatccaacaacatcatcgacgaatgaattatctgccgctgagccaacaatggtcacttcgacacatgccactaagacaatacaggctcaaacacaagatcctcccacgaagcacaagaagtccagtttcttcactaaactgttcaagaaaaaaagcagccgttagcagttgtttactcgaattttgcaatcaaccctaatttttgaagcctggttttagatttattctcttctttttcttcttgtgaacttcaattactaatgtaacttaatttttaatataacttttacagtttaataatattgatttttttcggtctggaccaatcgcgccgcatttctcactaatattactaacataccctcttctcattggctcggtacccctttcgtgacccgcattttttgttttctttgttagcccgaatgtctcacaatgaagatgtaaaattaagattatatatgaaaaattgatacaaaacaaaatagttcaaataatcaagttaaagcgacatcactctcaatttttgctttttttaggtttatagaatagaaatataacagaacgaagcctttagactctttttttttattggtatattgaacaaagaaactttttttttgtcctcccatatctcgtgcacacaaatattatggatgccggtgtgactgaaagtggactaaatgtgactctcaccattcggcttcttatgcacggaaaggaagtaggaagcatcattgggaagaaaggggagtcggttaagaggatccgcgaggagagtggcgcgcggatcaacatctcggaggggaattgtccggagagaatcatcactctgaccggccccaccaatgccatctttaaggctttcgctatgatcatcgacaagctggaggaagatatcaacagctccatgaccaacagtaccgcggccagcaggcccccggtcaccctgaggctggtggtgccggccacccagtgcggctccctgattgggaaaggcgggtgtaagatcaaagagatccgcgagagtacgggggcgcaggtccaggtggcgggggatatgctgcccaactccaccgagcgggccatcaccatcgctggcgtgccgcagtctgtcaccgagtgtgtcaagcagatttgcctggtcatgctggagacgctctcccagtctccgcaagggagagtcatgaccattccgtaccagcccatgccggccagctccccagtcatctgcgcgggcggccaagatcggtgcagcgacgctgcgggctacccccatgccacccatgacctggagggaccacctctagatgcctactcgattcaaggacaacacaccatttctccgctcgatctggccaagctgaaccaggtggcaagacaacagtctcactttgccatgatgcacggcgggaccggattcgccggaattgactccagctctccagaggtgaaaggctattgggcaagtttggatgcatctactcaaaccacccatgaactcaccattccaaataacttaattggctgcataatcgggcgccaaggcgccaacattaatgagatccgccagatgtccggggcccagatcaaaattgccaacccagtggaaggctcctctggtaggcaggttactatcactggctctgctgccagtattagtctggcccagtatctaatcaatgccaggctttcctctgagaagggcatggggtgcagctagtagcgtgttacgcacccaaactttttatgaaagtctttgtttataatgatgaggtttataaatatatagtggagcaaagattaatcactaaatcaagaagcagtaccagtattttttctatatcaagtagtgataatggaaatagcccaaatttggcttccgtcgacacatagaacgtttgagagacattatcaccatcaagcatcgagccgcccaaacctaaccgtataagttttttcacgtttttgatttttccttgcacacttcgatattactctcacgataaaagggccgaagaga

SEQ ID NO: 2 - DNA, 3593 bp, Artificial Sequence
  Composed of TCO89p-TCO89-TCO89t. This sequence includes the promoter, the gene sequence and the terminator for target of rapamycin complex 1 subunit TCO89 protein (Saccharomyces cerevisiae (strain ATCC 204508 / S288c) (Baker′s yeast)).
taaaagagtttccaaaccagcaagaaagggcaacagaactcgtcgaagcaatacaagttcagacaccaaccaaaatagaaggagtgctgatataggtaccgacaaaccagtaaagcccagattaccccctcaaaggacctcattaaacgaaatgagaagaagggtatccgctattttggagttcatttctagaactcaatgggaattgagtgaagatcagtctgatcgagaggaatttgtacgattcgtggaaaaccagcatttcgtagaaaaagttgatacgatttacaacggttataatgaaagtctatcaatgatggacgacctgactagagagttactactatgggagaaaaaatattcaaataacactaatgccattcaataaacgcaaaacactgcaatattattctcaaccaaagtataactgtaatgaggcgaacaaacacatctatacatatatatacatctatatggatataaaaacgactaattcaacgttgtttttatcaaccgagcttactcttgtacgggtaaccgcaaggatagctagttgcggatggtatagcgatttggctggcacgatgattaaggaatccaaacatctaatggactagcacattctatcgatttacgggtcaggtaaacatagatattgggatatatcatatatccttactgagtaactataattatggttcatcgaggaaggactttgaagtcagacactgatgtaacatctcttaatgcgtcaacagtatcacaccagtcaaagccatttagacagttttcgactaggtcgagagcaaagagtaacgcaagcttcaaaggtttgcgtagagttttaacacatgatggcaccctggataatgattattttaataagcacaacgtttctcagaaatgcaagagttctgatgcacttttcagaaagcgaacgattagtgggttgaatatgacagctttaacaagagtaaagtccaatcaaggaaaaagatcagcatcctttcatagtccggtgcataatacgctgctcagtccaaagaacagcagtcattctaatactggaactgctggtttcggcctgaaaccacgaagaagtaaaagtacccaatctgttctgagtcttcgagatgcgcaagaatctaaaaagagtgaatctactactgacgaggaggtggaatgtttttcggaagacaacattgaagatggaaaggtgaataatgataaagtaatagccgagcatgttatgcctgaagaaaaaaagaatgtgcagcaattaaatcagaatgaattacaatccccggattcaatagatgaacaagaagaagataaatcaggtactgatggaaaggaaaatcatagagctgtatccttaccattacctcatttatcttccaataactatttcggagaatcaagccattctatagaacatcagaaagatggagaaacatctccaagctcaattgaaacaaaactgaatgcaacaagtgtaatcaatgaagaggggcaatcaaaggtgacgaaggaagctgatattgatgacttgtccagccattctcaaaatttgagggcctcattggttaaagcgggcgataatatatcagaagcaccatatgataaagaaaaaaaaattcttgatgttggtaataccttagctgcacataaaagtaatcaaaaaccaagtcattcagatgaacagtttgatcaggaagatcacattgatgcccctaggagtaattcatcaagaaaaagcgactcgagctttatgtctcttaggagacaaagttctaaacaacacaaattattaaacgaagaagaagatctaatcaagcctgatgatatttcttccgctggtaccaaggatattgaagggcatagcttactggaaaattatgcgcctaatatgattctctcccagtcgactggagttgaacgtagatttgaaaattcatcatccatccaaaattcgcttgggaatgaaattcatgactcgggtgagcatatggcttcaggtgatacttttaatgaactggatgatggcaaattgcgcaagagcaagaaaaatggtggaagatctcaacttggccaaaatataccgaactctcagtctactttccccaccattgctaacatcggtagtaaagataataatgtaccacagcacaacttttcgacctccatatcgagtttaaccaataatttgaggagagctgctcctgaaagcttccatggttcaagaatgaataatatttttcacaagaaaggtaatcagaatctacttctgagatccaacgatctcaacaaaaattctgcagccccggcctctccattgtccaacgaacatattacatctagtacgaactccggtagcgatgcaaacagacaatccaactcaggtgccaaatttaatagcttcgcccagttccttaaatcagatgggattgatgcagaatcaagaacacaaagaaaattatggttgcagagggagaattctattatggacttaagttcacaaaatgacggtagtgactctatctttatggcaggaaacattgatgcgaaaagggagtttgagagaatatcccatgaatactctaatgtaaaaagattttacaacccattagatgaagcattgttgagagtacaacctataataacgggaaatgcaaataatatcaggaaaaaaagccataacgatgctcagtcaatcgcacattctagcagtgatacagatcataaggatgaggacgatttgctctttactaactatgacaaaaaatttgatgatctttatccacatcttgcaagtgcaaagattcaggcagtgttgtccggtatatggaaaagcgaaagttacttatttaacaaggatgttaatccaatcaacaagaataggacaacgagtacaaaccacagcgttggccacactgcttcacagaatgcacgtaacttgctgaggggcccgatgggttccagcacgactttgcaccaccaacgcgtcattaactctctgcagccgactacgagggcagtgaatcgcaggatggaaaatgtgggctacatgcatacacagccacaacaaaggtgaaaacaaccaacacgaagcacacagtttaaaagagtagctaatgctttcgaggtaaaacgcgaagttcgtagagagcgaatatgtttggacacttaaggaacatactatgtttatgcactataaaaggacatgtacttctatacgagctaacgaggcgtatttgtataacccggttaacgtaataaatgataaattatcgaacaaaaagaaagaaaaacgttgaatgcaaccaccgcaaatttagcgatttcgctggattccgtatctcttaaaaaatggcattagtaaatataccttaaacgtgtctattcttttagcaattttttggtaagtattcctcacggactataaatactatcggtcagaatcactacattcaaattaatcttgttttaccgagtctctgacgtttcatttacaagcttgtccttacaaaaaacctattttattactttagtccattttcctttcaagttagatattgttttcccttcactta

SEQ ID NO: 3 - DNA, 2261 bp, Artificial Sequence
  Composed of GPDp-peptide#1-Aga1-Cyc1t. This sequence includes the gene coding for A-agglutinin anchorage subunit protein (Saccaromyces cerevisiae) and the gene coding for the single letter amino acid code MHGKTQATSGTIQS.
atactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggattctcaaaaaactaatccatctgattctcaaaaaactaatccatctgattctcaaaaaactaatccatctgattctcaaaaaactaatccatctgattctcaaaaaactaatccatctgattctcaaaaaactaatccatctgattctcaaaaaactaatccatctgattctcaaaaaactaatccatcttaaactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagcttcgcagtttacactctcatc

SEQ ID NO: 4 - DNA, 2363 bp, Artificial Sequence
  Composed of GPDp-peptide#2-Aga1-Cyc1t.This sequence includes the gene coding for A-agglutinin anchorage subunit protein (Saccaromyces cerevisiae) and the gene coding for the single letter amino acid code DSQKTNPS.
atactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgatgcatggtaaaactcaagctacttctggtactattcaatctatgcatggtaaaactcaagctacttctggtactattcaatctatgcatggtaaaactcaagctacttctggtactattcaatctatgcatggtaaaactcaagctacttctggtactattcaatctatgcatggtaaaactcaagctacttctggtactattcaatctatgcatggtaaaactcaagctacttctggtactattcaatctatgcatggtaaaactcaagctacttctggtactattcaatcttaaactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagcttcgcagtttacactctcatc

SEQ ID NO: 5 - DNA, 552 bp, Artificial Sequence
  FTH synthesis. FTH is the gene coding for ferritin heavy chain protein (Homo sapiens (Human)).
atgacaactgcttctacatcacaagttagacaaaattatcatcaagattctgaagctgcaattaatagacaaattaatttggaattatacgcttcatacgtttatttgtcaatgtcttattactttgatagagatgatgttgctttgaaaaattttgctaaatactttttgcatcaatctcatgaagaaagagaacatgctgaaaaattgatgaaattacaaaatcaaagaggtggtagaattttcttgcaagatattaaaaagccagattgtgatgattgggaatcaggtttaaatgcaatggaatgtgctttacatttggaaaagaatgttaatcaatctttgttagaattgcataaattggctactgataaaaatgatccacatttgtgtgattttattgaaactcattatttgaatgaacaagttaaagctattaaagaattgggtgaccatgttactaatttgagaaaaatgggtgcaccagaatcaggtttggctgaatacttatttgataaacatactttgggtgactcagataatgaatcataa

SEQ ID NO: 6 - DNA, 552 bp, Artificial Sequence; identical to SEQ ID NO: 5
  FTL synthesis. FTL is the gene coding for ferritin light chain protein (Homo sapiens (Human)).

SEQ ID NO: 7 - DNA, 1071 bp, Artificial Sequence
  PCBP1 synthesis. PCBP1 is the gene coding for poly(RC) binding protein 1 (Homo sapiens (Human)).
atggatgctggtgttacagaatctggtttgaatgttacattgactattagattgttaatgcatggtaaagaagttggttcaattattggtaaaaagggtgaatctgttaaaagaattagagaagaatctggtgctagaattaatatttctgaaggtaattgtccagaaagaattattactttaacaggtccaactaatgctatttttaaagcatttgctatgattattgataaattggaagaagatattaattcatctatgactaattcaacagctgcatcaagaccaccagttactttgagattagttgttccagcaacacaatgtggttctttgattggtaaaggtggttgtaaaattaaagaaattagagaatctactggtgcacaagttcaagttgcaggtgacatgttgccaaattctactgaaagagcaattacaattgctggtgttccacaatcagttactgaatgtgttaaacaaatttgtttagttatgttggaaacattgtctcaatcaccacaaggtagagttatgactattccataccaaccaatgccagcatcttcaccagttatttgtgctggtggtcaagatagatgttcagatgctgcaggttatccacatgcaacacatgatttggaaggtccaccattggatgcttattcaattcaaggtcaacatactatttcaccattagatttggctaaattgaatcaagttgctagacaacaatcacattttgctatgatgcatggtggtacaggttttgctggtattgattcatcttcaccagaagttaaaggttattgggcttctttagatgcttctactcaaacaactcatgaattgactattccaaataatttgattggttgtattattggtagacaaggtgcaaatattaatgaaattagacaaatgtctggtgctcaaattaaaattgcaaatccagttgaaggttcatctggtagacaagttacaattactggttctgctgcatctatttcattggcacaatacttaattaatgcaagattgtcatctgaaaaaggtatgggttgttcttaa

SEQ ID NO: 8 - DNA, 255 bp, Artificial Sequence
  Peptide 1 synthesis. Gene coding for MHGKTQATSGTIQS (single-letter amino acid).
atggattctcaaaagacaaatccatcagattctcaaaagactaatccatcagattctcaaaagacaaatccatctgattctcaaaagactaatccatctgattcacaaaagactaatccatcagattcacaaaagacaaatccatctgattcacaaaagacaaatccatcagattcacaaaagactaatccatctggtggtggtggttctggtggtggtggttctggtggtggtggttctgcttctggtggtggt

SEQ ID NO: 9 - DNA, 354 bp, Artificial Sequence
  Peptide 2 synthesis. Gene coding for DSQKTNPS (single-letter amino acid code).
atgcatggtaaaacacaagctacttcaggtactattcaatctatgcatggtaaaacacaagcaacatctggtacaattcaatctatgcatggtaaaactcaagcaacttctggtactattcaatctatgcatggtaaaactcaagctacttctggtacaattcaatcaatgcatggtaaaactcaagctacatctggtactattcaatcaatgcatggtaaaacacaagctacttcaggtacaattcaatcaatgcatggtaaaacacaagcaacatcaggtactattcaatctggtggtggtggttctggtggtggtggttctggtggtggtggttctgcttctggtggtggt

SEQ ID NO: 10 - DNA, 10587 bp, Artificial Sequence
  Composed of pRS316-FTL-FTH-Pcbp1. This sequence includes the genes coding for ferritin light chain protein (FTL), ferritin heavy chain protein (FTH) and poly(RC) binding protein (Pcbp1) (Homo sapiens). pRS316 is a yeast centrosome vector with a URA3
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctccaccgcggtggcggccgcaacccctcagcgtcaggaagacgccacggatccaacaacatcatcgacgaatgaattatctgccgctgagccaacaatggtcacttcgacacatgccactaagacaatacaggctcaaacacaagatcctcccacgaagcacaagaagtccagtttcttcactaaactgttcaagaaaaaaagcagccgttagcagttgtttactcgaattttgcaatcaaccctaatttttgaagcctggttttagatttattctcttctttttcttcttgtgaacttcaattactaatgtaacttaatttttaatataacttttacagtttaataatattgatttttttcggtctggaccaatcgcgccgcatttctcactaatattactaacataccctcttctcattggctcggtacccctttcgtgacccgcattttttgttttctttgttagcccgaatgtctcacaatgaagatgtaaaattaagattatatatgaaaaattgatacaaaacaaaatagttcaaataatcaagttaaagcgacatcactctcaatttttgctttttttaggtttatagaatagaaatataacagaacgaagcctttagactctttttttttattggtatattgaacaaagaaactttttttttgtcctcccatatctcgtgcacacaaatattatgtcatctcaaattagacaaaattactctactgatgttgaagctgcagttaattctttggttaatttgtacttacaagcatcatacacatatttgtctttaggtttttactttgatagagatgatgttgctttagaaggtgtttctcatttctttagagaattggctgaagaaaagagagaaggttacgaaagattgttaaaaatgcaaaatcaaagaggtggtagagctttgtttcaagatattaaaaagccagctgaagatgaatggggtaaaacaccagatgctatgaaagcagctatggcattggaaaagaaattgaatcaagcattgttagatttgcatgctttaggttctgctagaacagatccacatttgtgtgatttcttggaaactcatttcttggatgaagaagttaaattgattaaaaagatgggtgaccatttgactaatttgcatagattgggtggtccagaagctggtttgggtgaatacttgtttgaaagattaactttgaaacatgattaagtgtaaagtaacaacactatacatatttattgtaaagaaatttgggattgagaagctttgctatatacttggaactgggctggattttctcgacatacatttcttttacgttttaatttgtttctatattctcctctttaaatttatttatattaatgaatttcaaactagtttctttttctattgccaagaggcccatcaggtgatccatgataacctttattcgcattgaatcgtttttcattggatctaattcgtcatttggtcgccgcctttctttgctccttctgcttttttctttttctctttgactatcaggtcataagatcttctcctccattatgcccaagtgtttttcttttttttgcctttatataaattacaaaatacatacatatacttctcgtgcagtatactggaatcagctttataccatatgaagcacaacttggatcaggaccgctcttcattaaaaatcgaagaattatctaaataattaatgtttttaatgctgatttcctataatattaaccggtaacccctcagcgtcaggaagacgccacggatccaacaacatcatcgacgaatgaattatctgccgctgagccaacaatggtcacttcgacacatgccactaagacaatacaggctcaaacacaagatcctcccacgaagcacaagaagtccagtttcttcactaaactgttcaagaaaaaaagcagccgttagcagttgtttactcgaattttgcaatcaaccctaatttttgaagcctggttttagatttattctcttctttttcttcttgtgaacttcaattactaatgtaacttaatttttaatataacttttacagtttaataatattgatttttttcggtctggaccaatcgcgccgcatttctcactaatattactaacataccctcttctcattggctcggtacccctttcgtgacccgcattttttgttttctttgttagcccgaatgtctcacaatgaagatgtaaaattaagattatatatgaaaaattgatacaaaacaaaatagttcaaataatcaagttaaagcgacatcactctcaatttttgctttttttaggtttatagaatagaaatataacagaacgaagcctttagactctttttttttattggtatattgaacaaagaaactttttttttgtcctcccatatctcgtgcacacaaatattatgacaactgcttctacatcacaagttagacaaaattatcatcaagattctgaagctgcaattaatagacaaattaatttggaattatacgcttcatacgtttatttgtcaatgtcttattactttgatagagatgatgttgctttgaaaaattttgctaaatactttttgcatcaatctcatgaagaaagagaacatgctgaaaaattgatgaaattacaaaatcaaagaggtggtagaattttcttgcaagatattaaaaagccagattgtgatgattgggaatcaggtttaaatgcaatggaatgtgctttacatttggaaaagaatgttaatcaatctttgttagaattgcataaattggctactgataaaaatgatccacatttgtgtgattttattgaaactcattatttgaatgaacaagttaaagctattaaagaattgggtgaccatgttactaatttgagaaaaatgggtgcaccagaatcaggtttggctgaatacttatttgataaacatactttgggtgactcagataatgaatcataagtgtaaagtaacaacactatacatatttattgtaaagaaatttgggattgagaagctttgctatatacttggaactgggctggattttctcgacatacatttcttttacgttttaatttgtttctatattctcctctttaaatttatttatattaatgaatttcaaactagtttctttttctattgccaagaggcccatcaggtgatccatgataacctttattcgcattgaatcgtttttcattggatctaattcgtcatttggtcgccgcctttctttgctccttctgcttttttctttttctctttgactatcaggtcataagatcttctcctccattatgcccaagtgtttttcttttttttgcctttatataaattacaaaatacatacatatacttctcgtgcagtatactggaatcagctttataccatatgaagcacaacttggatcaggaccgctcttcattaaaaatcgaagaattatctaaataattaaatccgctctaaccgaaaaggaaggagttaggaattcaacccctcagcgtcaggaagacgccacggatccaacaacatcatcgacgaatgaattatctgccgctgagccaacaatggtcacttcgacacatgccactaagacaatacaggctcaaacacaagatcctcccacgaagcacaagaagtccagtttcttcactaaactgttcaagaaaaaaagcagccgttagcagttgtttactcgaattttgcaatcaaccctaatttttgaagcctggttttagatttattctcttctttttcttcttgtgaacttcaattactaatgtaacttaatttttaatataacttttacagtttaataatattgatttttttcggtctggaccaatcgcgccgcatttctcactaatattactaacataccctcttctcattggctcggtacccctttcgtgacccgcattttttgttttctttgttagcccgaatgtctcacaatgaagatgtaaaattaagattatatatgaaaaattgatacaaaacaaaatagttcaaataatcaagttaaagcgacatcactctcaatttttgctttttttaggtttatagaatagaaatataacagaacgaagcctttagactctttttttttattggtatattgaacaaagaaactttttttttgtcctcccatatctcgtgcacacaaatattatggatgctggtgttacagaatctggtttgaatgttacattgactattagattgttaatgcatggtaaagaagttggttcaattattggtaaaaagggtgaatctgttaaaagaattagagaagaatctggtgctagaattaatatttctgaaggtaattgtccagaaagaattattactttaacaggtccaactaatgctatttttaaagcatttgctatgattattgataaattggaagaagatattaattcatctatgactaattcaacagctgcatcaagaccaccagttactttgagattagttgttccagcaacacaatgtggttctttgattggtaaaggtggttgtaaaattaaagaaattagagaatctactggtgcacaagttcaagttgcaggtgacatgttgccaaattctactgaaagagcaattacaattgctggtgttccacaatcagttactgaatgtgttaaacaaatttgtttagttatgttggaaacattgtctcaatcaccacaaggtagagttatgactattccataccaaccaatgccagcatcttcaccagttatttgtgctggtggtcaagatagatgttcagatgctgcaggttatccacatgcaacacatgatttggaaggtccaccattggatgcttattcaattcaaggtcaacatactatttcaccattagatttggctaaattgaatcaagttgctagacaacaatcacattttgctatgatgcatggtggtacaggttttgctggtattgattcatcttcaccagaagttaaaggttattgggcttctttagatgcttctactcaaacaactcatgaattgactattccaaataatttgattggttgtattattggtagacaaggtgcaaatattaatgaaattagacaaatgtctggtgctcaaattaaaattgcaaatccagttgaaggttcatctggtagacaagttacaattactggttctgctgcatctatttcattggcacaatacttaattaatgcaagattgtcatctgaaaaaggtatgggttgttcttaagtgtaaagtaacaacactatacatatttattgtaaagaaatttgggattgagaagctttgctatatacttggaactgggctggattttctcgacatacatttcttttacgttttaatttgtttctatattctcctctttaaatttatttatattaatgaatttcaaactagtttctttttctattgccaagaggcccatcaggtgatccatgataacctttattcgcattgaatcgtttttcattggatctaattcgtcatttggtcgccgcctttctttgctccttctgcttttttctttttctctttgactatcaggtcataagatcttctcctccattatgcccaagtgtttttcttttttttgcctttatataaattacaaaatacatacatatacttctcgtgcagtatactggaatcagctttataccatatgaagcacaacttggatcaggaccgctcttcattaaaaatcgaagaattatctaaataattaactcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 11 - DNA, 9390 bp, Artificial Sequence
  Composed of pRS423-TC089. pRS423 is a yeast episomal vector with a HIS3 marker and an MCS derived from pBLUESCRIPT II. TC089 is the gene coding for target of rapamycin complex 1 subunit TCO89 protein (Saccharomyces cerevisiae (strain ATCC 204508 / S288c)).
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgattaaaagagtttccaaaccagcaagaaagggcaacagaactcgtcgaagcaatacaagttcagacaccaaccaaaatagaaggagtgctgatataggtaccgacaaaccagtaaagcccagattaccccctcaaaggacctcattaaacgaaatgagaagaagggtatccgctattttggagttcatttctagaactcaatgggaattgagtgaagatcagtctgatcgagaggaatttgtacgattcgtggaaaaccagcatttcgtagaaaaagttgatacgatttacaacggttataatgaaagtctatcaatgatggacgacctgactagagagttactactatgggagaaaaaatattcaaataacactaatgccattcaataaacgcaaaacactgcaatattattctcaaccaaagtataactgtaatgaggcgaacaaacacatctatacatatatatacatctatatggatataaaaacgactaattcaacgttgtttttatcaaccgagcttactcttgtacgggtaaccgcaaggatagctagttgcggatggtatagcgatttggctggcacgatgattaaggaatccaaacatctaatggactagcacattctatcgatttacgggtcaggtaaacatagatattgggatatatcatatatccttactgagtaactataattatggttcatcgaggaaggactttgaagtcagacactgatgtaacatctcttaatgcgtcaacagtatcacaccagtcaaagccatttagacagttttcgactaggtcgagagcaaagagtaacgcaagcttcaaaggtttgcgtagagttttaacacatgatggcaccctggataatgattattttaataagcacaacgtttctcagaaatgcaagagttctgatgcacttttcagaaagcgaacgattagtgggttgaatatgacagctttaacaagagtaaagtccaatcaaggaaaaagatcagcatcctttcatagtccggtgcataatacgctgctcagtccaaagaacagcagtcattctaatactggaactgctggtttcggcctgaaaccacgaagaagtaaaagtacccaatctgttctgagtcttcgagatgcgcaagaatctaaaaagagtgaatctactactgacgaggaggtggaatgtttttcggaagacaacattgaagatggaaaggtgaataatgataaagtaatagccgagcatgttatgcctgaagaaaaaaagaatgtgcagcaattaaatcagaatgaattacaatccccggattcaatagatgaacaagaagaagataaatcaggtactgatggaaaggaaaatcatagagctgtatccttaccattacctcatttatcttccaataactatttcggagaatcaagccattctatagaacatcagaaagatggagaaacatctccaagctcaattgaaacaaaactgaatgcaacaagtgtaatcaatgaagaggggcaatcaaaggtgacgaaggaagctgatattgatgacttgtccagccattctcaaaatttgagggcctcattggttaaagcgggcgataatatatcagaagcaccatatgataaagaaaaaaaaattcttgatgttggtaataccttagctgcacataaaagtaatcaaaaaccaagtcattcagatgaacagtttgatcaggaagatcacattgatgcccctaggagtaattcatcaagaaaaagcgactcgagctttatgtctcttaggagacaaagttctaaacaacacaaattattaaacgaagaagaagatctaatcaagcctgatgatatttcttccgctggtaccaaggatattgaagggcatagcttactggaaaattatgcgcctaatatgattctctcccagtcgactggagttgaacgtagatttgaaaattcatcatccatccaaaattcgcttgggaatgaaattcatgactcgggtgagcatatggcttcaggtgatacttttaatgaactggatgatggcaaattgcgcaagagcaagaaaaatggtggaagatctcaacttggccaaaatataccgaactctcagtctactttccccaccattgctaacatcggtagtaaagataataatgtaccacagcacaacttttcgacctccatatcgagtttaaccaataatttgaggagagctgctcctgaaagcttccatggttcaagaatgaataatatttttcacaagaaaggtaatcagaatctacttctgagatccaacgatctcaacaaaaattctgcagccccggcctctccattgtccaacgaacatattacatctagtacgaactccggtagcgatgcaaacagacaatccaactcaggtgccaaatttaatagcttcgcccagttccttaaatcagatgggattgatgcagaatcaagaacacaaagaaaattatggttgcagagggagaattctattatggacttaagttcacaaaatgacggtagtgactctatctttatggcaggaaacattgatgcgaaaagggagtttgagagaatatcccatgaatactctaatgtaaaaagattttacaacccattagatgaagcattgttgagagtacaacctataataacgggaaatgcaaataatatcaggaaaaaaagccataacgatgctcagtcaatcgcacattctagcagtgatacagatcataaggatgaggacgatttgctctttactaactatgacaaaaaatttgatgatctttatccacatcttgcaagtgcaaagattcaggcagtgttgtccggtatatggaaaagcgaaagttacttatttaacaaggatgttaatccaatcaacaagaataggacaacgagtacaaaccacagcgttggccacactgcttcacagaatgcacgtaacttgctgaggggcccgatgggttccagcacgactttgcaccaccaacgcgtcattaactctctgcagccgactacgagggcagtgaatcgcaggatggaaaatgtgggctacatgcatacacagccacaacaaaggtgaaaacaaccaacacgaagcacacagtttaaaagagtagctaatgctttcgaggtaaaacgcgaagttcgtagagagcgaatatgtttggacacttaaggaacatactatgtttatgcactataaaaggacatgtacttctatacgagctaacgaggcgtatttgtataacccggttaacgtaataaatgataaattatcgaacaaaaagaaagaaaaacgttgaatgcaaccaccgcaaatttagcgatttcgctggattccgtatctcttaaaaaatggcattagtaaatataccttaaacgtgtctattcttttagcaattttttggtaagtattcctcacggactataaatactatcggtcagaatcactacattcaaattaatcttgttttaccgagtctctgacgtttcatttacaagcttgtccttacaaaaaacctattttattactttagtccattttcctttcaagttagatattgttttcccttcacttaatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 12 - DNA, 9104 bp, Artificial Sequence
  Composed of pRS425-peptide 1. pRS425 is a yeast episomal vector with a LEU3 marker and an MCS derived from pBLUESCRIPT II. Peptide 1 is the gene coding for the single amino acid chain MHGKTQATSGTIQS.
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggattctcaaaagacaaatccatcagattctcaaaagactaatccatcagattctcaaaagacaaatccatctgattctcaaaagactaatccatctgattcacaaaagactaatccatcagattcacaaaagacaaatccatctgattcacaaaagacaaatccatcagattcacaaaagactaatccatctggtggtggtggttctggtggtggtggttctggtggtggtggttctgcttctggtggtggtactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagcttcgcagtttacactctcatcgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 13 - DNA, 9203 bp, Artificial Sequence
  Composed of pRS425-peptide 2. pRS425 is a yeast episomal vector with a LEU3 marker and an MCS derived from pBLUESCRIPT II. Peptide 2 is the gene coding for the single amino acid chain DSQKTNPS.
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgcatggtaaaacacaagctacttcaggtactattcaatctatgcatggtaaaacacaagcaacatctggtacaattcaatctatgcatggtaaaactcaagcaacttctggtactattcaatctatgcatggtaaaactcaagctacttctggtacaattcaatcaatgcatggtaaaactcaagctacatctggtactattcaatcaatgcatggtaaaacacaagctacttcaggtacaattcaatcaatgcatggtaaaacacaagcaacatcaggtactattcaatctggtggtggtggttctggtggtggtggttctggtggtggtggttctgcttctggtggtggtactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagcttcgcagtttacactctcatcgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 14 - protein, 12 aa, Artificial Sequence
  Selectively binds to Au3+, Au, Pd, Pd4+, Pt2+ and Ag. Display platform: M13 phage, streptavidin coated glass slide, SPOT synthesized peptide array and chemical synthesis.
AYSSGAPPMPPF

SEQ ID NO: 15 - protein, 12 aa, Artificial Sequence
  Selectively binds to Au3+. Display platform: streptavidin coated glass slide.
ASSSGAPPMPPF

SEQ ID NO: 16 - protein, 8 aa, Artificial Sequence
  Selectively binds to Au3+, Au and Pd. Display platform: streptavidin coated glass slide and simulation.
DYKDDDDK

SEQ ID NO: 17 - protein, 21 aa, Artificial Sequence
  Selectively binds to Au3+, Pd4+, Pt 2+ and Au nanoparticles. Display platform: chemical synthesis and streptavidin coated glass slides.
DYKDDDDKPAYSSGAPPMPPF

SEQ ID NO: 18 - protein, 20 aa, Artificial Sequence
  Selectively binds to Au3+, Pd4+ and Pt2+. Display platform: streptavidin coated glass slides and chemical synthesis.
AYSSGAPPMPPFPDYKDDDK

SEQ ID NO: 19 - protein, 14 aa, Artificial Sequence
  Selectively binds to Au powder. Display platform: E. coli.
ALVPTAHRIDGNMH

SEQ ID NO: 20 - protein, 14 aa, Artificial Sequence
  Selectively binds to Au powder. Display platform: E. coli.
ALPRGVYKIDSNMH

SEQ ID NO: 21 - protein, 28 aa, Artificial Sequence
  Selectively binds to Au powder. Display platform: E. coli.
PGMKASKSMRNQATPGMPSSLDLTWQAT

SEQ ID NO: 22 - protein, 28 aa, Artificial Sequence
  Selectively binds to Au powder. Display platform: E. coli.
PGMKMRLSGAKEATPGMSTTVAGLLQAT

SEQ ID NO: 23 - protein, 28 aa, Artificial Sequence
  Selectively binds to Au powder. Display platform: E. coli.
PGMIHVQKTAVQATPGMVNLTSPVKQAT

SEQ ID NO: 24 - protein, 28 aa, Artificial Sequence
  Selectively binds to Au powder. Display platform: E. coli.
AIDSPAGCISFSMHMHGKTQATSGTIQS

SEQ ID NO: 25 - protein, 14 aa, Artificial Sequence
  Selectively binds to Au powder. Display platform: E. coli, E. coli fermentation, SPOT synthesized peptide array, M13K07 helper phage, Fmoc synthesis, Au coated Si AFM probe and chemical synthesis.
MHGKTQATSGTIQS

SEQ ID NO: 26 - protein, 147 aa, Artificial Sequence
  Selectively binds to Au. Display platform: E. coli.
SKTSLGCQKPLYMGREMRMLTSKTSLGCQKPLYMGREMRMLTSKTSLQQSGASLQGSEKLTNGSKTSLQQSGASLQGSEKLTNGSKTSLQQSGASLQGSEKLTNGSKTSLQQSGASLQGSEKLTNGSKTSLQQSGASLQGSEKLTNG

SEQ ID NO: 27 - protein, 147 aa, Artificial Sequence
  Selectively binds to Au. Display platform: E. coli.
QATSEKLVRGMEGASLHPAKTQATSEKLVRGMEGASLHPAKTQATSEKLVRGMEGASLHPAKTQATSEKLVRGMEGASLHPAKTQATSEKLVRGMEGASLHPAKTQATSEKLVRGMEGASLHPAKTQATSEKLVRGMEGASLHPAKT

SEQ ID NO: 28 - protein, 357 aa, Artificial Sequence
  Selectively binds to Au. Display platform: E. coli.
SKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKISKTSTNNFGGMMPGGDESTKI

SEQ ID NO: 29 - protein, 231 aa, Artificial Sequence
  Selectively binds to Au. Display platform: E. coli.
QATSEMQRQMGIRVGPEQDKTQATSEMQRQMGIRVGPEQDKTQATSEMQRQMGIRVGPEQDKTQATSEMQRQMGIRVGPEQDKTQATSEMQRQMGIRVGPEQDKTQATSEMQRQMGIRVGPEQDKTQATSEMQRQMGIRVGPEQDKTQATSEMQRQMGIRVGPEQDKTQATSEMQRQMGIRVGPEQDKTQATSEMQRQMGIRVGPEQDKTQATSEMQRQMGIRVGPEQDKT

SEQ ID NO: 30 - protein, 147 aa, Artificial Sequence
  Selectively binds to Au. Display platform: E. coli.
QATSGSERMGHQSGTVHPGKTQATSGSERMGHQSGTVHPGKTQATSGSERMGHQSGTVHPGKTQATSGSERMGHQSGTVHPGKTQATSGSERMGHQSGTVHPGKTQATSGSERMGHQSGTVHPGKTQATSGSERMGHQSGTVHPGKT

SEQ ID NO: 31 - protein, 12 aa, Artificial Sequence
  Selectively binds to Au. Display platform: phage display and M13 virus.
LKAHLPPSRLPS

SEQ ID NO: 32 - protein, 12 aa, Artificial Sequence
  Selectively binds to Au. Display platform: phage display.
KPHTPHNHPSHH

SEQ ID NO: 33 - protein, 58 aa, Artificial Sequence
  Selectively binds to Au. Display platform: Saccharomyces cerevisiae.
PGLVKPSQTLSLTCAISGDSVSSNSAGWTWIRQSPSRGLEWLGRTYYKSKWYYDMQYL

SEQ ID NO: 34 - protein, 58 aa, Artificial Sequence
  Selectively binds to Au. Display platform: Saccharomyces cerevisiae.
PGLVKPSQTLSLTCAISGDSVSSNRAAWNWIRQSPSRGLEWLGRTYHRSKWGYDMRYL

SEQ ID NO: 35 - protein, 58 aa, Artificial Sequence
  Selectively binds Au. Display platform: Saccharomyces cerevisiae.
PGLVKPSQTLSLTCAISGDSVSGNTAAWNWIRQSPSRGLEWLGRTYYRSKWHYDMRHL

SEQ ID NO: 36 - protein, 7 aa, Artificial Sequence
  Selectively binds for Au, ZnS, CdS, Au coated slides and AuPt nanoparticles. Display platform: Saccharomyces cerevisiae and SPOT synthesized peptide array.
KHKHWHW

SEQ ID NO: 37 - protein, 7 aa, Artificial Sequence
  Selectively binds to Au. Display platform: Saccharomyces cerevisiae and SPOT synthesized peptide array.
RMRMKMK

SEQ ID NO: 38 - protein, 12 aa, Artificial Sequence
  Selectively binds to gold foil, , Au, Au & Ag QCM sensor surfaces. Display platform: E. coli.
WAGAKRLVLRRE

SEQ ID NO: 39 - protein, 18 aa, Artificial Sequence
  Selectively binds to Au. Display platform: E. coli.
CGPWAGAKRLVLRREGPC

SEQ ID NO: 40 - protein, 12 aa, Artificial Sequence
  Selectively binds to Au, Au nanoparticles and Au & Ag QCM sensor surfaces. Display platform: E. coli, SPOT syntehsized peptide array and chemical synthesis.
WALRRSIRRQSY

SEQ ID NO: 41 - protein, 18 aa, Artificial Sequence
  Selectively binds to Au. Display platform: E. coli.
CGPWALRRSIRRQSYGPC

SEQ ID NO: 42 - protein, 21 aa, Artificial Sequence
  Selectively binds to Au and gold powder. Display platform: M13 peptide library.
SKTSLGQSGQSLQGSEKLTNG

SEQ ID NO: 43 - protein, 21 aa, Artificial Sequence
  Selectively binds to chlorauric acid and Au. Display platform: M13 peptide library.
QATSEKLVRGMEGASLHPAKT

SEQ ID NO: 44 - protein, 13 aa, Artificial Sequence
  Selectively binds to gold foil and Au nanoparticles. Display platform: SPOT synthesized peptide array and chemical synthesis.
WAGAKRLVLRRGE

SEQ ID NO: 45 - protein, 17 aa, Artificial Sequence
  Selectively binds to Au and AuCl3. Display platform: SPOT synthesized peptide array.
LGQSGQSLQGSEKLTNG

SEQ ID NO: 46 - protein, 15 aa, Artificial Sequence
  Selectively binds to AuCl3. Display platform: SPOT synthesized peptide array.
EKLVRGMEGASLHPA

SEQ ID NO: 47 - protein, 10 aa, Artificial Sequence
  Selectively binds to Au nanoparticles. Display platform: SPOT synthesized peptide array.
HFSSWETQQG

SEQ ID NO: 48 - protein, 10 aa, Artificial Sequence
  Selectively binds to Au nanoparticles. Display platform: SPOT syntehsized peptide array.
WTHRDASTPW

SEQ ID NO: 49 - protein, 10 aa, Artificial Sequence
  Selectively binds to Au nanoparticles. Display platform: SPOT synthesized peptide array.
WYEKWQKANW

SEQ ID NO: 50 - protein, 10 aa, Artificial Sequence
  Selectively binds to Au nanoparticles. Display platform: SPOT synthesized  peptide array.
WMETKWQARA

SEQ ID NO: 51 - protein, 10 aa, Artificial Sequence
  Selectively binds to Au nanoparticles. Display platform: SPOT synthesized peptide array.
GTWSEHQNGW

SEQ ID NO: 52 - protein, 10 aa, Artificial Sequence
  Selectively binds to Au nanoparticles. Display platform: SPOT synthesized peptide array.
ETWSMQQHEW

SEQ ID NO: 53 - protein, 10 aa, Artificial Sequence
  Selectively binds to Au nanoparticles. Display platform: SPOT synthesized peptide array.
WRAGQAQMQW

SEQ ID NO: 54 - protein, 10 aa, Artificial Sequence
  Selectively binds to Au nanoparticles. Display platform: SPOT synthesized peptide array.
WKPWMEPQHS

SEQ ID NO: 55 - protein, 10 aa, Artificial Sequence
  Selectively binds to Au nanoparticles. Display platform: SPOT synthesized peptide array.
AMQQQWEMSQ

SEQ ID NO: 56 - protein, 10 aa, Artificial Sequence
  Selectively binds to Au nanoparticles. Display platform: SPOT synthesized peptide array.
RWQIEEHFAP

SEQ ID NO: 57 - protein, 10 aa, Artificial Sequence
  Selectively binds to Au nanoparticles. Display platform: SPOT synthesized peptide array.
PEESQEGWMA

SEQ ID NO: 58 - protein, 10 aa, Artificial Sequence
  Selectively binds to Au nanoparticles. Display platform: SPOT synthesized peptide array.
TGEWGMQGIH

SEQ ID NO: 59 - protein, 10 aa, Artificial Sequence
  Selectively binds to Au nanoparticles. Display platform: SPOT synthesized peptide array.
EEPHWEEMAA

SEQ ID NO: 60 - protein, 10 aa, Artificial Sequence
  Selectively binds to Au nanoparticles. Display platform: SPOT synthesized peptide array.
WWKVANIHSK

SEQ ID NO: 61 - protein, 10 aa, Artificial Sequence
  Selectively binds to Au nanoparticles. Display platform: SPOT synthesized peptide array,
RHWHSWTWEI

SEQ ID NO: 62 - protein, 10 aa, Artificial Sequence
  Selectively binds to Au nanoparticles. Display platform: SPOT synthesized peptide array.
MNWKWGLESM

SEQ ID NO: 63 - protein, 10 aa, Artificial Sequence
  Selectively binds to Au nanoparticles. Display platform: SPOT synthesized peptide array.
NWTAKWTQTH

SEQ ID NO: 64 - protein, 10 aa, Artificial Sequence
  Selectively binds to Au nanoparticles. Display platform: SPOT synthesized peptide array.
HWIKIPPWMW

SEQ ID NO: 65 - protein, 10 aa, Artificial Sequence
  Selectively binds to Au nanoparticles. Display platform: SPOT synthesized peptide array.
HWKQKVHWWG

SEQ ID NO: 66 - protein, 10 aa, Artificial Sequence
  Selectively binds to Au nanoparticles. Display platform: SPOT synthesized peptide array.
WHKWWTHGHW

SEQ ID NO: 67 - protein, 10 aa, Artificial Sequence
  Selectively binds to Au nanoparticles. Display platform: SPOT synthesized peptide array.
KYWQMWMSWK

SEQ ID NO: 68 - protein, 10 aa, Artificial Sequence
  Selectively binds to Au nanoparticles. Display platform: SPOT synthesized peptide array.
KWQWKQAGAQ

SEQ ID NO: 69 - protein, 10 aa, Artificial Sequence
  Selectively binds to Au nanoparticles. Display platform: SPOT synthesized peptide array.
EHQQWKETWH

SEQ ID NO: 70 - protein, 10 aa, Artificial Sequence
  Selectively binds to Au nanoparticles. Display platform: SPOT synthesized peptide array.
GQWQWMDAGW

SEQ ID NO: 71 - protein, 10 aa, Artificial Sequence
  Selectively binds to Au nanoparticles. Display platform: SPOT synthesized peptide array.
QWTWKIQVMK

SEQ ID NO: 72 - protein, 10 aa, Artificial Sequence
  Selectively binds to Au nanoparticles. Display platform: SPOT synthesized peptide array.
HWKGEMHTDF

SEQ ID NO: 73 - protein, 10 aa, Artificial Sequence
  Selectively binds to Au nanoparticles. Display platform: SPOT synthesized peptide array.
PEEGPHSLWH

SEQ ID NO: 74 - protein, 10 aa, Artificial Sequence
  Selectively binds to Au nanoparticles. Display platform: SPOT synthesized peptide array.
EWVEAMGGHT

SEQ ID NO: 75 - protein, 10 aa, Artificial Sequence
  Selectively binds to Au nanoparticles. Display platform: SPOT synthesized peptide array.
WPAMGWNMEQ

SEQ ID NO: 76 - protein, 10 aa, Artificial Sequence
  Selectively binds to Au nanoparticles. Display platform: SPOT synthesized peptide array.
KWAIWEMKGH

SEQ ID NO: 77 - protein, 10 aa, Artificial Sequence
  Selectively binds to Au nanoparticles. Display platform: SPOT synthesized peptide array.
GETWETHYSE

SEQ ID NO: 78 - protein, 10 aa, Artificial Sequence
  Selectively binds to Au nanoparticles. Display platform: SPOT synthesized peptide array.
WVHKRLNWTV

SEQ ID NO: 79 - protein, 10 aa, Artificial Sequence
  Selectively binds to Au nanoparticles. Display platform: SPOT synthesized peptide array.
WSWPKVKSFW

SEQ ID NO: 80 - protein, 10 aa, Artificial Sequence
  Selectively binds to Au nanoparticles. Display platform: SPOT synthesized peptide array.
MLGWMHQSWQ

SEQ ID NO: 81 - protein, 10 aa, Artificial Sequence
  Selectively binds to Au nanoparticles. Display platform: SPOT synthesized peptide array.
NWKWQMKWTQ

SEQ ID NO: 82 - protein, 10 aa, Artificial Sequence
  Selectively binds to Au nanoparticles. Display platform: SPOT synthesized peptide array.
EWHVKWSEAI

SEQ ID NO: 83 - protein, 10 aa, Artificial Sequence
  Selectively binds to Au nanoparticles. Display platform: SPOT synthesized peptide array.
LAGVPMHWYT

SEQ ID NO: 84 - protein, 10 aa, Artificial Sequence
  Selectively binds to Au nanoparticles. Display platform: SPOT synthesized peptide array.
QEHLSEMWGE

SEQ ID NO: 85 - protein, 10 aa, Artificial Sequence
  Selectively binds to Au nanoparticles. Display platform: SPOT synthesized peptide array.
ASHQWAWKWE

SEQ ID NO: 86 - protein, 10 aa, Artificial Sequence
  Selectively binds to Au nanoparticles. Display platform: SPOT synthesized peptide array.
WSEETEMWPL

SEQ ID NO: 87 - protein, 10 aa, Artificial Sequence
  Selectively binds to Au nanoparticles. Display platform: SPOT synthesized peptide array.
DMVWHESWGI

SEQ ID NO: 88 - protein, 10 aa, Artificial Sequence
  Selectively binds to Au nanoparticles. Display platform: SPOT synthesized peptide array.
WWLQKWHGSH

SEQ ID NO: 89 - protein, 10 aa, Artificial Sequence
  Selectively binds to Au nanoparticles. Display platform: SPOT synthesized peptide array.
ENHSWGGGGA

SEQ ID NO: 90 - protein, 10 aa, Artificial Sequence
  Selectively binds to Au nanoparticles. Display platform: SPOT synthesized peptide array.
QRHSWGGGEA

SEQ ID NO: 91 - protein, 10 aa, Artificial Sequence
  Selectively binds to Au nanoparticles. Display platform: SPOT synthesized peptide array.
WKATWAKYEK

SEQ ID NO: 92 - protein, 10 aa, Artificial Sequence
  Selectively binds to Au nanoparticles. Display platform: SPOT synthesized peptide array.
TWHIMWRHAW

SEQ ID NO: 93 - protein, 10 aa, Artificial Sequence
  Selectively binds to Au nanoparticles. Display platform: SPOT synthesized peptide array.
WEAKEWLHNW

SEQ ID NO: 94 - protein, 10 aa, Artificial Sequence
  Selectively binds to Au nanoparticles. Display platform: SPOT synthesized peptide array.
NKGGGWQGPE

SEQ ID NO: 95 - protein, 10 aa, Artificial Sequence
  Selectively binds to Au nanoparticles. Display platform: SPOT synthesized peptide array.
WGWKWEHSEA

SEQ ID NO: 96 - protein, 12 aa, Artificial Sequence
  Selectively binds to Au and Pd. Display platform: M13 phage and simulation.
NFMSLPRLGHMH

SEQ ID NO: 97 - protein, 12 aa, Artificial Sequence
  Selectively binds to Au, Pd and Pd films deposited on silicon. Display platform: simulation and E. coli.
TSNAVHPTLRHL

SEQ ID NO: 98 - protein, 10 aa, Artificial Sequence
  Selectively binds to Au and Pd. Display platform: simulation.
GGGGGGGGGG

SEQ ID NO: 99 - protein, 10 aa, Artificial Sequence
  Selectively binds to Au and Pd. Display platform: simulation.
PPPPPPPPPP

SEQ ID NO: 100 - protein, 7 aa, Artificial Sequence
  Selectively binds to Pt. Display platform: M13 PD library.
DRTSTWR

SEQ ID NO: 101 - protein, 7 aa, Artificial Sequence
  Selectively binds to Pt. Display platform: M13 PD library.
QSVTSTK

SEQ ID NO: 102 - protein, 7 aa, Artificial Sequence
  Selectively binds to Pt. Display platform: M13 PD library.
SSSHLNK

SEQ ID NO: 103 - protein, 7 aa, Artificial Sequence
  Selectively binds to Pd. Display platform: M13 PD library.
SVTQNKY

SEQ ID NO: 104 - protein, 7 aa, Artificial Sequence
  Selectively binds to Pd. Display platform: M13 PD library.
SPHPGPY

SEQ ID NO: 105 - protein, 7 aa, Artificial Sequence
  Selectively binds to Pd. Display platform: M13 PD library.
HAPTPML

SEQ ID NO: 106 - protein, 9 aa, Artificial Sequence
  Selectively binds to Au, Ag, Ni, Au nanoparticles, ZnS, TiO2 and Ag2S. Display platform: chemical synthesis.
AHHAHHAAD

SEQ ID NO: 107 - protein, 4 aa, Artificial Sequence
  Selectively binds to Au nanoparticles. Display platform: chemical synthesis.
CALN

SEQ ID NO: 108 - protein, 5 aa, Artificial Sequence
  Selectively binds to Au nanoparticles. Display platform: chemical synthesis.
CALNN

SEQ ID NO: 109 - protein, 6 aa, Artificial Sequence
  Selectively binds to Au nanoparticles. Display platform: chemical synthesis.
CCALNN

SEQ ID NO: 110 - protein, 5 aa, Artificial Sequence
  Selectively binds to Au nanoparticles. Display platform: chemical synthesis.
KALNN

SEQ ID NO: 111 - protein, 5 aa, Artificial Sequence
  Selectively binds to Au nanoparticles. Display platform: chemical synthesis.
AALNN

SEQ ID NO: 112 - protein, 5 aa, Artificial Sequence
  Selectively binds to Au nanoparticles. Display platform: chemical synthesis.
NNLAC

SEQ ID NO: 113 - protein, 5 aa, Artificial Sequence
  Selectively binds to Au nanoparticles. Display platform: chemical synthesis.
CILNN

SEQ ID NO: 114 - protein, 5 aa, Artificial Sequence
  Selectively binds to Au nanoparticles. Display platform: chemical synthesis.
CLLNN

SEQ ID NO: 115 - protein, 5 aa, Artificial Sequence
  Selectively binds to Au nanoparticles. Display platform: chemical synthesis.
CVLNN

SEQ ID NO: 116 - protein, 5 aa, Artificial Sequence
  Selectively binds to Au nanoparticles. Display platform: chemical synthesis.
CFLNN

SEQ ID NO: 117 - protein, 5 aa, Artificial Sequence
  Selectively binds to Au nanoparticles. Display platform: chemical synthesis.
CAANN

SEQ ID NO: 118 - protein, 5 aa, Artificial Sequence
  Selectively binds to Au nanoparticles. Display platform: chemical synthesis.
CAINN

SEQ ID NO: 119 - protein, 5 aa, Artificial Sequence
  Selectively binds to Au nanoparticles. Display platform: chemical synthesis.
CAVNN

SEQ ID NO: 120 - protein, 5 aa, Artificial Sequence
  Selectively binds to Au nanoparticles. Display platform: chemical synthesis.
CLANN

SEQ ID NO: 121 - protein, 5 aa, Artificial Sequence
  Selectively binds to Au nanoparticles. Display platform: chemical synthesis.
CKLNN

SEQ ID NO: 122 - protein, 5 aa, Artificial Sequence
  Selectively binds to Au nanoparticles. Display platform: chemical synthesis.
CAFNN

SEQ ID NO: 123 - protein, 5 aa, Artificial Sequence
  Selectively binds to Au nanoparticles. Display platform: chemical synthesis.
CDLNN

SEQ ID NO: 124 - protein, 5 aa, Artificial Sequence
  Selectively binds to Au nanoparticles. Display platform: chemical synthesis.
CTLNN

SEQ ID NO: 125 - protein, 4 aa, Artificial Sequence
  Selectively binds to Au nanoparticles. Display platform: chemical synthesis.
CNNN

SEQ ID NO: 126 - protein, 5 aa, Artificial Sequence
  Selectively binds to Au nanoparticles. Display platform: chemical synthesis.
CAKNN

SEQ ID NO: 127 - protein, 5 aa, Artificial Sequence
  Selectively binds to Au nanoparticles. Display platform: chemical synthesis.
CADNN

SEQ ID NO: 128 - protein, 5 aa, Artificial Sequence
  Selectively binds to Au nanoparticles. Display platform: chemical synthesis.
CATNN

SEQ ID NO: 129 - protein, 5 aa, Artificial Sequence
  Selectively binds to Au nanoparticles. Display platform: chemical synthesis.
CANNN

SEQ ID NO: 130 - protein, 5 aa, Artificial Sequence
  Selectively binds to Au nanoparticles. Display platform: chemical synthesis.
CDDNN

SEQ ID NO: 131 - protein, 5 aa, Artificial Sequence
  Selectively binds to Au nanoparticles. Display platform: chemical synthesis.
CKKNN

SEQ ID NO: 132 - protein, 5 aa, Artificial Sequence
  Selectively binds to Au nanoparticles. Display platform: chemical synthesis.
CTTNN

SEQ ID NO: 133 - protein, 5 aa, Artificial Sequence
  Selectively binds to Au nanoparticles. Display platform: chemical synthesis.
CTSNN

SEQ ID NO: 134 - protein, 5 aa, Artificial Sequence
  Selectively binds to Au nanoparticles. Display platform: chemical synthesis.
CALLS

SEQ ID NO: 135 - protein, 5 aa, Artificial Sequence
  Selectively binds to Au nanoparticles. Display platform: chemical synthesis.
CALLD

SEQ ID NO: 136 - protein, 5 aa, Artificial Sequence
  Selectively binds to Au nanoparticles. Display platform: chemical synthesis.
CALLK

SEQ ID NO: 137 - protein, 5 aa, Artificial Sequence
  Selectively binds to Au nanoparticles. Display platform: chemical synthesis.
CALLR

SEQ ID NO: 138 - protein, 5 aa, Artificial Sequence
  Selectively binds to Au nanoparticles. Display platform: chemical synthesis.
CALNS

SEQ ID NO: 139 - protein, 5 aa, Artificial Sequence
  Selectively binds to Au nanoparticles. Display platform: chemical synthesis.
CALND

SEQ ID NO: 140 - protein, 5 aa, Artificial Sequence
  Selectively binds to Au nanoparticles. Display platform: chemical synthesis.
CALNK

SEQ ID NO: 141 - protein, 5 aa, Artificial Sequence
  Selectively binds to Au nanoparticles. Display platform: chemical synthesis.
CALNR

SEQ ID NO: 142 - protein, 6 aa, Artificial Sequence
  Selectively binds to Au nanoparticles. Display platform: chemical synthesis.
NCALSS

SEQ ID NO: 143 - protein, 5 aa, Artificial Sequence
  Selectively binds to Au nanoparticles. Display platform: chemical synthesis.
CALSR

SEQ ID NO: 144 - protein, 5 aa, Artificial Sequence
  Selectively binds to Au nanoparticles. Display platform: chemical synthesis.
CALKS

SEQ ID NO: 145 - protein, 5 aa, Artificial Sequence
  Selectively binds to Au nanoparticles. Display platform: chemical synthesis.
CTTTT

SEQ ID NO: 146 - protein, 5 aa, Artificial Sequence
  Selectively binds to Au nanoparticles. Display platform: chemical synthesis.
CHRIS

SEQ ID NO: 147 - protein, 5 aa, Artificial Sequence
  Selectively binds to Au nanoparticles. Display platform: chemical synthesis.
CVVIT

SEQ ID NO: 148 - protein, 11 aa, Artificial Sequence
  Selectively binds to Au nanoparticles. Display platform: chemical synthesis.
CAALPDGLAAC

SEQ ID NO: 149 - protein, 5 aa, Artificial Sequence
  Selectively binds to Au nanoparticles. Display platform: chemical synthesis.
CALSD

SEQ ID NO: 150 - protein, 5 aa, Artificial Sequence
  Selectively binds to Au nanoparticles. Display platform: chemical synthesis.
CALKK

SEQ ID NO: 151 - protein, 13 aa, Artificial Sequence
  Selectively binds to Au nanoparticles. Display platform: chemical synthesis.
CVVITPDGTIVVC

SEQ ID NO: 152 - protein, 5 aa, Artificial Sequence
  Selectively binds to Au nanoparticles. Display platform: chemical synthesis.
CALKD

SEQ ID NO: 153 - protein, 5 aa, Artificial Sequence
  Selectively binds to Au nanoparticles. Display platform: chemical synthesis.
CALSK

SEQ ID NO: 154 - protein, 5 aa, Artificial Sequence
  Selectively binds to Au nanoparticles. Display platform: chemical synthesis.
CALSS

SEQ ID NO: 155 - protein, 9 aa, Artificial Sequence
  Selectively binds to Au nanoparticles. Display platform: chemical synthesis.
NNLACALNN

SEQ ID NO: 156 - protein, 10 aa, Artificial Sequence
  Selectively binds to Au nanoparticles. Display platform: chemical synthesis.
NNLACCALNN

SEQ ID NO: 157 - protein, 6 aa, Artificial Sequence
  Selectively binds to Au nanoparticles. Display platform: chemical synthesis.
CCVVVK

SEQ ID NO: 158 - protein, 6 aa, Artificial Sequence
  Selectively binds to Au nanoparticles. Display platform: chemical synthesis.
CCVVVT

SEQ ID NO: 159 - protein, 15 aa, Artificial Sequence
  Selectively binds to Au nanoparticles. Display platform: chemical synthesis.
CALNNGGWSHPQFEK

SEQ ID NO: 160 - protein, 8 aa, Artificial Sequence
  Selectively binds to Au and Au nanoparticles. Display platform: Fmoc synthesis (genescript).
VSGSSPDS

SEQ ID NO: 161 - protein, 7 aa, Artificial Sequence
  Selectively binds to Pt and 2 nm of Pt coated directly onto Au. Display platform: M13 phage and Fmoc synthesis.
PTSTGQA

SEQ ID NO: 162 - protein, 7 aa, Artificial Sequence
  Selectively binds to Pt and Pt(100). Display platform: M13 (Ph.D. -7, NEB)
TLTTLTN

SEQ ID NO: 163 - protein, 7 aa, Artificial Sequence
  Selectively binds to Pt and Pt(111). Display platform: M13 (Ph.D. -7, NEB)
SSFPQPN

SEQ ID NO: 164 - protein, 11 aa, Artificial Sequence
  Selectively binds to Pt. Display platform: CSBio 336s automated peptide synthesizer (SBio, USA) on Wang resin.
CSQSVTSTKSC

SEQ ID NO: 165 - protein, 12 aa, Artificial Sequence
  Selectively binds to Au. Display platform: phage library and SPOT synthesized peptide array.
TGTSVLIATPYV

SEQ ID NO: 166 - protein, 12 aa, Artificial Sequence
  Selectively binds to Au and Si 4 nm SiOx spatter coated on a gold SPR chip. Display platform: chemical synthesis.
PPPWLPYMPPWS

SEQ ID NO: 167 - protein, 5 aa, Artificial Sequence
  Selectively binds to Au. Display platform: CSBio 336s automated peptide synthesizer (SBio, USA) on Wang resin.
GRGDS

SEQ ID NO: 168 - protein, 5 aa, Artificial Sequence
  Selectively binds to Au. Display platform: Fmoc synthesis.
IKVAV

SEQ ID NO: 169 - protein, 38 aa, Artificial Sequence
  Selectively binds to Au nanoparticles. Display platform: chemical synthesis.
CGGEVSALEKEVSALEKEVSALEKEVSALEKEVSALEK

SEQ ID NO: 170 - protein, 12 aa, Artificial Sequence
  Selectively binds to Au & Ag QCOM sensor surfaces. Display platform: FliTrx bacterial surface library.
TGIFKSARAMRN

SEQ ID NO: 171 - protein, 12 aa, Artificial Sequence
  Selectively binds to Au  & Ag QCM sensor surfaces. Display platform: FliTrx bacterial surface library.
EQLGVRKELRGV

SEQ ID NO: 172 - protein, 21 aa, Artificial Sequence
  Selectively binds to Au, Ag, ZnS and Pt. Display platform: streptavidin coated polystyrene beads.
QATSIGVEKLAGMAESKPTKT

SEQ ID NO: 173 - protein, 16 aa, Artificial Sequence
  Selectively binds to Au, TiO2, Al3O4 and SiO. Display platform: chemical synthesis.
AGSWLRDIWTWLQSAL

SEQ ID NO: 174 - protein, 12 aa, Artificial Sequence
  Selectively binds to Pd nanoparticles. Display platform: mutations of SEQ ID NO. 105.
TSNAVHPTLRAL

SEQ ID NO: 175 - protein, 12 aa, Artificial Sequence
  Selectively binds to Pd nanoparticles. Display platform: mutations of SEQ ID NO. 105.
TSNAVAPTLRHL

SEQ ID NO: 176 - protein, 12 aa, Artificial Sequence
  Selectively binds to Pd nanoparticles. Display platform: mutations of SEQ ID NO. 105.
TSNAVAPTLRAL

SEQ ID NO: 177 - protein, 13 aa, Artificial Sequence
  Selectively binds to PdAu nanoparticles. Display platform: chemical synthesis and through the combination of SEQ ID NO. 105 and SEQ ID NO. 39.
WAGAKRHPTLRHL

SEQ ID NO: 178 - protein, 12 aa, Artificial Sequence
  Selectively binds to Ag and Ag nanoparticles. Display platform: M13 phage.
NPSSLFRYLPSD

SEQ ID NO: 179 - protein, 12 aa, Artificial Sequence
  Selectively binds to Ag. Display platform: M13 phage.
SLATQPPRTPPV

SEQ ID NO: 180 - protein, 12 aa, Artificial Sequence
  Selectively binds to Ag. Display platform: M13 phage.
KFLQFVCLGVGP

SEQ ID NO: 181 - protein, 12 aa, Artificial Sequence
  Selectively binds to Ag. Display platform: M13 phage.
AVLMQKYHQLGP

SEQ ID NO: 182 - protein, 12 aa, Artificial Sequence
  Selectively binds to Ag. Display platform: M13 phage.
IRPAIHIIPISH

SEQ ID NO: 183 - protein, 12 aa, Artificial Sequence
  Selectively binds to Ag. Display platform: M13 phage.
NVIRASPPDTSY

SEQ ID NO: 184 - protein, 12 aa, Artificial Sequence
  Selectively binds to Ag. Display platform: M13 phage.
LAMPNTQADAPF

SEQ ID NO: 185 - protein, 12 aa, Artificial Sequence
  Selectively binds to Ag. Display platform: M13 phage.
QQNVPASGTCSI

SEQ ID NO: 186 - protein, 12 aa, Artificial Sequence
  Selectively binds to Ag. Display platform: M13 phage.
NAMPGMVAWLCR

SEQ ID NO: 187 - protein, 12 aa, Artificial Sequence
  Selectively binds to Ag. Display platform: M13 phage.
HNTSPSPIILTP

SEQ ID NO: 188 - protein, 12 aa, Artificial Sequence
  Selectively binds to Ag. Display platform: M13 phage.
ASQTLLLPVPPL

SEQ ID NO: 189 - protein, 12 aa, Artificial Sequence
  Selectively binds to Ag. Display platform: M13 phage.
YNKDRYEMQAPP

SEQ ID NO: 190 - protein, 12 aa, Artificial Sequence
  Selectively binds to Ag. Display platform: M13 phage.
TLLLLAFVHTRH

SEQ ID NO: 191 - protein, 12 aa, Artificial Sequence
  Selectively binds to Ag. Display platform: M13 phage.
PWATAVSGCFAP

SEQ ID NO: 192 - protein, 12 aa, Artificial Sequence
  Selectively binds to Ag. Display platform: M13 phage.
SPLLYATTSNQS

SEQ ID NO: 193 - protein, 12 aa, Artificial Sequence
  Selectively binds to Ag. Display platform: M13 phage.
WSWRSPTPHVVT

SEQ ID NO: 194 - protein, 8 aa, Artificial Sequence
  Selectively binds to Ag+. Display platform: SPOT synthesized peptide array.
NYYRKYRD

SEQ ID NO: 195 - protein, 8 aa, Artificial Sequence
  Selectively binds to Ag+. Display platform: SPOT synthesized peptide array.
YGQAWYKK

SEQ ID NO: 196 - protein, 8 aa, Artificial Sequence
  Selectively binds to Ag+. Display platform: SPOT synthesized peptide array.
KGKNKRRR

SEQ ID NO: 197 - protein, 8 aa, Artificial Sequence
  Selectively binds to Ag+. Display platform: SPOT synthesized peptide array.
QRRRKAWG

SEQ ID NO: 198 - protein, 8 aa, Artificial Sequence
  Selectively binds to Ag+. Display platform: SPOT synthesized peptide array.
YKRWKSKD

SEQ ID NO: 199 - protein, 8 aa, Artificial Sequence
  Selectively binds to Ag+. Display platform: SPOT synthesized peptide array.
NYRAKAPK

SEQ ID NO: 200 - protein, 8 aa, Artificial Sequence
  Selectively binds to Ag+. Display platform: SPOT synthesized peptide array.
TKKGRYQK

SEQ ID NO: 201 - protein, 8 aa, Artificial Sequence
  Selectively binds to Ag+. Display platform: SPOT synthesized peptide array.
RKYNWKKE

SEQ ID NO: 202 - protein, 8 aa, Artificial Sequence
  Selectively binds to Ag+. Display platform: SPOT synthesized peptide array.
AYWWGRAR

SEQ ID NO: 203 - protein, 12 aa, Artificial Sequence
  Selectively binds to nonspecific metals, Co and FePt. Display platform: M13 phage and M13 phage (12 - mer).
SVSVGMKPSPRP

SEQ ID NO: 204 - protein, 12 aa, Artificial Sequence
  Selectively binds to 100nm Ti particles (Q-sense, QSX-310) and titanium bisammonium lactatodihydroxide. Display platform: chemical synthesis and M13 phage.
GHTHYHAVRTQT

SEQ ID NO: 205 - protein, 12 aa, Artificial Sequence
  Selectively binds to 100 nm Ti particles (Q-sense, QSX-310) and titanium bisammonium lactatodihydroxide. Display platform: chemical synthesis and M13 phage.
QPYLFATDSLIK

SEQ ID NO: 206 - protein, 13 aa, Artificial Sequence
  Selectively binds to acid washed calcite and acid washed aragonite. Display platform: M13 phage (NEB) and chemical synthesis.
DVFSSFNLKHMRG

SEQ ID NO: 207 - protein, 13 aa, Artificial Sequence
  Selectively binds to acid washed calcite and acid washed aragonite. Display platform: M13 pphage (NEB) and chemical synthesis.
HTQNMRMYEPWFG

SEQ ID NO: 208 - protein, 12 aa, Artificial Sequence
  Selectively binds to aluminium 2024. Display platform: M13 phage.
NNRPEPSPVVPH

SEQ ID NO: 209 - protein, 12 aa, Artificial Sequence
  Selectively binds to aluminium 2024. Display platform: M13 phage.
SPLDGKNIPLGH

SEQ ID NO: 210 - protein, 12 aa, Artificial Sequence
  Selectively binds to aluminium 2024. Display platform: M13 phage.
TLWSQGRSAYPV

SEQ ID NO: 211 - protein, 12 aa, Artificial Sequence
  Selectively binds to aluminium 2024. Display platform: M13 phage.
VPSSGPQDTRTT

SEQ ID NO: 212 - protein, 12 aa, Artificial Sequence
  Selectively binds to aluminium 2024. Display platform: M13 phage.
WPAPAIWHAPTL

SEQ ID NO: 213 - protein, 12 aa, Artificial Sequence
  Selectively binds to aluminium 2024. Display platform: M13 phage.
YSPDPRPWSSRY

SEQ ID NO: 214 - protein, 7 aa, Artificial Sequence
  Selectively binds to C graphene and the edges of graphene. Display platform: phage (7 mer).
EPLQLKM

SEQ ID NO: 215 - protein, 12 aa, Artificial Sequence
  Selectively binds to C graphene sheets and HOPG (graphite). Display platform: chemical synthesis.
GAMHLPWHMGTL

SEQ ID NO: 216 - protein, 8 aa, Artificial Sequence
  Selectively binds to C single walled carbon nanotubes.. Display platform: pVIII library.
DSPHTELP

SEQ ID NO: 217 - protein, 12 aa, Artificial Sequence
  Selectively binds to C single walled carbon nanotubes. Display platform: M13 (Ph.D. -12, NEB).
DYFSSPYYEQLF

SEQ ID NO: 218 - protein, 12 aa, Artificial Sequence
  Selectively binds to C single walled carbon nanotubes, carbon nanotubes and central plane of graphene. Display platform: combinatorial phage peptide display library.
HSSYWYAFNNKT

SEQ ID NO: 219 - protein, 4 aa, Artificial Sequence
  Selectively binds to Ca hydroxyapatite. Display platform: phage display.
KLSW

SEQ ID NO: 220 - protein, 12 aa, Artificial Sequence
  Selectively binds to Ca hydroxyapatite. Display platform: M13 (Ph.D. -12, NEB).
NPYHPTIPQSVH

SEQ ID NO: 221 - protein, 12 aa, Artificial Sequence
  Selectively binds to Ca hydroxyapatite. Display platform: M13 (Ph.D. -12, NEB).
TVSRPTAPYVTP

SEQ ID NO: 222 - protein, 12 aa, Artificial Sequence
  Selectively binds to CaCO3. Display platform: M13 phage library (NEB).
DVFSSFNLKHMR

SEQ ID NO: 223 - protein, 12 aa, Artificial Sequence
  Selectively binds to CaCO3. Display platform: M13 phage library (NEB).
HTQNMRMYEPWF

SEQ ID NO: 224 - protein, 9 aa, Artificial Sequence
  Selectively binds to cadmium sulphide. Display platform: phage display.
CTYSRKHKC

SEQ ID NO: 225 - protein, 4 aa, Artificial Sequence
  Selectively binds to calcite powder. Display platform: Fmoc synthesis.
MLIL

SEQ ID NO: 226 - protein, 4 aa, Artificial Sequence
  Selectively binds to calcite powder. Display platform: Fmoc synthesis.
NTNS

SEQ ID NO: 227 - protein, 4 aa, Artificial Sequence
  Selectively binds to calcite powder. Display synthesis: Fmoc synthesis.
PICL

SEQ ID NO: 228 - protein, 4 aa, Artificial Sequence
  Selectively binds to calcite powder. Display platform: Fmoc synthesis.
PWFF

SEQ ID NO: 229 - protein, 4 aa, Artificial Sequence
  Selectively binds to calcite powder. Display platform: Fmoc synthesis.
PWFW

SEQ ID NO: 230 - protein, 4 aa, Artificial Sequence
  Selectively binds to calcite powder. Display platform: Fmoc synthesis.
QSQN

SEQ ID NO: 231 - protein, 4 aa, Artificial Sequence
  Selectively binds to calcite powder. Display platform: Fmoc synthesis.
QSTN

SEQ ID NO: 232 - protein, 4 aa, Artificial Sequence
  Selectively binds to calcite powder. Display platform: Fmoc synthesis.
STTC

SEQ ID NO: 233 - protein, 4 aa, Artificial Sequence
  Selectively bind to calcite powder. Display platform: Fmoc synthesis.
TQNY

SEQ ID NO: 234 - protein, 4 aa, Artificial Sequence
  Selectively bind to calcite powder. Display platform: Fmoc synthesis.
TTNN

SEQ ID NO: 235 - protein, 4 aa, Artificial Sequence
  Selectively binds to calcite powder. Display platform: Fmoc synthesis.
SSYN

SEQ ID NO: 236 - protein, 12 aa, Artificial Sequence
  Selectively binds to calcium phosphate. Display platform: FliTrx cell surface display system (Invitrogen).
KDVVVGVPGGQD

SEQ ID NO: 237 - protein, 12 aa, Artificial Sequence
  Selectively binds to carbon nanotubes. Display platform: M13 phage (NEB), Si-C particles.
HHWHHWCMPHKT

SEQ ID NO: 238 - protein, 12 aa, Artificial Sequence
  Selectively binds to carbon nanotubes. Display platformL M13 phage (NEB), Si-C particles.
HNWYHWWMPHNT

SEQ ID NO: 239 - protein, 12 aa, Artificial Sequence
  Selectively binds to carbon nanotubes. Display platform: phage peptide library.
HTSYWYAFNTKT

SEQ ID NO: 240 - protein, 12 aa, Artificial Sequence
  Selectively binds to carbon nanotubes. Display platform: M13 phage (NEB) and Si-C particles.
HWKHPWGAWDTL

SEQ ID NO: 241 - protein, 12 aa, Artificial Sequence
  Selectively binds to carbon nanotubes. Display platform: M13 phage (NEB) and Si-C particles.
HWSAWWIRSNQS

SEQ ID NO: 242 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4 and YOX(Y2O3:Eu3+). Display platform: f88.4 phage (disulfide- constrained hybrid pVIII: LX-4 library from Dr. Jamie Scott).
ACQYPLCS

SEQ ID NO: 243 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4 and YOX(Y2O3:Eu3+). Display platform: f88.4 phage (disulfide- constrained hybrid pVIII; LX-4 library from Dr. Jamie Scott).
RAQTPLCS

SEQ ID NO: 244 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4 and YOX(Y2O3:Eu3+). Display platform: f88.4 phage (disulfide- constrained hybrid pVIII; LX-4 library from Dr. Jamie Scott).
RCAYPLCS

SEQ ID NO: 245 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4, YOX(Y2O3:Eu3+) and lanthanide phosphates. Display platform: f88.4 phage (disulfide-constrained hybrid pVIII; LX-4 library from Dr. Jamie
RCLRSHCG

SEQ ID NO: 246 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4, YOX(Y2O3:Eu3+) and lanthanide phosphates. Display platform: f88.4 phage (disulfide-constrained hybrid pVIII; LX-4 library from Dr. Jamie
RCPRFSCW

SEQ ID NO: 247 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4, YOX(Y2O3:Eu3+).Display platform: f88.4 phage (disulfide- constrained hybrid pVIII; LX-4 library from Dr. Jamie Scott).
RCQAPLCS

SEQ ID NO: 248 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4 and YOX(Y2O3:Eu3+). Display platform: f88.4 phage (disulfide- constrained hybrid pVIII; LX-4 library from Dr. Jamie Scott).
RCQYALCS

SEQ ID NO: 249 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4 and YOX(Y2O3:Eu3+). Display platform: f88.4 phage (disulfide- constrained hybrid pVIII; LX-4 library from Dr. Jamie Scott).
RCQYPLAS

SEQ ID NO: 250 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4 and YOX(Y2O3:Eu3+). Display platform: f88.4 phage (disulfide- constrained hybrid pVIII; LX-4 library from Dr. Jamie Scott).
RCQYPLCA

SEQ ID NO: 251 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4, YOX(Y2O3:Eu3+) and lanthanide phosphates. Display platform: f88.4 phage (disulfide-constrained hybrid pVIII; LX-4 library from Dr. Jamie
RCQYPLCS

SEQ ID NO: 252 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4, YOX(Y2O3:Eu3+) and lanthanide phosphates. Display platform: f88.4 phage (disulfide-constrained hybrid pVIII; LX-4 library from Dr. Jamie
RCQYSPCH

SEQ ID NO: 253 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4, YOX(Y2O3:Eu3+) and lanthanide phosphates. Display platform: f88.4 phage (disulfide-constrained hybrid pVIII; LX-4 library from Dr. Jamie
SCFRPTCP

SEQ ID NO: 254 - protein, 8 aa, Artificial Sequence
  Selectively binds to CAT(CeMgAl11O19:Tb3+), LAP(LaPO4:Ce3+, Tb3+), SiO2, BAM(BaMgAl10O17:Eu2+), LaPO4, YOX(Y2O3:Eu3+) and lanthanide phosphates. Display platform: Phage VIII, f88.4 phage (disulfide-constrained hybrid pVIII; LX-4 library from Dr. Jamie
SCKTVFCY

SEQ ID NO: 255 - protein, 9 aa, Artificial Sequence
  Selectively binds to Cd, Cd2+, Cu2+ and Zn2+. Display platform: E. coli and chemical synthesis.
GCGCPCGCG

SEQ ID NO: 256 - protein, 12 aa, Artificial Sequence
  Selectively binds to Cd. Display platform: E. coli.
GHHPHGGHHPHG

SEQ ID NO: 257 - protein, 6 aa, Artificial Sequence
  Selectively binds to Cd, CdCl2, Co2+, Cu2+, Cd2+, and Zn2+. Display platform: E. coli, Caulobacter crescentus and Saccharomyces cerevisiae.
HHHHHH

SEQ ID NO: 258 - protein, 6 aa, Artificial Sequence
  Selectively binds to Cd. Display platform: E. coli.
HSQKVF

SEQ ID NO: 259 - protein, 6 aa, Artificial Sequence
  Selectively binds to Cd2+, Cu2+, and Zn2+. Display platform: E. coli and chemical synthesis.
GHHPHG

SEQ ID NO: 260 - protein, 15 aa, Artificial Sequence
  Selectively binds to Cd2+ and Hg2+. Display platform: E. coli.
CGCCGCGCCGCGCCG

SEQ ID NO: 261 - protein, 18 aa, Artificial Sequence
  Selectively binds to CdCl2, CdSO4 and ZnCl2. Display platform: Saccharomyces cerevisiae immobilized on cytopore microcarrier beads and Saccharomyces cerevisiae.
SGCGCPCGCGCGCPCGCG

SEQ ID NO: 262 - protein, 17 aa, Artificial Sequence
  Selectively binds to CdCl2 and ZnCl2. Display platform: Saccharomyces cerevisiae.
SGHHPHGHHPHGHHPHG

SEQ ID NO: 263 - protein, 9 aa, Artificial Sequence
  Selectively binds to CdS. Display platform: M13 phage.
CTYSRLHLC

SEQ ID NO: 264 - protein, 15 aa, Artificial Sequence
  Selectively binds to CdS. Display platform: Saccharomyces cerevisiae.
DVHHHGRHGAEHADI

SEQ ID NO: 265 - protein, 15 aa, Artificial Sequence
  Selectively binds to CdS. Display platform: Saccharomyces cerevisiae.
DVHHHGRHGAEQAEI

SEQ ID NO: 266 - protein, 19 aa, Artificial Sequence
  Selectively binds to CdS. Display platform: Saccharomyces cerevisiae.
HDYRGHIHGHSQHGTEQPD

SEQ ID NO: 267 - protein, 12 aa, Artificial Sequence
  Selectively binds to Cds. Display platform: M13 phage.
PWIPTPRPTFTG

SEQ ID NO: 268 - protein, 51 aa, Artificial Sequence
  Selectively binds to CdS. Display platform: Saccharomyces cerevisiae.
QVQLQQSGPGLVKPSQTLSLTCAISGDSVSSNSAAWNWIRQSPSRGLEWQG

SEQ ID NO: 269 - protein, 84 aa, Artificial Sequence
  Selectively binds to CdS. Display platform: Saccharomyces cerevisiae.
QVQLQQSGPGLVKPSQTLSLTCAISGDSVSSNSAAWNWIRQSPSRGLEWQGHDYRGHIHGHSQHGTEQPDIRRHGRLLLCERCN

SEQ ID NO: 270 - protein, 70 aa, Artificial Sequence
  Selectively binds to CdS. Display platform: Saccharomyces cerevisiae.
QVQLQQSGPGLVKPSQTLSLTCAISGDSVSSNSAAWNWIRQSPSRGLEWQGHDYRGHIHGHSQHGTEQPD

SEQ ID NO: 271 - protein, 49 aa, Artificial Sequence
  Selectively binds to CdS. Display platform: Saccharomyces cerevisiae.
QVQLVQSGAEVKKPGASVKVSCKAPGYTFTGYDLHWVRQAPGQGLEWMG

SEQ ID NO: 272 - protein, 87 aa, Artificial Sequence
  Selectively binds to CdS. Display paltform: Saccharomyces cerevisiae.
QVQLVQSGAEVKKPGASVKVSCKAPGYTFTGYDLHWVRQAPGQGLEWMGRINPSSGATNYAQRFQGRVTMTRDVHHHGRHGAEHADI

SEQ ID NO: 273 - protein, 73 aa, Artificial Sequence
  Selectively binds to CdS. Display platform: Saccharomyces cerevisiae.
QVQLVQSGAEVKKPGASVKVSCKAPGYTFTGYDLHWVRQAPGQGLEWMGRINPSSGATNYAQRFQGRVTMTRD

SEQ ID NO: 274 - protein, 49 aa, Artificial Sequence
  Selectively binds to CdS. Display platform: Saccharomyces cerevisiae.
QVQLVQSGAEVKKPGSSVKVSCKASGDTFSSYAINWVRQAPGQGLEWMG

SEQ ID NO: 275 - protein, 87 aa, Artificial Sequence
  Selectively binds to CdS. Display platform: Saccharomyces cerevisiae.
QVQLVQSGAEVKKPGSSVKVSCKASGDTFSSYAINWVRQAPGQGLEWMGRINPNSGATNYAQRFQGRVTMTRDVHHHGRHGAEQAEI

SEQ ID NO: 276 - protein, 73 aa, Artificial Sequence
  Selectively binds to CdS. Display platform: Saccharomyces cerevisiae.
QVQLVQSGAEVKKPGSSVKVSCKASGDTFSSYAINWVRQAPGQGLEWMGRINPNSGATNYAQRFQGRVTMTRD

SEQ ID NO: 277 - protein, 12 aa, Artificial Sequence
  Selectively binds to CdS. Display platform: M13 phage.
SLTPLTTSHLRS

SEQ ID NO: 278 - protein, 12 aa, Artificial Sequence
  Selectively binds to Co. Display platform: M13 phage.
ESIPALAGLSDK

SEQ ID NO: 279 - protein, 12 aa, Artificial Sequence
  Selectively binds to Co. Display platform: M13 phage.
GTSTFNSVPVRD

SEQ ID NO: 280 - protein, 12 aa, Artificial Sequence
  Selectively binds to Co. Display platform: M13 phage.
GVLNAAQTWALS

SEQ ID NO: 281 - protein, 12 aa, Artificial Sequence
  Selectively binds to Co. Display platform: M13 phage.
HAMRPQVHPNYA

SEQ ID NO: 282 - protein, 12 aa, Artificial Sequence
  Selectively binds to Co. Display platform: M13 phage.
HETNPPATIMPH

SEQ ID NO: 283 - protein, 12 aa, Artificial Sequence
  Selectively binds to Co. Display platform: M13 phage.
HPPTDGMVPSPP

SEQ ID NO: 284 - protein, 12 aa, Artificial Sequence
  Selectively binds to Co. Display platform: M13 phage.
HSVRWLLPGAHP

SEQ ID NO: 285 - protein, 12 aa, Artificial Sequence
  Selectively binds to Co. Display platform: M13 phage.
HYPTLPLGSSTY

SEQ ID NO: 286 - protein, 12 aa, Artificial Sequence
  Selectively binds to Co. Display platform: M13 phage.
KLHSSPHTPLVQ

SEQ ID NO: 287 - protein, 12 aa, Artificial Sequence
  Selectively binds to Co. Display platform: M13 phage.
QLLPLTPSLLQA

SEQ ID NO: 288 - protein, 12 aa, Artificial Sequence
  Selectively binds to Co. Display platform: M13 phage.
QNFLQVIRNAPR

SEQ ID NO: 289 - protein, 12 aa, Artificial Sequence
  Selectively binds to Co. Display platform: M13 phage.
QYKHHPQKAAHI

SEQ ID NO: 290 - protein, 12 aa, Artificial Sequence
  Selectively binds to Co. Display platform: M13 phage.
SAPNLNALSAAS

SEQ ID NO: 291 - protein, 12 aa, Artificial Sequence
  Selectively binds to Co. Display platform: M13 phage.
SLTQTVTPWAFY

SEQ ID NO: 292 - protein, 12 aa, Artificial Sequence
  Selectively binds to Co. Display platform: M13 phage.
SPLQVLPYQGYV

SEQ ID NO: 293 - protein, 12 aa, Artificial Sequence
  Selectively binds to Co. Display platform: M13 phage.
TFPSHLATSTQP

SEQ ID NO: 294 - protein, 12 aa, Artificial Sequence
  Selectively binds to Co. Display platform: M13 phage.
TGDVSNNPNVTL

SEQ ID NO: 295 - protein, 12 aa, Artificial Sequence
  Selectively binds to Co. Display platform: M13 phage.
TNLDDSYPLHHL

SEQ ID NO: 296 - protein, 12 aa, Artificial Sequence
  Selectively binds to Co. Display platform: M13 phage.
TPNSDALLTPAL

SEQ ID NO: 297 - protein, 12 aa, Artificial Sequence
  Selectively binds to Co. Display platformL M13 phage.
TQQTDSRPPVLL

SEQ ID NO: 298 - protein, 12 aa, Artificial Sequence
  Selectively binds to Co. Display platform: M13 phage.
TWQPFGMRPSDP

SEQ ID NO: 299 - protein, 12 aa, Artificial Sequence
  Selectively binds to Co. Display platform: M13 phage.
VPTNVQLQTPRS

SEQ ID NO: 300 - protein, 12 aa, Artificial Sequence
  Selectively binds to Co. Display platform: M13 phage.
WASAAWLVHSTI

SEQ ID NO: 301 - protein, 12 aa, Artificial Sequence
  Selectively binds to Co. Display platform: M13 phage.
YGNQTPYWYPHR

SEQ ID NO: 302 - protein, 6 aa, Artificial Sequence
  Selectively binds to Co2+, Cu2+, Cd2+ and Zn2+. Display platform: Saccharomyces cerevisiae.
CCCCCC

SEQ ID NO: 303 - protein, 6 aa, Artificial Sequence
  Selectively binds to Co2+. Cu2+, Cd2+ and Zn2+. Display platform: Saccharomyces cerevisiae.
DDDDDD

SEQ ID NO: 304 - protein, 6 aa, Artificial Sequence
  Selectively binds to  Co2+, Cu2+, Cd2+ and Zn2+. Display platform: Saccharomyces cerevisiae.
DEDEDE

SEQ ID NO: 305 - protein, 6 aa, Artificial Sequence
  Selectively binds to Co2+, Cu2+, Cd2+ and Zn2+. Display platform: Saccharomyces cerevisiae.
GGGGGG

SEQ ID NO: 306 - protein, 14 aa, Artificial Sequence
  Selectively binds to Cr. Display platform: E. coli.
ALRRDVNCIGASMH

SEQ ID NO: 307 - protein, 14 aa, Artificial Sequence
  Selectively binds to Cr. Display platform: E. coli.
PGMDHQKPLGKQAT

SEQ ID NO: 308 - protein, 14 aa, Artificial Sequence
  Selectively binds to Cr. Display platform: E. coli.
PGMDRQQHQSKQAT

SEQ ID NO: 309 - protein, 14 aa, Artificial Sequence
  Selectively binds to Cr. Display platform: E. coli.
PGMYNQHQKTKEAT

SEQ ID NO: 310 - protein, 9 aa, Artificial Sequence
  Selectively binds to Cr2O3. Display platformL E. coli K-12.
RIRHRLVGQ

SEQ ID NO: 311 - protein, 9 aa, Artificial Sequence
  Selectively binds to Cr2O3. Display platform: E. coli K-12.
VVRPKAATN

SEQ ID NO: 312 - protein, 12 aa, Artificial Sequence
  Selectively binds to Cu nanoparticles and Ni nanoparticles. Display platform: chemical synthesis (Applied Biosystems Peptide Synthesizer 432A).
HGGGHGHGGGHG

SEQ ID NO: 313 - protein, 12 aa, Artificial Sequence
  Selectively binds to Cu2O class I. Display platform: E. coli.
ADRTRGRIRGNC

SEQ ID NO: 314 - protein, 12 aa, Artificial Sequence
  Selectively binds to Cu2O class I. Display paltform: E. coli.
RHTDGLRRIAAR

SEQ ID NO: 315 - protein, 12 aa, Artificial Sequence
  Selectively binds to Cu2O class I. Display paltform: E. coli.
RPRRSAARGSEG

SEQ ID NO: 316 - protein, 12 aa, Artificial Sequence
  Selectively binds to Cu2O class I. Display platform: E. coli.
RTRRQGGDVSRD

SEQ ID NO: 317 - protein, 12 aa, Artificial Sequence
  Selectively binds to Cu2O class II. Display platform: E. coli.
EKWGMHQECYRH

SEQ ID NO: 318 - protein, 12 aa, Artificial Sequence
  Selectively binds to Cu2O class II. Display platform: E. coli.
NTVWRLNSSCGM

SEQ ID NO: 319 - protein, 12 aa, Artificial Sequence
  Selectively binds to  Cu2O class II. Display platform: E. coli.
TMEPRWWCNPIN

SEQ ID NO: 320 - protein, 12 aa, Artificial Sequence
  Selectively binds to Cu2O, ZnO class II and ZnO. Display platform: E. coli.
MRHSSSGEPRLL

SEQ ID NO: 321 - protein, 12 aa, Artificial Sequence
  Selectively binds to Cu2O, ZnO class II and ZnO. Display platform: E. coli.
PAGLQVGFAVEV

SEQ ID NO: 322 - protein, 12 aa, Artificial Sequence
  Selectively binds to Cu2O,, ZnO class I and ZnO. Display platform: E. coli.
PASRVEKNGVRR

SEQ ID NO: 323 - protein, 12 aa, Artificial Sequence
  Selectively binds to Cu2O, ZnO class I and ZnO. Display platform: E. coli.
RIGHGRQIRKPL

SEQ ID NO: 324 - protein, 12 aa, Artificial Sequence
  Selectively binds to Cu2O, ZnO class II and ZnO. Display platform: E. coli.
RTDDGVAGRTWL

SEQ ID NO: 325 - protein, 12 aa, Artificial Sequence
  Selectively binds to Cu2O and ZnO. Display platform: E. coli.
VRTRDDARTHRK

SEQ ID NO: 326 - protein, 14 aa, Artificial Sequence
  Selectively binds to CuFeS2 chalcopyrite. Display platform: M13 phage.
DKKKCDGKRCSWPS

SEQ ID NO: 327 - protein, 8 aa, Artificial Sequence
  Selectively binds to CuFeS2 chalcopyrite. Display platform: M13 phage.
DPIKHTSG

SEQ ID NO: 328 - protein, 8 aa, Artificial Sequence
  Selectively binds to CuFeS2 chalcopyrite. Display platform: M13 phage.
DSQKTNPS

SEQ ID NO: 329 - protein, 14 aa, Artificial Sequence
  Selectively binds to CuFeS2 chalcopyrite. Display platform: M13 phage.
EKDRCTKNTCKPPA

SEQ ID NO: 330 - protein, 14 aa, Artificial Sequence
  Selectively binds to CuFeS2 chalcopyrite. Display platform: M13 phage.
EKKKCGTMACPYRA

SEQ ID NO: 331 - protein, 14 aa, Artificial Sequence
  Selectively binds to CuFeS2 chalcopyrite. Display platform: M13 phage.
ERSGCHKKACPKTP

SEQ ID NO: 332 - protein, 14 aa, Artificial Sequence
  Selectively binds to CuFeS2 chalcopyrite. Display platform: M13 phage.
GKCSCKEKQCRTTL

SEQ ID NO: 333 - protein, 14 aa, Artificial Sequence
  Selectively binds to CuFeS2 chalcopyrite. Display platform: M13 phage.
GKKKCPNKSCTSLF

SEQ ID NO: 334 - protein, 14 aa, Artificial Sequence
  Selectively binds to CuFeS2 chalcopyrite. Display platform: M13 phage.
HKTQCNPRACTRRH

SEQ ID NO: 335 - protein, 14 aa, Artificial Sequence
  Selectively binds to CuFeS2. Display platform: M13 phage.
KDHDCHRAQCRPNL

SEQ ID NO: 336 - protein, 14 aa, Artificial Sequence
  Selectively binds to CuFeS2 chalcopyrite. Display platform: M13 phage.
KKTNCKHDSCRTCQ

SEQ ID NO: 337 - protein, 14 aa, Artificial Sequence
  Selectively binds to CuFeS2 chalcopyrite. Display platform: M13 phage.
KNEKCAHHKCYLYP

SEQ ID NO: 338 - protein, 14 aa, Artificial Sequence
  Selectively binds to CuFeS2 chalcopyrite. Display platform: M13 phage.
KNKRCSQGCCINNG

SEQ ID NO: 339 - protein, 14 aa, Artificial Sequence
  Selectively binds to CuFeS2 chalcopyrite. Display platform: M13 phage.
KSKSCEAMQCNKYY

SEQ ID NO: 340 - protein, 14 aa, Artificial Sequence
  Selectively binds to CuFeS2 chalcopyrite. Display platform: M13 phage.
KSRHCSQIQCGDKV

SEQ ID NO: 341 - protein, 14 aa, Artificial Sequence
  Selectively binds to CuFeS2 chalcopyrite. Display paltform: M13 phage.
QRNKCHHNTCVKML

SEQ ID NO: 342 - protein, 14 aa, Artificial Sequence
  Selectively binds to CuFeS2 chalcopyrite. Display platform: M13 phage.
RKKKCKGNCCYTPQ

SEQ ID NO: 343 - protein, 12 aa, Artificial Sequence
  Selectively binds to dolomite. Display platform: M13 phage (Ph.D. -12, NEB).
ADYFTARPGPIT

SEQ ID NO: 344 - protein, 12 aa, Artificial Sequence
  Selectively binds to dolomite. Display platform: M13 phage (Ph.D. - 12, NEB).
ANDGLATRPRDL

SEQ ID NO: 345 - protein, 12 aa, Artificial Sequence
  Selectively binds to dolomite. Display platform: M13 phage (Ph.D. - 12, NEB).
APKGLTNTSQLM

SEQ ID NO: 346 - protein, 12 aa, Artificial Sequence
  Selectively binds to dolomite. Display platform: M13 phage (Ph.D. -12, NEB).
APVAHAFPQAMM

SEQ ID NO: 347 - protein, 12 aa, Artificial Sequence
  Selectively binds to dolomite. Display platform: M13 phage (Ph.D. -12, NEB).
DTNFVKAPRQPN

SEQ ID NO: 348 - protein, 12 aa, Artificial Sequence
  Selectively binds to dolomite. Display platform: M13 phage (Ph.D. -12, NEB).
EFQTPLRANVSF

SEQ ID NO: 349 - protein, 12 aa, Artificial Sequence
  Selectively binds to dolomite. Display platform: M13 phage (Ph.D. -12, NEB).
GFAHHSWAPDRA

SEQ ID NO: 350 - protein, 12 aa, Artificial Sequence
  Selectively binds to dolomite. Display platform: M13 phage (Ph.D. -12, NEB).
GFASDPSSSPWT

SEQ ID NO: 351 - protein, 12 aa, Artificial Sequence
  Selectively binds to dolomite. Display platform: M13 phage (Ph.D. -12, NEB).
GMELHSKLPIYR

SEQ ID NO: 352 - protein, 12 aa, Artificial Sequence
  Selectively binds to dolomite. Display platform: M13 phage (Ph.D. -12, NEB).
GMELHSKLPTYR

SEQ ID NO: 353 - protein, 12 aa, Artificial Sequence
  Selectively binds to dolomite. Display platform: M13 phage (Ph.D. -12, NEB).
HLGGSIARIPEQ

SEQ ID NO: 354 - protein, 12 aa, Artificial Sequence
  Selectively binds to dolomite. Display platform: M13 phage (Ph. D. -12, NEB).
HTEPANWYPHTH

SEQ ID NO: 355 - protein, 12 aa, Artificial Sequence
  Selectively binds to dolomite. Display platform:M13 phage (Ph.D. -12, NEB).
HYTEASFDIRTR

SEQ ID NO: 356 - protein, 12 aa, Artificial Sequence
  Selectively binds to dolomite. Display platform: M13 phage (Ph.D. -12, NEB).
LPSRVQELWWPA

SEQ ID NO: 357 - protein, 12 aa, Artificial Sequence
  Selectively binds to dolomite. Display platform: M13 phage (Ph.D. -12, NEB).
LPTMMNNNWNQR

SEQ ID NO: 358 - protein, 12 aa, Artificial Sequence
  Selectively binds to dolomite. Display platform: M13 phage (Ph.D. -12, NEB).
MNDTKWAAPQGL

SEQ ID NO: 359 - protein, 12 aa, Artificial Sequence
  Selectively binds to dolomite. Display platform: M13 phage (Ph.D. -12, NEB).
MPNPHLALPHGS

SEQ ID NO: 360 - protein, 12 aa, Artificial Sequence
  Selectively binds to dolomite. Display platform: M13 phage (Ph.D. -12, NEB).
NFDELTMPNYRT

SEQ ID NO: 361 - protein, 12 aa, Artificial Sequence
  Selectively binds to dolomite. Display platform: M13 phage (Ph.D. -12, NEB).
NIQTTHLFPLPR

SEQ ID NO: 362 - protein, 12 aa, Artificial Sequence
  Selectively binds to dolomite. Display platform: M13 phage (Ph.D. -12, NEB).
NPIPDTRNHRLV

SEQ ID NO: 363 - protein, 12 aa, Artificial Sequence
  Selectively binds to dolomite. Display platform: M13 phage (Ph.D. -12, NEB).
NQNYDAEQLITP

SEQ ID NO: 364 - protein, 12 aa, Artificial Sequence
  Selectively binds to dolomite. Display platform: M13 phage (Ph.D. -12, NEB).
QHHTLSTAPYLY

SEQ ID NO: 365 - protein, 12 aa, Artificial Sequence
  Selectively binds to dolomite. Display platform: M13 phage (Ph.D. -12, NEB).
QIPNAVDLYWSP

SEQ ID NO: 366 - protein, 12 aa, Artificial Sequence
  Selectively binds to dolomite. Display platform: M13 phage (Ph.D. -12, NEB).
QLTVDNNHQGND

SEQ ID NO: 367 - protein, 12 aa, Artificial Sequence
  Selectively binds to M13 phage (Ph.D. -12, NEB).
QQNYLTQNIGRA

SEQ ID NO: 368 - protein, 12 aa, Artificial Sequence
  Selectively binds to dolomite. Display platform: M13 phage (Ph.D. -12, NEB).
QTLPLPLTIAHP

SEQ ID NO: 369 - protein, 12 aa, Artificial Sequence
  Selectively binds to dolomite. Display platform: M13 phage (Ph.D. -12, NEB).
SLNCSLASSACR

SEQ ID NO: 370 - protein, 12 aa, Artificial Sequence
  Selectively binds to dolomite. Display platform: M13 phage (Ph.D. -12, NEB).
SNITPQTSTPSL

SEQ ID NO: 371 - protein, 12 aa, Artificial Sequence
  Selectively binds to dolomite. Display platform: M13 phage (Ph.D. -12, NEB).
SPNIGIAKNMLY

SEQ ID NO: 372 - protein, 12 aa, Artificial Sequence
  Selectively binds to dolomite. Display platform: M13 phage (Ph.D. -12, NEB).
SPNPPANAVITN

SEQ ID NO: 373 - protein, 12 aa, Artificial Sequence
  Selectively binds to dolomite. Display platform: M13 phage (Ph.D. -12, NEB).
SPNPPANAVTTN

SEQ ID NO: 374 - protein, 12 aa, Artificial Sequence
  Selectively binds to dolomite. Display platform: M13 phage (Ph.D. -12, NEB).
STDMSPSPMSHS

SEQ ID NO: 375 - protein, 12 aa, Artificial Sequence
  Selectively binds to dolomite. Display platform: M13 phage (Ph.D. -12, NEB).
TANWHPARTLLT

SEQ ID NO: 376 - protein, 12 aa, Artificial Sequence
  Selectively binds to dolomite. Display platform: M13 phage (Ph.D. -12, NEB).
THYTRGLSPFSL

SEQ ID NO: 377 - protein, 12 aa, Artificial Sequence
  Selectively binds to dolomite. Display platform: M13 phage (Ph.D. -12, NEB).
TSENNYAVESFH

SEQ ID NO: 378 - protein, 12 aa, Artificial Sequence
  Selectively binds to dolomite. Display platform: M13 phage (Ph.D. -12, NEB).
TVLHNKSPDQSQ

SEQ ID NO: 379 - protein, 12 aa, Artificial Sequence
  Selectively binds to dolomite. Display platform: M13 phage (Ph.D. -12, NEB).
VDIHSGTWPLSY

SEQ ID NO: 380 - protein, 12 aa, Artificial Sequence
  Selectively binds to dolomite. Display platform: M13 phage (Ph.D. -12, NEB).
YVAHEVINLHHT

SEQ ID NO: 381 - protein, 12 aa, Artificial Sequence
  Selectively binds to dolomite. Display platform: M13 phage.
  SITE (7)..(7): Xaa can be any naturally occurring amino acid
FHRAPWXYLGNY

SEQ ID NO: 382 - protein, 12 aa, Artificial Sequence
  Selectively binds to enargite. Display platform: M13 phage.
FPHHKQRYVDPL

SEQ ID NO: 383 - protein, 12 aa, Artificial Sequence
  Selectively binds to enargite. Display platform: M13 phage.
FPWYKWRLPDVS

SEQ ID NO: 384 - protein, 12 aa, Artificial Sequence
  Selectively binds to enargite. Display platform: M13 phage.
GMKPWFYSNWKG

SEQ ID NO: 385 - protein, 12 aa, Artificial Sequence
  Selectively binds to enargite. Display platform: M13 phage.
GMLHWSYSIFNP

SEQ ID NO: 386 - protein, 12 aa, Artificial Sequence
  Selectively binds to enargite. Display platform: M13 phage.
HTSSLWHLFRST

SEQ ID NO: 387 - protein, 12 aa, Artificial Sequence
  Selectively binds to enargite. Display platform: M13 phage.
IPLHSLHVKWGK

SEQ ID NO: 388 - protein, 12 aa, Artificial Sequence
  Selectively binds to enargite. Display platform: M13 phage.
IPWHRPAQVMHL

SEQ ID NO: 389 - protein, 12 aa, Artificial Sequence
  Selectively binds to enargite. Display platform: M13 phage.
KFSTHPWHSYSP

SEQ ID NO: 390 - protein, 12 aa, Artificial Sequence
  Selectively binds to enargite. Display platform: M13 phage.
LPWHWAPNMYRS

SEQ ID NO: 391 - protein, 12 aa, Artificial Sequence
  Selectively binds to enargite. Display platform: M13 phage.
MGKPAPRYLGNN

SEQ ID NO: 392 - protein, 12 aa, Artificial Sequence
  Selectively bnids to enargite. Display platform: M13 phage.
MGKSTLRYTTIV

SEQ ID NO: 393 - protein, 12 aa, Artificial Sequence
  Selectively binds to enargite. Display platform: M13 phage.
MHKPTVHIKGPT

SEQ ID NO: 394 - protein, 14 aa, Artificial Sequence
  Selectively binds to zeolites. Display platform: E. coli.
MDHGKYRQKQATPG

SEQ ID NO: 395 - protein, 9 aa, Artificial Sequence
  Selectively binds to Fe2O3. Display platform: E. coli.
RRTVKHHVN

SEQ ID NO: 396 - protein, 12 aa, Artificial Sequence
  Selectively binds to Fe, Fe3O4, Zn and ZnO. Display platform: M13 phage.
AGYPLSENFYYP

SEQ ID NO: 397 - protein, 12 aa, Artificial Sequence
  Selectively binds to Fe, Fe3O4, Zn and ZnO. Display platform: M13 phage.
FHPRLQQDHWLH

SEQ ID NO: 398 - protein, 12 aa, Artificial Sequence
  Selectively binds to Fe, Fe3O4, Zn and ZnO. Display platform: M13 phage.
GLHTSATNLYLH

SEQ ID NO: 399 - protein, 12 aa, Artificial Sequence
  Selectively binds to Fe, Fe3O4, Zn and ZnO. Display platform: M13 phage.
WQDFGAVRSTRS

SEQ ID NO: 400 - protein, 12 aa, Artificial Sequence
  Selectively binds to FePt. Display platform: M13 phage (12-mer).
HNKHLPSTQPLA

SEQ ID NO: 401 - protein, 12 aa, Artificial Sequence
  Selectively binds to FePt. Display platform: M13 phage (12-mer).
KSLSRHDHIHHH

SEQ ID NO: 402 - protein, 12 aa, Artificial Sequence
  Selectively binds to FePt. Display platform: M13 phage (12-mer).
VISNHRESSRPL

SEQ ID NO: 403 - protein, 12 aa, Artificial Sequence
  Selectively binds to francolite. Display platform: M13 phage (Ph.D. -12, NEB).
WSITTYHDRAIV

SEQ ID NO: 404 - protein, 12 aa, Artificial Sequence
  Selectively binds to francolite. Display platform: M13 phage (Ph.D. -12, NEB).
WSYVPFARQVNQ

SEQ ID NO: 405 - protein, 12 aa, Artificial Sequence
  Selectively binds to francolite. Display platform: M13 phage (Ph.D. -12, NEB).
HMPHHVSNLQLH

SEQ ID NO: 406 - protein, 12 aa, Artificial Sequence
  Selectively binds to francolite. Display platform: M13 phage.
GSNGIWFNLAHR

SEQ ID NO: 407 - protein, 12 aa, Artificial Sequence
  Selectively binds to francolite. Display platform: M13 phage (Ph.D. -12, NEB).
YSQPTLWALTSR

SEQ ID NO: 408 - protein, 12 aa, Artificial Sequence
  Selectively binds to francolite. Display platformL M13 phage (Ph.D. -12, NEB).
NIGHRVNSPFPQ

SEQ ID NO: 409 - protein, 12 aa, Artificial Sequence
  Selectively binds to francolite. Display platform: M13 phage (Ph.D. -12, NEB).
APRLLSDNTYNV

SEQ ID NO: 410 - protein, 12 aa, Artificial Sequence
  Selectively binds to francolite. Display platform: M13 phage (Ph.D. -12, NEB).
AHLEDITVHDGS

SEQ ID NO: 411 - protein, 12 aa, Artificial Sequence
  Selectively binds to francolite. Display platform: M13 phage (Ph.D. -12, NEB).
TNNTFWFPAEFG

SEQ ID NO: 412 - protein, 12 aa, Artificial Sequence
  Selectively binds to francolite. Display platform: M13 phage (Ph.D. -12, NEB).
TNSNWTPFWPLP

SEQ ID NO: 413 - protein, 12 aa, Artificial Sequence
  Selectively binds to francolite. Display platform: M13 phage (Ph.D. -12, NEB).
TSPPQVAYPTLS

SEQ ID NO: 414 - protein, 12 aa, Artificial Sequence
  Selectively binds to francolite. Display platform: M13 phage (Ph.D. -12, NEB).
SHVGNPYISATL

SEQ ID NO: 415 - protein, 12 aa, Artificial Sequence
  Selectively binds to francolite. Display platform: M13 phage (Ph.D. -12, NEB).
SSMTHQHARVDT

SEQ ID NO: 416 - protein, 12 aa, Artificial Sequence
  Selectively binds to francolite. Display platform: M13 phage (Ph.D. -12, NEB).
ASLQHTALLNQN

SEQ ID NO: 417 - protein, 12 aa, Artificial Sequence
  Selectively binds to francolite. Display platform: M13 phage (Ph.D. -12, NEB).
EHWQDNWMRWIT

SEQ ID NO: 418 - protein, 12 aa, Artificial Sequence
  Selectively binds to francolite. Display platform: M13 phage (Ph.D. -12, NEB).
EKISDYAWPWRT

SEQ ID NO: 419 - protein, 12 aa, Artificial Sequence
  Selectively binds to francolite. Display platform: M13 phage (Ph.D. -12, NEB).
HYGVQAPHNSNS

SEQ ID NO: 420 - protein, 12 aa, Artificial Sequence
  Selectively binds to francolite. Display platform: M13 phage (Ph.D. -12, NEB).
DHRSISAFPNPP

SEQ ID NO: 421 - protein, 12 aa, Artificial Sequence
  Selectively binds to francolite. Display platform: M13 phage (Ph.D. -12, NEB).
MEQFQSAGNPGW

SEQ ID NO: 422 - protein, 12 aa, Artificial Sequence
  Selectively binds to francolite. Display platform: M13 phage (Ph.D. -12, NEB).
MEQTYPSSHRPG

SEQ ID NO: 423 - protein, 12 aa, Artificial Sequence
  Selectively binds to francolite. Display platform: M13 phage (Ph.D. -12, NEB).
YIGSQTNERYSP

SEQ ID NO: 424 - protein, 12 aa, Artificial Sequence
  Selectively binds to francolite. Display platform: M13 phage (Ph.D. -12, NEB).
YQSTRTHAEASP

SEQ ID NO: 425 - protein, 12 aa, Artificial Sequence
  Selectively binds to francolite and Zn. Display platform: M13 phage (Ph.D. -12, NEB) and M13 bacteriophage.
TKNMLSLPVGPG

SEQ ID NO: 426 - protein, 12 aa, Artificial Sequence
  Selectively binds to francolite. Display platform: M13 phage (Ph.D. -12, NEB).
HHHQTLRPAPFA

SEQ ID NO: 427 - protein, 12 aa, Artificial Sequence
  Selectively binds to francolite. Display platform: M13 phage (Ph.D. -12, NEB).
AVPHRVGGLHSL

SEQ ID NO: 428 - protein, 12 aa, Artificial Sequence
  Selectively binds to francolite. Display platform: M13 phage (Ph.D. -12, NEB).
HSVQTYARPLPS

SEQ ID NO: 429 - protein, 12 aa, Artificial Sequence
  Selectively binds to francolite. Display platform: M13 phage (Ph.D. -12, NEB).
QNLINWPPPRFS

SEQ ID NO: 430 - protein, 12 aa, Artificial Sequence
  Selectively binds to francolite. Display platform: M13 phage (Ph.D. -12, NEB).
HGLTVQRPEQMM

SEQ ID NO: 431 - protein, 12 aa, Artificial Sequence
  Selectively binds to francolite. Display platform: M13 phage (Ph.D. -12, NEB).
VSHSEYNRAATY

SEQ ID NO: 432 - protein, 12 aa, Artificial Sequence
  Selectively binds to francolite. Display platform: M13 phage (Ph.D. -12, NEB).
ASDNRTMVLMFP

SEQ ID NO: 433 - protein, 12 aa, Artificial Sequence
  Selectively binds to francolite. Display platform: M13 phage (Ph.D. -12, NEB).
GHVVTNSVWMLP

SEQ ID NO: 434 - protein, 12 aa, Artificial Sequence
  Selectively binds to francolite. Display platform: M13 phage (Ph.D. -12, NEB).
IDYSAPSRYANS

SEQ ID NO: 435 - protein, 12 aa, Artificial Sequence
  Selectively binds to francolite. Display platform: M13 phage (Ph.D. -12, NEB).
QGYTMFVAAEPL

SEQ ID NO: 436 - protein, 12 aa, Artificial Sequence
  Selectively binds to francolite. Display platform: M13 phage (Ph.D. -12, NEB).
DPFPQRVNYLKR

SEQ ID NO: 437 - protein, 12 aa, Artificial Sequence
  Selectively binds to francolite. Display platform: M13 phage (Ph.D. -12, NEB).
YSLPRHLVSLPP

SEQ ID NO: 438 - protein, 12 aa, Artificial Sequence
  Selectively binds to francolite. Display platform: M13 phage (Ph.D. -12, NEB).
AASFQHSATANL

SEQ ID NO: 439 - protein, 12 aa, Artificial Sequence
  Selectively binds to francolite. Display platform: M13 phage (Ph.D. -12, NEB).
HYNPEMPSSHNA

SEQ ID NO: 440 - protein, 12 aa, Artificial Sequence
  Selectively binds to francolite. Display platform: M13 phage (Ph.D. -12, NEB).
HSMPHMGTYLIT

SEQ ID NO: 441 - protein, 12 aa, Artificial Sequence
  Selectively binds to francolite. Display platform: M13 phage (Ph.D. -12, NEB).
TITPSYLLAHGP

SEQ ID NO: 442 - protein, 12 aa, Artificial Sequence
  Selectively binds to francolite and dolomite. Display platform: M13 phage (Ph.D. -12, NEB).
AQINLDNHARWF

SEQ ID NO: 443 - protein, 11 aa, Artificial Sequence
  Selectively binds to francolite and dolomite. Display platform: M13 phage (Ph.D. -12, NEB).
DIRTEPNTSNS

SEQ ID NO: 444 - protein, 12 aa, Artificial Sequence
  Selectively binds to francolite and dolomite. Display platform: M13 phage (Ph.D. -12, NEB).
DLFYDANNVHAG

SEQ ID NO: 445 - protein, 12 aa, Artificial Sequence
  Selectively binds to francolite and dolomite. Display platform: M13 phage (Ph.D. -12, NEB).
DRAPLIPFASQH

SEQ ID NO: 446 - protein, 12 aa, Artificial Sequence
  Selectively binds to francolite and dolomite. Display platform: M13 phage (Ph.D. -12, NEB).
EHWQDNWMRWTT

SEQ ID NO: 447 - protein, 12 aa, Artificial Sequence
  Selectively binds to francolite and dolomite. Display platform: M13 phage (Ph.D. -12, NEB).
FANTSSPVVHPF

SEQ ID NO: 448 - protein, 12 aa, Artificial Sequence
  Selectively binds to francolite and dolomite. Display platform: M13 phage (Ph.D. -12, NEB).
GDDVNTMRARPL

SEQ ID NO: 449 - protein, 12 aa, Artificial Sequence
  Selectively binds to francolite and dolomite. Display platform: M13 phage (Ph.D. -12, NEB).
LAPVRPIFSMEV

SEQ ID NO: 450 - protein, 12 aa, Artificial Sequence
  Selectively binds to francolite and dolomite. Display platform: M13 phage (Ph.D. -12, NEB).
LSASSPTTTATW

SEQ ID NO: 451 - protein, 12 aa, Artificial Sequence
  Selectively binds to francolite and dolomite. Display platform: M13 phage (Ph.D. -12, NEB).
MEQFQSAGNPGN

SEQ ID NO: 452 - protein, 12 aa, Artificial Sequence
  Selectively binds to francolite and dolomite. Display platform: M13 phage (Ph.D. -12, NEB).
QQYVAYPIMKAL

SEQ ID NO: 453 - protein, 12 aa, Artificial Sequence
  Selectively binds to francolite and dolomite. Display platform: M13 phage (Ph.D. -12, NEB).
SAHGTSTGVPWP

SEQ ID NO: 454 - protein, 12 aa, Artificial Sequence
  Selectively binds to francolite and dolomite. Display platform: M13 phage (Ph.D. -12, NEB).
SPTSLLPTQAHY

SEQ ID NO: 455 - protein, 12 aa, Artificial Sequence
  Selectively binds to francolite and dolomite. Display platform: M13 phage (Ph.D. -12, NEB).
TNHTFWFPAEFG

SEQ ID NO: 456 - protein, 12 aa, Artificial Sequence
  Selectively binds to francolite and dolomite. Display platform: M13 phage (Ph.D. -12, NEB).
TPPPSEITTSPP

SEQ ID NO: 457 - protein, 12 aa, Artificial Sequence
  Selectively binds to francolite and dolomite. Display platform: M13 phage (Ph.D. -12, NEB).
VHFRIATPYFSP

SEQ ID NO: 458 - protein, 12 aa, Artificial Sequence
  Selectively binds to francolite and dolomite. Display platform: M13 phage (Ph.D. -12, NEB).
YLAHSSNNKILF

SEQ ID NO: 459 - protein, 12 aa, Artificial Sequence
  Selectively binds to francolite and dolomite. Display platform: M13 phage (Ph.D. -12, NEB).
YNLTPLPKGNAM

SEQ ID NO: 460 - protein, 12 aa, Artificial Sequence
  Selectively binds to GaAs (100), GaAs (111)A, GaAs (111)B, InP (100), Si (100) single crystal semi conductors and GaAs. Display platform: M13 phage.
AQNPSDNNTHTH

SEQ ID NO: 461 - protein, 12 aa, Artificial Sequence
  Selectively binds to GaAs(100), GaAs(111)A, GaAs(111)B, InP(100), Si(100) single-crystal semi-conductors and GaAs. Display platform: M13 phage.
RLELAIPLQGSG

SEQ ID NO: 462 - protein, 12 aa, Artificial Sequence
  Selectively binds to GaAs. Display platform: M13 phage.
TPPRPIQYNHTS

SEQ ID NO: 463 - protein, 12 aa, Artificial Sequence
  Selectively binds to GaAs(100), GaAs(111)A, GaAs(111)B, InP(100) and Si(100) single-crystal semi-conductors. Display platform: M13 phage.
AASPTQSMSQAP

SEQ ID NO: 464 - protein, 12 aa, Artificial Sequence
  Selectively binds to GaAs(100), GaAs(111)A, GaAs(111)B, InP(100) and Si(100) single-crystal semi-conductors. Display platform: M13 phage.
ARYDLSIPSSES

SEQ ID NO: 465 - protein, 12 aa, Artificial Sequence
  Selectively binds to GaAs(100), GaAs(111)A, GaAs(111)B, InP(100) and Si(100) single-crystal semi-conductors. Display platform: M13 phage.
ASSSRSHFGQTD

SEQ ID NO: 466 - protein, 12 aa, Artificial Sequence
  Selectively binds to GaAs(100), GaAs(111)A, GaAs(111)B, InP(100) and Si(100) single-crystal semi-conductors. Display platform: M13 phage.
GTLANQQIFLSS

SEQ ID NO: 467 - protein, 12 aa, Artificial Sequence
  Selectively binds to GaAs(100), GaAs(111)A, GaAs(111)B, InP(100) and Si(100) single-crystal semi-conductors. Display platform: M13 phage.
HGNPLPMTPFPG

SEQ ID NO: 468 - protein, 12 aa, Artificial Sequence
  Selectively binds to GaAs(100), GaAs(111)A, GaAs(111)B, InP(100) and Si(100) single-crystal semi-conductors. Display platform: M13 phage.
SSLQLPENSFPH

SEQ ID NO: 469 - protein, 12 aa, Artificial Sequence
  Selectively binds to GaAs(100), GaAs(111)A, GaAs(111)B, InP(100) and Si(100) single-crystal semi-conductors. Display platform: M13 phage.
VTSPDSTTGAMA

SEQ ID NO: 470 - protein, 12 aa, Artificial Sequence
  Selectively binds to GaAs(100), GaAs(111)A, GaAs(111)B, InP(100) and Si(100) single-crystal semi-conductors. Display platform: M13 phage.
WAHAPQLASSST

SEQ ID NO: 471 - protein, 12 aa, Artificial Sequence
  Selectively binds to tetramethoxygermanium. Display platform: phage (12 mer).
SLKMPHWPHLLP

SEQ ID NO: 472 - protein, 12 aa, Artificial Sequence
  Selectively binds to tetramethoxygermanium. Display platform: phage (12 mer) and chemical synthesis.
TGHQSPGAYAAH

SEQ ID NO: 473 - protein, 12 aa, Artificial Sequence
  Selectively binds to tetramethoxygermanium. Display platform: phage (12 mer) and chemical synthesis.
TSLYTDRPSTPL

SEQ ID NO: 474 - protein, 11 aa, Artificial Sequence
  Selectively binds to hematite nanoparticles. Display platform: custom engineered fluorescent protein.
STVQTISPSNH

SEQ ID NO: 475 - protein, 8 aa, Artificial Sequence
  Selectively binds to IrO2. Display platform: M13 virus library.
AGETQQAM

SEQ ID NO: 476 - protein, 7 aa, Artificial Sequence
  Selectively binds to isotactic poly(methyl methacrylate). Display platform: E. coli.
ELWRPTR

SEQ ID NO: 477 - protein, 11 aa, Artificial Sequence
  Selectively binds to Ln oxide and upconversion nanocrystals. Display platform: Ph.D. -C7C phage library.
ACTARSPWICG

SEQ ID NO: 478 - protein, 13 aa, Artificial Sequence
  Selectively binds to MgF2. Display platform: bio-panning.
GEYDYACGVVGYE

SEQ ID NO: 479 - protein, 13 aa, Artificial Sequence
  Selectively binds to MgF2. Display platform: bio-panning.
GGLNQVLRIPSFI

SEQ ID NO: 480 - protein, 13 aa, Artificial Sequence
  Selectively binds to MgF2. Display platform: bio-panning.
GMIVDHLPIQVNT

SEQ ID NO: 481 - protein, 13 aa, Artificial Sequence
  Selectively binds to MgF2. Display platform: bio-panning.
GSPKHNLDMVKMM

SEQ ID NO: 482 - protein, 13 aa, Artificial Sequence
  Selectively binds to MgF2. Display platform: bio-panning.
GSYPKASLALLAP

SEQ ID NO: 483 - protein, 13 aa, Artificial Sequence
  Selectively binds to MgF2. Display platform: bio-panning.
GTQAIRVHTISSQ

SEQ ID NO: 484 - protein, 13 aa, Artificial Sequence
  Selectively binds to MgF2. Display platform: bio-panning.
GTQYYAYSTTQKS

SEQ ID NO: 485 - protein, 12 aa, Artificial Sequence
  Selectively binds to mild steel 1010. Display platform: M13 phage (Ph.d. -12, NEB).
ATIHDAFYSAPE

SEQ ID NO: 486 - protein, 12 aa, Artificial Sequence
  Selectively binds to mild steel 1010. Display platform: M13 phage (Ph.D. -12, NEB).
NLNPNTASAMHV

SEQ ID NO: 487 - protein, 12 aa, Artificial Sequence
  Selectively binds to mild steel 1010. Display platform: M13 phage (Ph.D. -12, NEB).
NLTIASYPSMVV

SEQ ID NO: 488 - protein, 12 aa, Artificial Sequence
  Selectively binds to mild steel 1010. Display platform: M13 phage (Ph.D. -12, NEB).
QMDISLGRWSSM

SEQ ID NO: 489 - protein, 12 aa, Artificial Sequence
  Selectively binds to mild steel 1010. Display platform: M13 phage (Ph.D. -12, NEB).
QSHYRHISPAQV

SEQ ID NO: 490 - protein, 12 aa, Artificial Sequence
  Selectively binds to mild steel 1010. Display platform: M13 phage (Ph.D. -12, NEB).
YMKQIPAGRTNP

SEQ ID NO: 491 - protein, 12 aa, Artificial Sequence
  Selectively binds to MoS2. Display platform: M13 phage.
DRWVARDPASIF

SEQ ID NO: 492 - protein, 12 aa, Artificial Sequence
  Selectively binds to MoS2. Display platform: M13 phage.
GVIHRNDQWTAP

SEQ ID NO: 493 - protein, 12 aa, Artificial Sequence
  Selectively binds to MoS2. Display platform: M13 phage.
SVMNTSTKDAIE

SEQ ID NO: 494 - protein, 91 aa, Artificial Sequence
  Selectively binds to natural zeolites, synthetic zeolites and silica. Display platform: E. coli.
VKTQATSREEPPRLPSKHRPGVKTQATSREEPPRLPSKHRPGVKTQATSREEPPRLPSKHRPGVKTQATSREEPPRLPSKHRPGVKTQTAS

SEQ ID NO: 495 - protein, 7 aa, Artificial Sequence
  Selectively binds to Ni3B amorphous and Ni3B crystalline. Display platform: M13 phage (Ph.D. -7, NEB).
ANHQSAN

SEQ ID NO: 496 - protein, 7 aa, Artificial Sequence
  Selectively binds to Ni3B amorphous. Display platform: M13 phage (Ph.D. -7, NEB).
GALPNNL

SEQ ID NO: 497 - protein, 7 aa, Artificial Sequence
  Selectively binds to Ni3B amorphous. Display platform: M13 phage (Ph.D. -7, NEB).
GNRLSAD

SEQ ID NO: 498 - protein, 7 aa, Artificial Sequence
  Selectively binds to Ni3B amorphous. Display platform: M13 phage (Ph.D. -7, NEB).
HVQYWQF

SEQ ID NO: 499 - protein, 7 aa, Artificial Sequence
  Selectively binds to Ni3B amorphous. Display platform: M13 phage (Ph.D. -7, NEB).
LGFREKE

SEQ ID NO: 500 - protein, 7 aa, Artificial Sequence
  Selectively binds to Ni3B amorphous. Display platform: M13 phage (Ph.D. -7, NEB).
NTVIYQK

SEQ ID NO: 501 - protein, 7 aa, Artificial Sequence
  Selectively binds to Ni3B amorphous. Display platform: M13 phage (Ph.D. -7, NEB).
NVNSTSF

SEQ ID NO: 502 - protein, 7 aa, Artificial Sequence
  Selectively binds to Ni3B amorphous. Display platform: M13 phage (Ph.D. -7, NEB).
RLLNPWI

SEQ ID NO: 503 - protein, 7 aa, Artificial Sequence
  Selectively binds to Ni3B amorphous. Display platform: M13 phage (Ph.D. -7, NEB).
SEIVDNH

SEQ ID NO: 504 - protein, 7 aa, Artificial Sequence
  Selectively binds to Ni3B amorphous. Display platform: M13 phage (Ph.D. -7, NEB).
SLAVSRS

SEQ ID NO: 505 - protein, 7 aa, Artificial Sequence
  Selectively binds to Ni3B amorphous. Display platform: M13 phage (Ph.D -7, NEB).
SPDTVQK

SEQ ID NO: 506 - protein, 7 aa, Artificial Sequence
  Selectively binds to Ni3B amorphous. Display platform: M13 phage (Ph.D. -7, NEB).
TNLTLAS

SEQ ID NO: 507 - protein, 7 aa, Artificial Sequence
  Selectively binds to Ni3B amorphous. Display platform: M13 phage (Ph.D. -7, NEB).
TNSSFHK

SEQ ID NO: 508 - protein, 7 aa, Artificial Sequence
  Selectively binds to Ni3B amorphous. Selectively binds to M13 phage (Ph.D. -7, NEB).
TQVYHPM

SEQ ID NO: 509 - protein, 7 aa, Artificial Sequence
  Selectively binds to Ni3B amorphous. Display platform: M13 phage (Ph.D. -7, NEB).
VSVNSRT

SEQ ID NO: 510 - protein, 7 aa, Artificial Sequence
  Selectively binds to Ni3B amorphous. Display platform: M13 phage (Ph.D. -7, NEB).
AGLPKHQ

SEQ ID NO: 511 - protein, 7 aa, Artificial Sequence
  Selectively binds to Ni3B crystalline. Display platform: M13 phage (Ph.D. -7, NEB).
ATSTAHA

SEQ ID NO: 512 - protein, 7 aa, Artificial Sequence
  Selectively binds to Ni3B crystalline. Display platform: M13 phage (Ph.D. -7, NEB).
DPYNRIN

SEQ ID NO: 513 - protein, 7 aa, Artificial Sequence
  Selectively binds to Ni3B crystalline. Display platform: M13 phage (Ph.D. -7, NEB).
ELTQISS

SEQ ID NO: 514 - protein, 7 aa, Artificial Sequence
  Selectively binds to Ni3B crystalline. Display platform: M13 phage (Ph.D. -7, NEB).
ETFPARG

SEQ ID NO: 515 - protein, 7 aa, Artificial Sequence
  Selectively binds to Ni3B crystalline. Display platform: M13 phage (Ph.D. -7, NEB).
GASATRT

SEQ ID NO: 516 - protein, 7 aa, Artificial Sequence
  Selectively binds to Ni3B crystalline. Display platform: M13 phage (Ph.D. -7, NEB).
GDHSRHK

SEQ ID NO: 517 - protein, 7 aa, Artificial Sequence
  Selectively binds to Ni3B crystalline. Display platform: M13 phage (Ph.D. -7, NEB).
GDPKAAR

SEQ ID NO: 518 - protein, 7 aa, Artificial Sequence
  Selectively binds to Ni3B crystalline. Display platform: M13 phage (Ph.D. -7, NEB).
GPVNHQL

SEQ ID NO: 519 - protein, 7 aa, Artificial Sequence
  Selectively binds to Ni3B crystalline. Display platform: M13 phage (Ph.D. -7, NEB).
HAMRTEP

SEQ ID NO: 520 - protein, 7 aa, Artificial Sequence
  Selectively binds to Ni3B crystalline. Display platform: M13 phage (Ph.D. -7, NEB).
LEQTPMF

SEQ ID NO: 521 - protein, 7 aa, Artificial Sequence
  Selectively binds to Ni3B crystalline. Display platform: M13 phage (Ph.D. -7, NEB).
LNHVLPA

SEQ ID NO: 522 - protein, 7 aa, Artificial Sequence
  Selectively binds to Ni3B crystalline. Display platform: M13 phage (Ph.D. -7, NEB).
MNHAESY

SEQ ID NO: 523 - protein, 7 aa, Artificial Sequence
  Selectively binds to Ni3B crystalline. Display platform: M13 phage (Ph.D. -7, NEB).
RTFDAIS

SEQ ID NO: 524 - protein, 7 aa, Artificial Sequence
  Selectively binds to Ni3B crystalline. Display platform: M13 phage (Ph.D. -7, NEB).
SASKVHN

SEQ ID NO: 525 - protein, 7 aa, Artificial Sequence
  Selectively binds to Ni3B crystalline. Display platform: M13 phage (Ph.d. -7, NEB).
SDPQTHT

SEQ ID NO: 526 - protein, 7 aa, Artificial Sequence
  Selectively binds to Ni3B crystalline. Display platform: M13 phage (Ph.D. -7, NEB).
SPPKSNA

SEQ ID NO: 527 - protein, 7 aa, Artificial Sequence
  Selectively binds to Ni3B crystalline. Display platform: M13 phage (Ph.D. -7, NEB).
SPSTHWK

SEQ ID NO: 528 - protein, 7 aa, Artificial Sequence
  Selectively binds to Ni3B crystalline. Display platform: M13 phage (Ph.D. -7, NEB).
STFNSRV

SEQ ID NO: 529 - protein, 7 aa, Artificial Sequence
  Selectively binds to Ni3B crystalline. Display platform: M13 phage (Ph.D. -7, NEB).
SYTKLHL

SEQ ID NO: 530 - protein, 7 aa, Artificial Sequence
  Selectively binds to Ni3B crystalline. Display platform: M13 phage (Ph.D. -7, NEB).
TPPLLSP

SEQ ID NO: 531 - protein, 7 aa, Artificial Sequence
  Selectively binds to Ni3B crystalline. Display platform: M13 phage (Ph.D. -7, NEB).
VHTNPSR

SEQ ID NO: 532 - protein, 7 aa, Artificial Sequence
  Selectively binds to Ni3B crystalline. Display platform: M13 phage (Ph.D. -7, NEB).
VPIPYLP

SEQ ID NO: 533 - protein, 7 aa, Artificial Sequence
  Selectively binds to Ni3B crystalline. Display platform: M13 phage (Ph.D. -7, NEB).
VPSLTPT

SEQ ID NO: 534 - protein, 7 aa, Artificial Sequence
  Selectively binds to Ni3B crystalline. Display platform: M13 phage (Ph.D. -7, NEB)>
WNAKYTL

SEQ ID NO: 535 - protein, 7 aa, Artificial Sequence
  Selectively binds to Ni3B crystalline. Display platform: M13 phage (Ph.D. -7, NEB).
YELVLPK

SEQ ID NO: 536 - protein, 6 aa, Artificial Sequence
  Selectively binds to Ni3B crystalline. Display platform: M13 phage (Ph.D. -7, NEB).
YQWPAR

SEQ ID NO: 537 - protein, 12 aa, Artificial Sequence
  Selectively binds to particulate matter with hgih metal content. Display platform: peptide array containing a library of 85 sequences pre-designed with R based on Alvin, 2017, with WQDFGAVRSTRS = 100% binding.
GYHHSTYYTHNH

SEQ ID NO: 538 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
HHLFHAVYLNHY

SEQ ID NO: 539 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
HHLHWPHHHSYT

SEQ ID NO: 540 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
HHTSGHHSLTLT

SEQ ID NO: 541 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
HLVNRLRYPHVH

SEQ ID NO: 542 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
LTAHSHHHYHYA

SEQ ID NO: 543 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
LTHVLVHFYYHH

SEQ ID NO: 544 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
NGYYPHSHSYHQ

SEQ ID NO: 545 - protein, 12 aa, Artificial Sequence
  Selectively binds to particulate matter with a high metal content. Display platform: peptide array containing a library of 85 sequences pre-designed with R based on Alvin, 2017, with WQDFGAVRSTRS = 100% binding.
NHVNTNYYPTLH

SEQ ID NO: 546 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
NHYYSHTHTYHG

SEQ ID NO: 547 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
WHHYFRTTHLTT

SEQ ID NO: 548 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding.
YRAYHYLSYRDT

SEQ ID NO: 549 - protein, 12 aa, Artificial Sequence
  Selectively binds to particulate matter with high metal content. Display platform: peptide array containing a library of 85 sequences pre-designed with R based on Alvin, 2017, with WQDFGAVRSTRS = 100% binding
YTYTGLHLLASH

SEQ ID NO: 550 - protein, 11 aa, Artificial Sequence
  Selectively binds to Pb(NO3)2, Cd(NO3)2 and ZnCl2. Display platform: Saccharomyces cerevisiae.
MDCPTEEALIR

SEQ ID NO: 551 - protein, 7 aa, Artificial Sequence
  Selectively binds to Pb2+. Display platform: phage display library.
ISLLHST

SEQ ID NO: 552 - protein, 7 aa, Artificial Sequence
  Selectively binds to PbS. Display platform: M13 phage.
QNPIHTH

SEQ ID NO: 553 - protein, 23 aa, Artificial Sequence
  Selectively binds to polycrystalline quartz. Display platform: E. coli.
DAQTNSSSGGGEQLGVRKELRGV

SEQ ID NO: 554 - protein, 23 aa, Artificial Sequence
  Selectively binds to polycrystalline quartz. Display platform: E. coli.
DAQTNSSSGGGNPSSLFRYLPSD

SEQ ID NO: 555 - protein, 24 aa, Artificial Sequence
  Selectively binds to polycrystalline quartz. Display platform: E. coli.
DAQTNSSSNNNNNNNNNNLGIEGR

SEQ ID NO: 556 - protein, 7 aa, Artificial Sequence
  Selectively binds to polystyrene. Display platform: phage display library.
RRETAWA

SEQ ID NO: 557 - protein, 34 aa, Artificial Sequence
  Selectively binds to REE leachate and TbCl3 hydrate salts. Display platform: Caulobacter crescentus and E. coli.
FIDTNNDGWIEGDELLAFIDTNNDGWIEGDELLA

SEQ ID NO: 558 - protein, 7 aa, Artificial Sequence
  Selectively binds to sheet silicates mica. Display platform: Si3N4 AFM cantilever with silicon tip.
QPASSRY

SEQ ID NO: 559 - protein, 7 aa, Artificial Sequence
  Selectively binds to sheet silicates mica. Display platform: Si3N4 AFM cantilever with silicon tip.
APASSRY

SEQ ID NO: 560 - protein, 7 aa, Artificial Sequence
  Selectively binds to sheet silicates mica. Display platform: Si3N4 AFM cantilever with silicon tip.
QAASSRY

SEQ ID NO: 561 - protein, 7 aa, Artificial Sequence
  Selectively binds to sheet silicates mica. Display platform: Si3N4 AFM cantilever with silicon tip.
QPAASRY

SEQ ID NO: 562 - protein, 7 aa, Artificial Sequence
  Selectively binds to sheet silicates mica. Display platform: Si3N4 AFM cantilever with silicon tip.
QPASARY

SEQ ID NO: 563 - protein, 7 aa, Artificial Sequence
  Selectively binds to sheet silicates mica. Display platform: Si3N4 AFM cantilever with silicon tip.
QPASSAY

SEQ ID NO: 564 - protein, 7 aa, Artificial Sequence
  Selectively binds to sheet silicates mica. Display platform: Si3N4 AFM cantilever with silicon tip.
QPASSRA

SEQ ID NO: 565 - protein, 12 aa, Artificial Sequence
  Selectively binds to Si 4nm SiOx spatter coated on a gold SPR chip. Display platform: chemical synthesis.
CINQEGAGSKDK

SEQ ID NO: 566 - protein, 15 aa, Artificial Sequence
  Selectively binds to Si 4 nm SiOx spatter coated on a gold SPR chip. Display platform: chemical synthesis.
EVRKEVVAVARNTVI

SEQ ID NO: 567 - protein, 12 aa, Artificial Sequence
  Selectively binds to Si 4 nm SiOx spatter coated ona  gold SPR chip. Display platform: chemical synthesis.
LPDWWPPPQLYH

SEQ ID NO: 568 - protein, 12 aa, Artificial Sequence
  Selectively binds to Si 4 nm SiOx spatter coated on a gold SPR chip. Display platform: chemical synthesis.
LPWLPSWHQHLS

SEQ ID NO: 569 - protein, 12 aa, Artificial Sequence
  Selectively binds to Si 4 nm SiOx spatter coated on a gold SPR chip. Display platform: chemical synthesis.
LQWLGPQSPQWP

SEQ ID NO: 570 - protein, 12 aa, Artificial Sequence
  Selectively binds to Si 4 nm SiOx spatter coated on a gold SPR chip. Display platform: chemical synthesis.
LSPFWPLAPPWH

SEQ ID NO: 571 - protein, 12 aa, Artificial Sequence
  Selectively binds to Si 4 nm spatter coated on a gold SPR chip. Display platform: chemical synthesis.
RKEDKAEDTKKK

SEQ ID NO: 572 - protein, 12 aa, Artificial Sequence
  Selectively binds to Si 4 nm SiOx spatter coated on a gold SPR chip. Display platform: phage library and chemical synthesis.
RLNPPSQMDPPF

SEQ ID NO: 573 - protein, 12 aa, Artificial Sequence
  Selectively binds to Si 4 nm SiOx spatter coated on a gold SPR chip. Display platform: chemical synthesis.
SPPRLLPWLRMP

SEQ ID NO: 574 - protein, 12 aa, Artificial Sequence
  Selectively binds to Si 4 nm SiOx spatter coated on a gold SPR chip. Display platform: chemical synthesis.
VSVKTTKMTVVD

SEQ ID NO: 575 - protein, 12 aa, Artificial Sequence
  Selectively binds to Si glass. Display platform: custom engineered fluorescent proteins and sfGFP.
DSARGFKKPGKR

SEQ ID NO: 576 - protein, 12 aa, Artificial Sequence
  Selectively binds to Si glass. Display platform: custom engineered fluorescent protein.
RTYLPLPWMAAL

SEQ ID NO: 577 - protein, 12 aa, Artificial Sequence
  Selectively binds to silica, SiO2. Display platform: M13 phage.
HPPMNASHPHMH

SEQ ID NO: 578 - protein, 12 aa, Artificial Sequence
  Selectively binds to silica, silicic acid and SiO2. Display platform: M13 (Ph.D. -12, NEB).
MSPHPHPRHHHT

SEQ ID NO: 579 - protein, 6 aa, Artificial Sequence
  Selectively binds to silica, SiO2 nanoparticles, Ti organometallic compounds and Ti. Display platform: Fmoc synthesis and E. coli fermentation.
RKLPDA

SEQ ID NO: 580 - protein, 18 aa, Artificial Sequence
  Selectively binds to silica and silicic acid. Display platform: M13 (Ph.D. -12, NEB).
SSKKSGSYSGSKGSRRIL

SEQ ID NO: 581 - protein, 12 aa, Artificial Sequence
  Selectively binds to silicic acid. Display platform: M13 (Ph.D. -12, NEB).
APHHHHPHHLSR

SEQ ID NO: 582 - protein, 12 aa, Artificial Sequence
  Selectively bidns to silicic acid. Display platform: M13 (Ph.D. -12, NEB).
APPGHHHWHIHH

SEQ ID NO: 583 - protein, 12 aa, Artificial Sequence
  Selectively binds to silicic acid and SiO2. Display platform: M13 (Ph.D. -12, NEB).
KPSHHHHHTGAN

SEQ ID NO: 584 - protein, 12 aa, Artificial Sequence
  Selectively binds to silicic acid. Display platform: M13 (Ph.D. -12, NEB).
LPHHHHLHTKLP

SEQ ID NO: 585 - protein, 12 aa, Artificial Sequence
  Selectively binds to silicic acid. Display platform: M13 (Ph.D. -12, NEB).
MSASSYASFSWS

SEQ ID NO: 586 - protein, 12 aa, Artificial Sequence
  Selectively binds to silicic acid. Display platform: M13 (Ph.D. -12, NEB).
MSPHHMHHSHGH

SEQ ID NO: 587 - protein, 12 aa, Artificial Sequence
  Selectively binds to silicic acid and SiO2. Display platform: M13 (Ph.D. -12, NEB).
RGRRRRLSCRLL

SEQ ID NO: 588 - protein, 12 aa, Artificial Sequence
  Selectively binds to silicic acid. Display platform: M13 (Ph.D. -12, NEB).
TVASNSGLRPAS

SEQ ID NO: 589 - protein, 12 aa, Artificial Sequence
  Selectively binds to single crystal graphite. Display platform: M13 phage (NEB) and Si-C particles.
WPHHPHAAHTIR

SEQ ID NO: 590 - protein, 11 aa, Artificial Sequence
  Selectively binds to SiO4 quartz. Display platform: chemical synthesis.
PPWLPYMPPWS

SEQ ID NO: 591 - protein, 13 aa, Artificial Sequence
  Selectively binds to streptavidin binder. Display platform: M13 phage pIII library.
SWDPYSHLLQHPQ

SEQ ID NO: 592 - protein, 4 aa, Artificial Sequence
  Selectively binds to strontium titanante. Display platform: M13 bacteriophage.
AEEE

SEQ ID NO: 593 - protein, 21 aa, Artificial Sequence
  Selectively binds to Tn3+. Display platform: 280-320 micron Tentagel microbeads.
AACGDYNADGWIEFEELAACA

SEQ ID NO: 594 - protein, 20 aa, Artificial Sequence
  Selectively binds to Tb3+. Display platform: 280-320 micron TentaGel microbeads.
AACGDYNADGWIEFEELACA

SEQ ID NO: 595 - protein, 17 aa, Artificial Sequence
  Selectively binds to Tb3+. Display platform: 280-320 micron TentaGel beads.
AADWNKDGWYEGPEAAA

SEQ ID NO: 596 - protein, 17 aa, Artificial Sequence
  Selectively binds to Tb3+. Display platform: 280-320 micron TentaGel microbeads.
  SITE (4)..(4): Xaa can be any naturally occurring amino acid
AADXNKDGWYEGPEYYY

SEQ ID NO: 597 - protein, 14 aa, Artificial Sequence
  Selectively binds to Tb3+ and TbCl. Display platform: 280-320 micron TentaGel microbeads and plasmids designed to express LBT-tags on cells.
  MOD_RES (1)..(1): ACETYLATION
GDYNADGWIEFEEL

SEQ ID NO: 598 - protein, 16 aa, Artificial Sequence
  Selectively binds to Tb3+. Display platform: 280-320 micron TentaGel microbeads.
  MOD_RES (1)..(1): ACETYLATION
GGDYNADGWIEFEELL

SEQ ID NO: 599 - protein, 21 aa, Artificial Sequence
  Selectively binds to Tb3+. Display platform: 280-320 micron TentaGel microbeads.
ACAAGDYNADGWIEFEELACA

SEQ ID NO: 600 - protein, 21 aa, Artificial Sequence
  Selectively binds to Tb3+. Display platform: 280-320 micron TentaGel microbeads.
ACAGDYNADGWIEFEELAACA

SEQ ID NO: 601 - protein, 20 aa, Artificial Sequence
  Selectively binds to Tb3+. Display platform: 280-320 micron TentaGel microbeads.
ACAGDYNADGWIEFEELACA

SEQ ID NO: 602 - protein, 20 aa, Artificial Sequence
  Selectively binds to Tb3+. Display platform: 280-320 micron TentaGel microbeads.
ACAGDYNADGWIEFEELCAA

SEQ ID NO: 603 - protein, 17 aa, Artificial Sequence
  Selectively binds to Tb3+. Display platform: 280-320 micron TentaGel microbeads.
ACVDWNNDGWYEGDECA

SEQ ID NO: 604 - protein, 18 aa, Artificial Sequence
  Selectively binds to Tb3+. Display platform: 280-320 micron TentaGel microbeads.
AYADTNNDGWYEGDELEA

SEQ ID NO: 605 - protein, 16 aa, Artificial Sequence
  Selectively binds to Tb3+. Display platform: 280-320 micron TentaGel microbeads.
CGDYNADGWIEFEELC

SEQ ID NO: 606 - protein, 11 aa, Artificial Sequence
  Selectively binds to Tb3+. Display platform: 280-320 micron TentaGel microbeads.
  SITE (2)..(2): Xaa can be any naturally occurring amino acid
  SITE (4)..(4): Xaa can be any naturally occurring amino acid
  SITE (6)..(7): Xaa can be any naturally occurring amino acid
  SITE (9)..(10): Xaa can be any naturally occurring amino acid
DXNXDXXEXXE

SEQ ID NO: 607 - protein, 18 aa, Artificial Sequence
  Selectively binds to Tb3+. Display platform: 280-320 micron TentaGel microbeads.
DYDDTNNDGWYEGDELLA

SEQ ID NO: 608 - protein, 18 aa, Artificial Sequence
  Selectively binds to Tb3+. Display platform: 280-320 micron TentaGel microbeads.
EYEDTNNDGWYEGDELNA

SEQ ID NO: 609 - protein, 17 aa, Artificial Sequence
  Selectively binds to Tb3+. Display platform: 280-320 micron TentaGel microbeads.
FIDFNGDGWWEDDELLA

SEQ ID NO: 610 - protein, 17 aa, Artificial Sequence
  Selectively binds to Tb3+. Display platform: 280-320 micron TentaGel microbeads.
FIDTNNDGWFEGDEFLA

SEQ ID NO: 611 - protein, 17 aa, Artificial Sequence
  Selectively binds to Tb3+ and TbCl3 hydrate salts. Display platform: 280-320 micron TentaGel microbeads, E. coli and chemical synthesis.
FIDTNNDGWIEGDELLA

SEQ ID NO: 612 - protein, 20 aa, Artificial Sequence
  Selectively binds to Tb3+. Display platform: 280-320 micron TentaGel microbeads.
FIDTNNDGWIEGDELLLEEG

SEQ ID NO: 613 - protein, 17 aa, Artificial Sequence
  Selectively binds to Tb3+. Display platform: 280-320 micron TentaGel microbeads.
FIDTNNDGWWEGDELLA

SEQ ID NO: 614 - protein, 17 aa, Artificial Sequence
  Selectively binds to Tb3+. Display platform: 280-320 micron TentaGel microbeads.
FIDTNNDGWYEGDELLA

SEQ ID NO: 615 - protein, 17 aa, Artificial Sequence
  Selectively binds to Tb3+. Display platform: 280-320 micron TentaGel microbeads.
  SITE (2)..(2): Xaa can be any naturally occurring amino acid
  SITE (9)..(10): Xaa can be any naturally occurring amino acid
FXDFNRDGXXENNELLA

SEQ ID NO: 616 - protein, 17 aa, Artificial Sequence
  Selectively binds to Tb3+. Display platform: advanced ChemTech 396 synthesizer.
IFDKNADGFIDIEELGE

SEQ ID NO: 617 - protein, 17 aa, Artificial Sequence
  Selectively binds to Tb3+. Display platform: 280-320 micron TentaGel microbeads.
IIDTNNDGWIEGDELLA

SEQ ID NO: 618 - protein, 15 aa, Artificial Sequence
  Selectively binds to Tb3+. Display platform: 280-320 micron TentaGel microbeads.
LADYNKDGWYDGGDL

SEQ ID NO: 619 - protein, 17 aa, Artificial Sequence
  Selectively binds to Tb3+. Display platform: 280-320 micron TentaGel microbeads.
  SITE (4)..(4): Xaa can be any naturally occurring amino acid
LLDXNKDGWYEGPEWWA

SEQ ID NO: 620 - protein, 17 aa, Artificial Sequence
  Selectively binds to Tb3+. Display platform: 280-320 micron TentaGel microbeads.
LLDYNKDGWYEGPELLL

SEQ ID NO: 621 - protein, 17 aa, Artificial Sequence
  Selectively binds to Tb3+. Display platform: 280-320 micron TentaGel microbeads.
MIDTNNDGWMEGDEMLA

SEQ ID NO: 622 - protein, 19 aa, Artificial Sequence
  Selectively binds to Tb3+. Display platform: 280-320 micron TentaGel microbeads.
NYIDTNNDGWYEGDELQAH

SEQ ID NO: 623 - protein, 18 aa, Artificial Sequence
  Selectively binds to Tb3+. Display platform: 280-320 micron TentaGel microbeads.
SYNDTNNDGWYEGDELYA

SEQ ID NO: 624 - protein, 15 aa, Artificial Sequence
  Selectively binds to Tb3+. Display platform 280-320 micron TentaGel microbeads.
TGDYNKDGWYEPPET

SEQ ID NO: 625 - protein, 15 aa, Artificial Sequence
  Selectively binds to Tb3+. Display platform: 280-320 micron TentaGel microbeads.
  SITE (12)..(13): Xaa can be any naturally occurring amino acid
TWDYNKDGWYSXXST

SEQ ID NO: 626 - protein, 18 aa, Artificial Sequence
  Selectively binds to Tb3+. Display platform: 280-320 micron TentaGel microbeads.
TYQDTNNDGWYEGDELLA

SEQ ID NO: 627 - protein, 17 aa, Artificial Sequence
  Selectively binds to Tb3+. Display platform: 280-320 micron TentaGel microbeads.
  SITE (4)..(4): Xaa can be any naturally occurring amino acid
VVDXNKDGWYEGPEPPT

SEQ ID NO: 628 - protein, 17 aa, Artificial Sequence
  Selectively binds to Tb3+. Display platform: 280-320 micron TentaGel microbeads.
  SITE (4)..(4): Xaa can be any naturally occurring amino acid
VVDXNKDGWYEGPETTT

SEQ ID NO: 629 - protein, 15 aa, Artificial Sequence
  Selectively binds to Tb3+. Display platform: 280-320 micron TentaGel microbeads.
VWDYNKDGWYNGPNV

SEQ ID NO: 630 - protein, 15 aa, Artificial Sequence
  Selectively binds to Tb3+. Display platform: 280-320 micron TentaGel microbeads.
VYDYNKDGWYEGPEL

SEQ ID NO: 631 - protein, 15 aa, Artificial Sequence
  Selectively binds to Tb3+. Display platform: 280-320 micron TentaGel microbeads.
VYDYNKDGWYEPPEV

SEQ ID NO: 632 - protein, 15 aa, Artificial Sequence
  Selectively binds to Tb3+. Display platform: 280-320 micron TentaGel microbeads.
VYDYNKDGWYQGPQL

SEQ ID NO: 633 - protein, 15 aa, Artificial Sequence
  Selectively binds to Tb3+. Display platform: 280-320 micron TentaGel microbeads.
VYDYNKDGWYSGPSL

SEQ ID NO: 634 - protein, 17 aa, Artificial Sequence
  Selectively binds to Tb3+. Display platform: 280-320 micron TentaGel microbeads.
WIDTNNDGWTEGDEWLA

SEQ ID NO: 635 - protein, 17 aa, Artificial Sequence
  Selectively binds to Tb3+. Display platform: 280-320 micron TentaGel microbeads.
WIDWNKDGYYESSELLA

SEQ ID NO: 636 - protein, 17 aa, Artificial Sequence
  Selectively binds to Tb3+. Display platform: 280-320 micron TentaGel microbeads.
  SITE (4)..(4): Xaa can be any naturally occurring amino acid
WPDXNKDGWYEGPETTV

SEQ ID NO: 637 - protein, 17 aa, Artificial Sequence
  Selectively binds to Tb3+. Display platform: 280-320 micron TentaGel microbeads.
WVDGNKDGYYEEGELLA

SEQ ID NO: 638 - protein, 17 aa, Artificial Sequence
  Selectively binds to Tb3+. Display platform: 280-320 micron TentaGel microbeads.
WVDWNKDGWYEGPELLA

SEQ ID NO: 639 - protein, 17 aa, Artificial Sequence
  Selectively binds to Tb3+. Display platform: 280-320 micron TentaGel microbeads.
WWDYNKDGWYEGPELLL

SEQ ID NO: 640 - protein, 17 aa, Artificial Sequence
  Selectively binds to Tb3+. Display platform: 280-320 micron TentaGel microbeads.
  SITE (1)..(1): Xaa can be any naturally occurring amino acid
  SITE (16)..(17): Xaa can be any naturally occurring amino acid
XGDYNKDGWYEELELXX

SEQ ID NO: 641 - protein, 17 aa, Artificial Sequence
  Selectively binds to Tb3+. Display platform: 280-320 micron TentaGel microbeads.
  SITE (1)..(1): Xaa can be any naturally occurring amino acid
  SITE (4)..(4): Xaa can be any naturally occurring amino acid
  SITE (17)..(17): Xaa can be any naturally occurring amino acid
XPDXBKDGWYEGPEAAX

SEQ ID NO: 642 - protein, 17 aa, Artificial Sequence
  Selectively binds to Tb3+. Display platform: 280-320 micron TentaGel microbeads.
  SITE (1)..(1): Xaa can be any naturally occurring amino acid
  SITE (4)..(4): Xaa can be any naturally occurring amino acid
XVDXNKDGWYEGPEYYA

SEQ ID NO: 643 - protein, 17 aa, Artificial Sequence
  Selectively binds to Tb3+. Display platform: 280-320 micron TentaGel microbeads.
  SITE (1)..(2): Xaa can be any naturally occurring amino acid
  SITE (6)..(6): Xaa can be any naturally occurring amino acid
  SITE (9)..(10): Xaa can be any naturally occurring amino acid
  SITE (13)..(13): Xaa can be any naturally occurring amino acid
XXDFNXDGXXEPXELLA

SEQ ID NO: 644 - protein, 17 aa, Artificial Sequence
  Selectively binds to Tb3+. Display platform: 280-320 micron TentaGel microbeads.
  SITE (1)..(2): Xaa can be any naturally occurring amino acid
  SITE (9)..(10): Xaa can be any naturally occurring amino acid
  SITE (13)..(13): Xaa can be any naturally occurring amino acid
XXDGNGDGXXEEXELLA

SEQ ID NO: 645 - protein, 17 aa, Artificial Sequence
  Selectively binds to Tb3+. Display platform: 280-320 micron TentaGel microbeads.
  SITE (1)..(2): Xaa can be any naturally occurring amino acid
  SITE (6)..(6): Xaa can be any naturally occurring amino acid
  SITE (9)..(10): Xaa can be any naturally occurring amino acid
  SITE (12)..(13): Xaa can be any naturally occurring amino acid
XXDLNXDGXXEXXELLA

SEQ ID NO: 646 - protein, 17 aa, Artificial Sequence
  Selectively binds to Tb3+. Display platform: 280-320 micron TentaGel microbeads.
  SITE (1)..(2): Xaa can be any naturally occurring amino acid
  SITE (9)..(10): Xaa can be any naturally occurring amino acid
XXDPNPDGXXEDDELLA

SEQ ID NO: 647 - protein, 17 aa, Artificial Sequence
  Selectively binds to Tb3+. Display platform: 280-320 micron TentaGel microbeads.
  SITE (1)..(2): Xaa can be any naturally occurring amino acid
  SITE (4)..(4): Xaa can be any naturally occurring amino acid
  SITE (17)..(17): Xaa can be any naturally occurring amino acid
XXDXNKDGWYEGPEPPX

SEQ ID NO: 648 - protein, 17 aa, Artificial Sequence
  Selectively binds to Tb3+. Display platform: 280-320 micron TentaGel microbeads.
  SITE (1)..(2): Xaa can be any naturally occurring amino acid
  SITE (4)..(4): Xaa can be any naturally occurring amino acid
  SITE (15)..(17): Xaa can be any naturally occurring amino acid
XXDXNKDGWYEGPEXXX

SEQ ID NO: 649 - protein, 17 aa, Artificial Sequence
  Selectively binds to Tb3+. Display platform: 280-320 micron TentaGel microbeads.
  SITE (1)..(2): Xaa can be any naturally occurring amino acid
  SITE (4)..(4): Xaa can be any naturally occurring amino acid
  SITE (6)..(6): Xaa can be any naturally occurring amino acid
  SITE (9)..(10): Xaa can be any naturally occurring amino acid
  SITE (12)..(13): Xaa can be any naturally occurring amino acid
XXDXNXDGXXEXXELLA

SEQ ID NO: 650 - protein, 15 aa, Artificial Sequence
  Selectively binds to Tb3+. Display platform: 280-320 micron TentaGel microbeads.
  SITE (1)..(2): Xaa can be any naturally occurring amino acid
  SITE (12)..(13): Xaa can be any naturally occurring amino acid
  SITE (15)..(15): Xaa can be any naturally occurring amino acid
XXDYNKDGWYNXXNX

SEQ ID NO: 651 - protein, 15 aa, Artificial Sequence
  Selectively binds to Tb3+. Display platform: 280-320 micron TentaGel microbeads.
  SITE (1)..(2): Xaa can be any naturally occurring amino acid
  SITE (12)..(13): Xaa can be any naturally occurring amino acid
  SITE (15)..(15): Xaa can be any naturally occurring amino acid
XXDYNKDGWYQXXQX

SEQ ID NO: 652 - protein, 17 aa, Artificial Sequence
  Selectively binds to Tb3+. Display platform: 280-320 micron TentaGel microbeads.
  SITE (1)..(1): Xaa can be any naturally occurring amino acid
  SITE (4)..(4): Xaa can be any naturally occurring amino acid
XYDXNKDGWYEGPEWWA

SEQ ID NO: 653 - protein, 17 aa, Artificial Sequence
  Selectively binds to Tb3+. Display platform: 280-320 micron TentaGel microbeads.
YIDFNGDGWYEGDELLA

SEQ ID NO: 654 - protein, 17 aa, Artificial Sequence
  Selectively binds to Tb3+. Display platform: 280-320 micron TentaGel microbeads.
YIDFNNDGWYEGDELLA

SEQ ID NO: 655 - protein, 17 aa, Artificial Sequence
  Selectively binds to Tb3+. Display platform: 280-320 micron TentaGel microbeads.
YIDLNNDGWYEGDELLA

SEQ ID NO: 656 - protein, 17 aa, Artificial Sequence
  Selectively binds to Tb3+. Display platform: 280-320 micron TentaGel microbeads.
YIDLNNDGWYEGNELLA

SEQ ID NO: 657 - protein, 17 aa, Artificial Sequence
  Selectively binds to Tb3+. Display platform: 280-320 micron TentaGel microbeads.
  SITE (6)..(6): Xaa can be any naturally occurring amino acid
YIDLNXDGWYEGDELLA

SEQ ID NO: 658 - protein, 17 aa, Artificial Sequence
  Selectively binds to Tb3+. Display platform: 280-320 micron TentaGel microbeads.
YIDPNPDGWTENPELLA

SEQ ID NO: 659 - protein, 17 aa, Artificial Sequence
  Selectively binds to Tb3+. Display platform: 280-320 micron TentaGel micron TentaGel microbeads.
YIDTNNDGWVEGDEYLA

SEQ ID NO: 660 - protein, 17 aa, Artificial Sequence
  Selectively binds to Tb3+ and TbCl3 hydrate salts. Display platform: E. coli, chemical synthesis and 280-320 micron TentaGel microbeads.
YIDTNNDGWYEGDELLA

SEQ ID NO: 661 - protein, 20 aa, Artificial Sequence
  Selectively binds to Tb3+. Display platform: 280-320 micron TentaGel microbeads.
YIDTNNDGWYEGDELLAAAA

SEQ ID NO: 662 - protein, 20 aa, Artificial Sequence
  Selectively binds to Tb3+. Display platform: 280-320 micron TentaGel microbeads.
YIDTNNDGWYEGDELLKEEG

SEQ ID NO: 663 - protein, 20 aa, Artificial Sequence
  Selectively binds to Tb3+. Display platform: 280-320 micron TentaGel microbeads.
YIDTNNDGWYEGDELLLEEG

SEQ ID NO: 664 - protein, 20 aa, Artificial Sequence
  Selectively binds to Tb3+. Display platform: 280-320 micron TentaGel microbeads.
YIDTNNDGWYEGDELLLEER

SEQ ID NO: 665 - protein, 20 aa, Artificial Sequence
  Selectively binds to Tb3+. Display platform: 280-320 micron TentaGel microbeads.
YIDTNNDGWYEGDELLLKKK

SEQ ID NO: 666 - protein, 20 aa, Artificial Sequence
  Selectively binds to Tb3+. Display platform: 280-320 micron TentaGel microbeads.
YIDTNNDGWYEGDELLLLLL

SEQ ID NO: 667 - protein, 17 aa, Artificial Sequence
  Selectively binds to Tb3+. Display platform: 280-320 micron TentaGel microbeads.
YIDWNNDGWYEGDELLA

SEQ ID NO: 668 - protein, 17 aa, Artificial Sequence
  Selectively binds to Tb3+. Display platform: 280-320 micron TentaGel microbeads.
YIDWNRDGWYESSELLA

SEQ ID NO: 669 - protein, 17 aa, Artificial Sequence
  Selectively binds to Tb3+. Display platform: 280-320 micron TentaGel microbeads.
  SITE (4)..(4): Xaa can be any naturally occurring amino acid
YIDXNNDGWYEGDELLA

SEQ ID NO: 670 - protein, 17 aa, Artificial Sequence
  Selectively binds to Tb3+. Display platform: 280-320 micron TentaGel microbeads.
YIDYNNDGWYEGDELLA

SEQ ID NO: 671 - protein, 17 aa, Artificial Sequence
  Selectively binds to Tb3+. Display platform: 280-320 micron TentaGel microbeads.
YVDYNKDGWYEGPELLA

SEQ ID NO: 672 - protein, 17 aa, Artificial Sequence
  Selectively binds to Tb3+. Display platform: 280-320 micron TentaGel microbeads.
YVDYNNDGWWEGGELLA

SEQ ID NO: 673 - protein, 17 aa, Artificial Sequence
  Selectively binds to Tb3+. Display platform: 280-320 micron TentaGel microbeads.
  SITE (4)..(4): Xaa can be any naturally occurring amino acid
YWDXNKDGWYEGPEWWA

SEQ ID NO: 674 - protein, 17 aa, Artificial Sequence
  Selectively binds to Tb3+. Display platform: 280-320 micorn TentaGel microbeads.
YYDTNNDGWYEGDELLA

SEQ ID NO: 675 - protein, 17 aa, Artificial Sequence
  Selectively binds to Tb3+. Display platform: 280-320 micron TentaGel microbeads.
YYDWNKDGWYEGPEVVV

SEQ ID NO: 676 - protein, 18 aa, Artificial Sequence
  Selectively binds to Tb3+. Display platform: 280-320 micron TentaGel microbeads.
YYTDTNNDGWYEGDELLA

SEQ ID NO: 677 - protein, 19 aa, Artificial Sequence
  Selectively binds to TbCl. Display platform: chemical synthesis and plasmids designed so cells express LBT-tags.
AACDYNKDGWYEELEAACA

SEQ ID NO: 678 - protein, 18 aa, Artificial Sequence
  Selectively binds to TbCl. Display platform: chemical synthesis and plasmids designed so cells express LBT-tags.
AACDYNKDGWYEELEACA

SEQ ID NO: 679 - protein, 14 aa, Artificial Sequence
  Selectively binds to TbCl. Display platform: chemical synthesis and plasmids designed so cells express LBT-tags.
  MOD_RES (1)..(1): ACETYLATION
GDYNKDGWYEELEL

SEQ ID NO: 680 - protein, 20 aa, Artificial Sequence
  Selectively binds to TbCl. Display platform: chemical synthesis and plasmids designed so cells express LBT-tags.
ACAADYNKDGWYEELEAACA

SEQ ID NO: 681 - protein, 19 aa, Artificial Sequence
  Selectively binds to TbCl. Display platform: chemical synthesis and plasmids designed so cells express LBT-tags.
ACAADYNKDGWYEELEACA

SEQ ID NO: 682 - protein, 19 aa, Artificial Sequence
  Selectively binds to TbCl. Display platform: chemical synthesis and plasmids designed so cells express LBT-tags.
ACAADYNKDGWYEELECAA

SEQ ID NO: 683 - protein, 19 aa, Artificial Sequence
  Selectively binds to TbCl. Display platform: chemical synthesis and plasmids designed so cells express LBT-tags.
ACADYNKDGWYEELEAACA

SEQ ID NO: 684 - protein, 18 aa, Artificial Sequence
  Selectively binds to TbCl. Display platform: chemical synthesis and plasmids designed so cells express LBT-tags.
ACADYNKDGWYEELEACA

SEQ ID NO: 685 - protein, 18 aa, Artificial Sequence
  Selectively binds to TbCl. Display platform: chemical synthesis and plasmids designed so cells express LBT-tags.
ACADYNKDGWYEELECAA

SEQ ID NO: 686 - protein, 14 aa, Artificial Sequence
  Selectively binds to TbCl. Display platform: chemical synthesis and plasmids designed so cells express LBT-tags.
CDYNKDGWYEELEC

SEQ ID NO: 687 - protein, 35 aa, Artificial Sequence
  Selectively binds to TbCl3 hydrate salts. Display platform: E. coli and chemical synthesis.
GPGYIDTNNDGWIEGDELYIDTNNDGWIEGDELLA

SEQ ID NO: 688 - protein, 33 aa, Artificial Sequence
  Selectively binds to TbCl3 hydrate salts. Display platform: E. coli and chemical synthesis.
GYIDTNNDGWIEGDELYIDTNNDGWIEGDELLA

SEQ ID NO: 689 - protein, 17 aa, Artificial Sequence
  Selectively binds to TbCl3 hydrate salts. Display platform: E. coli and chemical synthesis.
YIDTNNDGWIEGDELLA

SEQ ID NO: 690 - protein, 32 aa, Artificial Sequence
  Selectively binds to TbCl3 hydrate salts. Display platform: E. coli and chemical synthesis.
YIDTNNDGWIEGDELYIDTNNDGWIEGDELLA

SEQ ID NO: 691 - protein, 12 aa, Artificial Sequence
  Selectively binds to TbCl. Display platform: chemical synthesis and plasmids designed so cells express LBT-tags.
DYNKDGWYEELE

SEQ ID NO: 692 - protein, 12 aa, Artificial Sequence
  Selectively binds to Ti (<150 micron pure Ti particles). Display platform: M13 phage.
LCANNTTSVHPP

SEQ ID NO: 693 - protein, 12 aa, Artificial Sequence
  Selectively binds to Ti (<150 micron pure Ti particles). Display platform: M13 phage.
LDTTNVSGPMSS

SEQ ID NO: 694 - protein, 12 aa, Artificial Sequence
  Selectively binds to Ti (<150 micron pure Ti particles). Display platform: M13 phage.
LPSQLLSQVNLT

SEQ ID NO: 695 - protein, 12 aa, Artificial Sequence
  Selectively binds to Ti (<150 micron pure Ti particles). Display platform: M13 phage.
MQMEGKPTLTLR

SEQ ID NO: 696 - protein, 12 aa, Artificial Sequence
  Selectively binds to Ti (<150 micron pure Ti particles). Display platform: M13 phage.
QDMIRTSALMLQ

SEQ ID NO: 697 - protein, 12 aa, Artificial Sequence
  Selectively binds to Ti (<150 micron pure Ti particles). Display platform: M13 phage.
RKLPDAPGMHTW

SEQ ID NO: 698 - protein, 12 aa, Artificial Sequence
  Selectively binds to Ti (<150 micron pure Ti particles). Display platform: M13 phage.
SCHVWYDSCSSP

SEQ ID NO: 699 - protein, 12 aa, Artificial Sequence
  Selectively binds to Ti (<150 micron pure Ti particles). Display platform: M13 phage.
SDPNQDWRRTTP

SEQ ID NO: 700 - protein, 12 aa, Artificial Sequence
  Selectively binds to Ti (<150 micron pure Ti particles). Display platform: M13 phage.
STLKNPINLLAN

SEQ ID NO: 701 - protein, 12 aa, Artificial Sequence
  Selectively binds to Ti (<150 micron pure Ti particles). Display platform: M13 phage.
SYRLPVYLHALL

SEQ ID NO: 702 - protein, 12 aa, Artificial Sequence
  Selectively binds to Ti (implant-grade Ti). Display platform: solid phase peptide synthesis on Wang resin.
RPRENRGRERGL

SEQ ID NO: 703 - protein, 12 aa, Artificial Sequence
  Selectively binds to Ti (implant-grade Ti. Display platform: solid phase peptide synthesis on Wang resin.
SRPNGYGGSESS

SEQ ID NO: 704 - protein, 12 aa, Artificial Sequence
  Selectively binds to Ti (implant grade Ti). Display platform: FliTrx bacterial cell surface display system (Invitrogen).
VGRVTSPRPQGR

SEQ ID NO: 705 - protein, 18 aa, Artificial Sequence
  Selectively binds to TiBALDH. Display platform: chemical synthesis.
SSKKSGSYSGSKGKRRIL

SEQ ID NO: 706 - protein, 15 aa, Artificial Sequence
  Selectively binds to toner. Display platform: custom proteins.
GGGSGVYKVAYDWQH

SEQ ID NO: 707 - protein, 21 aa, Artificial Sequence
  Selectively binds to zeolites. Display platform: E. coli.
VKTQATSREEPPRLPSKHRPG

SEQ ID NO: 708 - protein, 12 aa, Artificial Sequence
  Selectively binds to zinc oxide. Display platform: M13 (Ph.D. -12, NEB).
EAHVMHKVAPRP

SEQ ID NO: 709 - protein, 5 aa, Artificial Sequence
  Selectively binds to zinc oxide. Display platform: chemical synthesis.
RPHRK

SEQ ID NO: 710 - protein, 6 aa, Artificial Sequence
  Selectively binds to Zn. Display platform: E. coli.
SYHHHH

SEQ ID NO: 711 - protein, 20 aa, Artificial Sequence
  Selectively binds to Zn2+. Display platform: influenza virus.
VCATCEQIANSQHRSHRQMV

SEQ ID NO: 712 - protein, 20 aa, Artificial Sequence
  Selectively binds to ZnO. Display platform: E. coli K-12.
NTRMTARQHRSANHKSTQRA

SEQ ID NO: 713 - protein, 9 aa, Artificial Sequence
  Selectively binds to ZnO. Display platform: peptide library in FimH.
YDSRSMRPH

SEQ ID NO: 714 - protein, 12 aa, Artificial Sequence
  Selectively binds to ZnO particles. Display platform: M13 (Ph.D. -12, NEB).
ATHTNQTHALYR

SEQ ID NO: 715 - protein, 12 aa, Artificial Sequence
  Selectively binds to ZnO particles. Display platform: M13 (Ph.D. -12, NEB).
DSGRYSMTNHYS

SEQ ID NO: 716 - protein, 12 aa, Artificial Sequence
  Selectively binds to ZnO. Display platform: M13 (Ph.D. -12, NEB).
QNTATAVSRLSP

SEQ ID NO: 717 - protein, 12 aa, Artificial Sequence
  Selectively binds to ZnO particles. Display platform: M13 (Ph.D. -12, NEB).
VSNHKALDYPTR

SEQ ID NO: 718 - protein, 12 aa, Artificial Sequence
  Selectively binds to ZnS. Display platform: sfGFP.
AGDSSGVDSRSV

SEQ ID NO: 719 - protein, 9 aa, Artificial Sequence
  Selectively binds to ZnS. Display platform: M13 phage.
CFPMRSNQC

SEQ ID NO: 720 - protein, 18 aa, Artificial Sequence
  Selectively binds to ZnS. Display platform: FliTrx cell surface display system (Invitrogen).
CGPAGDSSGVDSRSVGPC

SEQ ID NO: 721 - protein, 9 aa, Artificial Sequence
  Selectively binds to ZnS. Display platform: M13 phage.
CHMAPRWQC

SEQ ID NO: 722 - protein, 9 aa, Artificial Sequence
  Selectively binds to ZnS. Display platform: M13 phage.
CLQNRQSQC

SEQ ID NO: 723 - protein, 9 aa, Artificial Sequence
  Selectively binds to ZnS. Display platform: M13 phage.
CNHQMPMQC

SEQ ID NO: 724 - protein, 9 aa, Artificial Sequence
  Selectively binds to ZnS. Display platform: M13 phage.
CNKHQPMHC

SEQ ID NO: 725 - protein, 9 aa, Artificial Sequence
  Selectively binds to ZnS. Display platform: M13 phage.
CNNKVPVLC

SEQ ID NO: 726 - protein, 9 aa, Artificial Sequence
  Selectively binds to ZnS. Display platform: M13 phage and M13 phage (7 mer).
CNNPMHQNC

SEQ ID NO: 727 - protein, 9 aa, Artificial Sequence
  Selectively binds to ZnS. Display platform: M13 phage.
CNQLSTRPC

SEQ ID NO: 728 - protein, 9 aa, Artificial Sequence
  Selectively binds to ZnS. Display platform: M13 phage.
CNRQAVNAC

SEQ ID NO: 729 - protein, 9 aa, Artificial Sequence
  Selectively binds to ZnS. Display platform: M13 phage.
CPPQPNRQC

SEQ ID NO: 730 - protein, 9 aa, Artificial Sequence
  Selectively binds to ZnS. Display platform: M13 phage.
CQLQRQWNC

SEQ ID NO: 731 - protein, 9 aa, Artificial Sequence
  Selectively binds to ZnS. Display platform: M13 phage.
CQNPMQTFC

SEQ ID NO: 732 - protein, 9 aa, Artificial Sequence
  Selectively binds to ZnS. Display platform: M13 phage.
CQSMPHNRC

SEQ ID NO: 733 - protein, 9 aa, Artificial Sequence
  Selectively binds to ZnS. Display platform: M13 phage.
CQVNSAHQC

SEQ ID NO: 734 - protein, 12 aa, Artificial Sequence
  Selectively binds to ZnS. Display platform: M13 phage.
LPPAWAMQVHTA

SEQ ID NO: 735 - protein, 12 aa, Artificial Sequence
  Selectively binds to ZnS. Display platform: M13 phage.
LPRAFMGHAPGS

SEQ ID NO: 736 - protein, 12 aa, Artificial Sequence
  Selectively binds to ZnS. Display platform: M13 phage.
LRRSSEAHNSIV

SEQ ID NO: 737 - protein, 7 aa, Artificial Sequence
  Selectively binds to ZnS. Display platform: M13 phage and M13 phage (7 mer).
NNPMHQN

SEQ ID NO: 738 - protein, 7 aa, Artificial Sequence
  Selectively binds to ZnS. Display platform: Saccharomyces cerevisiae.
PHPHTHT

SEQ ID NO: 739 - protein, 12 aa, Artificial Sequence
  Selectively binds to ZnS. Display platform: M13 phage.
PRPSPKMGVSVS

SEQ ID NO: 740 - protein, 12 aa, Artificial Sequence
  Selectively binds to ZnS. Display platform: M13 phage.
RRQDVHLPSRTL

SEQ ID NO: 741 - protein, 12 aa, Artificial Sequence
  Selectively binds to ZnS. Display platform: M13 phage.
TRHMASRTEAHL

SEQ ID NO: 742 - protein, 7 aa, Artificial Sequence
  Selectively binds to ZnS sphalerite. Display platform: M13 phage.
QPKGPKQ

SEQ ID NO: 743 - protein, 7 aa, Artificial Sequence
  Selectively binds to ZnS sphalerite and CuFeS2 chalcopyrite. Display platform: M13 phage.
TPTTYKV

SEQ ID NO: 744 - protein, 7 aa, Artificial Sequence
  Selectively binds to ZnS sphalerite. Display platform: M13 phage.
KPLLMGS

SEQ ID NO: 745 - protein, 8 aa, Artificial Sequence
  Selectively binds to Co2+ and Ni2+. Display platform: Rink resin and Wang resin.
DAHKSEVA

SEQ ID NO: 746 - protein, 4 aa, Artificial Sequence
  Selectively binds to Co2+ and Ni2+. Display platform: Rink resin and Wang resin.
DAHK

SEQ ID NO: 747 - protein, 7 aa, Artificial Sequence
  Selectively binds to cobalt. Display platform: E. coli.
DRTISNK

SEQ ID NO: 748 - protein, 7 aa, Artificial Sequence
  Selectively binds to cobalt. Display platform: E. coli.
QNPGNTL

SEQ ID NO: 749 - protein, 7 aa, Artificial Sequence
  Selectively binds to cobalt. Display platform: E. coli.
SSSVVTH

SEQ ID NO: 750 - protein, 7 aa, Artificial Sequence
  Selectively binds to cobalt. Display platform: E. coli.
DAKDLNS

SEQ ID NO: 751 - protein, 7 aa, Artificial Sequence
  Selectively binds to cobalt. Display platform: E. coli.
DNDTKAS

SEQ ID NO: 752 - protein, 7 aa, Artificial Sequence
  Selectively binds to cobalt. Display platform: E. coli.
GLTDTSN

SEQ ID NO: 753 - protein, 7 aa, Artificial Sequence
  Selectively binds to cobalt. Display platform: E. coli.
KTSTHAI

SEQ ID NO: 754 - protein, 7 aa, Artificial Sequence
  Selectively binds to cobalt. Display platform: E. coli.
MRDSKML

SEQ ID NO: 755 - protein, 7 aa, Artificial Sequence
  Selectively binds to cobalt. Display platform: E. coli.
STISKAK

SEQ ID NO: 756 - protein, 7 aa, Artificial Sequence
  Selectively binds to cobalt. Display platform: E. coli.
TGQGGEY

SEQ ID NO: 757 - protein, 7 aa, Artificial Sequence
  Selectively binds to cobalt. Display platform: E. coli.
TKTQTHA

SEQ ID NO: 758 - protein, 7 aa, Artificial Sequence
  Selectively binds to cobalt. Display platform: E. coli.
TNHSAYH

SEQ ID NO: 759 - protein, 7 aa, Artificial Sequence
  Selectively binds to cobalt. Display platform: E. coli.
TQMLGQL

SEQ ID NO: 760 - protein, 7 aa, Artificial Sequence
  Selectively binds to cobalt. Display platform: E. coli.
VSPNKEA

SEQ ID NO: 761 - protein, 4 aa, Artificial Sequence
  Selectively binds to cobalt. Display platform: M13 virus.
EEEE

SEQ ID NO: 762 - protein, 7 aa, Artificial Sequence
  Selectively binds to cobalt and nickel. Display platform: E. coli.
MSTGLSS

SEQ ID NO: 763 - protein, 7 aa, Artificial Sequence
  Selectively binds to cobalt and nickel. Display platform: E. coli.
VPILEGT

SEQ ID NO: 764 - protein, 7 aa, Artificial Sequence
  Selectively binds to cobalt and nickel. Display platform: E. coli.
SGTGASY

SEQ ID NO: 765 - protein, 7 aa, Artificial Sequence
  Selectively binds to cobalt and nickel. Display platform: E. coli.
TASQNFY

SEQ ID NO: 766 - protein, 13 aa, Artificial Sequence
  Selectively binds to CoO. Display platform: E. coli K-12.
RSGRMQRRVAHRS

SEQ ID NO: 767 - protein, 13 aa, Artificial Sequence
  Selectively binds to CoO. Display platform: E. coli K-12.
RSLGKDRPHFHRS

SEQ ID NO: 768 - protein, 24 aa, Artificial Sequence
  Selectively binds to CoO and ZnO. Display platform: E. coli K-12.
RSRGLRNILMLRSYDSRSMRPHRS

SEQ ID NO: 769 - protein, 24 aa, Artificial Sequence
  Selectively binds to CoO. Display platform: E. coli K-12.
RSEPRRATQAPRSKPQKNEPAPRS

SEQ ID NO: 770 - protein, 33 aa, Artificial Sequence
  Selectively binds to CoO. Display platform: E. coli K-12.
RSLGAVSSLFRSQKIMQTDIVRSKGVRPAQRRS

SEQ ID NO: 771 - protein, 13 aa, Artificial Sequence
  Selectively binds to Cr2O3. Display platform: E. coli K-12.
RSVVRPKAATNRS

SEQ ID NO: 772 - protein, 13 aa, Artificial Sequence
  Selectively binds to Cr2O3. Display platform: E. coli K-12.
RSRIRHRLVGQRS

SEQ ID NO: 773 - protein, 24 aa, Artificial Sequence
  Selectively binds to Cr2O3. Display platform: E. coli K-12.
RSVKDGSATAKRSVANFETPRVRS

SEQ ID NO: 774 - protein, 24 aa, Artificial Sequence
  Selectively binds to Cr2O3. Display platform: E. coli K-12.
RSAPQTGRPNNRSLPLGNRDMQRS

SEQ ID NO: 775 - protein, 12 aa, Artificial Sequence
  Selectively binds to Fe2O3. Display platform: M13 bacteriophage.
QMDTSTSLAPSR

SEQ ID NO: 776 - protein, 12 aa, Artificial Sequence
  Selectively binds to Fe2O3. Display platform: M13 bacteriophage.
VPFTLQTRSLSD

SEQ ID NO: 777 - protein, 12 aa, Artificial Sequence
  Selectively binds to Fe2O3. Display platform: M13 bacteriophage.
TVTPSNISFTPS

SEQ ID NO: 778 - protein, 12 aa, Artificial Sequence
  Selectively binds to Fe2O3. Display platform: M13 bacteriophage.
ASTLINPLSISL

SEQ ID NO: 779 - protein, 12 aa, Artificial Sequence
  Selectively binds to Fe2O3. Display platform: M13 bacteriophage.
AGSTASVTPAKH

SEQ ID NO: 780 - protein, 12 aa, Artificial Sequence
  Selectively binds to Fe2O3. Display platform: M13 bacteriophage.
QMANSVMPLSWT

SEQ ID NO: 781 - protein, 11 aa, Artificial Sequence
  Selectively binds to Fe2O3. Display platform: M13 bacteriophage.
YAHSHDKYHPN

SEQ ID NO: 782 - protein, 12 aa, Artificial Sequence
  Selectively binds to Fe2O3. Display platform: M13 bacteriophage.
NQSPHSTYTLKP

SEQ ID NO: 783 - protein, 12 aa, Artificial Sequence
  Selectively binds to Fe2O3. Display platform: M13 bacteriophage.
HNYPQSYRPPIV

SEQ ID NO: 784 - protein, 12 aa, Artificial Sequence
  Selectively binds to Fe2O3. Display platform: M13 bacteriophage.
TDNNTTATVSPS

SEQ ID NO: 785 - protein, 12 aa, Artificial Sequence
  Selectively binds to Fe2O3. Display platform: M13 bacteriophage.
TMNNTTATVSPS

SEQ ID NO: 786 - protein, 12 aa, Artificial Sequence
  Selectively binds to Fe2O3. Display platform: M13 bacteriophage.
FQKQTNQSVSVS

SEQ ID NO: 787 - protein, 12 aa, Artificial Sequence
  Selectively binds to Fe2O3. Display platform: M13 bacteriophage.
VHMTPTNLTPNL

SEQ ID NO: 788 - protein, 11 aa, Artificial Sequence
  Selectively binds to Fe2O3. Display platform: M13 bacteriophage.
TFSYHNSNSPT

SEQ ID NO: 789 - protein, 12 aa, Artificial Sequence
  Selectively binds to Fe2O3. Display platform: M13 bacteriophage.
VPDHQVSYTLSR

SEQ ID NO: 790 - protein, 12 aa, Artificial Sequence
  Selectively binds to Fe2O3. Display platform: M13 bacteriophage.
IFHSHASLSPNS

SEQ ID NO: 791 - protein, 12 aa, Artificial Sequence
  Selectively binds to Fe2O3. Display platform: M13 bacteriophage.
ADNANVSTLHPT

SEQ ID NO: 792 - protein, 12 aa, Artificial Sequence
  Selectively binds to Fe2O3. Display platform: M13 bacteriophage.
VNQQPSSAFSPS

SEQ ID NO: 793 - protein, 12 aa, Artificial Sequence
  Selectively binds to Fe2O3. Display platform: M13 bacteriophage.
LSTVQTLSPSNH

SEQ ID NO: 794 - protein, 12 aa, Artificial Sequence
  Selectively binds to Fe2O3. Display platform: M13 bacteriophage.
DMNHTKSSYNPS

SEQ ID NO: 795 - protein, 9 aa, Artificial Sequence
  Selectively binds to hydroxyapatite. Display platform: solid phase synthesis on Wang resin.
CMLPHHGAC

SEQ ID NO: 796 - protein, 12 aa, Artificial Sequence
  Selectively binds to L1CoPt. Display platform: M13 phage.
KTHEIHSPLLHK

SEQ ID NO: 797 - protein, 16 aa, Artificial Sequence
  Selectively binds to lanthanide phosphate (LaPO4: Ce3+, Tb3+). Display platform: E. coli.
TSTQCPSHIRACLKKR

SEQ ID NO: 798 - protein, 16 aa, Artificial Sequence
  Selectively binds to lanthanide phosphate (LaPO4: Ce3+, Tb3+). Display platform: E. coli.
DQSTCGRKAQRCPAYP

SEQ ID NO: 799 - protein, 16 aa, Artificial Sequence
  Selectively binds to lanthanide phosphate (LaPO4: Ce3+, Tb3+). Display platform: E. coli.
HTYPCYQTTPTCVPTS

SEQ ID NO: 800 - protein, 16 aa, Artificial Sequence
  Selectively binds to lanthanide phosphate (LaPO4: Ce3+, Tb3+). Display platform: E. coli.
DHNQCKQSPRMCIPPL

SEQ ID NO: 801 - protein, 16 aa, Artificial Sequence
  Selectively binds to lanthanide phosphate (LaPO4: Ce3+, Tb3+). Display platform: E. coli.
LKFTCSTYGGLCKADT

SEQ ID NO: 802 - protein, 16 aa, Artificial Sequence
  Selectively binds to lanthanide phosphate (LaPO4: Ce3+, Tb3+). Display platform: E. coli.
RQLMCNHRPPNCTKCH

SEQ ID NO: 803 - protein, 13 aa, Artificial Sequence
  Selectively binds to MnO2. Display platform: E. coli K-12.
RSHHMLRRRNTRS

SEQ ID NO: 804 - protein, 13 aa, Artificial Sequence
  Selectively binds to MnO2. Display platform: E. coli K-12.
RSHINASQRVARS

SEQ ID NO: 805 - protein, 24 aa, Artificial Sequence
  Selectively binds to MnO2. Display platform: E. coli K-12.
RSCPRLGVWFYRSLSVGDGFVRRS

SEQ ID NO: 806 - protein, 35 aa, Artificial Sequence
  Selectively binds to MnO2. Display platform: E. coli K-12.
RSTSGPSRVMTRSIILRIGTLDRSCLKVFHMGWRS

SEQ ID NO: 807 - protein, 35 aa, Artificial Sequence
  Selectively binds to MnO2. Display platform: E. coli K-12.
RSITPILHDHRRSSVRPMVAHRRSPTLYFPAASRS

SEQ ID NO: 808 - protein, 7 aa, Artificial Sequence
  Selectively binds to nickel. Display platform: E. coli.
NTGSPYE

SEQ ID NO: 809 - protein, 7 aa, Artificial Sequence
  Selectively binds to nickel. Display platform: E. coli.
GSRSAQT

SEQ ID NO: 810 - protein, 7 aa, Artificial Sequence
  Selectively binds to nickel. Display platform: E. coli.
GTKGSLN

SEQ ID NO: 811 - protein, 7 aa, Artificial Sequence
  Selectively binds to nickel. Display platform: E. coli.
GYSSFNR

SEQ ID NO: 812 - protein, 7 aa, Artificial Sequence
  Selectively binds to nickel. Display platform: E. coli.
HHPVANT

SEQ ID NO: 813 - protein, 7 aa, Artificial Sequence
  Selectively binds to nickel. Display platform: E. coli.
HNETQKM

SEQ ID NO: 814 - protein, 7 aa, Artificial Sequence
  Selectively binds to nickel. Display platform: E. coli.
KDTSRSA

SEQ ID NO: 815 - protein, 7 aa, Artificial Sequence
  Selectively binds to nickel. Display platform: E. coli.
NAKHHPR

SEQ ID NO: 816 - protein, 7 aa, Artificial Sequence
  Selectively binds to nickel. Display platform: E. coli.
NGRAVNY

SEQ ID NO: 817 - protein, 7 aa, Artificial Sequence
  Selectively binds to nickel. Display platform: E. coli.
PGASVTY

SEQ ID NO: 818 - protein, 7 aa, Artificial Sequence
  Selectively binds to nickel. Display platform: E. coli.
RAEGTSE

SEQ ID NO: 819 - protein, 7 aa, Artificial Sequence
  Selectively binds to nickel. Display platform: E. coli.
RGATPMS

SEQ ID NO: 820 - protein, 7 aa, Artificial Sequence
  Selectively binds to nickel. Display platform: E. coli.
SLATDQK

SEQ ID NO: 821 - protein, 7 aa, Artificial Sequence
  Selectively binds to nickel. Display platform: E. coli.
SNNHSSM

SEQ ID NO: 822 - protein, 7 aa, Artificial Sequence
  Selectively binds to nickel. Display platform: E. coli.
STATPYK

SEQ ID NO: 823 - protein, 7 aa, Artificial Sequence
  Selectively binds to nickel. Display platform: E. coli.
TKTDVHF

SEQ ID NO: 824 - protein, 7 aa, Artificial Sequence
  Selectively binds to nickel. Display platform: E. coli.
TSVLNNT

SEQ ID NO: 825 - protein, 12 aa, Artificial Sequence
  Selectively binds to O. Display platform: M13 bacteriophage.
QWGWNMPLVEAQ

SEQ ID NO: 826 - protein, 12 aa, Artificial Sequence
  Selectively binds to O. Display platform: M13 bacteriophage.
HSHLHIHSGIQA

SEQ ID NO: 827 - protein, 12 aa, Artificial Sequence
  Selectively binds to O. Display platform: M13 bacteriophage.
NHVHRMHATPAY

SEQ ID NO: 828 - protein, 12 aa, Artificial Sequence
  Selectively binds to O. Display platform: M13 bacteriophage.
HYQHNTHHPSRW

SEQ ID NO: 829 - protein, 12 aa, Artificial Sequence
  Selectively binds to O. Display platform: M13 bacteriophage.
HSSPHFSRTWAS

SEQ ID NO: 830 - protein, 12 aa, Artificial Sequence
  Selectively binds to O. Display platform: M13 bacteriophage.
HHRTLSPSVSIL

SEQ ID NO: 831 - protein, 12 aa, Artificial Sequence
  Selectively binds to O. Display platform: M13 bacteriophage.
HSSPHFSRHGLL

SEQ ID NO: 832 - protein, 11 aa, Artificial Sequence
  Selectively binds to O. Display platform: M13 bacteriophage.
NTIHHRHHMPP

SEQ ID NO: 833 - protein, 12 aa, Artificial Sequence
  Selectively binds to O. Display platform: M13 bacteriophage.
SSGLRHSHHQHP

SEQ ID NO: 834 - protein, 12 aa, Artificial Sequence
  Selectively binds to O. Display platform: M13 bacteriophage.
GHIHSMRHHRPT

SEQ ID NO: 835 - protein, 13 aa, Artificial Sequence
  Selectively binds to PbO2. Display platform: E. coli K-12.
RSVQNDRIVAGRS

SEQ ID NO: 836 - protein, 13 aa, Artificial Sequence
  Selectively binds to PbO2. Display platform: E. coli K-12.
RSYPPFHNNDHRS

SEQ ID NO: 837 - protein, 24 aa, Artificial Sequence
  Selectively binds to PbO2 and ZnO. Display platform: E. coli K-12.
RSNTRMTARQHRSANHKSTQRARS

SEQ ID NO: 838 - protein, 24 aa, Artificial Sequence
  Selectively binds to PbO2. Display platform: E. coli K-12.
RSLAIDGTDVQRSKPLARSSGARS

SEQ ID NO: 839 - protein, 35 aa, Artificial Sequence
  Selectively binds to PbO2. Display platform: E. coli K-12.
RSPSPIRVPHHRSTAIPNRQLIRSQIRIHAMGHRS

SEQ ID NO: 840 - protein, 35 aa, Artificial Sequence
  Selectively binds to PbO2. Display platform: E. coli K-12.
RSRRVRDIHLGRSVQHRLGQPLRSLHQQSSPTLRS

SEQ ID NO: 841 - protein, 44 aa, Artificial Sequence
  Selectively binds to PbO2. Display platform: E. coli K-12.
RSRTPLAPVPVRSWHIGSRTIARSFNGITIGDRSYIPEHWYWRS

SEQ ID NO: 842 - protein, 12 aa, Artificial Sequence
  Selectively binds to SiO2. Display platform: phage.
HTKHSHTSPPPL

SEQ ID NO: 843 - protein, 12 aa, Artificial Sequence
  Selectively binds to SiO2. Display platform: phage.
HVSHFHASRHER

SEQ ID NO: 844 - protein, 12 aa, Artificial Sequence
  Selectively binds to SiO2. Display platform: phage.
HLASGHSIHYRT

SEQ ID NO: 845 - protein, 12 aa, Artificial Sequence
  Selectively binds to SiO2. Display platform: phage.
HQAHNHTHPSSL

SEQ ID NO: 846 - protein, 12 aa, Artificial Sequence
  Selectively binds to SiO2. Display platform: phage.
HGSKANHPHIRA

SEQ ID NO: 847 - protein, 12 aa, Artificial Sequence
  Selectively binds to SiO2. Display platform: phage.
HTPSNHRHTHNW

SEQ ID NO: 848 - protein, 12 aa, Artificial Sequence
  Selectively binds to SiO2. Display platform: phage.
HAPHTHMRSWSA

SEQ ID NO: 849 - protein, 12 aa, Artificial Sequence
  Selectively binds to SiO2. Display platform: phage.
HVSHHATGHTHT

SEQ ID NO: 850 - protein, 12 aa, Artificial Sequence
  Selectively binds to SiO2. Display platform: phage.
HKLPSASRHHFH

SEQ ID NO: 851 - protein, 12 aa, Artificial Sequence
  Selectively binds to SiO2. Display platform: phage.
HTTPSHLHPHSR

SEQ ID NO: 852 - protein, 12 aa, Artificial Sequence
  Selectively binds to SiO2. Display platform: phage.
DPSTHQHPPHKH

SEQ ID NO: 853 - protein, 12 aa, Artificial Sequence
  Selectively binds to SiO2. Display platform: phage.
SPQHTHHARIKN

SEQ ID NO: 854 - protein, 12 aa, Artificial Sequence
  Selectively binds to SiO2. DIsplay platform: phage.
HINHHHDTPSYR

SEQ ID NO: 855 - protein, 12 aa, Artificial Sequence
  Selectively binds to SiO2. Display platform: phage.
HPGVHSHPSPTP

SEQ ID NO: 856 - protein, 12 aa, Artificial Sequence
  Selectively binds to SiO2. Display platform: phage.
TVVQTYSMVTRA

SEQ ID NO: 857 - protein, 12 aa, Artificial Sequence
  Selectively binds to SiO2. Display platform: phage.
FSYRQSPPPPLY

SEQ ID NO: 858 - protein, 12 aa, Artificial Sequence
  Selectively binds to SiO2. Display platform: phage.
IMQNSISSPEML

SEQ ID NO: 859 - protein, 9 aa, Artificial Sequence
  Selectively binds to SiO2 and TiO2. Display platform: M13 phage.
CHKKPSKSC

SEQ ID NO: 860 - protein, 9 aa, Artificial Sequence
  Selectively binds to SiO2 and TiO2. Display platform: M13 phage.
CTKRNNKRC

SEQ ID NO: 861 - protein, 9 aa, Artificial Sequence
  Selectively binds to SiO2. Display platform: M13 phage.
CRRWESKRC

SEQ ID NO: 862 - protein, 35 aa, Artificial Sequence
  Selectively binds to synthetic sapphire. Display platform: Saccharomyces cerevisiae.
SGGGGSGGGGSGGGGSASQGGGGKMRAWGHPIWNW

SEQ ID NO: 863 - protein, 37 aa, Artificial Sequence
  Selectively binds to synthetic sapphire. Display platform: Saccharomyces cerevisiae.
SGGGGSGGGGSGGGGSASQGGGGSGTKHGKRSRCYNL

SEQ ID NO: 864 - protein, 37 aa, Artificial Sequence
  Selectively binds to synthetic sapphire. Display platform: Saccharomyces cerevisiae.
SGGGGSGGGGSGGGGSASQGGGGSGRTAKRKWKHTRD

SEQ ID NO: 865 - protein, 37 aa, Artificial Sequence
  Selectively binds to synthetic sapphire. Display platform: Saccharomyces cerevisiae.
SGGGGSGGGGSGGGGSASQGGGGSGKRHKQKTSRMGK

SEQ ID NO: 866 - protein, 37 aa, Artificial Sequence
  Selectively binds to synthetic sapphire. Display platform: Saccharomyces cerevisiae.
SGGGGSGGGGSGGGGSASQGGGGSGKRSKKCLRKNGS

SEQ ID NO: 867 - protein, 37 aa, Artificial Sequence
  Selectively binds to synthetic sapphire. Display platform: Saccharomyces cerevisiae.
  SITE (27)..(27): Xaa can be any naturally occurring amino acid
  SITE (29)..(29): Xaa can be any naturally occurring amino acid
  SITE (31)..(31): Xaa can be any naturally occurring amino acid
  SITE (33)..(33): Xaa can be any naturally occurring amino acid
  SITE (35)..(35): Xaa can be any naturally occurring amino acid
  SITE (37)..(37): Xaa can be any naturally occurring amino acid
SGGGGSGGGGSGGGGSASQGGGGSGGXGXGXGXGXGX

SEQ ID NO: 868 - protein, 37 aa, Artificial Sequence
  Selectively binds to synthetic sapphire. Display platform: Saccharomyces cerevisiae.
  SITE (28)..(29): Xaa can be any naturally occurring amino acid
  SITE (32)..(33): Xaa can be any naturally occurring amino acid
  SITE (36)..(37): Xaa can be any naturally occurring amino acid
SGGGGSGGGGSGGGGSASQGGGGSGGGXXGGXXGGXX

SEQ ID NO: 869 - protein, 37 aa, Artificial Sequence
  Selectively binds to synthetic sapphire. Display platform: Saccharomyces cerevisiae.
  SITE (29)..(31): Xaa can be any naturally occurring amino acid
  SITE (35)..(37): Xaa can be any naturally occurring amino acid
SGGGGSGGGGSGGGGSASQGGGGSGGGGXXXGGGXXX

SEQ ID NO: 870 - protein, 39 aa, Artificial Sequence
  Selectively binds to synthetic sapphire. Display platform: Saccharomyces cerevisiae.
SGGGGSGGGGSGGGGSASQGGGGSGCGKGKGKGKGKGKC

SEQ ID NO: 871 - protein, 37 aa, Artificial Sequence
  Selectively binds to synthetic sapphire. Display platform: Saccharomyces cerevisiae.
SGGGGSGGGGSGGGGSASQGGGGSGGKPKGKPKGKPK

SEQ ID NO: 872 - protein, 14 aa, Artificial Sequence
  Selectively binds to terbium doped cerium-magnesium aluminate. Display platform: E. coli.
KKQKCRTDACVTQM

SEQ ID NO: 873 - protein, 14 aa, Artificial Sequence
  Selectively binds to terbium doped cerium-magnesium aluminate. Display platform: E. coli.
NEKKCKGARCTIVT

SEQ ID NO: 874 - protein, 14 aa, Artificial Sequence
  Selectively binds to terbium doped cerium-magnesium aluminate. Display platform: E. coli.
ATPKCKKKSCMTTQ

SEQ ID NO: 875 - protein, 14 aa, Artificial Sequence
  Selectively binds to terbium doped cerium-magnesium aluminate. Display platform: E. coli.
VDKKCKSDDCGAWH

SEQ ID NO: 876 - protein, 14 aa, Artificial Sequence
  Selectively binds to terbium doped cerium-magnesium aluminate. Display platform: E. coli.
HDKKCKRQPCVLAN

SEQ ID NO: 877 - protein, 14 aa, Artificial Sequence
  Selectively binds to terbium doped cerium-magnesium aluminate. Display platform: E. coli.
FDKKCKSNKCLEVR

SEQ ID NO: 878 - protein, 14 aa, Artificial Sequence
  Selectively binds to terbium doped cerium-magnesium aluminate. Display platform: E. coli.
PKKKCHPEPCQTCG

SEQ ID NO: 879 - protein, 14 aa, Artificial Sequence
  Selectively binds to terbium doped cerium-magnesium aluminate. Display platform: E. coli.
KTEHCKKRKCPLDM

SEQ ID NO: 880 - protein, 14 aa, Artificial Sequence
  Selectively binds to terbium doped cerium-magnesium aluminate. Display platform: E. coli.
ETKKCTTGPCKVVT

SEQ ID NO: 881 - protein, 14 aa, Artificial Sequence
  Selectively binds to terbium doped cerium-magnesium aluminate. Display platform: E. coli.
KKKKCKKKICTTHT

SEQ ID NO: 882 - protein, 14 aa, Artificial Sequence
  Selectively binds to terbium doped cerium-magnesium aluminate. Display platform: E. coli.
KKKKCKKNTCKNHT

SEQ ID NO: 883 - protein, 10 aa, Artificial Sequence
  Selectively binds to TiO2. Display platform: M13 phage.
CDQQTNSEFC

SEQ ID NO: 884 - protein, 16 aa, Artificial Sequence
  Selectively binds to titanium. Display platform: bacteriophage.
SHKHPVTPRFFVVESK

SEQ ID NO: 885 - protein, 15 aa, Artificial Sequence
  Selectively binds to titanium. Display platform: bacteriophage.
SGGGVTPRFFVVESK

SEQ ID NO: 886 - protein, 14 aa, Artificial Sequence
  Selectively binds to titanium. Display platform: bacteriophage.
SHKHGGHKHGSSGK

SEQ ID NO: 887 - protein, 19 aa, Artificial Sequence
  Selectively binds to titanium. Display platform: bacteriophage.
SHKHGGHKHGGHKHGSSGK

SEQ ID NO: 888 - protein, 7 aa, Artificial Sequence
  Selectively binds to titanium. Display platform: phage.
ATWVSPY

SEQ ID NO: 889 - protein, 7 aa, Artificial Sequence
  Selectively binds to titanium. Display platform: phage.
AHSMGTG

SEQ ID NO: 890 - protein, 7 aa, Artificial Sequence
  Selectively binds to titanium. Display platform: phage.
FSSQMRY

SEQ ID NO: 891 - protein, 7 aa, Artificial Sequence
  Selectively binds to titanium. Display platform: phage.
GVGLPHT

SEQ ID NO: 892 - protein, 7 aa, Artificial Sequence
  Selectively binds to titanium. Display platform: phage.
QIEPLAL

SEQ ID NO: 893 - protein, 7 aa, Artificial Sequence
  Selectively binds to titanium. Display platform: phage.
RIVLPTY

SEQ ID NO: 894 - protein, 7 aa, Artificial Sequence
  Selectively binds to titanium. Display platform: phage.
VQQVALL

SEQ ID NO: 895 - protein, 7 aa, Artificial Sequence
  Selectively binds to titanium. Display platform: phage.
IVLPVPY

SEQ ID NO: 896 - protein, 7 aa, Artificial Sequence
  Selectively binds to titanium. Display platform: phage.
GHWTRLA

SEQ ID NO: 897 - protein, 7 aa, Artificial Sequence
  Selectively binds to titanium. Display platform: phage.
NLPLHST

SEQ ID NO: 898 - protein, 19 aa, Artificial Sequence
  Selectively binds to titanium. Display platform: M13 phage.
SCFWFLRWSLFIVLFTCCS

SEQ ID NO: 899 - protein, 19 aa, Artificial Sequence
  Selectively binds to titanium. Display platform: M13 phage.
SCESVDCFADSRMAKVSMS

SEQ ID NO: 900 - protein, 19 aa, Artificial Sequence
  Selectively binds to titanium. Display platform: M13 phage.
SCVGFFCITGSDVASVNSS

SEQ ID NO: 901 - protein, 19 aa, Artificial Sequence
  Selectivley binds to titanium. Display platform: M13 phage.
SCSDCLKSVDFIPSSLASS

SEQ ID NO: 902 - protein, 19 aa, Artificial Sequence
  Selectively binds to titanium. Display platform: M13 phage.
SCAFDCPSSVARSPGEWSS

SEQ ID NO: 903 - protein, 19 aa, Artificial Sequence
  Selectively binds to titanium. Display platform: M13 phage.
SCMLFSSVFDCGMLISDLS

SEQ ID NO: 904 - protein, 19 aa, Artificial Sequence
  Selectively binds to titanium. Display platform: M13 phage.
SCVDYVMHADSPGPDGLNS

SEQ ID NO: 905 - protein, 19 aa, Artificial Sequence
  Selectively binds to titanium. Display platform: M13 phage.
SCSENFMFNMYGTGVCTES

SEQ ID NO: 906 - protein, 19 aa, Artificial Sequence
  Selectively binds to titanium. Display platform: M13 phage.
SCSSFEVSEMFTCAVSSYS

SEQ ID NO: 907 - protein, 19 aa, Artificial Sequence
  Selectively binds to titanium. Display platform: M13 phage.
SCGLNFPLCSFVDFAQDAS

SEQ ID NO: 908 - protein, 12 aa, Artificial Sequence
  Selectively binds to Zn. Display platform: M13 bacteriophage.
ERSWTLDSALSM

SEQ ID NO: 909 - protein, 12 aa, Artificial Sequence
  Selectively binds to Zn. Display platform: M13 bacteriophage.
SNNDLSPLQTSH

SEQ ID NO: 910 - protein, 12 aa, Artificial Sequence
  Selectively binds to Zn. Display platform: M13 bacteriophage.
DSSNPIFWRPSS

SEQ ID NO: 911 - protein, 12 aa, Artificial Sequence
  Selectively binds to Zn. Display platform: M13 bacteriophage.
SILSTMSPHGAT

SEQ ID NO: 912 - protein, 12 aa, Artificial Sequence
  Selectively binds to Zn. Display platform: M13 bacteriophage.
SHALPLTWSTAA

SEQ ID NO: 913 - protein, 12 aa, Artificial Sequence
  Selectively binds to Zn. Display platform: M13 bacteriophage.
HVSIHRTTHHEM

SEQ ID NO: 914 - protein, 12 aa, Artificial Sequence
  Selectively binds to Zn. Display platform: M13 bacteriophage.
MKPDKAIRLDLL

SEQ ID NO: 915 - protein, 12 aa, Artificial Sequence
  Selectively binds to Zn. Display platform: M13 bacteriophage.
HYPTAKFHAERL

SEQ ID NO: 916 - protein, 12 aa, Artificial Sequence
  Selectively binds to Zn. Display platform: M13 bacteriophage.
FNTGSQMHQKFP

SEQ ID NO: 917 - protein, 12 aa, Artificial Sequence
  Selectively binds to Zn. Display platform: M13 bacteriophage.
HHTHRVDVHQTR

SEQ ID NO: 918 - protein, 12 aa, Artificial Sequence
  Selectively binds to Zn. Display platform: M13 bacteriophage.
FGLTAPRSASIL

SEQ ID NO: 919 - protein, 12 aa, Artificial Sequence
  Selectively binds to Zn. Display platform: M13 bacteriophage.
APRLPQSLLPQL

SEQ ID NO: 920 - protein, 12 aa, Artificial Sequence
  Selectively binds to ZnO. Display platform: M13 bacteriophage.
LLADTTHHRPWT

SEQ ID NO: 921 - protein, 12 aa, Artificial Sequence
  Selectively binds to ZnO. Display platform: M13 bacteriophage.
HSSHHQPKGTNP

SEQ ID NO: 922 - protein, 12 aa, Artificial Sequence
  Selectively binds to ZnO. Display platform: M13 bacteriophage.
HHGHSPTSPQVR

SEQ ID NO: 923 - protein, 12 aa, Artificial Sequence
  Selectively binds to ZnO. Display platform: M13 bacteriophage.
SHNHPPRHTAHS

SEQ ID NO: 924 - protein, 12 aa, Artificial Sequence
  Selectively binds to ZnO. Display platform: M13 bacteriophage.
HSKLNNRHHALL

SEQ ID NO: 925 - protein, 12 aa, Artificial Sequence
  Selectively binds to ZnO. Display platform: M13 bacteriophage.
HTKPHHTPTQRA

SEQ ID NO: 926 - protein, 24 aa, Artificial Sequence
  Selectively binds to ZnO. Display platform: E. coli K-12.
RSVFLPSILGWRSRLDDQGVAARS

SEQ ID NO: 927 - protein, 24 aa, Artificial Sequence
  Selectively binds to ZnO. Display platform: E. coli K-12.
RSTRNKHTTARRSVAPGIGEPSRS

SEQ ID NO: 928 - protein, 24 aa, Artificial Sequence
  Selectively binds to ZnO. Display platform: E. coli K-12.
RSIMHVRLRARRSARHMKDADPRS

SEQ ID NO: 929 - protein, 24 aa, Artificial Sequence
  Selectively binds to ZnO. Display platform: E. coli K-12.
RSPIIIRSRINRSHGRTKATPARS

SEQ ID NO: 930 - protein, 13 aa, Artificial Sequence
  Selectively binds to ZnO. Display platform: E. coli K-12.
RSTRRGTHNKDRS

SEQ ID NO: 931 - protein, 13 aa, Artificial Sequence
  Selectively binds to ZnO. Display platform: E. coli K-12.
RSTVPKRHPKDRS

SEQ ID NO: 932 - protein, 13 aa, Artificial Sequence
  Selectively binds to ZnO. Display platform: E. coli K-12.
PSIAKKTHNKWRS

SEQ ID NO: 933 - protein, 13 aa, Artificial Sequence
  Selectively binds to ZnO. Display platform: E. coli K-12.
RSYDSRSMRPHRS

SEQ ID NO: 934 - protein, 13 aa, Artificial Sequence
  Selectively binds to ZnO. Display platform: E. coli K-12.
RSTASRHTEPHRS

(19) United States
(12) Patent Application Publication
(10) Pub. No.: US 2022/0315935 A1
(43) Pub. Date: Oct. 6, 2022

(54) ENGINEERED YEAST FOR NONMAGNETIC FINES RECOVERY

(71) Applicant: Robert Crandall Greene, Vancouver (CA)

(72) Inventor: Robert Crandall Greene, Vancouver (CA)

(21) Appl. No.: 17/637,347

(22) PCT Filed: Aug. 16, 2020
(86) PCT No.: PCT/CA2020/051123

§ 371 Date: Feb. 22, 2022

Related U.S. Application Data
(60) Provisional application No. 62/890,597, filed on Aug. 22, 2019.

(51) Int. Cl. C12N 15/81; C22B 11/00; C07K 7/08; C07K 14/00

(52) U.S. Cl. CPC C12N 15/81; C22B 11/04; C07K 7/08; C07K 14/00

(57) ABSTRACT

The disclosure provides a magnetic reagent comprised of a recombinant yeast cell having the following genetic modifications: impairment of the CCC1 gene; addition of at least one copy of a human ferritin gene complex; addition of at least one copy of a TCO89 gene; and addition of at least one copy of a mineral- or metal ion-adsorbing target peptide, wherein the magnetic susceptibility or mass magnetization of said magnetic reagent is greater than it would be for a native yeast.

### BACKGROUND

### Field

The subject matter disclosed generally relates to harvesting microscopic mineral and mineral ore components from liquid using an engineered microorganism to instill magnetic qualities in nonmagnetic metals.

### Related Prior Art

Copper and gold are two of the most economically valuable mineral resources. While much un-mined copper and gold remain to meet increasing demands, reserve deposits have increased in mineralogical complexity.

For example, porphyry mineral deposits containing the largest reserves of copper have an average grade of 0.25%, and in addition usually contain arsenic in higher concentrations than the copper deposits mined before the 20th century. Low-grade deposits must be extensively milled to release ore from gangue (the valueless material from which copper has already been mined). Copper sulfide grains milled to less than 30 μm in diameter are hard to recover by flotation, and about 15% of this size fraction is lost to tailings, where it is oxidized and contributes to environmentally unfriendly acid rock drainage. Furthermore, copper concentrates containing arsenic at concentrations of greater than 2000 ppm cannot be smelted without releasing gaseous arsenic compounds, also an unwanted environmental consequence. Gold fines, for example, are typically recovered using cyanide leaching or mercury amalgamation.

In addition to these concerns, common flotation reagents cannot even distinguish between arsenic-bearing enargite (Cu₃AsS₄) and chalcopyrite (CuFeS₂).

To address all these issues, high gradient magnetic separation (HGMS) has been studied for its ability to pull copper mineral fines from slurries (Svoboda, J; Guest, R N et al, (1988)), however the low magnetic susceptibility of copper (and gold) have not surprisingly limited the industrial use of HGMS.

While a ferromagnetic metal like iron is strongly attracted to magnets; paramagnetic and diamagnetic metals like copper, carbon, gold, silver, lead and bismuth are either weakly magnetic or repel magnets.

Recently, metal binding peptides (MBP) have been discovered to selectively bind to certain minerals (THOTA; PERRY, 2017). These peptides can be used to avoid use of toxic mineral processing reagents, collect the ultra-fine mineral fraction usually lost during froth flotation, and selectively separate ore from gangue minerals.

Peptides that bind to chalcopyrite (a copper mineral) and not to silicate in gangue were attached to iron oxide nanoparticles (Greene, Robert Crandall, 2017 (GREENE, 2017) coated with aminopropylsilane via a polyethylene glycol cross-linker (HWANG; UNIVERSITY, 1989). The nanoparticles were then used to magnetize and concentrate chalcopyrite.

The metal binding peptides are expensive to produce in adequate quantities, so are not being used commercially.

Cell magnetization was first observed by attraction towards a magnet when normally diamagnetic yeast Saccharomyces cerevisiae were grown with ferric citrate. The magnetization was further enhanced by genetic modification of iron homeostasis and introduction of ferritin. (NISHIDA K, 2012)

A process is needed to economically extract high value minerals from low quality slurries, using mineral specific technologies that do not poison the environment, while able to take advantage of existing infrastructure such as HGMS.

### SUMMARY

According to an embodiment, there is provided a magnetic reagent comprised of a recombinant yeast cell having genetic modifications including

i) impairment of the yeast CCC1 gene; ii) addition of at least one copy of a human ferritin gene complex; iii) addition of at least one copy of a TCO89 gene; and iv) addition of at least one copy of a mineral- or metal ion-adsorbing target peptide, with the result that the magnetic susceptibility or mass magnetization of said magnetic reagent is increased.

In embodiments, the magnetic susceptibility or mass magnetization increase is to greater than 4.5-5.5 emu/g. In embodiments, the increase is enough to cause the recombinant yeast to be attracted to metal.

In embodiments, the mineral- or metal ion-adsorbing target peptide is operably associated with an α-agglutinin anchor domain. In embodiments, the human ferritin gene complex referred to above is expressed on a plasmid.

In other embodiments, the TCO89 gene is expressed on a plasmid.

In some embodiments, the expression of a mineral- or metal ion-adsorbing target peptide attached to the α-agglutinin anchor domain is mediated by a plasmid.

In still other embodiments, the plasmid is selected from the group consisting of pRS316, pRS423 and pRS425. In still other embodiments, the yeast cell is derived from Saccharomyces cerevisiae.

In embodiments, the yeast is Saccharomyces cerevisiae knockout strain BY4742. In other embodiments, the genetic modifications comprise an amino acid sequence at least 99% identical to any one of SEQ ID NOs: 14 to 947.

In some embodiments, the mineral- or metal ion-adsorbing target peptide includes an amino acid sequence at least 95% identical to any one of SEQ ID NOs: 14 to 947.

In other embodiments, the mineral- or metal ion-adsorbing target peptide includes an amino acid sequence identical to any one of SEQ ID NOs: 14 to 947. In some embodiments, the mineral- or metal ion-adsorbing target peptide comprises the amino acid sequence DSQKTNPS. In other embodiments, the mineral- or metal ion-adsorbing target peptide includes the amino acid sequence MHGKTQATSGTIQS.

According to another embodiment, there is provided a method of extracting metals from ore slurries using a magnetic reagent.

In some embodiments, the metal is copper. In others, the metal is gold. In still others, the metal is silver. In still others, the metal is an contaminant.

According to another embodiment, there is provided a recombinant yeast cell for use as a mining reagent.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 is a plasmid map corresponding to U1260DF290-17 bearing human ferritin gene complex FTh-FTL-PCBP1 on a PRS316 plasmid;

FIG. 2 is a plasmid map corresponding to U1260DF290-5 bearing KanMX4 gene and the ccc1 gene knocked out of a BY4742 plasmid;

FIG. 3 is a plasmid map corresponding to U1260DF290-4 bearing additional copy or copies of the TCO89 gene on a PRS423 plasmid;

FIG. 4 is a plasmid map corresponding to U1260DF290-12 bearing a metal binding peptide (peptide No. 1) on a PRS425 plasmid;

FIG. 5 is a plasmid map corresponding to U1260DF290-12 bearing a metal binding peptide (peptide No. 2) on a PRS425 plasmid;

FIG. 6 is three electron microscope images of modified Strain #0, #1 and #2 of S. cerevisiae respectively, according to the disclosure, taken at exposure=800 ms, gain=1, bin=1, gamma=1, no sharpening, normal contrast, HV=80.0 kV. Direct magnifications, from left to right, were: 80,000× for Strain #0, 60,000× for Strain #1, and 50,000× for Strain #2. Nanocrystals are indicated by arrows;

FIG. 7 is a series of photographs at 20× magnification in reflected, plane polarized light of (clockwise from top left): Control, Strain #0 on gold (18% coverage), Strain #1 on gold (0.85% coverage), and Strain #2 on gold (12.12% coverage) coated cover slips imaged immediately upon removal from phosphate buffered saline containing 0.1% Tween-20 (PBST) solution;

FIG. 8 shows Yeast+ expressing a gold-binding peptide bound to the surface of a gold-coated microscope slide at 5× magnification in reflected, plane polarized light. The Yeast+ surface coverage is 36.96%. The image was captured on a Zeiss optical microscope at 5× magnification in reflected, plane polarized light. Surface coverage was calculated with Image J;

FIG. 9 shows a single cell of Yeast+ expressing a gold-binding peptide [MHGKTQATSGTIQS (SEQ ID No. 27)×7 (BROWN, 1997)] bound to a quartz control slide. The image showing 0% surface coverage was captured on a Zeiss optical microscope at 5× magnification in transmitted, plane polarized light; and

FIG. 10. shows the intact Yeast+ biofilm from the top left quadrant of FIG. 10 after 3 cycles of dehydration and rehydration with PBST. While the cells have clustered together during the drying process, intact Yeast+ cells remain present at high concentration, 9.09% surface coverage. The image was captured at 300× magnification with a Bauch & Lombe scanning electron microscope in backscatter mode. Surface coverage was calculated with Image J.

It will be noted that throughout the appended drawings, like features are identified by like reference numerals.

### DETAILED DESCRIPTION

As discussed above, separating ore minerals from waste minerals in a finely crushed and ground rock sample is a process that works when there are differences in the minerals' magnetic susceptibilities and that of accompanying materials. Diamagnetic minerals are not susceptible to magnetic separation of ores, because they are not attracted to magnets even slightly.

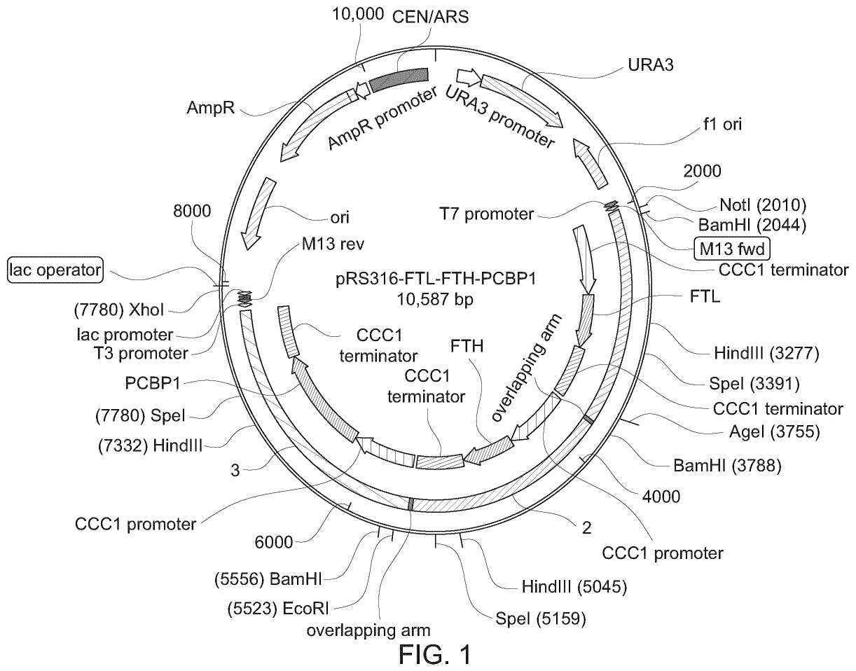

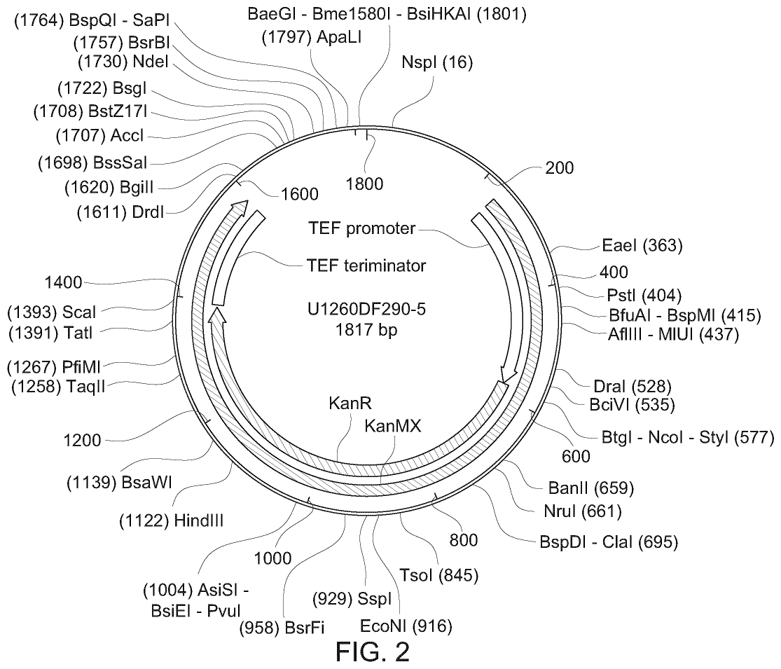

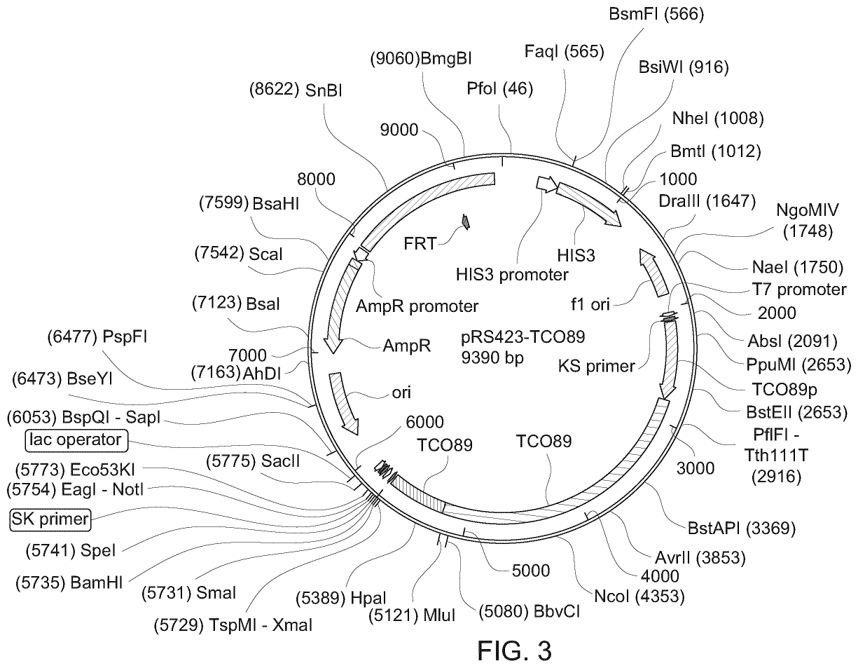

A standard yeast type such as S. cerevisiae with paramagnetic modifications is the starting material. In one modification, the native CCC1 gene, which is responsible for iron transport in and of the yeast's vacuole, is removed from the organism. The introduction into the yeast of a plasmid such as the one illustrated in FIG. 1 is used for this step. In a second modification, the human ferritin gene complex (FTH, FTL, PCBP1) is added to the yeast via a plasmid such as the one illustrated in FIG. 2, to enable the yeast to tolerate the uptake of high concentrations of iron citrate, which is transported into the cell and held in a gelatinous protein matrix by the human ferritin gene complex. Another plasmid such as the one illustrated in FIG. 3 is used to further modify the yeast with the addition of multiple copies of the yeast TCO89 gene to adjust the redox state within the yeast cell to the point that the iron ions held in the ferritin protein matrix will react with oxygen and crystallize into clusters of ferromagnetic iron oxide crystals.

The overall effect is to increase of the magnetic susceptibility of the yeast cells from the diamagnetic range into the paramagnetic range. At this stage, they can be concentrated in an HGMS unit.

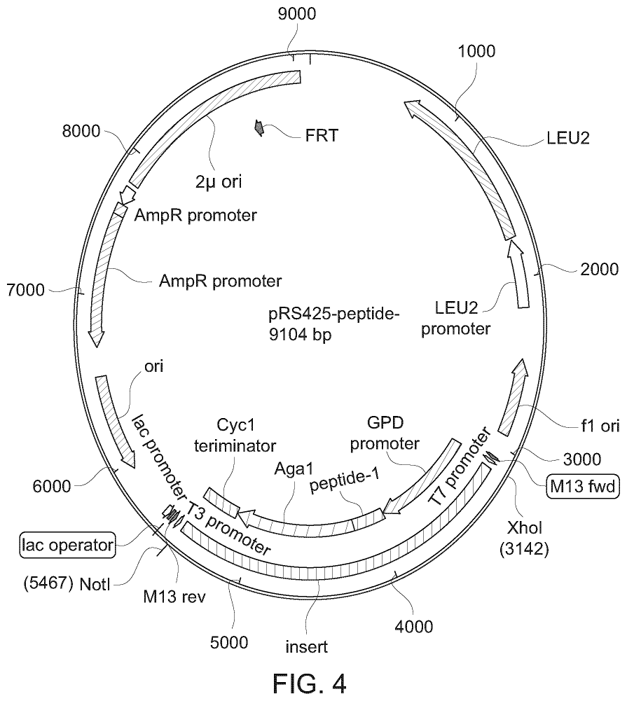

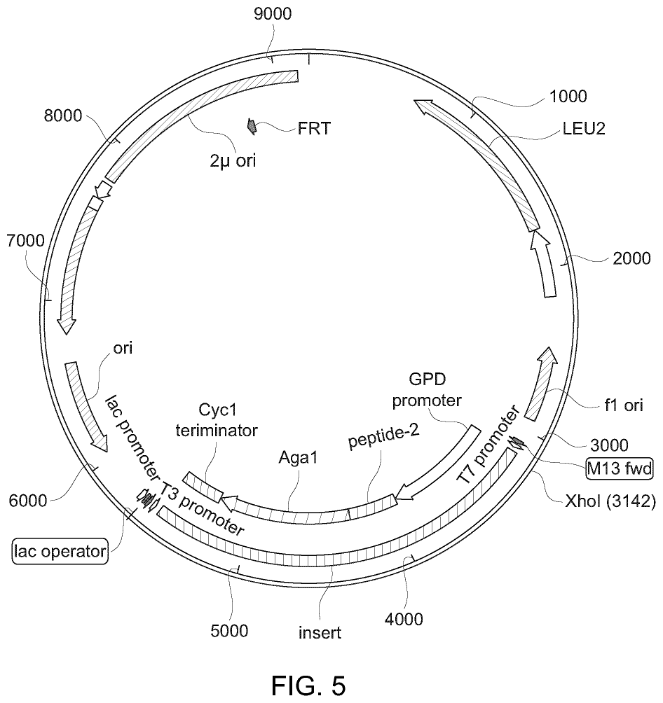

The attachment of a mineral-binding peptide, such as used in phage, to an alpha-agglutinin anchor protein, introduced by a plasmid such as the ones illustrated in FIG. 4 and FIG. 5, enables the paramagnetic yeast to selectively bind to whichever mineral phase the peptide targets, thereby increasing the magnetic susceptibility of that mineral phase to the point at which it could be concentrated in an HGMS unit.

Thus, the yeast according to embodiments of the disclosure will have both a magnetic nature as well as specificity for a mineral of interest. This combination is a cost effective and environmentally friendly regent for wet HGMS processes. An iron containing yeast expressing a specific metal binding protein such as those illustrated in Tables 2a-2d will bind to particular minerals, metals, metal ions, or mineral ore particles and will render them susceptible to magnetic separation.

The magnetic reagents according to embodiments of the disclosure may be applied to mineral processing and hydrometallurgical operations to harvest desired materials, and to environmental remediation application to remove toxic minerals and agents from liquid or dissolved media.

### Starting Materials

In embodiments of the disclosure, yeasts are used to express the desired peptides. Yeasts are eukaryotic, single-celled microorganisms classified as members of the fungus kingdom. Phage, the traditional source of metal binding peptides according to embodiments of the disclosure, simply cannot be grown in sufficient volumes to meet the demands of the mineral processing industry. Yeast can be readily magnetized and used in a mineral processing setting using existing equipment and techniques. Yeast are bigger, food safe, have a rigid cell wall, can tolerate high iron concentrations, are easy to grow in large reactors and are readily modified to display all sorts of peptide binders. Types of yeast that are useful in embodiments of the disclosure include Yeast of the species Saccharomyces spp, most preferably Saccharomyces cerevisiae. Other species of yeast such as Leucosporidium spp. or Candida spp. are alternative yeast hosts to use. Less common yeasts include Brettanomyces.

### Transformations

Transformations of S. cerevisiae were performed as described in Nishida et al. 2012 (NISHIDA K, 2012), in particular with respect to the TCO89 component and the ferritin component, unless otherwise described. The organisms were further transformed with selective peptides as described below.

As used herein, mineral-binding peptides or MBPs are peptides of less than 50 amino acids that preferentially bind to minerals. Examples include those identified by Curtis et al. (CURTIS; LEDERER; DUNBAR; MACGILLIVRAY, 2017) including an enargite-selective peptide with the sequence MHKPTVHIKGPT (SEQ ID No. 1) and a chalcopyrite-selective peptide with the sequence RKKKCKGNCCYTPQ (SEQ ID No. 2). Tables 2A to 2D list MBPs and their binding specificities which, in embodiments of the disclosure, are useful in the magnetic reagents formed by transformed yeast as herein described. Mineral-binding selectivity is generally confirmed by binding studies, zeta potential determination and immunochemistry.

### Definitions

Unless defined otherwise, all technical and scientific terms used herein have the same meaning as commonly understood to one of ordinary skill in the art recombinant yeast and ore separation. Although methods and materials similar or equivalent to those described herein can be used in the practice of the disclosed methods and compositions, exemplary methods, devices and materials are described herein.

The description of “a” or “an” item herein refers to a single item or multiple items.

High gradient magnetic separators (HGMS) are used to concentrate and recover very fine paramagnetic ore particles from froth flotation waste streams (see Svoboda, 1988), though widespread use for this specific application has been somewhat limited by the high operating costs arising from the energy needed to power the electromagnets.

It is understood that wherever embodiments are described herein with the language “comprising,” embodiments described in terms of “consisting of” and/or “consisting essentially of” are also provided. Thus, for example, reference to “a polynucleotide” includes a plurality of such polynucleotides and reference to “the microorganism” includes reference to one or more microorganisms.

A “plasmid” or “YAC” (yeast artificial chromosome) refers to an extrachromosomal element carrying genetic elements not part of the original chromosomes of the genome of the cell. Typically, the plasmid is a circular double-stranded DNA molecule. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with an appropriate 3′ untranslated sequence into a cell.

Preferably, the plasmids or vectors of the present disclosure are stable and self-replicating. In other embodiments, the plasmids are designed to replicate only a certain number of times before being lost during a replication. The plasmids used in embodiments of the disclosure are based on known templates with additions of the CCC1, TCO89, a ferritin complex, preferably human, and the metal-binding peptide in combination with an alpha-agglutinin anchor protein.

While the above-referenced genetic modifications are made by the addition of plasmid components to the yeast, alternate approaches such as synthesizing the components together on one plasmid, or complete or partial synthesis of the yeast genome with the desired components built in.

The term “integrated” as used herein refers to genetic elements that are placed into the genome of a host cell. For example, genetic elements can be placed into the chromosomes of the host cell rather than a vector such as a plasmid carried by the host cell. Methods for integrating genetic elements into the genome of a host cell are well known in the art and include homologous recombination.

The term “heterologous” refers to a polynucleotide, gene, polypeptide, or an enzyme not normally found in the host organism. The heterologous polynucleotide or gene can be introduced into the host organism by, e.g., gene transfer. A heterologous or exogenous polynucleotide, gene, polypeptide, or an enzyme can be derived from any source, e.g., eukaryotes, prokaryotes, viruses, or synthetic polynucleotide fragments.

The term “domain” refers to a part of a molecule or structure that shares common features, for example is hydrophobic, polar, globular, helical domains or properties, e.g., a DNA binding domain or an ATP binding domain.

A “nucleic acid,” “polynucleotide,” or “nucleic acid molecule” is a polymeric compound comprised of covalently linked subunits called nucleotides. Nucleic acid includes polyribonucleic acid (RNA) and polydeoxyribonucleic acid (DNA), both of which can be single-stranded or double-stranded. DNA includes cDNA, genomic DNA, synthetic DNA, and semi-synthetic DNA.

In double-stranded DNA molecules, sequences are described herein according to the normal convention of providing the sequence in the 5′ to 3′ direction along the non-transcribed strand of DNA (i.e., the strand having a sequence homologous to the mRNA).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, e.g., in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein (hereinafter “Maniatis”, entirely incorporated herein by reference).

Suitable nucleic acid sequences or fragments thereof (isolated polynucleotides of the present disclosure) encode peptides that are at least about 90 percent identical to the amino acid sequences reported herein, or at least about 90 percent, 91 percent, 92 percent, 93 percent, 94 percent, 95 percent, 96 percent, 97 percent, 98 percent, 99 percent, or 100 percent identical to the amino acid sequences reported herein.

A DNA or RNA “coding region” is a DNA or RNA molecule which is transcribed and/or translated into a polypeptide in a cell in vitro or in vivo when placed under the control of appropriate regulatory sequences. Regulatory regions include promoters, translation leader sequences, RNA processing site, effector binding site and stem-loop structure. The boundaries of the coding region are determined by a start codon at the 5′ (amino) terminus and a translation stop codon at the 3′ (carboxyl) terminus.

A polyadenylation signal and transcription termination sequence will usually be located 3′ to the coding region.

“Open reading frame” is abbreviated “ORF”, and means a length of nucleic acid, either DNA, cDNA or RNA, that comprises a translation start signal or initiation codon, such as an ATG or AUG, and a termination codon and can be potentially translated into a polypeptide sequence.

“Promoter” herein means a DNA fragment capable of controlling the expression of a coding sequence or functional RNA. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site, as well as protein binding domains (consensus sequences) for binding RNA polymerase.

A coding region is “under the control” of transcriptional and translational control elements in a cell when RNA polymerase transcribes the coding region into mRNA, which is then trans-RNA spliced (if the coding region contains introns) and translated into the protein encoded by the coding region.

The term “operably associated” refers to the association of nucleic acid sequences on a single nucleic acid fragment so that the function of one is affected by the other. For example, a promoter is operably associated with a coding region when it is capable of affecting the expression of that coding region (i.e., that the coding region is under the transcriptional control of the promoter).

The term “expression,” as used herein, refers to the transcription and stable accumulation of sense (mRNA) or antisense RNA derived from the nucleic acid fragment of the disclosure. Expression can also refer to translation of mRNA into a polypeptide.

The term magnetism refers to the degree of magnetism that that exists for a material described. The units of measurement shown for embodiments is emu/g. The term magnetism refers to the vector field that expresses the density of permanent or induced magnetic dipole moments in a magnetic material, also known as the (mass) magnetization. In the CGS (centimetre-gram-second) system of units, mass magnetization is given in emu/g, which is the magnetic moment divided by the mass of the material. In the SI (International System of Units) system of units, mass magnetization is given as Am²/kg.

One tesla (T) of magnetic field is equal to 104 Gauss, and one ampere (A) per meter is equal to 4π×10-3 oersted. The Gauss is the unit of magnetic flux density B and the equivalent of Mx/cm2, while the oersted is the unit of magnetizing field H. One tesla (T) is equal to 104 Gauss, and one ampere (A) per meter is equal to 4π×10-3 oersted.

### Operation

Mining is the field of extracting valuable metals or composites from the earth includes processes of breaking larger rocks into smaller pieces to free those valuable elements trapped inside. Starting with large rocks and large deposits, mining exploits smaller and smaller elements and attempts to extract as much value from them as possible.

High Gradient Magnetic Field Separation (HGMS) is one method used in mining to separate ore minerals from waste minerals in a finely crushed and ground rock sample is to take advantage of differences in the minerals' magnetic susceptibilities. Iron ore, for example, is strongly magnetic (1500-80000 emu/g) and is routinely separated from other minerals in the sample with a relatively low gradient magnetic field (1-3 kGauss). With the application of a high gradient magnetic field (2-20 kGauss), paramagnetic minerals (0.21-290 emu/g) can also be separated from other minerals, and from each other with great precision (METSEO, 2012).

In addition to highly controllable separations of minerals based on differences in magnetic susceptibility, “wet carousel” high gradient magnetic separators (HGMS) can also successfully concentrate paramagnetic particles from 1 mm to as small as 0.1 μm.

In the field, during wet HGMS, the contents of a holding tank containing minerals and ores as well as the magnetic reagent according to embodiments of the disclosure will be continuously passed through a wet carousel HGMS unit, where the ˜20% of very fine mineral (such as chalcopyrite) grains missed by the froth flotation process will be captured by the magnetic filaments within the unit, and separated from the nonmagnetic contents of the slurry which flow to a tailings pond or waste receptacle.

For gold extraction, the magnetic reagent according to embodiments of the disclosure (“MBP yeast”) will be added to the gold processing circuit after the gravity separation unit operation in a typical gold processing circuit. Any fines missed by the gravity concentrator will be selectively coated with MBP yeast in a stirred tank, magnetized, and separated in a wet carousel HGMS unit.

The bio-magnetic mineral separation system would operate continuously using existing industrial magnetic mineral processing equipment. No toxic reagents are necessary and, in the case of gold, the need for several toxic reagents is eliminated. Also, by removing mineral fines and metal ions from tailings streams, future acid mine drainage or environmental contamination is prevented. The yeast according to embodiments of the disclosure are biodegradable, and the process takes place in regular water at normal pH. Once the ore minerals have been recovered by the magnetic separator, the yeast will be ashed when the ore concentrates are smelted.

The yeast can be cultured off or on site using standard industrial bioreactors and they still function as mineral processing reagents even when dead, so they can be used in harsh conditions if necessary.

### Targets

Copper minerals exist in a variety of chemical forms. Table 1 lists the most common ones.

TABLE 1
Copper Minerals and Ions

| | | % of mineral |
|---|---|---|
| Mineral name | Chemical Formula | that is Copper |
| Chalcopyrite | CuFeS₂ | 34.5 |
| Chalcocite | Cu₂S | 79.8 |
| Covellite | CuS | 66.5 |
| Bornite | Cu₅FeS₄ | 63.3 |
| Tetrahedrite | Cu₃SbS₃ + x(Fe, Zn)₆Sb₂S₉ | 32-45 |
| Digenite | Cu₉S₅ | 78.1 |
| Malachite | CuCO₃•Cu(OH)₂ | 57.7 |
| Azurite | Cu₃(CO₃)₂(OH)₂ | 55.1 |
| Cuprite | Cu₂O | 88.8 |
| Chrysocolla | CuO•SiO₂•2H₂O | 37.9 |
| Tennantite | Cu₁₂AS₄S₁₃ | 51.6 |
| Other | Cu, CuO, Cu²⁺, Cu₂O | various |
| Enargite | Cu₃AsS₄ | 52 |

Another metal that is susceptible to extraction according to embodiments of the disclosure is Zinc. Zinc minerals include ZnS (sphalerite), a form of zinc sulfide, smithsonite (zinc carbonate), hemimorphite (zinc silicate), wurtzite (another zinc sulfide), and hydrozincite (basic zinc carbonate). Gold is a desirable, valuable metal, widespread but usually in tiny quantities. It exists as a stable anion, “Au− is considered paramagnetic. Au3+ is another gold ion. Gold minerals are rare but include Au(CN)₂—, AuCl, gold(III) chloride, Au₂Cl₆, gold(III) fluoride, AuCl₃, gold(III) bromide, monoiodide, gold(III) sulfide, gold(III) chloride, mercury alloys, AuCl₄—, CsAu, gold(II) sulfate, Au2(SO4)2, gold(II) complex, [AuXe₄], (Sb2F11)₂, gold pentafluoride, along with its derivative anion, AuF⁻⁶, and its difluorine complex, gold heptafluoride. Gold nanoparticles are also susceptible to binding and extraction using reagents and methods of the disclosure. Gold specific binding peptides are listed in Table 2A, along with Platinum and Palladium specific binding peptides.

TABLE 2A
Gold, Platinum, Palladium Binding Peptides

| Table 2A. Target Au, | Literature | | Peptide | |
|---|---|---|---|---|
| Pt, Pd Metal or mineral | ref. | SEQ ID NO. | Sequence to use | Display Platform |
| Au Ions (Au³⁺), Au, | A3, PEPA., | 14 | AYSSGAPPMPPF | M13 phage, |
| Pd, Palladium & | AG3 | | | streptavidin |
| Platinum Ions (Pd4+, | | | | coated glass slide, |
| Pt²⁺), Ag | | | | SPOT synthesized |
| | | | | peptide array, |
| | | | | chemically |
| | | | | synthesized |
| | | | | without display |
| | | | | platform |
| Au Ions (Au³⁺) | A3-S | 15 | ASSSGAPPMPPF | Streptavidin |
| | | | | coated glass slide |
| Au Ions (Au³⁺), Au, Pd | Flg | 16 | DYKDDDDK | Streptavidin |
| | | | | coated glass slide, |
| | | | | simulation |
| Au Ions (Au³⁺), | Flg-A3 | 17 | DYKDDDDKPAYSSG | Chemically |
| Palladium & | | | APPMPPF | synthesized |
| Platinum Ions (Pd⁴⁺, | | | | without display |
| Pt²⁺), Au | | | | platform, |
| nanoparticles | | | | streptavidin |
| | | | | coated glass slide |
| Au Ions (Au³⁺), | A3-Flg | 18 | AYSSGAPPMPPFPDY | Streptavidin |
| Palladium & | | | KDDDK | coated glass slide, |
| Platinum Ions (Pd⁴⁺, | | | | chemically |
| Pt²⁺) | | | | synthesized |
| | | | | without display |
| | | | | platform |
| Au powder | pSB3004 | 19 | ALVPTAHRIDGNMH | E. coli |
| Au powder | pSB3071 | 20 | ALPRGVYKIDSNMH | E. coli |
| Au powder | pSB3006 | 21 | PGMKASKSMRNQAT | E. coli |
| | | | PGMPSSLDLTWQAT | |
| Au powder | pSB3081 | 22 | PGMKMRLSGAKEAT | E. coli |
| | | | PGMSTTVAGLLQAT | |
| Au powder | pSB3073 | 23 | PGMIHVQKTAVQAT | E. coli |
| | | | PGMVNLTSPVKQAT | |
| Au powder | pSB3008 | 24 | AIDSPAGCISFSMH | E. coli |
| | | | MHGKTQATSGTIQS | |
| Au powder | pSB3127 | 25 | MHGKTQATSGTIQS¹ | E. coli, E. coli |
| | | | | fermentation, |
| | | | | SPOT synthesized |
| | | | | peptide array, |
| | | | | M13K07 helper |
| | | | | phage, Fmoc |
| | | | | synthesis, Au |
| | | | | coated Si AFM |
| | | | | probe, chemical |
| | | | | synthesis without |
| | | | | display platform |
| Au | RP1 | 26 | (SKTSLGCQKPLYMG | E. coli |
| | | | REMRMLT)₂₊(SKTSL | |
| | | | QQSGASLQGSEKLTN | |
| | | | G)₅ | |
| Au | RP2 | 27 | (QATSEKLVRGMEGA | E. coli |
| | | | SLHPAKT)₇ | |
| Au | RP4 | 28 | (SKTSTNNFGGMMPG | E. coli |
| | | | GDESTKI)₁₇ | |
| Au | RPS | 29 | (QATSEMQRQMGIRV | E. coli |
| | | | GPEQDKT)₁₁ | |
| Au | RP6 | 30 | (QATSGSERMGHQSG | E. coli |
| | | | TVHPGKT)₇ | |
| Au | Dan1, B1, | 31 | LKAHLPPSRLPS | Phage display, |
| | Au binding | | | M13 virus |
| | motif | | | |
| Au | Yu1 | 32 | KPHTPHNHPSHH | Phage display, |
| Au | d7-A02 | 33 | PGLVKPSQTLSLTCA | Saccharomyces |
| | | | ISGDSVSSNSAGWTW | cerevisiae |
| | | | IRQSPSRGLEWLGRT | |
| | | | YYKSKWYYDMQYL | |
| Au | d7-A12 | 34 | PGLVKPSQTLSLTCA | Saccharomyces |
| | | | ISGDSVSSNRAAWNW | cerevisiae |
| | | | IRQSPSRGLEWLGRT | |
| | | | YHRSKWGYDMRYL | |
| Au | d7-A01 | 35 | PGLVKPSQTLSLTCA | Saccharomyces |
| | | | ISGDSVSGNTAAWNW | cerevisiae |
| | | | IRQSPSRGLEWLGRT | |
| | | | YYRSKWHYDMRHL | |
| Au > ZnS > CdS, Au- | Z1 | 36 | KHKHWHW | Saccharomyces |
| coated slides | | | | cerevisiae, SPOT |
| (Evaporated Metals | | | | synthesized |
| Films Corp.), AuPt | | | | peptide array |
| alloy nanoparticles | | | | |
| Au | Z2 | 37 | RMRMKMK | Saccharomyces |
| | | | | cerevisiae, SPOT |
| | | | | synthesized |
| | | | | peptide array |
| AU 99.9% pure gold | 1-AuBP1, | 38 | WAGAKRLVLRRE | E. coli |
| foil, Au, Au & Ag | AuBP1 | | | |
| QCM sensor surfaces | | | | |
| Au | c-AuBP1 | 39 | CGPWAGAKRLVLRRE | E. coli |
| | | | GPC | |
| Au, Au nanoparticles, | 1-AuBP2, | 40 | WALRRSIRRQSY | E. coli, SPOT |
| Au & Ag QCM | AuBP2 | | | synthesized |
| sensor surfaces | | | | peptide array, |
| | | | | chemical |
| | | | | synthesis without |
| | | | | display platform |
| Au | c-AuBP2 | 41 | CGPWALRRSIRRQSY | E. coli |
| | | | GPC | |
| AU metallic gold | Au | 42 | SKTSLGQSGQSLQGS | M13 peptide |
| powder (Aldrich) | | | EKLTNG | library (7 amino |
| | | | | acids long) and |
| | | | | selected using CSD |
| Au, chloroauric acid | Au | 43 | QATSEKLVRGMEGAS | M13 peptide |
| | | | LHPAKT | library (7 amino |
| | | | | acids long) and |
| | | | | selected using |
| | | | | CSD |
| AU 99.9% pure gold | AuBP1 | 44 | WAGAKRLVLRRGE | SPOT synthesized |
| foil, Au nanoparticles | | | | peptide array, |
| (Sigma Canada) | | | | chemical |
| | | | | synthesis without |
| | | | | display platform |
| AU metallic gold, | Au2 | 45 | LGQSGQSLQGSEKLT | SPOT synthesized |
| AuCl₃ | | | NG | peptide array |
| AUCl₃ | Au3 | 46 | EKLVRGMEGASLHPA | SPOT synthesized |
| | | | | peptide array |
| Au nanoparticles | 1 | 47 | HFSSWETQQG | SPOT synthesized |
| | | | | peptide array |
| Au nanoparticles | 2 | 48 | WTHRDASTPW | SPOT synthesized |
| | | | | peptide array |
| Au nanoparticles | 3 | 49 | WYEKWQKANW | SPOT synthesized |
| | | | | peptide array |
| Au nanoparticles | 4 | 50 | WMETKWQARA | SPOT synthesized |
| | | | | peptide array |
| Au nanoparticles | 5 | 51 | GTWSEHQNGW | SPOT synthesized |
| | | | | peptide array |
| Au nanoparticles | 6 | 52 | ETWSMQQHEW | SPOT synthesized |
| | | | | peptide array |
| Au nanoparticles | 7 | 53 | WRAGQAQMQW | SPOT synthesized |
| | | | | peptide array |
| Au nanoparticles | 8 | 54 | WKPWMEPQHS | SPOT synthesized |
| | | | | peptide array |
| Au nanoparticles | 9 | 55 | AMQQQWEMSQ | SPOT synthesized |
| | | | | peptide array |
| Au nanoparticles | 10 | 56 | RWQIEEHFAP | SPOT synthesized |
| | | | | peptide array |
| Au nanoparticles | 11 | 57 | PEESQEGWMA | SPOT synthesized |
| | | | | peptide array |
| Au nanoparticles | 12 | 58 | TGEWGMQGIH | SPOT synthesized |
| | | | | peptide array |
| Au nanoparticles | 13 | 59 | EEPHWEEMAA | SPOT synthesized |
| | | | | peptide array |
| Au nanoparticles | 14 | 60 | WWKVANIHSK | SPOT synthesized |
| | | | | peptide array |
| Au nanoparticles | 15 | 61 | RHWHSWTWEI | SPOT synthesized |
| | | | | peptide array |
| Au nanoparticles | 16 | 62 | MNWKWGLESM | SPOT synthesized |
| | | | | peptide array |
| Au nanoparticles | 17 | 63 | NWTAKWTQTH | SPOT synthesized |
| | | | | peptide array |
| Au nanoparticles | 18 | 64 | HWIKIPPWMW | SPOT synthesized |
| | | | | peptide array |
| Au nanoparticles | 19 | 65 | HWKQKVHWWG | SPOT synthesized |
| | | | | peptide array |
| Au nanoparticles | 20 | 66 | WHKWWTHGHW | SPOT synthesized |
| | | | | peptide array |
| Au nanoparticles | 21 | 67 | KYWQMWMSWK | SPOT synthesized |
| | | | | peptide array |
| Au nanoparticles | 22 | 68 | KWQWKQAGAQ | SPOT synthesized |
| | | | | peptide array |
| Au nanoparticles | 23 | 69 | EHQQWKETWH | SPOT synthesized |
| | | | | peptide array |
| Au nanoparticles | 24 | 70 | GQWQWMDAGW | SPOT synthesized |
| | | | | peptide array |
| Au nanoparticles | 25 | 71 | QWTWKIQVMK | SPOT synthesized |
| | | | | peptide array |
| Au nanoparticles | 26 | 72 | HWKGEMHTDF | SPOT synthesized |
| | | | | peptide array |
| Au nanoparticles | 27 | 73 | PEEGPHSLWH | SPOT synthesized |
| | | | | peptide array |
| Au nanoparticles | 28 | 74 | EWVEAMGGHT | SPOT synthesized |
| | | | | peptide array |
| Au nanoparticles | 29 | 75 | WPAMGWNMEQ | SPOT synthesized |
| | | | | peptide array |
| Au nanoparticles | 30 | 76 | KWAIWEMKGH | SPOT synthesized |
| | | | | peptide array |
| Au nanoparticles | 31 | 77 | GETWETHYSE | SPOT synthesized |
| | | | | peptide array |
| Au nanoparticles | 32 | 78 | WVHKRLNWTV | SPOT synthesized |
| | | | | peptide array |
| Au nanoparticles | 33 | 79 | WSWPKVKSFW | SPOT synthesized |
| | | | | peptide array |
| Au nanoparticles | 34 | 80 | MLGWMHQSWQ | SPOT synthesized |
| | | | | peptide array |
| Au nanoparticles | 35 | 81 | NWKWQMKWTQ | SPOT synthesized |
| | | | | peptide array |
| Au nanoparticles | 36 | 82 | EWHVKWSEAI | SPOT synthesized |
| | | | | peptide array |
| Au nanoparticles | 37 | 83 | LAGVPMHWYT | SPOT synthesized |
| | | | | peptide array |
| Au nanoparticles | 38 | 84 | QEHLSEMWGE | SPOT synthesized |
| | | | | peptide array |
| Au nanoparticles | 39 | 85 | ASHQWAWKWE | SPOT synthesized |
| | | | | peptide array |
| Au nanoparticles | 40 | 86 | WSEETEMWPL | SPOT synthesized |
| | | | | peptide array |
| Au nanoparticles | 41 | 87 | DMVWHESWGI | SPOT synthesized |
| | | | | peptide array |
| Au nanoparticles | 42 | 88 | WWLQKWHGSH | SPOT synthesized |
| | | | | peptide array |
| Au nanoparticles | 43 | 89 | ENHSWGGGGA | SPOT synthesized |
| | | | | peptide array |
| Au nanoparticles | 44 | 90 | QRHSWGGGEA | SPOT synthesized |
| | | | | peptide array |
| Au nanoparticles | 45 | 91 | WKATWAKYEK | SPOT synthesized |
| | | | | peptide array |
| Au nanoparticles | 46 | 92 | TWHIMWRHAW | SPOT synthesized |
| | | | | peptide array |
| Au nanoparticles | 47 | 93 | WEAKEWLHNW | SPOT synthesized |
| | | | | peptide array |
| Au nanoparticles | 48 | 94 | NKGGGWQGPE | SPOT synthesized |
| | | | | peptide array |
| Au nanoparticles | 49 | 95 | WGWKWEHSEA | SPOT synthesized |
| | | | | peptide array |
| Au, Pd | Pd2 | 96 | NFMSLPRLGHMH | M13 phage, |
| | | | | simulation |
| Au, Pd, Pd films | Pd4 | 97 | TSNAVHPTLRHL | Simulation, E. |
| deposited on silicon | | | | coli, M13 phage |
| Au, Pd | Gly₁₀ | 98 | GGGGGGGGGG | Simulation |
| Au, Pd | Pro₁₀ | 99 | PPPPPPPPPP | Simulation |
| Pt | Pt | 100 | DRTSTWR | M13 PD library |
| | Pt | 101 | QSVTSTK | M13 PD library |
| | Pt | 102 | SSSHLNK | M13 PD library |
| Pd | Pd | 103 | SVTQNKY | M13 PD library |
| | Pd | 104 | SPHPGPY | M13 PD library |
| | Pd | 105 | HAPTPML | M13 PD library |
| Au⁰, Ag⁰, Ni, Au | HRE | 106 | AHHAHHAAD | Chemical |
| nanoparticles, ZnS, | | | | synthesis without |
| TiO₂, Ag₂S | | | | display platform |
| AU nanoparticles | | Null 2 | CA | Chemical |
| | | | | synthesis without |
| | | | | display platform |
| AU nanoparticles | | Null 3 | CAL | Chemical |
| | | | | synthesis without |
| | | | | display platform |
| AU nanoparticles | | 107 | CALN | Chemical |
| | | | | synthesis without |
| | | | | display platform |
| AU nanoparticles, Au | | 108 | CALNN | Chemical |
| nanoparticles | | | | synthesis without |
| | | | | display platform |
| AU nanoparticles | | 109 | CCALNN | Chemical |
| | | | | synthesis without |
| | | | | display platform |
| Au nanoparticles | | 110 | KALNN | Chemical |
| | | | | synthesis without |
| | | | | display platform |
| Au nanoparticles | | 111 | AALNN | Chemical |
| | | | | synthesis without |
| | | | | display platform |
| Au nanoparticles | | 112 | NNLAC | Chemical |
| | | | | synthesis without |
| | | | | display platform |
| Au nanoparticles | | 113 | CILNN | Chemical |
| | | | | synthesis without |
| | | | | display platform |
| Au nanoparticles | | 114 | CLLNN | Chemical |
| | | | | synthesis without |
| | | | | display platform |
| Au nanoparticles | | 115 | CVLNN | Chemical |
| | | | | synthesis without |
| | | | | display platform |
| Au nanoparticles | | 116 | CFLNN | Chemical |
| | | | | synthesis without |
| | | | | display platform |
| Au nanoparticles | | 117 | CAANN | Chemical |
| | | | | synthesis without |
| | | | | display platform |
| Au nanoparticles | | 118 | CAINN | Chemical |
| | | | | synthesis without |
| | | | | display platform |
| Au nanoparticles | | 119 | CAVNN | Chemical |
| | | | | synthesis without |
| | | | | display platform |
| Au nanoparticles | | 120 | CLANN | Chemical |
| | | | | synthesis without |
| | | | | display platform |
| Au nanoparticles | | 121 | CKLNN | Chemical |
| | | | | synthesis without |
| | | | | display platform |
| Au nanoparticles | | 122 | CAFNN | Chemical |
| | | | | synthesis without |
| | | | | display platform |
| Au nanoparticles | | 123 | CDLNN | Chemical |
| | | | | synthesis without |
| | | | | display platform |
| Au nanoparticles | | 124 | CTLNN | Chemical |
| | | | | synthesis without |
| | | | | display platform |
| Au nanoparticles | | 125 | CNNN | Chemical |
| | | | | synthesis without |
| | | | | display platform |
| Au nanoparticles | | 126 | CAKNN | Chemical |
| | | | | synthesis without |
| | | | | display platform |
| Au nanoparticles | | 127 | CADNN | Chemical |
| | | | | synthesis without |
| | | | | display platform |
| Au nanoparticles | | 128 | CATNN | Chemical |
| | | | | synthesis without |
| | | | | display platform |
| Au nanoparticles | | 129 | CANNN | Chemical |
| | | | | synthesis without |
| | | | | display platform |
| Au nanoparticles | | 130 | CDDNN | Chemical |
| | | | | synthesis without |
| | | | | display platform |
| Au nanoparticles | | 131 | CKKNN | Chemical |
| | | | | synthesis without |
| | | | | display platform |
| Au nanoparticles | | 132 | CTTNN | Chemical |
| | | | | synthesis without |
| | | | | display platform |
| Au nanoparticles | | 133 | CTSNN | Chemical |
| | | | | synthesis without |
| | | | | display platform |
| Au nanoparticles | | 134 | CALLS | Chemical |
| | | | | synthesis without |
| | | | | display platform |
| Au nanoparticles | | 135 | CALLD | Chemical |
| | | | | synthesis without |
| | | | | display platform |
| Au nanoparticles | | 136 | CALLK | Chemical |
| | | | | synthesis without |
| | | | | display platform |
| Au nanoparticles | | 137 | CALLR | Chemical |
| | | | | synthesis without |
| | | | | display platform |
| Au nanoparticles | | 138 | CALNS | Chemical |
| | | | | synthesis without |
| | | | | display platform |
| Au nanoparticles | | 139 | CALND | Chemical |
| | | | | synthesis without |
| | | | | display platform |
| Au nanoparticles | | 140 | CALNK | Chemical |
| | | | | synthesis without |
| | | | | display platform |
| Au nanoparticles | | 141 | CALNR | Chemical |
| | | | | synthesis without |
| | | | | display platform |
| Au nanoparticles | | 142 | NCALSS | Chemical |
| | | | | synthesis without |
| | | | | displayplatform |
| Au nanoparticles | — | 143 | CALSR | Chemical |
| | | | | synthesis without |
| | | | | display platform |
| Au nanoparticles | | 144 | CALKS | Chemical |
| | | | | synthesis without |
| | | | | display platform |
| Au nanoparticles | | 145 | CTTTT | Chemical |
| | | | | synthesis without |
| | | | | display platform |
| Au nanoparticles | | 146 | CHRIS | Chemical |
| | | | | synthesis without |
| | | | | display platform |
| Au nanoparticles | | 147 | CVVIT | Chemical |
| | | | | synthesis without |
| | | | | display platform |
| Au nanoparticles | | 148 | CAALPDGLAAC | Chemical |
| | | | | synthesis without |
| | | | | display platform |
| Au nanoparticles | | 149 | CALSD | Chemical |
| | | | | synthesis without |
| | | | | display platform |
| Au nanoparticles | | 150 | CALKK | Chemical |
| | | | | synthesis without |
| | | | | display platform |
| Au nanoparticles | | 151 | CVVITPDGTIVVC | Chemical |
| | | | | synthesis without |
| | | | | display platform |
| Au nanoparticles | | 152 | CALKD | Chemical |
| | | | | synthesis without |
| | | | | display platform |
| Au nanoparticles | | 153 | CALSK | Chemical |
| | | | | synthesis without |
| | | | | display platform |
| Au nanoparticles | | 154 | CALSS | Chemical |
| | | | | synthesis without |
| | | | | display platform |
| Au nanoparticles | | 155 | NNLACALNN | Chemical |
| | | | | synthesis without |
| | | | | display platform |
| Au nanoparticles | | 156 | NNLACCALNN | Chemical |
| | | | | synthesis without |
| | | | | display platform |
| Au nanoparticles | | 157 | CCVVVK | Chemical |
| | | | | synthesis without |
| | | | | display platform |
| Au nanoparticles | | 158 | CCVVVT | Chemical |
| | | | | synthesis without |
| | | | | display platform |
| Au nanoparticles | | 159 | CALNNGGWSHPQFE | Chemical |
| | | | K | synthesis without |
| | | | | display platform |
| AU, Au nanoparticles | P8#9, #9s1 | 160 | VSGSSPDS | Fmoc synthesis |
| | | | | (Genescript) |
| Pt, Pt 2 nm of Pt | PtBP1 | 161 | PTSTGQA | M13 phage, Fmoc |
| coated directly onto | | | | synthesis |
| Au | | | | |
| Pt, Pt(100) | T7 | 162 | TLTTLTN | M13 (Ph.D. −7, |
| | | | | NEB) |
| Pt, Pt(111) | S7 | 163 | SSFPQPN | M13 (Ph.D. −7, |
| | | | | NEB) |
| Pt | | 164 | CSQSVTSTKSC | CSBio 336s |
| | | | | automated peptide |
| | | | | synthesizer (SBio, |
| | | | | USA) on Wang |
| | | | | resin |
| Au | Midas2 | 165 | TGTSVLIATPYV | Chemical |
| | | | | synthesis |
| | | | | (originally |
| | | | | isolated from a |
| | | | | phage library), |
| | | | | SPOT synthesized |
| | | | | peptide array |
| Au, Si 4 nm SiOx | QBP1, 51 | 166 | PPPWLPYMPPWS | Chemical |
| spatter coated on a | | | | synthesis without |
| gold SPR | chip | | | display platform |
| Au | | 167 | GRGDS | CSBio 336s |
| | | | | automated peptide |
| | | | | synthesizer (SBio, |
| | | | | USA) on Want |
| | | | | resin |
| Au | | 168 | IKVAV | Fmoc synthesis |
| Au nanoparticles | E5 | 169 | CGGEVSALEKEVSAL | Chemical |
| | | | EKEVSALEKEVSALE | synthesis |
| | | | KEVSALEK | |
| Au & Ag QCM | AgBP1 | 170 | TGIFKSARAMRN | FliTrx bacterial |
| sensor surfaces | | | | surface library |
| Au & Ag QCM | AgBP2 | 171 | EQLGVRKELRGV | FliTrx bacterial |
| sensor surfaces | | | | surface library |
| Au > Ag = ZnS >> Pt | 6GB- & | 172 | QATSIGVEKLAGMAE | Streptavidin |
| | 11GB-AP | | SKPTKT | coated |
| | | | | polystyrene beads |
| Au > TiO₂ > Al₃O₄ > SiO₄ | | 173 | AGSWLRDIWTWLQSA | Chemical |
| | | | L | synthesis without |
| | | | | display platform, |
| Pd nanoparticles | A11 | 174 | TSNAVHPTLRAL | Mutating Pd4 |
| | | | | (Seq ID no. 105- |
| | | | | originally isolated |
| | | | | via phage display) |
| Pd nanoparticles | A6 | 175 | TSNAVAPTLRHL | Mutating Pd4 |
| | | | | (Seq ID no. 105- |
| | | | | originally isolated |
| | | | | via phage display) |
| Pd nanoparticles | A6, 11 | 176 | TSNAVAPTLRAL | Mutating Pd4 |
| | | | | (Seq ID no. 105- |
| | | | | originally isolated |
| | | | | via phage display) |
| PdAu bimetallic | H1 | 177 | WAGAKRHPTLRHL | Chemical |
| nanopartices | | | | synthesis without |
| | | | | display platform |
| | | | | (combining Pd4 |
| | | | | and AuBP1 (seq |
| | | | | ID no. 105 and 39) |

¹This is the MBP sequence for Strain #2

Silver is another desirable metal, and is diamagnetic. Examples are Ag, Ag nanoparticles, AgO, and Ag₂S. Metal binding peptides useful in silver extraction are shown in Table 2B.

TABLE 2B
Silver in Metal or Mineral Form Binding Peptides

| Table 2B | | | | |
|---|---|---|---|---|
| Target Silver | Literature Name | SEQ | Peptide | |
| Metal or mineral | (if assigned) | ID No. | Sequence to use | Display Platform |
| Silver (Ag), Ag | AG4 | 178 | NPSSLFRYLPSD | M13 phage |
| nanoparticles | | | | |
| Silver (Ag) | AG5 | 179 | SLATQPPRTPPV | M13 phage |
| Silver (Ag) | AG-P1 | 180 | KFLQFVCLGVGP | M13 phage |
| | AG-P2 | 181 | AVLMQKYHQLGP | M13 phage |
| | AG-P3 | 182 | IRPAIHIIPISH | M13 phage |
| | AG-P4 | 183 | NVIRASPPDTSY | M13 phage |
| | AG-P5 | 184 | LAMPNTQADAPF | M13 phage |
| | AG-P6 | 185 | QQNVPASGTCSI | M13 phage |
| | AG-P10 | 186 | NAMPGMVAWLCR | M13 phage |
| | AG-P11 | 187 | HNTSPSPIILTP | M13 phage |
| | AG-P12 | 188 | ASQTLLLPVPPL | M13 phage |
| | AG-P14 | 189 | YNKDRYEMQAPP | M13 phage |
| | AG-P18 | 190 | TLLLLAFVHTRH | M13 phage |
| | AG-P27 | 191 | PWATAVSGCFAP | M13 phage |
| | AG-P28 | 192 | SPLLYATTSNQS | M13 phage |
| | AG-P35 | 193 | WSWRSPTPHVVT | M13 phage |
| Ag⁺ | I | 194 | NYYRKYRD | SPOT |
| | | | | synthesized |
| | | | | peptide array |
| Ag⁺ | II | 195 | YGQAWYKK | SPOT |
| | | | | synthesized |
| | | | | peptide array |
| Ag⁺ | III | 196 | KGKNKRRR | SPOT |
| | | | | synthesized |
| | | | | peptide array |
| Ag⁺ | IV | 197 | QRRRKAWG | SPOT |
| | | | | synthesized |
| | | | | peptide array |
| Ag⁺ | V | 198 | YKRWKSKD | SPOT |
| | | | | synthesized |
| | | | | peptide array |
| Ag⁺ | VI | 199 | NYRAKAPK | SPOT |
| | | | | synthesized |
| | | | | peptide array |
| Ag⁺ | VII | 200 | TKKGRYQK | SPOT |
| | | | | synthesized |
| | | | | peptide array |
| Ag⁺ | VIII | 201 | RKYNWKKE | SPOT |
| | | | | synthesized |
| | | | | peptide array |
| Ag⁺ | IX | 202 | AYWWGRAR | SPOT |
| | | | | synthesized |
| | | | | peptide array |

Carbon, for example CO₃, or CaMg(CO₃)² (dolomite) can be a useful target in some applications.

Cadmium minerals and metal ions include Cd(NO₃)₂, CdCl₂, CdS, and CdSO₄.

Aluminum is paramagnetic but is also a useful target for mining and recycling applications.

Other metals and metal ions to which metal binding peptides have been generated include: B, CeMgAl, Co²⁺, Cobalt (Co), Cr, Cr₂O₃, Fe₂O, Fluoride, Francolite, Gallium arsenide, Germanium, Graphene, Hydroxyapatite, InP, LaPO₄, LaPO4:Ce³⁺, Tb³⁺, Magnesium, MgF₂, Molybdenite (MoS₂), Ni, Ni₃B, Palladium & Platinum Ions (Pd⁴⁺, Pt²⁺), Pb(NO₃)₂, Pb²⁺, PbS, Pd, Pt, PtO, SiO₄ (Quartz), Silicon, SiO₂, Ti, Titanium bisammonium lactatodihydroxide, and TiO₂.

Table 2C contains a list of additional metal binding peptides as well as their targets.

| Table 2C. | Literature | | | |
|---|---|---|---|---|
| Target Metal | Name if | SEQ | Peptide | |
| or mineral | assigned | ID No. | Sequence to use | Display Plaform |
| Nonspecific metal, | Co3-P1, L1₀ | 203 | SVSVGMKPSPRP | M13 phage, M13 |
| Co, FePt | | | | phage (12-mer) |
| 100 nm Ti particles | Ti-2 | 204 | GHTHYHAVRTQT | Chemical synthesis |
| (Q-sense, QSX-310), | | | | without display |
| Titanium | | | | platform, M13 |
| Bisammonium | | | | phage |
| 100 nm Ti particles | Ti-1 | 205 | QPYLFATDSLIK | Chemical synthesis |
| (Q-sense, QSX-310), | | | | without display |
| Titanium | | | | platform, M13 |
| Bisammonium | | | | phage |
| Lactatodihydroxide) | | | | |
| acid-washed calcite | CalcAcid 5 | 206 | DVFSSFNLKHMRG | M13 phage (NEB), |
| or aragonite | | | | chemical synthesis |
| | | | | without display |
| | | | | platform |
| acid-washed calcite | AragBasic | 207 | HTQNMRMYEPWFG | M13 phage (NEB), |
| or aragonite | 10 | | | chemical synthesis |
| | | | | without display |
| | | | | platform |
| aluminum 2024 | A1-S4 | 208 | NNRPEPSPVVPH | M13 phage |
| aluminum 2024 | A1-S5 | 209 | SPLDGKNIPLGH | M13 phage |
| aluminum 2024 | A1-S3 | 210 | TLWSQGRSAYPV | M13 phage |
| aluminum 2024 | A1-S1 | 211 | VPSSGPQDTRTT | M13 phage |
| aluminum 2024 | A1-S6 | 212 | WPAPAIWHAPTL | M13 phage |
| aluminum 2024 | A1-S2 | 213 | YSPDPRPWSSRY | M13 phage |
| C Graphene, edges of | GBP | 214 | EPLQLKM | Phage (7 mer) |
| graphene | | | | |
| C graphene sheets or | GBP | 215 | GAMHLPWHMGTL | Chemical synthesis |
| HOPG (graphite) | | | | without display |
| | | | | platform |
| C Single-walled | — | 216 | DSPHTELP | pVIII library |
| carbon nanotubes | | | | |
| C Single-walled | | 217 | DYFSSPYYEQLF | M13 (Ph.D. −12, NEB) |
| carbon nanotubes | | | | |
| C Single-walled | CBP | 218 | HSSYWYAFNNKT | combinatorial |
| carbon nanotubes, | | | | phage peptide |
| carbon nanotubes, | | | | display library. |
| central plane of | | | | |
| graphene | | | | |
| Ca hydroxyapatite | HAP | 219 | KLSW | Phage display |
| Ca Hydroxyapatite | | 220 | NPYHPTIPQSVH | M13 (Ph.D. −12, NEB) |
| Ca hydroxyapatite | HAP-1 | 221 | TVSRPTAPYVTP | M13 (Ph.D. −12, NEB) |
| CaCO₃ | CaCO3 | 222 | DVFSSFNLKHMR | M13 phage library |
| | | | | (NEB) |
| CaCO₃ | CaCO3 | 223 | HTQNMRMYEPWF | M13 phage library |
| | | | | (NEB) |
| Cadmium sulphide | — | 224 | CTYSRKHKC | Phage display |
| calcite powder | np4346 | 225 | MLIL | Fmoc synthesis |
| calcite powder | p266 | 226 | NTNS | Fmoc synthesis |
| calcite powder | np8688 | 227 | PICL | Fmoc synthesis |
| calcite powder | np6688 | 228 | PWFF | Fmoc synthesis |
| calcite powder | np4138 | 229 | PWFW | Fmoc synthesis |
| calcite powder | p37 | 230 | QSQN | Fmoc synthesis |
| calcite powder | p87 | 231 | QSTN | Fmoc synthesis |
| calcite powder | nu67 | 232 | STTC | Fmoc synthesis |
| calcite powder | p509 | 233 | TQNY | Fmoc synthesis |
| calcite powder | p19 | 234 | TTNN | Fmoc synthesis |
| calcite powder | p113 | 235 | SSYN | Fmoc synthesis |
| Calcium phosphate | | 236 | KDVVVGVPGGQD | FliTrx cell surface |
| | | | | display system |
| | | | | (Invitrogen) |
| carbon nanotubes | B2 | 237 | EIHWEIHWCMPHKT | M13 phage (NEB), |
| | | | | Si-C particles |
| carbon nanotubes | B4 | 238 | HNWYHWWMPHNT | M13 phage (NEB), |
| | | | | Si-C particles |
| carbon nanotubes | | 239 | HTSYWYAFNTKT | Phage peptide |
| | | | | library |
| carbon nanotubes | B1 | 240 | HWKHPWGAWDTL | M13 phage (NEB), |
| | | | | Si-C particles |
| carbon nanotubes | B3 | 241 | HWSAWWIRSNQS | M13 phage (NEB), |
| | | | | Si-C particles |
| CAT(CeMgAl₁₁O₁₉: | FL 464 R/A | 242 | ACQYPLCS | f88.4 phage |
| Tb³⁺) > LAP(LaPO₄: | | | | (disulfide- |
| Ce³⁺, Tb³⁺) > | | | | constrained hybrid |
| SiO₂ > BAM(BaMgAl₁₀O₁₇: | | | | pVIII; LX-4 library |
| Eu²⁺) > LaPO₄ > | | | | from Dr. Jamie |
| YOX(Y₂O₃: Eu³⁺) | | | | Scott) |
| CAT(CeMgAl₁₁O₁₉: | FL 464 Cl/A | 243 | RAQTPLCS | f88.4 phage |
| Tb³⁺) > LAP(LaPO₄: | | | | (disulfide- |
| Ce³⁺, Tb³⁺) > | | | | constrained hybrid |
| SiO₂ > BAM(BaMgAl₁₀O₁₇: | | | | pVIII; LX-4 library |
| Eu²⁺) > LaPO₄ > | | | | from Dr. Jamie |
| YOX(Y₂O₃: Eu³⁺) | | | | Scott) |
| CAT(CeMgAl₁₁O₁₉: | FL 464 Q/A | 244 | RCAYPLCS | f88.4 phage |
| Tb³⁺) > LAP(LaPO₄: | | | | (disulfide- |
| Ce³⁺, Tb³⁺) > | | | | constrained hybrid |
| SiO₂ > BAM(BaMgAl₁₀O₁₇: | | | | pVIII; LX-4 library |
| Eu²⁺) > LaPO₄ > | | | | from Dr. Jamie |
| YOX(Y₂O₃: Eu³⁺) | | | | Scott) |
| Lanthanide | FL 473 | 245 | RCLRSHCG | f88.4 phage |
| Phosphates, | | | | (disulfide- |
| CAT(CeMgAl₁₁O₁₉: | | | | constrained hybrid |
| Tb³⁺) > LAP(LaPO₄: | | | | pVIII; LX-4 library |
| Ce³⁺, Tb³⁺) > | | | | from Dr. Jamie |
| SiO₂ > BAM(BaMgAl₁₀O₁₇: | | | | Scott), phage VIII |
| Eu²⁺) > LaPO₄ > | | | | |
| YOX(Y₂O₃: Eu³⁺) | | | | |
| Lanthanide | FL 486 | 246 | RCPRFSCW | f88.4 phage |
| Phosphates, | | | | (disulfide- |
| CAT(CeMgAl₁₁O₁₉: | | | | constrained hybrid |
| Tb³⁺) > LAP(LaPO₄: | | | | pVIII; LX-4 library |
| Ce³⁺, Tb³⁺) > | | | | from Dr. Jamie |
| SiO₂ > BAM(BaMgAl₁₀O₁₇: | | | | Scott), phage VIII |
| Eu²⁺) > LaPO₄ > | | | | |
| YOX(Y₂O₃: Eu³⁺) | | | | |
| CAT(CeMgAl₁₁O₁₉: | FL 464 Y/A | 247 | RCQAPLCS | f88.4 phage |
| Tb³⁺) > LAP(LaPO₄: | | | | (disulfide- |
| Ce³⁺, Tb³⁺) > | | | | constrained hybrid |
| SiO₂ > BAM(BaMgAl₁₀O₁₇: | | | | pVIII; LX-4 library |
| Eu²⁺) > LaPO₄ > | | | | from Dr. Jamie |
| YOX(Y₂O₃: Eu³⁺) | | | | Scott) |
| CAT(CeMgAl₁₁O₁₉: | FL 464 P/A | 248 | RCQYALCS | f88.4 phage |
| Tb³⁺) > LAP(LaPO₄: | | | | (disulfide- |
| Ce³⁺, Tb³⁺) > | | | | constrained hybrid |
| SiO₂ > BAM(BaMgAl₁₀O₁₇: | | | | pVIII; LX-4 library |
| Eu²⁺) > LaPO₄ > | | | | from Dr. Jamie |
| YOX(Y₂O₃: Eu³⁺) | | | | Scott) |
| CAT(CeMgAl₁₁O₁₉: | FL 464 C2/A | 249 | RCQYPLAS | f88.4 phage |
| Tb³⁺) > LAP(LaPO₄: | | | | (disulfide- |
| Ce³⁺, Tb³⁺) > | | | | constrained hybrid |
| SiO₂ > BAM(BaMgAl₁₀O₁₇: | | | | pVIII; LX-4 library |
| Eu²⁺) > LaPO₄ > | | | | from Dr. Jamie |
| YOX(Y₂O₃: Eu³⁺) | | | | Scott) |
| CAT(CeMgAl₁₁O₁₉: | FL 464 S/A | 250 | RCQYPLCA | f88.4 phage |
| Tb³⁺) > LAP(LaPO₄: | | | | (disulfide- |
| Ce³⁺, Tb³⁺) > | | | | constrained hybrid |
| SiO₂ > BAM(BaMgAl₁₀O₁₇: | | | | pVIII; LX-4 library |
| Eu²⁺) > LaPO₄ > | | | | from Dr. Jamie |
| YOX(Y₂O₃: Eu³⁺) | | | | Scott) |
| Lanthanide | FL 464 | 251 | RCQYPLCS | E. coli, Phage VIII, |
| phosphate | | | | f88.4 phage |
| CAT(CeMgAl₁₁O₁₉: | | | | (disulfide- |
| Tb³⁺) > LAP(LaPO₄: | | | | constrained hybrid |
| Ce³⁺, Tb³⁺) > | | | | pVIII; LX-4 library |
| SiO₂ > BAM(BaMgAl₁₀O₁₇: | | | | from Dr. Jamie |
| Eu²⁺) > LaPO₄ > | | | | Scott) |
| YOX(Y₂O₃: Eu³⁺) | | | | |
| Lanthanide | FL 476 | 252 | RCQYSPCH | Phage VIII, f88.4 |
| Phosphates, | | | | phage (disulfide- |
| CAT(CeMgAl₁₁O₁₉: | | | | constrained hybrid |
| Tb³⁺) > LAP(LaPO₄: | | | | pVIII; LX-4 library |
| Ce³⁺, Tb³⁺) > | | | | from Dr. Jamie |
| SiO₂ > BAM(BaMgAl₁₀O₁₇: | | | | Scott) |
| Eu²⁺) > LaPO₄ > | | | | |
| YOX(Y₂O₃: Eu³⁺) | | | | |
| Lanthanide | FL 499 | 253 | SCFRPTCP | f88.4 phage |
| Phosphates, | | | | (disulfide- |
| CAT(CeMgAl₁₁O₁₉: | | | | constrained hybrid |
| Tb³⁺) > LAP(LaPO₄: | | | | pVIII; LX-4 library |
| Ce³⁺, Tb³⁺) > | | | | from Dr. Jamie |
| SiO₂ > BAM(BaMgAl₁₀O₁₇: | | | | Scott), phage VIII |
| Eu²⁺) > LaPO₄ > | | | | |
| YOX(Y₂O₃: Eu³⁺) | | | | |
| Lanthanide | FL 489 | 254 | SCKTVFCY | Phage VIII, f88.4 |
| Phosphates, | | | | phage (disulfide- |
| CAT(CeMgAl₁₁O₁₉: | | | | constrained hybrid |
| Tb³⁺) > LAP(LaPO₄: | | | | pVIII; LX-4 library |
| Ce³⁺, Tb³⁺) > | | | | from Dr. Jamie |
| SiO₂ > BAM(BaMgAl₁₀O₁₇: | | | | Scott) |
| Eu²⁺) > LaPO₄ > | | | | |
| YOX(Y₂O₃: Eu³⁺) | | | | |
| Cd, Cd²⁺, Cu²⁺, Zn²⁺ | CP | 255 | GCGCPCGCG | E. coli, chemical |
| | | | | synthesis without |
| | | | | display platform |
| Cd | HP | 256 | GHHPHGGHHPHG | E. coli |
| Cd, CdC₁₂, Co²⁺, | His6, H6 | 257 | HHHHHH | E. coli, |
| Cu²⁺, Cd²⁺, Zn² | | | | Caulobacter |
| | | | | crescentus, |
| | | | | Saccharomyces |
| | | | | cerevisiae |
| Cd | CdBP | 258 | HSQKVF | E. coli |
| Cd²⁺, Cu²⁺, Zn²⁺ | HP | 259 | GHHPHG | E. coli, chemical |
| | | | | synthesis without |
| | | | | display platform |
| Cd²⁺, Hg²⁺ | | 260 | CGCCGCGCCGCGCCG | E. coli |
| CdCl₂ or ZnCl₂, | CP2 | 261 | SGCGCPCGCGCGCPCG | Saccharomyces |
| CdSO₄ | | | CG | cerevisiae |
| | | | | immobilized on |
| | | | | cytopore |
| | | | | microcarrier beads, |
| | | | | Saccharomyces |
| | | | | cerevisiae |
| CdCl₂ or ZnCl₂ | HP3 | 262 | SGHHPHGHHPHGHHPH | Saccharomyces |
| | | | G | cerevisiae |
| CdS | J182 | 263 | CTYSRLHLC | M13 phage |
| CdS | d7-D07pep | 264 | DVHHHGRHGAEHADI | Saccharomyces |
| | | | | cerevisiae |
| CdS | d7-E01pep | 265 | DVHHHGRHGAEQAEI | Saccharomyces |
| | | | | cerevisiae |
| CdS | d7-D01pep | 266 | HDYRGHIHGHSQHGTE | Saccharomyces |
| | | | QPD | cerevisiae |
| CdS | E14 | 267 | PWIPTPRPTFTG | M13 phage |
| CdS | D01H | 268 | QVQLQQSGPGLVKPSQ | Saccharomyces |
| | | | TLSLTCAISGDSVSSN | cerevisiae |
| | | | SAAWNWIRQSPSRGLE | |
| | | | WQG | |
| CdS | D01 | 269 | QVQLQQSGPGLVKPSQ | Saccharomyces |
| | | | TLSLTCAISGDSVSSN | cerevisiae |
| | | | SAAWNWIRQSPSRGLE | |
| | | | WQGHDYRGHIHGHSQH | |
| | | | GTEQPDIRRHGRLLLC | |
| | | | ERCN* | |
| CdS | D0H | 270 | QVQLQQSGPGLVKPSQ | Saccharomyces |
| | | | TLSLTCAISGDSVSSN | cerevisiae |
| | | | SAAWNWIRQSPSRGLE | |
| | | | WQGHDYRGHIHGHSQH | |
| | | | GTEQP*D* | |
| CdS | D07R | 271 | QVQLVQSGAEVKKPGA | Saccharomyces |
| | | | SVKVSCKAPGYTFTGY | cerevisiae |
| | | | DLHWVRQAPGQGLEWM | |
| | | | G | |
| CdS | D07 | 272 | QVQLVQSGAEVKKPGA | Saccharomyces |
| | | | SVKVSCKAPGYTFTGY | cerevisiae |
| | | | DLHWVRQAPGQGLEWM | |
| | | | GRINPSSGATNYAQRF | |
| | | | QGRVTMTRDVHHHGRH | |
| | | | HGAEHADI* | |
| CdS | D07V | 273 | QVQLVQSGAEVKKPGA | Saccharomyces |
| | | | SVKVSCKAPGYTFTGY | cerevisiae |
| | | | DLHWVRQAPGQGLEWM | |
| | | | GRINPSSGATNYAQRF | |
| | | | QG*RVTMTRD* | |
| CdS | E01R | 274 | QVQLVQSGAEVKKPGS | Saccharomyces |
| | | | SVKVSCKASGDTFSSY | cerevisiae |
| | | | AINWVRQAPGQGLEWM | |
| | | | G | |
| CdS | E01 | 275 | QVQLVQSGAEVKKPGS | Saccharomyces |
| | | | SVKVSCKASGDTFSSY | cerevisiae |
| | | | AINWVRQAPGQGLEWM | |
| | | | GRINPNSGATNYAQRF | |
| | | | QGRVTMTRDVHHHGRH | |
| | | | GAEQAEI* | |
| CdS | E01V | 276 | QVQLVQSGAEVKKPGS | Saccharomyces |
| | | | SVKVSCKASGDTFSSY | cerevisiae |
| | | | AINWVRQAPGQGLEWM | |
| | | | GRINPNSGATNYAQRF | |
| | | | QG*RVTMTRD* | |
| CdS | J140 | 277 | SLTPLTTSHLRS | M13 phage |
| Co | Co1-P5 | 278 | ESIPALAGLSDK | M13 phage |
| Co | Co3-P12 | 279 | GTSTFNSVPVRD | M13 phage |
| Co | Co1-P6 | 280 | GVLNAAQTWALS | M13 phage |
| Co | Co1-P13 | 281 | HAMRPQVHPNYA | M13 phage |
| Co | Co1-P2 | 282 | HETNPPATIMPH | M13 phage |
| Co | Co1-P17 | 283 | HPPTDGMVPSPP | M13 phage |
| Co | Co1-P1 | 284 | HSVRWLLPGAHP | M13 phage |
| Co | Co1-P10 | 285 | HYPTLPLGSSTY | M13 phage |
| Co | Co2-P2 | 286 | KLHSSPHTPLVQ | M13 phage |
| Co | Co2-P17 | 287 | QLLPLTPSLLQA | M13 phage |
| Co | Co2-P13 | 288 | QNFLQVIRNAPR | M13 phage |
| Co | Co1-P15 | 289 | QYKHHPQKAAHI | M13 phage |
| Co | Co3-P13 | 290 | SAPNLNALSAAS | M13 phage |
| Co | Co2-P1 | 291 | SLTQTVTPWAFY | M13 phage |
| Co | Co1-P4 | 292 | SPLQVLPYQGYV | M13 phage |
| Co | Co2-P11 | 293 | TFPSHLATSTQP | M13 phage |
| Co | Co1-P21 | 294 | TGDVSNNPNVTL | M13 phage |
| Co | Co2-P7 | 295 | TNLDDSYPLHHL | M13 phage |
| Co | Co2-P6 | 296 | TPNSDALLTPAL | M13 phage |
| Co | Co2-P9 | 297 | TQQTDSRPPVLL | M13 phage |
| Co | Co1-P18 | 298 | TWQPFGMRPSDP | M13 phage |
| Co | Co3-P16 | 299 | VPTNVQLQTPRS | M13 phage |
| Co | Co1-P3 | 300 | WASAAWLVHSTI | M13 phage |
| Co | Co1-P16 | 301 | YGNQTPYWYPHR | M13 phage |
| Co²⁺, Cu²⁺, Cd²⁺, Zn² | C6 | 302 | CCCCCC | Saccharomyces |
| | | | | cerevisiae |
| Co²⁺, Cu²⁺, Cd²⁺, Zn² | D6 | 303 | DDDDDD | Saccharomyces |
| | | | | cerevisiae |
| Co²⁺, Cu²⁺, Cd²⁺, Zn² | DE3 | 304 | DEDEDE | Saccharomyces |
| | | | | cerevisiae |
| Co²⁺, Cu²⁺, Cd²⁺, Zn² | G6 | 305 | GGGGGG | Saccharomyces |
| | | | | cerevisiae |
| Cr | pSB3103 | 306 | ALRRDVNCIGASMH | E. coli |
| Cr | pSB3089 | 307 | PGMDHQKPLGKQAT | E. coli |
| Cr | pSB3084 | 308 | PGMDRQQHQSKQAT | E. coli |
| Cr | pSB3088 | 309 | PGMYNQHQKTKEAT | E. coli |
| Cr₂O₃ | Cr2O3 | 310 | RIRHRLVGQ | E. coli K−12 |
| Cr₂O₃ | Cr2O3 | 311 | VVRPKAATN | E. coli K−12 |
| Cu & Ni | HG12 | 312 | HGGGHGHGGGHG | Chemical synthesis |
| nanoparticles | | | | (Applied |
| | | | | Biosystems Peptide |
| | | | | Synthesizer 432A |
| Cu₂O Class I | CN46 | 313 | ADRTRGRIRGNC | E. coli |
| Cu₂O Class I | CN225 | 314 | RHTDGLRRIAAR | E. coli |
| Cu₂O Class I | CN86 | 315 | RPRRSAARGSEG | E. coli |
| Cu₂O Class I | CN85 | 316 | RTRRQGGDVSRD | E. coli |
| Cu₂O Class II | CN88 | 317 | EKWGMHQECYRH | E. coli |
| Cu₂O Class II | CN44 | 318 | NTVWRLNSSCGM | E. coli |
| Cu₂O Class II | CN93 | 319 | TMEPRWWCNPIN | E. coli |
| Cu₂O, ZnO, ZnO | CN146 | 320 | MRHSSSGEPRLL | E. coli |
| class II | | | | |
| Cu₂O, ZnO, ZnO | CN111 | 321 | PAGLQVGFAVEV | E. coli |
| class II | | | | |
| Cu₂O, ZnO, ZnO | CN120 | 322 | PASRVEKNGVRR | E. coli |
| Class I | | | | |
| Cu₂O, ZnO, ZnO | CN179 | 323 | RIGHGRQIRKPL | E. coli |
| class I | | | | |
| Cu₂O, ZnO, ZnO | CN185 | 324 | RTDDGVAGRTWL | E. coli |
| class II | | | | |
| Cu₂O, ZnO | CN155 | 325 | VRTRDDARTHRK | E. coli |
| CuFeS2 chalcopyrite | Fel4 | 326 | DKKKCDGKRCSWPS | M13 phage |
| CuFeS2 chalcopyrite | #3 | 327 | DPIKHTSG | M13 phage |
| CuFeS2 chalcopyrite | #12 | 328 | DSQKTNPS3 | M13 phage |
| CuFeS2 chalcopyrite | Fe4 | 329 | EKDRCTKNTCKPPA | M13 phage |
| CuFeS2 chalcopyrite | Fe18 | 330 | EKKKCGTMACPYRA | M13 phage |
| CuFeS2 chalcopyrite | Fe9 | 331 | ERSGCHKKACPKTP | M13 phage |
| CuFeS2 chalcopyrite | Fe13 | 332 | GKCSCKEKQCRTTL | M13 phage |
| CuFeS2 chalcopyrite | Fe6 | 333 | GKKKCPNKSCTSLF | M13 phage |
| CuFeS2 chalcopyrite | Fe1 | 334 | HKTQCNPRACTRRH | M13 phage |
| CuFeS2 chalcopyrite | Fe5 | 335 | KDHDCHRAQCRPNL | M13 phage |
| CuFeS2 chalcopyrite | Fe2 | 336 | KKTNCKHDSCRTCQ | M13 phage |
| CuFeS2 chalcopyrite | Fe11 | 337 | KNEKCAFIHKCYLYP | M13 phage |
| CuFeS2 chalcopyrite | Fe10 | 338 | KNKRCSQGCCINNG | M13 phage |
| CuFeS2 chalcopyrite | Fe7 | 339 | KSKSCEAMQCNKYY | M13 phage |
| CuFeS2 chalcopyrite | Fe15 | 340 | KSRHCSQIQCGDKV | M13 phage |
| CuFeS2 chalcopyrite | Fe3 | 341 | QRNKCHHNTCVKML | M13 phage |
| CuFeS2 chalcopyrite | Fe8 | 342 | RKKKCKGNCCYTPQ | M13 phage |
| Dolomite | dolomite | 343 | ADYFTARPGPIT | M13 phage (Ph.D. −12, |
| | Clone 1 | | | NEB) |
| Dolomite | dolomite | 344 | ANDGLATRPRDL | M13 phage (Ph.D. −12, |
| | Clone 5 | | | NEB) |
| Dolomite | dolomite | 345 | APKGLTNTSQLM | M13 phage (Ph.D. −12, |
| | Clone 9 | | | NEB) |
| Dolomite | Dolomite | 346 | APVAHAFPQAMM | M13 phage (Ph.D. −12, |
| | | | | NEB) |
| Dolomite | dolomite | 347 | DTNFVKAPRQPN | M13 phage (Ph.D. −12, |
| | Clone 6 | | | NEB) |
| Dolomite | Dolomite | 348 | EFQTPLRANVSF | M13 phage (Ph.D. −12, |
| | | | | NEB) |
| Dolomite | Dolomite | 349 | GFAHHSWAPDRA | M13 phage (Ph.D. −12, |
| | | | | NEB) |
| Dolomite | Dolomite | 350 | GFASDPSSSPWT | M13 phage (Ph.D. −12, |
| | | | | NEB) |
| Dolomite | dolomite | 351 | GMELHSKLPIYR | M13 phage (Ph.D. −12, |
| | Clone 7 | | | NEB) |
| Dolomite | Dolomite | 352 | GMELHSKLPTYR | M13 phage (Ph.D. −12, |
| | | | | NEB) |
| Dolomite | Dolomite | 353 | HLGGSIARIPEQ | M13 phage (Ph.D. −12, |
| | | | | NEB) |
| Dolomite | dolomite | 354 | HTEPANWYPHTH | M13 phage (Ph.D. −12, |
| | Clone 5 | | | NEB) |
| Dolomite | dolomite | 355 | HYTEASFDIRTR | M13 phage (Ph.D. −12, |
| | Clone 4 | | | NEB) |
| Dolomite | Dolomite | 356 | LPSRVQELWWPA | M13 phage (Ph.D. −12, |
| | | | | NEB) |
| Dolomite | Dolomite | 357 | LPTMMNNNWNQR | M13 phage (Ph.D. −12, |
| | | | | NEB) |
| Dolomite | Dolomite | 358 | MNDTKWAAPQGL | M13 phage (Ph.D. −12, |
| | | | | NEB) |
| Dolomite | dolomite | 359 | MPNPHLALPHGS | M13 phage (Ph.D. −12, |
| | Clone 2 | | | NEB) |
| Dolomite | Dolomite | 360 | NFDELTMPNYRT | M13 phage (Ph.D. −12, |
| | | | | NEB) |
| Dolomite | dolomite | 361 | NIQTTHLFPLPR | M13 phage (Ph.D. −12, |
| | Clone 6 | | | NEB) |
| Dolomite | Dolomite | 362 | NPIPDTRNHRLV | M13 phage (Ph.D. −12, |
| | | | | NEB) |
| Dolomite | Dolomite | 363 | NQNYDAEQLITP | M13 phage (Ph.D. −12, |
| | | | | NEB) |
| Dolomite | dolomite | 364 | QHHTLSTAPYLY | M13 phage (Ph.D. −12, |
| | Clone 1 | | | NEB) |
| Dolomite | Dolomite | 365 | QIPNAVDLYWSP | M13 phage (Ph.D. −12, |
| | | | | NEB) |
| Dolomite | dolomite | 366 | QLTVDNNHQGND | M13 phage (Ph.D. −12, |
| | Clone 3 | | | NEB) |
| Dolomite | dolomite | 367 | QQNYLTQNIGRA | M13 phage (Ph.D. −12, |
| | Clone 2 | | | NEB) |
| Dolomite | dolomite | 368 | QTLPLPLTIAHP | M13 phage (Ph.D. −12, |
| | Clone 4 | | | NEB) |
| Dolomite | Dolomite | 369 | SLNCSLASSACR | M13 phage (Ph.D. −12, |
| | | | | NEB) |
| Dolomite | Dolomite | 370 | SNITPQTSTPSL | M13 phage (Ph.D. −12, |
| | | | | NEB) |
| Dolomite | Dolomite | 371 | SPNIGIAKNMLY | M13 phage (Ph.D. −12, |
| | | | | NEB) |
| Dolomite | dolomite | 372 | SPNPPANAVITN | M13 phage (Ph.D. −12, |
| | Clone 3 | | | NEB) |
| Dolomite | Dolomite | 373 | SPNPPANAVTTN | M13 phage (Ph.D. −12, |
| | | | | NEB) |
| Dolomite | dolomite | 374 | STDMSPSPMSHS | M13 phage (Ph.D. −12, |
| | Clone 7 | | | NEB) |
| Dolomite | Dolomite | 375 | TANWHPARTLLT | M13 phage (Ph.D. −12, |
| | | | | NEB) |
| Dolomite | Dolomite | 376 | THYTRGLSPFSL | M13 phage (Ph.D. −12, |
| | | | | NEB) |
| Dolomite | dolomite | 377 | TSENNYAVESFH | M13 phage (Ph.D. −12, |
| | Clone 8 | | | NEB) |
| Dolomite | Dolomite | 378 | TVLHNKSPDQSQ | M13 phage (Ph.D. −12, |
| | | | | NEB) |
| Dolomite | Dolomite | 379 | VDIHSGTWPLSY | M13 phage (Ph.D. −12, |
| | | | | NEB) |
| Dolomite | Dolomite | 380 | YVAHEVINLHHT | M13 phage (Ph.D. −12, |
| | | | | NEB) |
| Enargite | R4#8 | 381 | FHRAPWJYLGNY | M13 phage |
| Enargite | R4#11 | 382 | FPFIHKQRYVDPL | M13 phage |
| Enargite | R5#2 | 383 | FPWYKWRLPDVS | M13 phage |
| Enargite | R4#2 | 384 | GMKPWFYSNWKG | M13 phage |
| Enargite | R4#4 | 385 | GMLHWSYSIFNP | M13 phage |
| Enargite | R3#5 | 386 | HTSSLWHLFRST | M13 phage |
| Enargite | R4#16 | 387 | IPLHSLHVKWGK | M13 phage |
| Enargite | R4410 | 388 | IPWHRPAQVMHL | M13 phage |
| Enargite | R3(2)415 | 389 | KFSTHPWHSYSP | M13 phage |
| Enargite | R3416 | 390 | LPWHWAPNMYRS | M13 phage |
| Enargite | R544 | 391 | MGKPAPRYLGNN | M13 phage |
| Enargite | R4(2)49 | 392 | MGKSTLRYTTIV | M13 phage |
| Enargite | R4412 | 393 | MHKPTVHIKGPT* | M13 phage |
| Zeolites | zeolites | 394 | MDHGKYRQKQATPG | E. coli |
| Fe Oxide, Fe₂O₃ | | 395 | RRTVKHHVN | E. coli |
| Fe, Fe3O4, Zn, ZnO | | 396 | AGYPLSENFYYP | M13 phage |
| Fe, Fe3O4, Zn, ZnO | | 397 | FHPRLQQDHWLH | M13 phage |
| Fe, Fe3O4, Zn, ZnO | | 398 | GLHTSATNLYLH | M13 phage |
| Fe, Fe3O4, Zn, ZnO | | 399 | WQDFGAVRSTRS | M13 phage |
| FePt | L10 | 400 | HNKHLPSTQPLA | M13 phage (12-mer) |
| FePt | L10 | 401 | KSLSRHDHIHHH | M13 phage (12-mer) |
| FePt | L10 | 402 | VISNHRESSRPL | M13 phage (12-mer) |
| Francolite (Four | 3 | 403 | WSITTYHDRAIV | M13 phage (Ph.D. −12, |
| Corner mine) | | | | NEB) |
| Francolite (Four | 7 | 404 | WSYVPFARQVNQ | M13 phage (Ph.D. −12, |
| Corner mine) | | | | NEB) |
| Francolite (Four | — | 405 | HMPHHVSNLQLH | M13 phage (Ph.D. −12, |
| Corner mine) | | | | NEB) |
| Francolite (Four | — | 406 | GSNGIWFNLAHR | M13 phage (Ph.D. −12, |
| Corner mine) | | | | NEB) |
| Francolite (Four | — | 407 | YSQPTLWALTSR | M13 phage (Ph.D. −12, |
| Corner mine) | | | | NEB) |
| Francolite (Four | — | 408 | NIGHRVNSPFPQ | M13 phage (Ph.D. −12, |
| Corner mine)10 | | | | NEB) |
| Francolite (Four | — | 409 | APRLLSDNTYNV | M13 phage (Ph.D. −12, |
| Corner mine) | | | | NEB) |
| Francolite (Four | — | 410 | AHLEDITVHDGS | M13 phage (Ph.D. −12, |
| Corner mine)12 | | | | NEB) |
| Francolite (Four | — | 411 | TNNTFWFPAEFG | M13 phage (Ph.D. −12, |
| Corner mine)2 | | | | NEB) |
| Francolite (Four | — | 412 | TNSNWTPFWPLP | M13 phage (Ph.D. −12, |
| Corner mine)2 | | | | NEB) |
| Francolite (Four | - | 413 | TSPPQVAYPTLS | M13 phage (Ph.D. −12, |
| Corner mine)3 | | | | 12, NEB |
| Francolite (Four | 4 | 414 | SHVGNPYISATL | M13 phage (Ph.D. −12, |
| Corner mine) | | | | 12, NEB |
| Francolite (Four | 4 | 415 | SSMTHQHARVDT | M13 phage (Ph.D. −12, |
| Corner mine) | | | | NEB) |
| Francolite (Four | 5 | 416 | ASLQHTALLNQNN | M13 phage (Ph.D. −12, |
| Corner mine) | | | | NEB) |
| Francolite (Four | 5 | 417 | EHWQDNWMRWIT | M13 phage (Ph.D. −12, |
| Corner mine) | | | | NEB) |
| Francolite (Four | 6 | 418 | EKISDYAWPWRT | M13 phage (Ph.D. −12, |
| Corner mine) | | | | NEB) |
| Francolite (Four | 6 | 419 | HYGVQAPHNSNS | M13 phage (Ph.D. −12, |
| Corner mine) | | | | NEB) |
| Francolite (Four | 7 | 420 | DHRSISAFPNPP | M13 phage (Ph.D. −12, |
| Corner mine) | | | | NEB) |
| Francolite (Four | 8 | 421 | MEQFQSAGNPGW | M13 phage (Ph.D. −12, |
| Corner mine) | | | | NEB) |
| Francolite (Four | 9 | 422 | MEQTYPSSHRPG | M13 phage (Ph.D. −12, |
| Corner mine) | | | | NEB) |
| Francolite (South | 1 | 423 | YIGSQTNERYSP | M13 phage (Ph.D. −12, |
| Fort Meade) | | | | NEB) |
| Francolite (South | 1 | 424 | YQSTRTHAEASP | M13 phage (Ph.D. −12, |
| Fort Meade) | | | | NEB) |
| Francolite (South | 10, 20 | 425 | TKNMLSLPVGPG | M13 phage (Ph.D. −12, |
| Fort Meade), Zn | | | | NEB), M13 |
| | | | | bacteriophage |
| Francolite (South | 11 | 426 | HHHQTLRPAPFA | M13 phage (Ph.D. −12, |
| Fort Meade) | | | | NEB) |
| Francolite (South | 12 | 427 | AVPHRVGGLHSL | M13 phage (Ph.D. −12, |
| Fort Meade) | | | | NEB) |
| Francolite (South | 2 | 428 | HSVQTYARPLPS | M13 phage (Ph.D. −12, |
| Fort Meade) | | | | 12, NEB |
| Francolite (South | 2 | 429 | QNLINWPPPRFS | M13 phage (Ph.D. −12, |
| Fort Meade) | | | | 12, NEB |
| Francolite (South | 3 | 430 | HGLTVQRPEQMM | M13 phage (Ph.D. −12, |
| Fort Meade) | | | | NEB) |
| Francolite (South | 3 | 431 | VSHSEYNRAATY | M13 phage (Ph.D. −12, |
| Fort Meade) | | | | NEB) |
| Francolite (South | 4 | 432 | ASDNRTMVLMFP | M13 phage (Ph.D. −12, |
| Fort Meade) | | | | NEB) |
| Francolite (South | 4 | 433 | GHVVTNSVWMLP | M13 phage (Ph.D. −12, |
| Fort Meade) | | | | NEB) |
| Francolite (South | 5 | 434 | IDYSAPSRYANS | M13 phage (Ph.D. - |
| Fort Meade) | | | | NEB) |
| Francolite (South | 5 | 435 | QGYTMFVAAEPL | M13 phage (Ph.D. −12, |
| Fort Meade) | | | | NEB) |
| Francolite (South | 6 | 436 | DPFPQRVNYLKR | M13 phage (Ph.D. −12, |
| Fort Meade) | | | | NEB) |
| Francolite (South | 6 | 437 | YSLPRHLVSLPP | M13 phage (Ph.D. −12, |
| Fort Meade) | | | | NEB) |
| Francolite (South | 7 | 438 | AASFQHSATANL | M13 phage (Ph.D. −12, |
| Fort Meade) | | | | NEB) |
| Francolite (South | 7 | 439 | HYNPEMPSSHNA | M13 phage (Ph.D. −12, |
| Fort Meade) | | | | NEB) |
| Francolite (South | 8 | 440 | HSMPHMGTYLIT | M13 phage (Ph.D. −12, |
| Fort Meade) | | | | NEB) |
| Francolite (South | 9 | 441 | TITPSYLLAHGP | M13 phage (Ph.D. −12, |
| Fort Meade) | | | | NEB) |
| Francolite > dolomite | — | 442 | AQINLDNHARWF | M13 phage (Ph.D. −12, |
| | | | | 12, NEB |
| Francolite > dolomite | — | 443 | DIRTEPNTSNS | M13 phage (Ph.D. −12, |
| | | | | NEB) |
| Francolite > dolomite | — | 444 | DLFYDANNVHAG | M13 phage (Ph.D. −12, |
| | | | | NEB) |
| Francolite > dolomite | — | 445 | DRAPLIPFASQH | M13 phage (Ph.D. −12, |
| | | | | NEB) |
| Francolite > dolomite | — | 446 | EHWQDNWMRWTT | M13 phage (Ph.D. −12, |
| | | | | NEB) |
| Francolite > dolomite | — | 447 | FANTSSPVVHPF | M13 phage (Ph.D. −12, |
| | | | | NEB) |
| Francolite > dolomite | — | 448 | GDDVNTMRARPL | M13 phage (Ph.D. −12, |
| | | | | NEB) |
| Francolite > dolomite | — | 449 | LAPVRPIFSMEV | M13 phage (Ph.D. −12, |
| | | | | NEB) |
| Francolite > dolomite | — | 450 | LSASSPTTTATW | M13 phage (Ph.D. −12, |
| | | | | NEB) |
| Francolite > dolomite | — | 451 | MEQFQSAGNPGN | M13 phage (Ph.D. −12, |
| | | | | NEB) |
| Francolite > dolomite | — | 452 | QQYVAYPIMKAL | M13 phage (Ph.D. −12, |
| | | | | NEB) |
| Francolite > dolomite | — | 453 | SAHGTSTGVPWP | M13 phage (Ph.D. −12, |
| | | | | NEB) |
| Francolite > dolomite | — | 454 | SPTSLLPTQAHY | M13 phage (Ph.D. −12, |
| | | | | NEB) |
| Francolite > dolomite | — | 455 | TNHTFWFPAEFG | M13 phage (Ph.D. −12, |
| | | | | NEB) |
| Francolite > dolomite | — | 456 | TPPPSEITTSPP | M13 phage (Ph.D. −12, |
| | | | | NEB) |
| Francolite > dolomite | — | 457 | VHFRIATPYFSP | M13 phage (Ph.D. −12, |
| | | | | NEB) |
| Francolite > dolomite | — | 458 | YLAHSSNNKILF | M13 phage (Ph.D. −12, |
| | | | | NEB) |
| Francolite > dolomite | — | 459 | YNLTPLPKGNAM | M13 phage (Ph.D. −12, |
| | | | | NEB) |
| GaAs, GaAs(100), | G12-3 | 460 | AQNPSDNNTHTH | M13 phage |
| GaAs(111)A, | | | | |
| GaAs(111)B, | | | | |
| InP(100), Si(100) | | | | |
| single-crystal semi- | | | | |
| conductors | | | | |
| GaAs, GaAs(100), | G1-3 | 461 | RLELAIPLQGSG | M13 phage |
| GaAs(111)A, | | | | |
| GaAs(111)B, | | | | |
| InP(100), Si(100) | | | | |
| single-crystal semi- | | | | |
| conductors | | | | |
| GaAs | G7-4 | 462 | TPPRPIQYNHTS | M13 phage |
| GaAs(100), | G12-5 | 463 | AASPTQSMSQAP | M13 phage |
| GaAs(111)A, | | | | |
| GaAs(111)B, | | | | |
| InP(100), Si(100) | | | | |
| single-crystal semi- | | | | |
| conductors | | | | |
| GaAs(100), | G14-3 | 464 | ARYDLSIPSSES | M13 phage |
| GaAs(111)A, | | | | |
| GaAs(111)B, | | | | |
| InP(100), Si(100) | | | | |
| single-crystal semi- | | | | |
| conductors | | | | |
| GaAs(100), | G1-4 | 465 | ASSSRSHFGQTD | M13 phage |
| GaAs(111)A, | | | | |
| GaAs(111)B, | | | | |
| InP(100), Si(100) | | | | |
| single-crystal semi- | | | | |
| conductors | | | | |
| GaAs(100), | G14-4 | 466 | GTLANQQIFLSS | M13 phage |
| GaAs(111)A, | | | | |
| GaAs(111)B, | | | | |
| InP(100), Si(100) | | | | |
| single-crystal semi- | | | | |
| conductors | | | | |
| GaAs(100), | G11-3 | 467 | HGNPLPMTPFPG | M13 phage |
| GaAs(111)A, | | | | |
| GaAs(111)B, | | | | |
| InP(100), Si(100) | | | | |
| single-crystal semi- | | | | |
| conductors | | | | |
| GaAs(100), | G15-5 | 468 | SSLQLPENSFPH | M13 phage |
| GaAs(111)A, | | | | |
| GaAs(111)B, | | | | |
| InP(100), Si(100) | | | | |
| single-crystal semi- | | | | |
| conductors | | | | |
| GaAs(100), | G13-5 | 469 | VTSPDSTTGAMA | M13 phage |
| GaAs(111)A, | | | | |
| GaAs(111)B, | | | | |
| InP(100), Si(100) | | | | |
| single-crystal semi- | | | | |
| conductors | | | | |
| GaAs(100), | G12-4 | 470 | WAHAPQLASSST | M13 phage |
| GaAs(111)A, | | | | |
| GaAs(111)B, | | | | |
| InP(100), Si(100) | | | | |
| single-crystal semi- | | | | |
| conductors | | | | |
| GE | Ge8 | 471 | SLKMPHWPHLLP | Phage (12 mer), |
| tetramethoxygermanium | | | | chemical synthesis |
| | | | | without display |
| | | | | platform |
| GE | Ge34 | 472 | TGHQSPGAYAAH | Phage (12 mer), |
| tetramethoxygermanium | | | | chemical synthesis |
| | | | | without display |
| | | | | platform |
| GE | Ge2 | 473 | TSLYTDRPSTPL | Phage (12 mer), |
| tetramethoxygermanium | | | | chemical synthesis |
| | | | | without display |
| | | | | platform |
| Hematite | Hem-tag | 474 | STVQTISPSNH | Custom engineered |
| nanoparticles | | | | fluorescent protein |
| IrO₂ | — | 475 | AGETQQAM | M13 virus library |
| isotactic poly(methyl | c02 | 476 | ELWRPTR | E. coli |
| methacrylate) | | | | |
| Ln Oxide & | — | 477 | ACTARSPWICG | Ph. D. -C7C phage |
| upconversion | library | | | |
| nanocrystals | | | | |
| Magnesium Fluoride | III | 478 | GEYDYACGVVGYE | Bio-panning |
| (MgF₂) | | | | |
| Magnesium Fluoride | VI | 479 | GGLNQVLRIPSFI | Bio-panning |
| (MgF₂) | | | | |
| Magnesium Fluoride | II | 480 | GMIVDHLPIQVNT | Bio-panning |
| (MgF₂) | | | | |
| Magnesium Fluoride | VII | 481 | GSPKHNLDMVKMM | Bio-panning |
| (MgF₂) | | | | |
| Magnesium Fluoride | V | 482 | GSYPKASLALLAP | Bio-panning |
| (MgF₂) | | | | |
| Magnesium Fluoride | IV | 483 | GTQAIRVHTISSQ | Bio-panning |
| (MgF2) | | | | |
| Magnesium Fluoride | I | 484 | GTQYYAYSTTQKS | Bio-panning |
| (MgF₂) | | | | |
| Mild Steel 1010 | MS-S1 | 485 | ATIHDAFYSAPE | M13 phage (Ph.D. −12, |
| | | | | NEB) |
| Mild Steel 1010 | MS-52 | 486 | NLNPNTASAMHV | M13 phage (Ph.D. −12, |
| | | | | NEB) |
| Mild Steel 1010 | MS-53 | 487 | NLTIASYPSMVV | M13 phage (Ph.D. −12, |
| | | | | NEB) |
| Mild Steel 1010 | MS-55 | 488 | QMDISLGRWSSM | M13 phage (Ph.D. −12, |
| | | | | NEB) |
| Mild Steel 1010 | MS-54 | 489 | QSHYRHISPAQV | M13 phage (Ph.D. −12, |
| | | | | NEB) |
| Mild Steel 1010 | MS-56 | 490 | YMKQIPAGRTNP | M13 phage (Ph.D. −12, |
| | | | | NEB) |
| Molybdenite (MoS₂) | P28 | 491 | DRWVARDPASIF | M13 phage |
| Molybdenite (MoS₂) | P15 | 492 | GVIHRNDQWTAP | M13 phage |
| Molybdenite (MoS₂) | P3 | 493 | SVMNTSTKDAIE | M13 phage |
| natural & synthetic | L | 494 | (VKTQATSREEPPRL | E. coli |
| zeolites, silica | | | PSKHRPG)₄VK | |
| | | | TQTAS | |
| Ni₃B Amorphous, | A6, C28 | 495 | ANHQSAN | M13 phage (Ph.D. −12, |
| Ni₃B Crystalline | | | | 7, NEB) |
| Ni₃B Amorphous | A2 | 496 | GALPNNL | M13 phage (Ph.D. −12, |
| | | | | 7, NEB) |
| Ni₃B Amorphous | A10 | 497 | GNRLSAD | M13 phage (Ph.D. −12, |
| | | | | 7, NEB) |
| Ni₃B Amorphous | A13 | 498 | HVQYWQF | M13 phage (Ph.D. −12, |
| | | | | 7, NEB) |
| Ni₃B Amorphous | A7 | 499 | LGFREKE | M13 phage (Ph.D. −12, |
| | | | | 7, NEB) |
| Ni₃B Amorphous | A12 | 500 | NTVIYQK | M13 phage (Ph.D. −12, |
| | | | | 7, NEB) |
| Ni₃B Amorphous | A8 | 501 | NVNSTSF | M13 phage (Ph.D. −12, |
| | | | | 7, NEB) |
| Ni₃B Amorphous | A15 | 502 | RLLNPWI | M13 phage (Ph.D. −12, |
| | | | | 7, NEB) |
| Ni₃B Amorphous | A4 | 503 | SEIVDNH | M13 phage (Ph.D. −12, |
| | | | | 7, NEB) |
| Ni₃B Amorphous | A3 | 504 | SLAVSRS | M13 phage (Ph.D. −12, |
| | | | | 7, NEB) |
| Ni₃B Amorphous | A9 | 505 | SPDTVQK | M13 phage (Ph.D. −12, |
| | | | | 7, NEB) |
| Ni₃B Amorphous | A1 | 506 | TNLTLAS | M13 phage (Ph.D. −12, |
| | | | | 7, NEB) |
| Ni₃B Amorphous | A5 | 507 | TNSSFHK | M13 phage (Ph.D. −12, |
| | | | | 7, NEB) |
| Ni₃B Amorphous | A11 | 508 | TQVYHPM | M13 phage (Ph.D. −12, |
| | | | | 7, NEB) |
| Ni₃B Amorphous | A14 | 509 | VSVNSRT | M13 phage (Ph.D. −12, |
| | | | | 7, NEB) |
| Ni₃B Crystalline | C14 | 510 | AGLPKHQ | M13 phage (Ph.D. −12, |
| | | | | 7, NEB) |
| Ni₃B Crystalline | C6 | 511 | ATSTAHA | M13 phage (Ph.D. −12, |
| Ni₃B Crystalline | C19 | 512 | DPYNRIN | M13 phage (Ph.D. −12, |
| | | | | 7, NEB) |
| Ni₃B Crystalline | C16 | 513 | ELTQISS | M13 phage (Ph.D. −12, |
| | | | | 7, NEB) |
| Ni₃B Crystalline | C5 | 514 | ETFPARG | M13 phage (Ph.D. −12, |
| | | | | 7, NEB) |
| Ni₃B Crystalline | C7 | 515 | GASATRT | M13 phage (Ph.D. −12, |
| | | | | 7, NEB) |
| Ni₃B Crystalline | C11 | 516 | GDHSRHK | M13 phage (Ph.D. −12, |
| | | | | 7, NEB) |
| Ni₃B Crystalline | C26 | 517 | GDPKAAR | M13 phage (Ph.D. −12, |
| | | | | 7, NEB) |
| Ni₃B Crystalline | C13 | 518 | GPVNHQL | M13 phage (Ph.D. −12, |
| | | | | 7, NEB) |
| Ni₃B Cystalline | C23 | 519 | HAMRTEP | M13 phage (Ph.D. −12, |
| | | | | 7, NEB) |
| Ni₃B Crystalline | C15 | 520 | LEQTPMF | M13 phage (Ph.D. −12, |
| | | | | 7, NEB) |
| Ni₃B Crystalline | C22 | 521 | LNHVLPA | M13 phage (Ph.D. −12, |
| | | | | 7, NEB) |
| Ni₃B Cystalline | C17 | 522 | MNHAESY | M13 phage (Ph.D. −12, |
| | | | | 7, NEB) |
| Ni₃B Crystalline | C20 | 523 | RTFDAIS | M13 phage (Ph.D. −12, |
| | | | | 7, NEB) |
| Ni₃B Crystalline | C9 | 524 | SASKVHN | M13 phage (Ph.D. −12, |
| | | | | 7, NEB) |
| Ni₃B Crystalline | C2 | 525 | SDPQTHT | M13 phage (Ph.D. −12, |
| | | | | 7, NEB) |
| Ni₃B Crystalline | C1 | 526 | SPPKSNA | M13 phage (Ph.D. −12, |
| | | | | 7, NEB) |
| Ni₃B Crystalline | C24 | 527 | SPSTHWK | M13 phage (Ph.D. −12, |
| Ni₃B Crystalline | C2 | 528 | STFNSRV | M13 phage (Ph.D. - |
| | | | | 7, NEB) |
| Ni₃B Crystalline | C10 | 529 | SYTKLHL | M13 phage (Ph.D. - |
| | | | | 7, NEB) |
| Ni₃B Crystalline | C3 | 530 | TPPLLSP | M13 phage (Ph.D. - |
| | | | | 7, NEB) |
| Ni₃B Crystalline | C8 | 531 | VHTNPSR | M13 phage (Ph.D. - |
| | | | | 7, NEB) |
| Ni₃B Crystalline | C4 | 532 | VPIPYLP | M13 phage (Ph.D. - |
| | | | | 7, NEB) |
| Ni₃B Crystalline | C18 | 533 | VPSLTPT | M13 phage (Ph.D. - |
| | | | | 7, NEB) |
| Ni₃B Crystalline | C25 | 534 | WNAKYTL | M13 phage (Ph.D. - |
| | | | | 7, NEB) |
| Ni₃B Crystalline | C21 | 535 | YELVLPK | M13 phage (Ph.D. - |
| | | | | 7, NEB) |
| Ni₃B Crystalline | C12 | 536 | YQWPAR | M13 phage (Ph.D. - |
| | | | | 7, NEB) |
| Particulate Matter | VI | 537 | GYFIHSTYYTHNH | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | V | 538 | HHLFHAVYLNHY | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | II | 539 | HHLHWPHHHSYT | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | XI | 540 | HHTSGHHSLTLT | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | VII | 541 | HLVNRLRYPHVH | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | X | 542 | LTAHSHHHYHYA | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | VIII | 543 | LTHVLVHFYYHH | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | I | 544 | NGYYPHSHSYHQ | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | XII | 545 | NHVNTNYYPTLH | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | III | 546 | NHYYSHTHTYHG | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | IV | 547 | WHEIYFRTTHLTT | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | XII | 548 | YRAYHYLSYRDT | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Particulate Matter | IX | 549 | YTYTGLHLLASH | peptide array |
| with High Metal | | | | containing a library |
| Content | | | | of 85 sequences |
| | | | | pre-designed with |
| | | | | R based on Alvin, |
| | | | | 2017, with |
| | | | | WQDFGAVRSTR |
| | | | | S = 100% binding |
| Pb(NO₃)₂ >> Cd(NO₃)₂ & | NP | 550 | MDCPTEEALIR | Saccharomyces |
| ZnCl₂ | | | | cerevisiae |
| Pb2 | TAR-1 | 551 | ISLLHST | Phage display |
| | | | | library |
| PbS | J72 | 552 | QNPIHTH | M13 phage |
| polycrystalline | MBP-AgBP2 | 553 | DAQTNSSSGGGEQLGV | E. coli |
| quartz | | | RKELRGV | |
| polycrystalline | MBP-Ag4 | 554 | DAQTNSSSGGGNPSSL | E. coli |
| quartz | | | FRYLPSD | |
| polycrystalline | MBP2 | 555 | DAQTNSSSNNNNNNNN | E. coli |
| quartz | | | NNLGIEGR | |
| polystyrene | - | 556 | RRETAWA | Phage display |
| library | | | | |
| REE leachate (Bull | sLBT3 2x | 557 | FIDTNNDGWIEGDELL | Caulobacter |
| Hill, Round Top | tandem copy | | AFIDTNNDGWIEGDEL | crescentus, E. coli |
| Mountain, etc . . .), | | | LA | |
| TbCl3 hydrate salts | | | | |
| (Sigma) | | | | |
| Sheet silicates mica | Peptide 1 | 558 | QPASSRY | Si3N4 AFM |
| (Ted Pella Inc.) | | | | cantilever with |
| | | | | silicon tip |
| Sheet silicates mica | Peptide 2 | 559 | APASSRY | Si3N4 AFM |
| (Ted Pella Inc.) | | | | cantilever with |
| | | | | silicon tip |
| Sheet silicates mica | Peptide 3 | 560 | QAASSRY | Si3N4 AFM |
| (Ted Pella Inc.) | | | | cantilever with |
| | | | | silicon tip |
| Sheet silicates mica | Peptide 4 | 561 | QPAASRY | Si3N4 AFM |
| (Ted Pella Inc.) | | | | cantilever with |
| | | | | silicon tip |
| Sheet silicates mica | Peptide 5 | 562 | QPASARY | Si3N4 AFM |
| (Ted Pella Inc.) | | | | cantilever with |
| | | | | silicon tip |
| Sheet silicates mica | Peptide 6 | 563 | QPASSAY | Si3N4 AFM |
| (Ted Pella Inc.) | | | | cantilever with |
| | | | | silicon tip |
| Sheet silicates mica | Peptide 7 | 564 | QPASSRA | Si3N4 AFM |
| (Ted Pella Inc.) | | | | cantilever with |
| | | | | silicon tip |
| Si 4 nm SiOx spatter | W3 | 565 | CINQEGAGSKDK | Chemical synthesis |
| coated on a gold SPR | | | | without display |
| chip | | | | platform |
| Si 4 nm SiOx spatter | W1 | 566 | EVRKEVVAVARNTVI | Chemical synthesis |
| coated on a gold SPR | | | | without display |
| chip | | | | platform |
| Si 4 nm SiOx spatter | S2 | 567 | LPDWWPPPQLYH | Chemical synthesis |
| coated on a gold SPR | | | | without display |
| chip | | | | platform |
| Si 4 nm SiOx spatter | S5 | 568 | LPWLPSWHQHLS | Chemical synthesis |
| coated on a gold SPR | | | | without display |
| chip | | | | platform |
| Si 4 nm SiOx spatter | S6 | 569 | LQWLGPQSPQWP | Chemical synthesis |
| coated on a gold SPR | | | | without display |
| chip | | | | platform |
| Si 4 nm SiOx spatter | S4 | 570 | LSPFWPLAPPWH | Chemical synthesis |
| coated on a gold SPR | | | | without display |
| chip | | | | platform |
| Si 4 nm SiOx spatter | W2 | 571 | RKEDKAEDTKKK | Chemical synthesis |
| coated on a gold SPR | | | | without display |
| chip | | | | platform |
| Si 4 nm SiOx spatter | DS202 | 572 | RLNPPSQMDPPF | Chemical synthesis |
| coated on a gold SPR | | | | (originally isolated |
| chip | | | | from a phage |
| | | | | library) |
| Si 4 nm SiOx spatter | S3 | 573 | SPPRLLPWLRMP | Chemical synthesis |
| coated on a gold SPR | | | | without display |
| chip | | | | platform |
| Si 4 nm SiOx spatter | WR | 574 | VSVKTTKMTVVD | Chemical synthesis |
| coated on a gold SPR | | | | without display |
| chip | | | | platform |
| Si Glass, Silica & | Car9 | 575 | DSARGFKKPGKR | Custom engineered |
| Carbon | | | | fluorescent protein, |
| Nanostructures | | | | sfGFP |
| Si glass | Car15 | 576 | RTYLPLPWMAAL | Custom engineered |
| | | | | fluorescent protein |
| Silica, SiO₂ | 1 | 577 | HPPMNASHPHMH | M13 phage |
| Silica, Silicic Acid, | Si4-1, SiO₂ | 578 | MSPHPHPREIHHT | M13 (Ph.D. −12, |
| SiO₂ | NEB) | | | |
| Silica, SiO₂ | TBP6, | 579 | RKLPDA | Fmoc synthesis, E. |
| nanoparticle, Ti | minTBP-1, ti | | | coli fermentation |
| organometallic Ti | | | | |
| compounds, | | | | |
| Titanium | | | | |
| Silica, Silicic Acid | R5 | 580 | SSKKSGSYSGSKGSR | M13 (Ph.D. −12, NEB) |
| | | | RIL | |
| Silicic Acid | Si4-8 | 581 | APHHHHPHHLSR | M13 (Ph.D. −12,NEB) |
| Silicic Acid | Si3-3 | 582 | APPGHHHWHIHH | M13 (Ph.D. −12, NEB) |
| Silicic Acid, SiO2 | Si3-8, SiO₂ | 583 | KPSHHHHHTGAN | M13 (Ph.D. −12, NEB) |
| Silicic Acid | Si4-7 | 584 | LPHHHHLHTKLP | M13 (Ph.D. −12, NEB) |
| Silicic Acid | Si3-4 | 585 | MSASSYASFSWS | M13 (Ph.D. −12, NEB) |
| Silicic Acid | Si4-3 | 586 | MSPHHMHHSHGH | M13 (Ph.D. −12, NEB) |
| Silicic Acid, SiO₂ | Si4-10, SiO₂ | 587 | RGRRRRLSCRLL | M13 (Ph.D. −12, NEB) |
| Silicic Acid | Ge4-1 | 588 | TVASNSGLRPAS | M13 (Ph.D. −12, NEB) |
| | | | | NEB) |
| Single-Crystal | — | 589 | WPFIHPHAAHTIR | M13 Phage (NEB) |
| Graphite | | | | or Si-C particles |
| SiO₄ Quartz | — | 590 | PPWLPYMPPWS | Chemical synthesis |
| | | | | without display |
| | | | | platform |
| Streptavidin-binder | — | 591 | SWDPYSHLLQHPQ | M13 phage pIII |
| | | | | library |
| Strontium Titanate | — | 592 | AEEE | M13 bacteriophage |
| Tb³⁺ | LBT-6 | 593 | AACGDYNADGWIEFEE | 280-320-micron |
| | | | LAACA | TentaGel |
| | | | | microbeads |
| Tb³⁺ | LBT-1 | 594 | AACGDYNADGWIEFEE | 280-320-micron |
| | | | LACA | TentaGel |
| | | | | microbeads |
| Tb³⁺ | Library 2 | 595 | AADWNKDGWYEGPEAA | 280-320-micron |
| | | | A | TentaGel |
| | | | | microbeads |
| Tb³⁺ | — | 596 | AADXNKDGWYEGPEYY | 280-320-micron |
| | | | Y | TentaGel |
| | | | | microbeads |
| Tb³⁺, TbCl | LBT-Ref, | 597 | Ac- | 280-320-micron |
| | REF | | GDYNADGWIEFEEL | TentaGel |
| | | | | microbeads, |
| | | | | chemical synthesis |
| | | | | (plasmids also |
| | | | | designed & LBT- |
| | | | | tags expressed on |
| | | | | cells) |
| Tb³⁺ | LBT-2 | 598 | Ac- | 280-320-micron |
| | | | GGDYNADGWIEFEELL | TentaGel |
| | | | | microbeads |
| Tb³⁺ | LBT-5 | 599 | ACAAGDYNADGWIEFE | 280-320-micron |
| | | | ELACA | TentaGel |
| | | | | microbeads |
| Tb³⁺ | LBT-4 | 600 | ACAGDYNADGWIEFEE | 280-320-micron |
| | | | LAACA | TentaGel |
| | | | | microbeads |
| Tb³⁺ | LBT-3 | 601 | ACAGDYNADGWIEFEE | 280-320-micron |
| | | | LACA | TentaGel |
| | | | | microbeads |
| Tb³⁺ | LBT-8 | 602 | ACAGDYNADGWIEFEE | 280-320-micron |
| | | | LCAA | TentaGel |
| | | | | microbeads |
| Tb³⁺ | Peptide 2 | 603 | ACVDWNNDGWYEGDEC | 280-320-micron |
| | | | A | TentaGel |
| | | | | microbeads |
| Tb³⁺ | Library 7 | 604 | AYADTNNDGWYEGDEL | 280-320-micron |
| | | | EA | TentaGel |
| | | | | microbeads |
| Tb³⁺ | LBT-7 | 605 | CGDYNADGWIEFEELC | 280-320-micron |
| | | | | TentaGel |
| | | | | microbeads |
| Tb³⁺ | EF-hand | 606 | DXNXDXXEXXE | 280-320-micron |
| | protein motif | | | TentaGel |
| | | | | microbeads |
| Tb³⁺ | — | 607 | DYDDTNNDGWYEGDEL | 280-320-micron |
| | | | LA | TentaGel |
| | | | | microbeads |
| Tb³⁺ | — | 608 | EYEDTNNDGWYEGDEL | 280-320-micron |
| | | | NA | TentaGel |
| | | | | microbeads |
| Tb³⁺ | Library 3 | 609 | FIDFNGDGWWEDDELL | 280-320-micron |
| | | | A | TentaGel |
| | | | | microbeads |
| Tb³⁺ | Library 6 | 610 | FIDTNNDGWFEGDEFL | 280-320-micron |
| | | | A | TentaGel |
| | | | | microbeads |
| Tb³⁺, TbCl₃ hydrate | LBT-14, | 611 | FIDTNNDGWIEGDELL | 280-320-micron |
| salts (Sigma) | sLBT3 | | A | TentaGel |
| | | | | microbeads, E. coli, |
| | | | | chemical synthesis |
| | | | | without display |
| | | | | platform |
| Tb³⁺ | LBT-15 | 612 | FIDTNNDGWIEGDELL | 280-320-micron |
| | | | LEEG | TentaGel |
| | | | | microbeads |
| Tb³⁺ | — | 613 | FIDTNNDGWWEGDELL | 280-320-micron |
| | | | A | TentaGel |
| | | | | microbeads |
| Tb³⁺ | — | 614 | FIDTNNDGWYEGDELL | 280-320-micron |
| | | | A | TentaGel |
| | | | | microbeads |
| Tb³⁺ | — | 615 | FXDFNRDGXXENNELL | 280-320-micron |
| | | | A | TentaGel |
| | | | | microbeads |
| Tb³⁺ | troponin C | 616 | IFDKNADGFIDIEELG | Advanced |
| | | | E | ChemTech 396 |
| | | | | synthesizer |
| Tb³⁺ | — | 617 | IIDTNNDGWIEGDELL | 280-320-micron |
| | | | A | TentaGel |
| | | | | microbeads |
| Tb³⁺ | 1st gen., | 618 | LADYNKDGWYDGGDL | 280-320-micron |
| | library 1 | | | TentaGel |
| | | | | microbeads |
| Tb³⁺ | — | 619 | LLDXNKDGWYEGPEWW | 280-320-micron |
| | | | A | TentaGel |
| | | | | microbeads |
| Tb³⁺ | — | 620 | LLDYNKDGWYEGPELL | 280-320-micron |
| | | | L | TentaGel |
| | | | | microbeads |
| Tb³⁺ | — | 621 | MIDTNNDGWMEGDEML | 280-320-micron |
| | | | A | TentaGel |
| | | | | microbeads |
| Tb³⁺ | — | 622 | NYIDTNNDGWYEGDEL | 280-320-micron |
| | | | QA | TentaGel |
| | | | | microbeads |
| Tb³⁺ | — | 623 | SYNDTNNDGWYEGDEL | 280-320-micron |
| | | | YA | TentaGel |
| | | | | microbeads |
| Tb³⁺ | — | 624 | TGDYNKDGWYEPPET | 280-320-micron |
| | | | | TentaGel |
| | | | | microbeads |
| Tb³⁺ | — | 625 | TWDYNKDGWYSXXST | 280-320-micron |
| | | | | TentaGel |
| | | | | microbeads |
| Tb³⁺ | — | 626 | TYQDTNNDGWYEGDEL | 280-320-micron |
| | | | LA | TentaGel |
| | | | | microbeads |
| Tb³⁺ | — | 627 | VVDXNKDGWYEGPEP | 280-320-micron |
| | | | PT | TentaGel |
| | | | | microbeads |
| Tb³⁺ | — | 628 | VVDXNKDGWYEGPETT | 280-320-micron |
| | | | T | TentaGel |
| | | | | microbeads |
| Tb³⁺ | — | 629 | VWDYNKDGWYNGPNV | 280-320-micron |
| | | | | TentaGel |
| | | | | microbeads |
| Tb³⁺ | LB T-9 | 630 | VYDYNKDGWYEGPEL | 280-320-micron |
| | | | | TentaGel |
| | | | | microbeads |
| Tb³⁺ | — | 631 | VYDYNKDGWYEPPEV | 280-320-micron |
| | | | | TentaGel |
| | | | | microbeads |
| Tb³⁺ | — | 632 | VYDYNKDGWYQGPQL | 280-320-micron |
| | | | | TentaGel |
| | | | | microbeads |
| Tb³⁺ | — | 633 | VYDYNKDGWYSGPSL | 280-320-micron |
| | | | | TentaGel |
| | | | | microbeads |
| Tb³⁺ | — | 634 | WIDTNNDGWTEGDEWL | 280-320-micron |
| | | | A | TentaGel |
| | | | | microbeads |
| Tb³⁺ | — | 635 | WIDWNKDGYYESSELL | 280-320-micron |
| | | | A | TentaGel |
| | | | | microbeads |
| Tb³⁺ | — | 636 | WPDXNKDGWYEGPETT | 280-320-micron |
| | | | V | TentaGel |
| | | | | microbeads |
| Tb³⁺ | — | 637 | WVDGNKDGYYEEGELL | 280-320-micron |
| | | | A | TentaGel |
| | | | | microbeads |
| Tb³⁺ | LBT-10a | 638 | WVDWNKDGWYEGPELL | 280-320-micron |
| | | | A | TentaGel |
| | | | | microbeads |
| Tb³⁺ | — | 639 | WWDYNKDGWYEGPELL | 280-320-micron |
| | | | L | TentaGel |
| | | | | microbeads |
| Tb³⁺ | LBT1 | 640 | XGDYNKDGWYEELELX | 280-320-micron |
| | | | X | TentaGel |
| | | | | microbeads |
| Tb³⁺ | — | 641 | XPDXBKDGWYEGPEAA | 280-320-micron |
| | | | X | TentaGel |
| | | | | microbeads |
| Tb³⁺ | — | 642 | XVDXNKDGWYEGPEYY | 280-320-micron |
| | | | A | TentaGel |
| | | | | microbeads |
| Tb³⁺ | — | 643 | XXDFNXDGXXEPXELL | 280-320-micron |
| | | | A | TentaGel |
| | | | | microbeads |
| Tb³⁺ | — | 644 | XXDGNGDGXXEEXELL | 280-320-micron |
| | | | A | TentaGel |
| | | | | microbeads |
| Tb³⁺ | — | 645 | XXDLNXDGXXEXXELL | 280-320-micron |
| | | | A | TentaGel |
| | | | | microbeads |
| Tb³⁺ | — | 646 | XXDPNPDGXXEDDELL | 280-320-micron |
| | | | A | TentaGel |
| | | | | microbeads |
| Tb³⁺ | — | 647 | XXDXNKDGWYEGPEP | 280-320-micron |
| | | | PX | TentaGel |
| | | | | microbeads |
| Tb³⁺ | 2nd gen. | 648 | XXDXNKDGWYEGPEXX | 280-320-micron |
| | | | X | TentaGel |
| | | | | microbeads |
| Tb³⁺ | 3rd gen. | 649 | XXDXNXDGXXEXXELL | 280-320-micron |
| | | | A | TentaGel |
| | | | | microbeads |
| Tb³⁺ | — | 650 | XXDYNKDGWYNXXNX | 280-320-micron |
| | | | | TentaGel |
| | | | | microbeads |
| Tb³⁺ | — | 651 | XXDYNKDGWYQXXQX | 280-320-micron |
| | | | | TentaGel |
| | | | | microbeads |
| Tb³⁺ | — | 652 | XYDXNKDGWYEGPEWW | 280-320-micron |
| | | | A | TentaGel |
| | | | | microbeads |
| Tb³⁺ | — | 653 | YIDFNGDGWYEGDELL | 280-320-micron |
| | | | A | TentaGel |
| | | | | microbeads |
| Tb³⁺ | LBT-11a | 654 | YIDFNNDGWYEGDELL | 280-320-micron |
| | | | A | TentaGel |
| | | | | microbeads |
| Tb³⁺ | LBT-11b | 655 | YIDLNNDGWYEGDELL | 280-320-micron |
| | | | A | TentaGel |
| | | | | microbeads |
| Tb³⁺ | — | 656 | YIDLNNDGWYEGNELL | 280-320-micron |
| | | | A | TentaGel |
| | | | | microbeads |
| Tb3+ | — | 657 | YIDLNXDGWYEGDELL | 280-320-micron |
| | | | A | TentaGel |
| | | | | microbeads |
| Tb³⁺ | — | 658 | YIDPNPDGWTENPELL | 280-320-micron |
| | | | A | TentaGel |
| | | | | microbeads |
| Tb³⁺ | — | 659 | YIDTNNDGWVEGDEYL | 280-320-micron |
| | | | A | TentaGel |
| | | | | microbeads |
| TbTb³⁺, TbCl₃ hydrate | LBT, LBT- | 660 | YIDTNNDGWYEGDELL | E. coli, chemical |
| salts (Sigma) | 12, peptide 1, | | A | synthesis without |
| | sLBT1 | | | display platform, |
| | | | | 280-320-micron |
| | | | | TentaGel |
| | | | | microbeads |
| Tb³⁺ | Library 5 | 661 | YIDTNNDGWYEGDELL | 280-320-micron |
| | | | AAAA | TentaGel |
| | | | | microbeads |
| Tb³⁺ | — | 662 | YIDTNNDGWYEGDELL | 280-320-micron |
| | | | KEEG | TentaGel |
| | | | | microbeads |
| Tb³⁺ | LBT-13 | 663 | YIDTNNDGWYEGDELL | 280-320-micron |
| | | | LEEG | TentaGel |
| | | | | microbeads |
| Tb³⁺ | — | 664 | YIDTNNDGWYEGDELL | 280-320-micron |
| | | | LEER | TentaGel |
| | | | | microbeads |
| Tb³⁺ | — | 665 | YIDTNNDGWYEGDELL | 280-320-micron |
| | | | LKKK | TentaGel |
| | | | | microbeads |
| Tb³⁺ | — | 666 | YIDTNNDGWYEGDELL | 280-320-micron |
| | | | LLLL | TentaGel |
| | | | | microbeads |
| Tb³⁺ | Consensus | 667 | YIDWNNDGWYEGDELL | 280-320-micron |
| | | | A | TentaGel |
| | | | | microbeads |
| Tb³⁺ | — | 668 | YIDWNRDGWYESSELL | 280-320-micron |
| | | | A | TentaGel |
| | | | | microbeads |
| Tb³⁺ | 4th gen, | 669 | YIDXNNDGWYEGDELL | 280-320-micron |
| | library 4 | | A | TentaGel |
| | | | | microbeads |
| Tb³⁺ | — | 670 | YIDYNNDGWYEGDELL | 280-320-micron |
| | | | A | TentaGel |
| | | | | microbeads |
| Tb³⁺ | LBT-10b | 671 | YVDYNKDGWYEGPELL | 280-320-micron |
| | | | A | TentaGel |
| | | | | microbeads |
| Tb³⁺ | — | 672 | YVDYNNDGWWEGGELL | 280-320-micron |
| | | | A | TentaGel |
| | | | | microbeads |
| Tb³⁺ | — | 673 | YWDXNKDGWYEGPEWW | 280-320-micron |
| | | | A | TentaGel |
| | | | | microbeads |
| Tb³⁺ | — | 674 | YYDTNNDGWYEGDELL | 280-320-micron |
| | | | A | TentaGel |
| | | | | microbeads |
| Tb³⁺ | — | 675 | YYDWNKDGWYEGPEVV | 280-320-micron |
| | | | V | TentaGel |
| | | | | microbeads |
| Tb³⁺ | — | 676 | YYTDTNNDGWYEGDEL | 280-320-micron |
| | | | LA | TentaGel |
| | | | | microbeads |
| TbCl | LBTC7 | 677 | AACDYNKDGWYEELEA | chemical synthesis |
| | | | ACA | (plasmids also |
| | | | | designed & LBT- |
| | | | | tags expressed on |
| | | | | cells) |
| TbCl | LBTC4 | 678 | AACDYNKDGWYEELEA | chemical synthesis |
| | | | CA | (plasmids also |
| | | | | designed & LBT- |
| | | | | tags expressed on |
| | | | | cells) |
| TbCl | LBT1 | 679 | Ac- | chemical synthesis |
| | | | GDYNKDGWYEELEL | (plasmids also |
| | | | | designed & LBT- |
| | | | | tags expressed on |
| | | | | cells) |
| TbCl | LBTC9 | 680 | ACAADYNKDGWYEELE | chemical synthesis |
| | | | AACA | (plasmids also |
| | | | | designed & LBT- |
| | | | | tags expressed on |
| | | | | cells) |
| TbCl | LBTC6 | 681 | ACAADYNKDGWYEELE | chemical synthesis |
| | | | ACA | (plasmids also |
| | | | | designed & LBT- |
| | | | | tags expressed on |
| | | | | cells) |
| TbCl | LBTC3 | 682 | ACAADYNKDGWYEELE | chemical synthesis |
| | | | CAA | (plasmids also |
| | | | | designed & LBT- |
| | | | | tags expressed on |
| | | | | cells) |
| TbCl | LBTC8 | 683 | ACADYNKDGWYEELEA | chemical synthesis |
| | | | ACA | (plasmids also |
| | | | | designed & LBT- |
| | | | | tags expressed on |
| | | | | cells) |
| TbCl | LBTC5 | 684 | ACADYNKDGWYEELEA | chemical synthesis |
| | | | CA | (plasmids also |
| | | | | designed & LBT- |
| | | | | tags expressed on |
| | | | | cells) |
| TbCl | LBTC2 | 685 | ACADYNKDGWYEELEC | chemical synthesis |
| | | | AA | (plasmids also |
| | | | | designed & LBT- |
| | | | | tags expressed on |
| | | | | cells) |
| TbCl | LBTC1 | 686 | CDYNKDGWYEELEC | chemical synthesis |
| | | | | (plasmids also |
| | | | | designed & LBT- |
| | | | | tags expressed on |
| | | | | cells) |
| TbCl₃ hydrate salts | dLBT3 | 687 | GPGYIDTNNDGWIEGD | E. coli, chemical |
| (Sigma) | | | ELYIDTNNDGWIEGDE | synthesis without |
| | | | LLA | display platform |
| TbCl₃ hydrate salts | dLBT2 | 688 | GYIDTNNDGWIEGDEL | E. coli, chemical |
| (Sigma) | | | YIDTNNDGWIEGDELL | synthesis without |
| | | | A | display platform |
| TbCl₃ hydrate salts | sLBT2 | 689 | YIDTNNDGWIEGDELL | E. coli, chemical |
| (Sigma) | | | A | synthesis without |
| | | | | display platform |
| TbCl₃ hydrate salts | dLBT1 | 690 | YIDTNNDGWIEGDELY | E. coli, chemical |
| (Sigma) | | | IDTNNDGWIEGDELL | synthesis without |
| | | | A | display platform |
| Terbium Chloride | LBT | 691 | DYNKDGWYEELE | chemical synthesis |
| TbCl | | | | (plasmids also |
| | | | | designed & LBT- |
| | | | | tags expressed on |
| | | | | cells) |
| Ti <150 micron pure | Ti−12-3-6 | 692 | LCANNTTSVHPP | M13 phage |
| Ti particles | | | | |
| 696(Sumitomo | | | | |
| Titanium Corp. | | | | |
| Hyogo) | | | | |
| Ti <150 micron pure | Ti−12-3-2 | 693 | LDTTNVSGPMSS | M13 phage |
| Ti particles | | | | |
| (Sumitomo Titanium | | | | |
| Corp. Hyogo) | | | | |
| Ti <150 micron pure | Ti−12-3-5 | 694 | LPSQLLSQVNLT | M13 phage |
| Ti particles | | | | |
| (Sumitomo Titanium | | | | |
| Corp. Hyogo) | | | | |
| Ti <150 micron pure | Ti−12-3-7 | 695 | MQMEGKPTLTLR | M13 phage |
| Ti particles | | | | |
| (Sumitomo Titanium | | | | |
| Corp. Hyogo) | | | | |
| Ti <150 micron pure | Ti−12-3-9 | 696 | QDMIRTSALMLQ | M13 phage |
| Ti particles | | | | |
| (Sumitomo Titanium | | | | |
| Corp. Hyogo) | | | | |
| Ti <150 micron pure | Ti−12-3-1 | 697 | RKLPDAPGMHTW | M13 phage |
| Ti particles | | | | |
| (Sumitomo Titanium | | | | |
| Corp. Hyogo) | | | | |
| Ti <150 micron pure | Ti−12-3-10 | 698 | SCHVWYDSCSSP | M13 phage |
| Ti particles | | | | |
| (Sumitomo Titanium | | | | |
| Corp. Hyogo) | | | | |
| Ti <150 micron pure | Ti−12-3-4 | 699 | SDPNQDWRRTTP | M13 phage |
| Ti particles | | | | |
| (Sumitomo Titanium | | | | |
| Corp. Hyogo) | | | | |
| Ti <150 micron pure | Ti−12-3-8 | 700 | STLKNPINLLAN | M13 phage |
| Ti particles | | | | |
| (Sumitomo Titanium | | | | |
| Corp. Hyogo) | | | | |
| Ti <150 micron pure | Ti−12-3-3 | 701 | SYRLPVYLHALL | M13 phage |
| Ti particles | | | | |
| (Sumitomo Titanium | | | | |
| Corp. Hyogo) | | | | |
| Ti implant-grade Ti | TiBP1, Ti | 702 | RPRENRGRERGL | Synthesized using a |
| sheets (cp Grade 1 & | | | | standard solid |
| 4, A. D. MacKay, | | | | phase peptide |
| New York), titanium | | | | synthesis technique |
| on Wang resin | | | | |
| Ti implant-grade Ti | TiBP2 | 703 | SRPNGYGGSESS | Synthesized using a |
| sheets (cp Grade 1 & | | | | standard solid |
| 4, A. D. MacKay, | | | | phase peptide |
| New York), titanium | | | | synthesis technique |
| on Wang resin | | | | |
| Ti implant-grade Ti | TiBP60 | 704 | VGRVTSPRPQGR | FliTrx bacterial cell |
| sheets (cp Grade 1 & | | | | surface display |
| 4, A. D. MacKay, | | | | system (Invitrogen) |
| New York) | | | | |
| TiBALDH | R5 | 705 | SSKKSGSYSGSKGKRR | Chemical synthesis |
| | | | IL | without display |
| | | | | platform |
| Toner | TBP | 706 | GGGSGVYKVAYDWQH | Custom protein |
| | | | | and/or cross linker |
| Zeolites | — | 707 | VKTQATSREEPPRLPS | E. coli |
| | | | KHRPG | |
| Zinc Oxide, ZnO | ZnO-1 | 708 | EAHVMHKVAPRP | M13 (Ph.D. −12, NEB) |
| particles (Cojundo | | | | |
| Chemical Laboratory | | | | |
| Co.) | | | | |
| Zinc Oxide | — | 709 | RPHRK | Chemical synthesis |
| Zn | — | 710 | SYHHHH | E. coli |
| Zn²⁺ | M1 | 711 | VCATCEQIANSQHRSH | Influenza virus |
| | | | RQMV | |
| ZNO | ZNO | 712 | NTRMTARQHRSANHKS | E. coli k−12 |
| | | | TQRA | |
| ZNO | ZNO | 713 | YDSRSMRPH | Peptide library in |
| | | | | FimH |
| ZnO particles | ZnO-3 | 714 | ATHTNQTHALYR | M13 (Ph.D. −12, |
| (Cojundo Chemical | | | | NEB) |
| Laboratory Co.) | | | | |
| ZnO particles | ZnO-5 | 715 | DSGRYSMTNHYS | M13 (Ph.D. −12, |
| (Cojundo Chemical | | | | NEB) |
| Laboratory Co.) | | | | |
| ZnO particles | ZnO-2 | 716 | QNTATAVSRLSP | M13 (Ph.D. −12, |
| (Cojundo Chemical | | | | NEB) |
| Laboratory Co.) | | | | |
| ZnO particles | ZnO-4 | 717 | VSNHKALDYPTR | M13 (Ph.D. −12, |
| (Cojundo Chemical | | | | NEB) |
| Laboratory Co.) | | | | |
| ZnS | CT43 | 718 | AGDSSGVDSRSV | sfGFP |
| ZnS | Clone 9 | 719 | CFPMRSNQC | M13 phage |
| ZnS | ZnS | 720 | CGPAGDSSGVDSRSVG | FliTrx cell surface |
| | | | PC | display system |
| | | | | (Invitrogen) |
| ZnS | Clone 13 | 721 | CHMAPRWQC | M13 phage |
| ZnS | Clone 5 | 722 | CLQNRQSQC | M13 phage |
| ZnS | Clone 11 | 723 | CNHQMPMQC | M13 phage |
| ZnS | Clone 1 | 724 | CNKHQPMHC | M13 phage |
| ZnS | Clone 4 | 725 | CNNKVPVLC | M13 phage |
| ZnS | 18/A7, Clone | 726 | CNNPMHQNC | M13 phage, M13 |
| | 16 | | | phage (7 mer) |
| ZnS | Clone 3 | 727 | CNQLSTRPC | M13 phage |
| ZnS | Clone 12 | 728 | CNRQAVNAC | M13 phage |
| ZnS | Clone 10 | 729 | CPPQPNRQC | M13 phage |
| ZnS | Clone 6 | 730 | CQLQRQWNC | M13 phage |
| ZnS | Clone 2 | 731 | CQNPMQTFC | M13 phage |
| ZnS | Clone 14 | 732 | CQSMPHNRC | M13 phage |
| ZnS | Clone 7 | 733 | CQVNSAHQC | M13 phage |
| ZnS | Z15 | 734 | LPPAWAMQVHTA | M13 phage |
| ZnS | Z10 | 735 | LPRAFMGHAPGS | M13 phage |
| ZnS | Z8 | 736 | LRRSSEAHNSIV | M13 phage |
| ZnS | ZnS | 737 | NNPMHQN | M13 phage, M13 |
| | | | | phage (7 mer) |
| ZnS | Z3 | 738 | PHPHTHT | Saccharomyces |
| | | | | cerevisiae |
| ZnS | Z35 | 739 | PRPSPKMGVSVS | M13 phage |
| ZnS | Z6 | 740 | RRQDVHLPSRTL | M13 phage |
| ZnS | Z11 | 741 | TRHMASRTEAHL | M13 phage |
| ZnS sphalerite | — | 742 | QPKGPKQ | M13 phage |
| ZnS sphalerite & | — | 743 | TPTTYKV | M13 phage |
| CuFeS2 chalcopyrite | | | | |
| ZnS sphalerite | — | 744 | KPLLMGS | M13 phage |
| Co²⁺, Ni²⁺ | — | 745 | DAHKSEVA | Rink resin, Wang |
| | | | | resin |
| Co²⁺, Ni²⁺ | — | 746 | DAHK | Rink resin, Wang |
| | | | | resin |
| Cobalt | SMC03 | 747 | DRTISNK | E. coli |
| Cobalt | SMC04 | 748 | QNPGNTL | E. coli |
| Cobalt | SMC06 | 749 | SSSVVTH | E. coli |
| Cobalt | SMC07 | 750 | DAKDLNS | E. coli |
| Cobalt | SMC08 | 751 | DNDTKAS | E. coli |
| Cobalt | SMC09 | 752 | GLTDTSN | E. coli |
| Cobalt | SMC10 | 753 | KTSTHAI | E. coli |
| Cobalt | SMC11 | 754 | MRDSKML | E. coli |
| Cobalt | SMC12 | 755 | STISKAK | E. coli |
| Cobalt | SMC14 | 756 | TGQGGEY | E. coli |
| Cobalt | SMC15 | 757 | TKTQTHA | E. coli |
| Cobalt | SMC16 | 758 | TNHSAYH | E. coli |
| Cobalt | SMC17 | 759 | TQMLGQL | E. coli |
| Cobalt | SMC18 | 760 | VSPNKEA | E. coli |
| Cobalt | Co | 761 | EEEE | M13 virus |
| | nucleating | | | |
| | motif | | | |
| Cobalt, Nickel | SMC01, | 762 | MSTGLSS | E. coli |
| | SMN02 | | | |
| Cobalt, Nickel | SMCO2, | 763 | VPILEGT | E. coli |
| | SMN21 | | | |
| Cobalt, Nickel | SMC05, | 764 | SGTGASY | E. coli |
| | SMN01 | | | |
| Cobalt, Nickel | SMC13, | 765 | TASQNFY | E. coli |
| | SMN04 | | | |
| CoO | — | 766 | RSGRMQRRVAH | E. coli K−12 |
| | | | RS | |
| CoO | — | 767 | RSLGKDRPHFHRS | E. coli K−12 |
| CoO, ZnO | pJKS18 | 768 | RSRGLRNILMLR | E. coli K−12 |
| | | | SYDSRSMRPHRS | |
| CoO | — | 769 | RSEPRRATQAPR | E. coli K−12 |
| | | | SKPQKNEPAPRS | |
| CoO | — | 770 | RSLGAVSSLFRS | E. coli K−12 |
| | | | QKIMQTDIVRSK | |
| | | | GVRPAQRRS | |
| Cr₂O₃ | — | 771 | RSVVRPKAATN | E. coli K−12 |
| | | | RS | |
| Cr₂O₃ | — | 772 | RSRIRHRLVGQRS | E. coli K−12 |
| Cr₂O₃ | — | 773 | RSVKDGSATAKRSVAN | E. coli K−12 |
| | | | FETPRVRS | |
| Cr₂O₃ | — | 774 | RSAPQTGRPNNRSLPL | E. coli K−12 |
| | | | GNRDMQRS | |
| Fe₂O₃ | 1 | 775 | QMDTSTSLAPSR | M13 bacteriophage |
| Fe₂O₃ | 2 | 776 | VPFTLQTRSLSD | M13 bacteriophage |
| Fe₂O₃ | 3 | 777 | TVTPSNISFTPS | M13 bacteriophage |
| Fe₂O₃ | 4 | 778 | ASTLINPLSISL | M13 bacteriophage |
| Fe₂O₃ | 5 | 779 | AGSTASVTPAKH | M13 bacteriophage |
| Fe₂O₃ | 6 | 780 | QMANSVMPLSWT | M13 bacteriophage |
| Fe₂O₃ | 7 | 781 | YAHSHDKYHPN | M13 bacteriophage |
| Fe₂O₃ | 8 | 782 | NQSPHSTYTLKP | M13 bacteriophage |
| Fe₂O₃ | 9 | 783 | HNYPQSYRPPIV | M13 bacteriophage |
| Fe₂O₃ | 10 | 784 | TDNNTTATVSPS | M13 bacteriophage |
| Fe₂O₃ | 11 | 785 | TMNNTTATVSPS | M13 bacteriophage |
| Fe₂O₃ | 12 | 786 | FQKQTNQSVSVS | M13 bacteriophage |
| Fe₂O₃ | 13 | 787 | VHMTPTNLTPNL | M13 bacteriophage |
| Fe₂O₃ | 14 | 788 | TFSYHNSNSPT | M13 bacteriophage |
| Fe₂O₃ | 15 | 789 | VPDHQVSYTLSR | M13 bacteriophage |
| Fe₂O₃ | 16 | 790 | IFHSHASLSPNS | M13 bacteriophage |
| Fe₂O₃ | 17 | 791 | ADNANVSTLHPT | M13 bacteriophage |
| Fe₂O₃ | 18 | 792 | VNQQPSSAFSPS | M13 bacteriophage |
| Fe₂O₃ | 19 | 793 | LSTVQTLSPSNH | M13 bacteriophage |
| Fe₂O₃ | 20 | 794 | DMNHTKSSYNPS | M13 bacteriophage |
| Hydroxyapatite | cHABP1 | 795 | CMLPHEIGAC | Produced by |
| | | | | standard solid |
| | | | | phase peptide |
| | | | | synthesis on Wang |
| | | | | resin |
| L1₀CoPt | | 796 | KTHEIHSPLLHK | M13 phage |
| Lanthanide | FL 606 | 797 | TSTQCPSHIRACL | E. coli |
| phosphate (LaPO₄: | | | KKR | |
| Ce³⁺, Tb³⁺) | | | | |
| Lanthanide | FL 591 | 798 | DQSTCGRKAQRCPAYP | E. coli |
| phosphate (LaPO₄: | | | | |
| Ce³⁺, Tb³⁺) | | | | |
| Lanthanide | FL 592 | 799 | HTYPCYQTTPTCVPTS | E. coli |
| phosphate (LaPO₄: | | | | |
| Ce³⁺, Tb³⁺) | | | | |
| Lanthanide | FL 594 | 800 | DHNQCKQSPRMCIPPL | E. coli |
| phosphate (LaPO₄: | | | | |
| Ce³⁺, Tb³⁺) | | | | |
| Lanthanide | FL 601 | 801 | LKFTCSTYGGLCKADT | E. coli |
| phosphate (LaPO₄: | | | | |
| Ce³⁺, Tb³⁺) | | | | |
| Lanthanide | FL 602 | 802 | RQLMCNHRPPNCTKCH | E. coli |
| phosphate (LaPO₄: | | | | |
| Ce³⁺, Tb³⁺) | | | | |
| MnO₂ | — | 803 | RSHHMLRRRNTRS | E. coli K−12 |
| MnO₂ | — | 804 | RSHINASQRVARS | E. coli K−12 |
| MnO₂ | — | 805 | RSCPRLGVWFYRSLSV | E. coli K−12 |
| | | | GDGFVRRS | |
| MnO₂ | — | 806 | RSTSGPSRVMTRSIIL | E. coli K−12 |
| | | | RIGTLDRSCLKVFHMGW | |
| | | | RS | |
| MnO₂ | — | 807 | RSITPILHDHRRSSVR | E. coli K−12 |
| | | | PMVAHRRSPTLYFPAA | |
| | | | SRS | |
| Nickel | SMN03 | 808 | NTGSPYE | E. coli |
| Nickel | SMN05 | 809 | GSRSAQT | E. coli |
| Nickel | SMN06 | 810 | GTKGSLN | E. coli |
| Nickel | SMN07 | 811 | GYSSFNR | E. coli |
| Nickel | SMN08 | 812 | HHPVANT | E. coli |
| Nickel | SMN09 | 813 | HNETQKM | E. coli |
| Nickel | SMN10 | 814 | KDTSRSA | E. coli |
| Nickel | SMN11 | 815 | NAKHHPR | E. coli |
| Nickel | SMN12 | 816 | NGRAVNY | E. coli |
| Nickel | SMN13 | 817 | PGASVTY | E. coli |
| Nickel | SMN14 | 818 | RAEGTSE | E. coli |
| Nickel | SMN15 | 819 | RGATPMS | E. coli |
| Nickel | SMN16 | 820 | SLATDQK | E. coli |
| Nickel | SMN17 | 821 | SNNHSSM | E. coli |
| Nickel | SMN18 | 822 | STATPYK | E. coli |
| Nickel | SMN19 | 823 | TKTDVHF | E. coli |
| Nickel | SMN20 | 824 | TSVLNNT | E. coli |
| O | 6 | 825 | QWGWNMPLVEAQ | M13 bacteriophage |
| O | 24 | 826 | HSHLHIHSGIQA | M13 bacteriophage |
| O | 12 | 827 | NHVHRMHATPAY | M13 bacteriophage |
| O | 15 | 828 | HYQHNTHHPSRW | M13 bacteriophage |
| O | 9 | 829 | HSSPHFSRTWAS | M13 bacteriophage |
| O | 36 | 830 | HHRTLSPSVSIL | M13 bacteriophage |
| O | 3 | 831 | HSSPHFSRHGLL | M13 bacteriophage |
| O | 5 | 832 | NTIHHRHHMPP | M13 bacteriophage |
| O | 70 | 833 | SSGLRHSHHQHP | M13 bacteriophage |
| O | 38 | 834 | GHIHSMRHHRPT | M13 bacteriophage |
| PbO₂ | — | 835 | RSVQNDRIVAGRS | E. coli K−12 |
| PbO₂ | — | 836 | RSYPPFHNNDHRS | E. coli K−12 |
| PbO₂, ZnO | pJKS9 | 837 | RSNTRMTARQHRSANH | E. coli K−12 |
| | | | KSTQRARS | |
| PbO₂ | — | 838 | RSLAIDGTDVQRSKPL | E. coli K−12 |
| | | | ARSSGARS | |
| PbO₂ | — | 839 | RSPSPIRVPHHRSTAI | E. coli K−12 |
| | | | PNRQLIRSQIRIHAMG | |
| | | | HRS | |
| PbO₂ | — | 840 | RSRRVRDIHLGRSVQH | E. coli K−12 |
| | | | RLGQPLRSLHQQSSPT | |
| | | | LRS | |
| PbO₂ | — | 841 | RSRTPLAPVPVRSWHI | E. coli K−12 |
| | | | GSRTIARSFNGITIGD | |
| | | | RSYIPEHWYWRS | |
| SiO₂ | 2 | 842 | HTKHSHTSPPPL | phage |
| SiO₂ | 3 | 843 | HVSHFHASRHER | phage |
| SiO₂ | 4 | 844 | HLASGHSIHYRT | phage |
| SiO₂ | 5 | 845 | HQAHNHTHPSSL | phage |
| SiO₂ | 6 | 846 | HGSKANHPHIRA | phage |
| SiO₂ | 7 | 847 | HTPSNHRHTHNW | phage |
| SiO₂ | 8 | 848 | HAPHTHMRSWSA | phage |
| SiO₂ | 9 | 849 | HVSHHATGHTHT | phage |
| SiO₂ | 10 | 850 | HKLPSASRHHFH | phage |
| SiO₂ | 11 | 851 | HTTPSHLHPHSR | phage |
| SiO₂ | 12 | 852 | DPSTHQHPPHKH | phage |
| SiO₂ | 13 | 853 | SPQHTHHARIKN | phage |
| SiO₂ | 14 | 854 | HINHHHDTPSYR | phage |
| SiO₂ | 15 | 855 | HPGVHSHPSPTP | phage |
| SiO₂ | 1 | 856 | TVVQTYSMVTRA | phage |
| SiO₂ | 2 | 857 | FSYRQSPPPPLY | phage |
| SiO₂ | 3 | 858 | IMQNSISSPEML | phage |
| SiO₂, TiO₂ | SiC1, TiC1 | 859 | CHKKPSKSC | M13 phage |
| SiO₂, TiO₂ | SiC19, TiC6 | 860 | CTKRNNKRC | M13 phage |
| SiO₂ | SiC11 | 861 | CRRWESKRC | M13 phage |
| Synthetic Sapphire | B04 | 862 | (SG₄)₃SASQG₄SG- | Saccharomyces |
| | | | KMRAWGHPIWNW | cerevisiae |
| Synthetic Sapphire | B09 | 863 | (SG₄)₃SASQG₄SG- | Saccharomyces |
| | | | TKHGKRSRCYNL | cerevisiae |
| Synthetic Sapphire | D02 | 864 | (SG₄)₃SASQG₄SG- | Saccharomyces |
| | | | RTAKRKWKHTRD | cerevisiae |
| Synthetic Sapphire | F02 | 865 | (SG₄)₃SASQG₄SG- | Saccharomyces |
| | | | KRHKQKTSRMGK | cerevisiae |
| Synthetic Sapphire | F12 | 866 | (SG₄)₃SASQG₄SG- | Saccharomyces |
| | | | KRSKKCLRKNGS | cerevisiae |
| Synthetic Sapphire | X1 | 867 | (SG₄)₃SASQG₄SG- | Saccharomyces |
| | | | GXGXGXGXGXGX | cerevisiae |
| Synthetic Sapphire | X2 | 868 | (SG₄)₃SASQG₄SG- | Saccharomyces |
| | | | GGXXGGXXGGXX | cerevisiae |
| Synthetic Sapphire | X3 | 869 | (SG₄)₃SASQG₄SG- | Saccharomyces |
| | | | GGGXXXGGGXXX | cerevisiae |
| Synthetic Sapphire | cK1 | 870 | (SG₄)₃SASQG₄SG- | Saccharomyces |
| | | | CGKGKGKGKGKGKC | cerevisiae |
| Synthetic Sapphire | KIP | 871 | (SG₄)₃SASQG₄SG- | Saccharomyces |
| | | | GKPKGKPKGKPK | cerevisiae |
| Terbium doped | — | 872 | KKQKCRTDACVTQM | E. coli |
| Cerium-Magnesium | | | | |
| Aluminate | | | | |
| Terbium doped | — | 873 | NEKKCKGARCTIVT | E. coli |
| Cerium-Magnesium | | | | |
| Aluminate | | | | |
| Terbium doped | — | 874 | ATPKCKKKSCMTTQ | E. coli |
| Cerium-Magnesium | | | | |
| Aluminate | | | | |
| Terbium doped | — | 875 | VDKKCKSDDCGAWH | E. coli |
| Cerium-Magnesium | | | | |
| Aluminate | | | | |
| Terbium doped | — | 876 | HDKKCKRQPCVLAN | E. coli |
| Cerium-Magnesium | | | | |
| Aluminate | | | | |
| Terbium doped | — | 877 | FDKKCKSNKCLEVR | E. coli |
| Cerium-Magnesium | | | | |
| Aluminate | | | | |
| Terbium doped | — | 878 | PKKKCHPEPCQTCG | E. coli |
| Cerium-Magnesium | | | | |
| Aluminate | | | | |
| Terbium doped | — | 879 | KTEHCKKRKCPLDM | E. coli |
| Cerium-Magnesium | | | | |
| Aluminate | | | | |
| Terbium doped | — | 880 | ETKKCTTGPCKVVT | E. coli |
| Cerium-Magnesium | | | | |
| Aluminate | | | | |
| Terbium doped | — | 881 | KKKKCKKKICTTHT | E. coli |
| Cerium-Magnesium | | | | |
| Aluminate | | | | |
| Terbium doped | — | 882 | KKKKCKKNTCKNHT | E. coli |
| Cerium-Magnesium | | | | |
| Aluminate | | | | |
| TiO₂ | TiC21 | 883 | CDQQTNSEFC | M13 phage |
| Titanium | 1 | 884 | SHKHPVTPRFFVVESK | bacteriophage |
| Titanium | 2 | 885 | SGGGVTPRFFVVESK | bacteriophage |
| Titanium | 3 | 886 | SHKHGGHKHGSSGK | bacteriophage |
| Titanium | 4 | 887 | SHKHGGHKHGGHKHGS | bacteriophage |
| | | | DGK | |
| Titanium | Ti-7-3-1 | 888 | ATWVSPY | phage |
| Titanium | Ti-7-3-2 | 889 | AHSMGTG | phage |
| Titanium | Ti-7-3-3 | 890 | FSSQMRY | phage |
| Titanium | Ti-7-3-4 | 891 | GVGLPHT | phage |
| Titanium | Ti-7-3-5 | 892 | QIEPLAL | phage |
| Titanium | Ti-7-3-6 | 893 | RIVLPTY | phage |
| Titanium | Ti-7-3-7 | 894 | VQQVALL | phage |
| Titanium | Ti-7-3-8 | 895 | IVLPVPY | phage |
| Titanium | Ti-7-3-9 | 896 | GHWTRLA | phage |
| Titanium | Ti-7-3-10 | 897 | NLPLHST | phage |
| Titanium | 1 | 898 | SCFWFLRWSLFIVLFT | M13 phage |
| | | | CCS | |
| Titanium | 2 | 899 | SCESVDCFADSRMAKV | M13 phage |
| | | | SMS | |
| Titanium | 3 | 900 | SCVGFFCITGSDVASV | M13 phage |
| | | | NSS | |
| Titanium | 4 | 901 | SCSDCLKSVDFIPSSL | M13 phage |
| | | | ASS | |
| Titanium | 5 | 902 | SCAFDCPSSVARSPGE | M13 phage |
| | | | WSS | |
| Titanium | 6 | 903 | SCMLFSSVFDCGMLIS | M13 phage |
| | | | DLS | |
| Titanium | 7 | 904 | SCVDYVMHADSPGPDG | M13 phage |
| | | | LNS | |
| Titanium | 8 | 905 | SCSENFMFNMYGTGVC | M13 phage |
| | | | TES | |
| Titanium | 9 | 906 | SCSSFEVSEMFTCAVS | M13 phage |
| | | | SYS | |
| Titanium | 10 | 907 | SCGLNFPLCSFVCADF | M13 phage |
| | | | AQDAS | |
| Zn | 46 | 908 | ERSWTLDSALSM | M13 bacteriophage |
| Zn | 83 | 909 | SNNDLSPLQTSH | M13 bacteriophage |
| Zn | 21 | 910 | DSSNPIFWRPSS | M13 bacteriophage |
| Zn | 19 | 911 | SILSTMSPHGAT | M13 bacteriophage |
| Zn | 26 | 912 | SHALPLTWSTAA | M13 bacteriophage |
| Zn | 32 | 913 | HVSIHRTTHHEM | M13 bacteriophage |
| Zn | 52 | 914 | MKPDKAIRLDLL | M13 bacteriophage |
| Zn | 23 | 915 | HYPTAKFHAERL | M13 bacteriophage |
| Zn | 22 | 916 | FNTGSQMHQKFP | M13 bacteriophage |
| Zn | 53 | 917 | HHTHRVDVHQTR | M13 bacteriophage |
| Zn | 29 | 918 | FGLTAPRSASIL | M13 bacteriophage |
| Zn | 58 | 919 | APRLPQSLLPQL | M13 bacteriophage |
| ZnO | 7 | 920 | LLADTTHEIRPWT | M13 bacteriophage |
| ZnO | 44 | 921 | HSSHEIQPKGTNP | M13 bacteriophage |
| ZnO | 31 | 922 | HEIGHSPTSPQVR | M13 bacteriophage |
| ZnO | 43 | 923 | SHNHPPRHTAHS | M13 bacteriophage |
| ZnO | 25 | 924 | HSKLNNRHHALL | M13 bacteriophage |
| ZnO | 45 | 925 | HTKPFIEITPTQRA | M13 bacteriophage |
| ZnO | pJKS10 | 926 | RSVFLPSILGWRSRLD | E. coli K−12 |
| | | | DQGVAARS | |
| ZnO | pJKS12 | 927 | RSTRNKHTTARRSVAP | E. coli K−12 |
| | | | GIGEPSRS | |
| ZnO | pJKS14 | 928 | RSIMHVRLRARRSARH | E. coli K−12 |
| | | | MKDADPRS | |
| ZnO | pJKS17 | 929 | RSPIIIRSRINRSHGR | E. coli K−12 |
| | | | TKATPARS | |
| ZnO | pJKS11 | 930 | RSTRRGTHNKDRS | E. coli K−12 |
| ZnO | pJKS16 | 931 | RSTVPKRHPKDRS | E. coli K−12 |
| ZnO | pJKS45 | 932 | PSIAKKTHNKWRS | E. coli K−12 |
| ZnO | pJKS15 | 933 | RSYDSRSMRPHRS | E. coli K−12 |
| ZnO | pJKS46 | 934 | RSTASRHTEPHRS | E. coli K−12 |

²This “CAT(. . .” series of peptides are for the extraction of lanthanide elements from the
powder in waste fluorescent light bulbs.
3This is the MBP sequence for Strain #1

Use of the Metal Binding Yeast in Situ. The created Yeast of the disclosure are brought onto the mining site from a centralized manufacturing facility or cultured on-site using standard industrial bioreactors. Since the yeast still function as mineral processing reagents even when dead (cells retain displayed peptides and internal iron oxide deposits) they can be used in harsh conditions. The bio-magnetic mineral separation system operates continuously using existing industrial magnetic mineral processing equipment (eg. HGMS carousel concentrators) and the created yeast of the disclosure. Once the ore minerals have been recovered by the magnetic separator, the yeast will be ashed or dissolved when the ore concentrates are smelted or leached respectively.

The Plasmids that are embodiments of the disclosure are characterised by the inclusion of certain custom sequences:

1. CCC1p-FTL-CCC1t-CCC1p-FTH-CCC1t-CCC1p-Pcbp1-Adh3t
SEQ. ID. NO. 1
AACCCCTCAGCGTCAGGAAGACGCCACGGATCCAACAACATCATCGACGAAT
GAATTATCTGCCGCTGAGCCAACAATGGTCACTTCGACACATGCCACTAAGA
CAATACAGGCTCAAACACAAGATCCTCCCACGAAGCACAAGAAGTCCAGTTT
CTTCACTAAACTGTTCAAGAAAAAAAGCAGCCGTTAGCAGTTGTTTACTCGA
ATTTTGCAATCAACCCTAATTTTTGAAGCCTGGTTTTAGATTTATTCTCTTCTT
TTTCTTCTTGTGAACTTCAATTACTAATGTAACTTAATTTTTAATATAACTTTT
ACAGTTTAATAATATTGATTTTTTTCGGTCTGGACCAATCGCGCCGCATTTCTC
ACTAATATTACTAACATACCCTCTTCTCATTGGCTCGGTACCCCTTTCGTGACC
CGCATTTTTTGTTTTCTTTGTTAGCCCGAATGTCTCACAATGAAGATGTAAAA
TTAAGATTATATATGAAAAATTGATACAAAACAAAATAGTTCAAATAATCAA
GTTAAAGCGACATCACTCTCAATTTTTGCTTTTTTTAGGTTTATAGAATAGAA
ATATAACAGAACGAAGCCTTTAGACTCTTTTTTTTTATTGGTATATTGAACAA
AGAAACTTTTTTTTTGTCCTCCCATATCTCGTGCACACAAATATTATGAGCTCC
CAGATTCGTCAGAATTATTCCACCGACGTGGAGGCAGCCGTCAACAGCCTGG
TCAATTTGTACCTGCAGGCCTCCTACACCTACCTCTCTCTGGGCTTCTATTTCG
ACCGCGATGATGTGGCTCTGGAAGGCGTGAGCCACTTCTTCCGCGAATTGGC
CGAGGAGAAGCGCGAGGGCTACGAGCGTCTCCTGAAGATGCAAAACCAGCG
TGGCGGCCGCGCTCTCTTCCAGGACATCAAGAAGCCAGCTGAAGATGAGTGG
GGTAAAACCCCAGACGCCATGAAAGCTGCCATGGCCCTGGAGAAAAAGCTG
AACCAGGCCCTTTTGGATCTTCATGCCCTGGGTTCTGCCCGCACGGACCCCCA
TCTCTGTGACTTCCTGGAGACTCACTTCCTAGATGAGGAAGTGAAGCTTATCA
AGAAGATGGGTGACCACCTGACCAACCTCCACAGGCTGGGTGGCCCGGAGG
CTGGGCTGGGCGAGTATCTCTTCGAAAGGCTCACTCTCAAGCACGACTAAGT
GTAAAGTAACAACACTATACATATTTATTGTAAAGAAATTTGGGATTGAGAA
GCTTTGCTATATACTTGGAACTGGGCTGGATTTTCTCGACATACATTTCTTTTA
CGTTTTAATTTGTTTCTATATTCTCCTCTTTAAATTTATTTATATTAATGAATTT
CAAACTAGTTTCTTTTTCTATTGCCAAGAGGCCCATCAGGTGATCCATGATAA
CCTTTATTCGCATTGAATCGTTTTTCATTGGATCTAATTCGTCATTTGGTCGCC
GCCTTTCTTTGCTCCTTCTGCTTTTTTCTTTTTCTCTTTGACTATCAGGTCATAA
GATCTTCTCCTCCATTATGCCCAAGTGTTTTTCTTTTTTTTGCCTTTATATAAAT
TACAAAATACATACATATACTTCTCGTGCAGTATACTGGAATCAGCTTTATAC
CATATGAAGCACAACTTGGATCAGGACCGCTCTTCATTAAAAATCGAAGAAT
TATCTAAATAATTAAAACCCCTCAGCGTCAGGAAGACGCCACGGATCCAACA
ACATCATCGACGAATGAATTATCTGCCGCTGAGCCAACAATGGTCACTTCGA
CACATGCCACTAAGACAATACAGGCTCAAACACAAGATCCTCCCACGAAGCA
CAAGAAGTCCAGTTTCTTCACTAAACTGTTCAAGAAAAAAAGCAGCCGTTAG
CAGTTGTTTACTCGAATTTTGCAATCAACCCTAATTTTTGAAGCCTGGTTTTAG
ATTTATTCTCTTCTTTTTCTTCTTGTGAACTTCAATTACTAATGTAACTTAATTT
TTAATATAACTTTTACAGTTTAATAATATTGATTTTTTTCGGTCTGGACCAATC
GCGCCGCATTTCTCACTAATATTACTAACATACCCTCTTCTCATTGGCTCGGT
ACCCCTTTCGTGACCCGCATTTTTTGTTTTCTTTGTTAGCCCGAATGTCTCACA
ATGAAGATGTAAAATTAAGATTATATATGAAAAATTGATACAAAACAAAATA
GTTCAAATAATCAAGTTAAAGCGACATCACTCTCAATTTTTGCTTTTTTTAGG
TTTATAGAATAGAAATATAACAGAACGAAGCCTTTAGACTCTTTTTTTTTATT
GGTATATTGAACAAAGAAACTTTTTTTTTGTCCTCCCATATCTCGTGCACACA
AATATTatgacgaccgcgtccacctcgcaggtgcgccagaactaccaccaggactcagaggccgccatcaaccgccag
atcaacctggagctctacgcctcctacgtttacctgtccatgtcttactactttgaccgcgatgatgtggctttgaagaactttgccaa
atactttcttcaccaatctcatgaggagagggaacatgctgagaaactgatgaagctgcagaaccaacgaggtggccgaatcttc
cttcaggatatcaagaaaccagactgtgatgactgggagagcgggctgaatgcaatggagtgtgcattacatttggaaaaaaatg
tgaatcagtcactactggaactgcacaaactggccactgacaaaaatgacccccatttgtgtgacttcattgagacacattacctga
atgagcaggtgaaagccatcaaagaattgggtgaccacgtgaccaacttgcgcaagatgggagcgcccgaatctggcttggc
ggaatatctctttgacaagcacaccctgggagacagtgataatgaaagctaaGTGTAAAGTAACAACACTAT
ACATATTTATTGTAAAGAAATTTGGGATTGAGAAGCTTTGCTATATACTTGGA
ACTGGGCTGGATTTTCTCGACATACATTTCTTTTACGTTTTAATTTGTTTCTAT
ATTCTCCTCTTTAAATTTATTTATATTAATGAATTTCAAACTAGTTTCTTTTTCT
ATTGCCAAGAGGCCCATCAGGTGATCCATGATAACCTTTATTCGCATTGAATC
GTTTTTCATTGGATCTAATTCGTCATTTGGTCGCCGCCTTTCTTTGCTCCTTCT
GCTTTTTTCTTTTTCTCTTTGACTATCAGGTCATAAGATCTTCTCCTCCATTATG
CCCAAGTGTTTTTCTTTTTTTTGCCTTTATATAAATTACAAAATACATACATAT
ACTTCTCGTGCAGTATACTGGAATCAGCTTTATACCATATGAAGCACAACTTG
GATCAGGACCGCTCTTCATTAAAAATCGAAGAATTATCTAAATAATTAAAAC
CCCTCAGCGTCAGGAAGACGCCACGGATCCAACAACATCATCGACGAATGAA
TTATCTGCCGCTGAGCCAACAATGGTCACTTCGACACATGCCACTAAGACAA
TACAGGCTCAAACACAAGATCCTCCCACGAAGCACAAGAAGTCCAGTTTCTT
CACTAAACTGTTCAAGAAAAAAAGCAGCCGTTAGCAGTTGTTTACTCGAATT
TTGCAATCAACCCTAATTTTTGAAGCCTGGTTTTAGATTTATTCTCTTCTTTTT
CTTCTTGTGAACTTCAATTACTAATGTAACTTAATTTTTAATATAACTTTTACA
GTTTAATAATATTGATTTTTTTCGGTCTGGACCAATCGCGCCGCATTTCTCACT
AATATTACTAACATACCCTCTTCTCATTGGCTCGGTACCCCTTTCGTGACCCG
CATTTTTTGTTTTCTTTGTTAGCCCGAATGTCTCACAATGAAGATGTAAAATTA
AGATTATATATGAAAAATTGATACAAAACAAAATAGTTCAAATAATCAAGTT
AAAGCGACATCACTCTCAATTTTTGCTTTTTTTAGGTTTATAGAATAGAAATA
TAACAGAACGAAGCCTTTAGACTCTTTTTTTTTATTGGTATATTGAACAAAGA
AACTTTTTTTTTGTCCTCCCATATCTCGTGCACACAAATATTATGGATGCCGGT
GTGACTGAAAGTGGACTAAATGTGACTCTCACCATTCGGCTTCTTATGCACGG
AAAGGAAGTAGGAAGCATCATTGGGAAGAAAGGGGAGTCGGTTAAGAGGAT
CCGCGAGGAGAGTGGCGCGCGGATCAACATCTCGGAGGGGAATTGTCCGGA
GAGAATCATCACTCTGACCGGCCCCACCAATGCCATCTTTAAGGCTTTCGCTA
TGATCATCGACAAGCTGGAGGAAGATATCAACAGCTCCATGACCAACAGTAC
CGCGGCCAGCAGGCCCCCGGTCACCCTGAGGCTGGTGGTGCCGGCCACCCAG
TGCGGCTCCCTGATTGGGAAAGGCGGGTGTAAGATCAAAGAGATCCGCGAGA
GTACGGGGGCGCAGGTCCAGGTGGCGGGGGATATGCTGCCCAACTCCACCGA
GCGGGCCATCACCATCGCTGGCGTGCCGCAGTCTGTCACCGAGTGTGTCAAG
CAGATTTGCCTGGTCATGCTGGAGACGCTCTCCCAGTCTCCGCAAGGGAGAG
TCATGACCATTCCGTACCAGCCCATGCCGGCCAGCTCCCCAGTCATCTGCGCG
GGCGGCCAAGATCGGTGCAGCGACGCTGCGGGCTACCCCCATGCCACCCATG
ACCTGGAGGGACCACCTCTAGATGCCTACTCGATTCAAGGACAACACACCAT
TTCTCCGCTCGATCTGGCCAAGCTGAACCAGGTGGCAAGACAACAGTCTCAC
TTTGCCATGATGCACGGCGGGACCGGATTCGCCGGAATTGACTCCAGCTCTC
CAGAGGTGAAAGGCTATTGGGCAAGTTTGGATGCATCTACTCAAACCACCCA
TGAACTCACCATTCCAAATAACTTAATTGGCTGCATAATCGGGCGCCAAGGC
GCCAACATTAATGAGATCCGCCAGATGTCCGGGGCCCAGATCAAAATTGCCA
ACCCAGTGGAAGGCTCCTCTGGTAGGCAGGTTACTATCACTGGCTCTGCTGCC
AGTATTAGTCTGGCCCAGTATCTAATCAATGCCAGGCTTTCCTCTGAGAAGGG
CATGGGGTGCAGCTAGTAGCGTGTTACGCACCCAAACTTTTTATGAAAGTCTT
TGTTTATAATGATGAGGTTTATAAATATATAGTGGAGCAAAGATTAATCACTA
AATCAAGAAGCAGTACCAGTATTTTTTCTATATCAAGTAGTGATAATGGAAA
TAGCCCAAATTTGGCTTCCGTCGACACATAGAACGTTTGAGAGACATTATCAC
CATCAAGCATCGAGCCGCCCAAACCTAACCGTATAAGTTTTTTCACGTTTTTG
ATTTTTCCTTGCACACTTCGATATTACTCTCACGATAAAAGGGCCGAAGAGA
TCO89 gene expression cassette
TCO89p->TCO89 TC089t
SEQ ID NO. 2
TAAAAGAGTTTCCAAACCAGCAAGAAAGGGCAACAGAACTCGTCGAAGCAA
TACAAGTTCAGACACCAACCAAAATAGAAGGAGTGCTGATATAGGTACCGAC
AAACCAGTAAAGCCCAGATTACCCCCTCAAAGGACCTCATTAAACGAAATGA
GAAGAAGGGTATCCGCTATTTTGGAGTTCATTTCTAGAACTCAATGGGAATTG
AGTGAAGATCAGTCTGATCGAGAGGAATTTGTACGATTCGTGGAAAACCAGC
ATTTCGTAGAAAAAGTTGATACGATTTACAACGGTTATAATGAAAGTCTATC
AATGATGGACGACCTGACTAGAGAGTTACTACTATGGGAGAAAAAATATTCA
AATAACACTAATGCCATTCAATAAACGCAAAACACTGCAATATTATTCTCAA
CCAAAGTATAACTGTAATGAGGCGAACAAACACATCTATACATATATATACA
TCTATATGGATATAAAAACGACTAATTCAACGTTGTTTTTATCAACCGAGCTT
ACTCTTGTACGGGTAACCGCAAGGATAGCTAGTTGCGGATGGTATAGCGATT
TGGCTGGCACGATGATTAAGGAATCCAAACATCTAATGGACTAGCACATTCT
ATCGATTTACGGGTCAGGTAAACATAGATATTGGGATATATCATATATCCTTA
CTGAGTAACTATAATTATGGTTCATCGAGGAAGGACTTTGAAGTCAGACACT
GATGTAACATCTCTTAATGCGTCAACAGTATCACACCAGTCAAAGCCATTTAG
ACAGTTTTCGACTAGGTCGAGAGCAAAGAGTAACGCAAGCTTCAAAGGTTTG
CGTAGAGTTTTAACACATGATGGCACCCTGGATAATGATTATTTTAATAAGCA
CAACGTTTCTCAGAAATGCAAGAGTTCTGATGCACTTTTCAGAAAGCGAACG
ATTAGTGGGTTGAATATGACAGCTTTAACAAGAGTAAAGTCCAATCAAGGAA
AAAGATCAGCATCCTTTCATAGTCCGGTGCATAATACGCTGCTCAGTCCAAA
GAACAGCAGTCATTCTAATACTGGAACTGCTGGTTTCGGCCTGAAACCACGA
AGAAGTAAAAGTACCCAATCTGTTCTGAGTCTTCGAGATGCGCAAGAATCTA
AAAAGAGTGAATCTACTACTGACGAGGAGGTGGAATGTTTTTCGGAAGACAA
CATTGAAGATGGAAAGGTGAATAATGATAAAGTAATAGCCGAGCATGTTATG
CCTGAAGAAAAAAAGAATGTGCAGCAATTAAATCAGAATGAATTACAATCCC
CGGATTCAATAGATGAACAAGAAGAAGATAAATCAGGTACTGATGGAAAGG
AAAATCATAGAGCTGTATCCTTACCATTACCTCATTTATCTTCCAATAACTAT
TTCGGAGAATCAAGCCATTCTATAGAACATCAGAAAGATGGAGAAACATCTC
CAAGCTCAATTGAAACAAAACTGAATGCAACAAGTGTAATCAATGAAGAGG
GGCAATCAAAGGTGACGAAGGAAGCTGATATTGATGACTTGTCCAGCCATTC
TCAAAATTTGAGGGCCTCATTGGTTAAAGCGGGCGATAATATATCAGAAGCA
CCATATGATAAAGAAAAAAAAATTCTTGATGTTGGTAATACCTTAGCTGCAC
ATAAAAGTAATCAAAAACCAAGTCATTCAGATGAACAGTTTGATCAGGAAGA
TCACATTGATGCCCCTAGGAGTAATTCATCAAGAAAAAGCGACTCGAGCTTT
ATGTCTCTTAGGAGACAAAGTTCTAAACAACACAAATTATTAAACGAAGAAG
AAGATCTAATCAAGCCTGATGATATTTCTTCCGCTGGTACCAAGGATATTGAA
GGGCATAGCTTACTGGAAAATTATGCGCCTAATATGATTCTCTCCCAGTCGAC
TGGAGTTGAACGTAGATTTGAAAATTCATCATCCATCCAAAATTCGCTTGGGA
ATGAAATTCATGACTCGGGTGAGCATATGGCTTCAGGTGATACTTTTAATGAA
CTGGATGATGGCAAATTGCGCAAGAGCAAGAAAAATGGTGGAAGATCTCAA
CTTGGCCAAAATATACCGAACTCTCAGTCTACTTTCCCCACCATTGCTAACAT
CGGTAGTAAAGATAATAATGTACCACAGCACAACTTTTCGACCTCCATATCG
AGTTTAACCAATAATTTGAGGAGAGCTGCTCCTGAAAGCTTCCATGGTTCAA
GAATGAATAATATTTTTCACAAGAAAGGTAATCAGAATCTACTTCTGAGATC
CAACGATCTCAACAAAAATTCTGCAGCCCCGGCCTCTCCATTGTCCAACGAA
CATATTACATCTAGTACGAACTCCGGTAGCGATGCAAACAGACAATCCAACT
CAGGTGCCAAATTTAATAGCTTCGCCCAGTTCCTTAAATCAGATGGGATTGAT
GCAGAATCAAGAACACAAAGAAAATTATGGTTGCAGAGGGAGAATTCTATTA
TGGACTTAAGTTCACAAAATGACGGTAGTGACTCTATCTTTATGGCAGGAAA
CATTGATGCGAAAAGGGAGTTTGAGAGAATATCCCATGAATACTCTAATGTA
AAAAGATTTTACAACCCATTAGATGAAGCATTGTTGAGAGTACAACCTATAA
TAACGGGAAATGCAAATAATATCAGGAAAAAAAGCCATAACGATGCTCAGT
CAATCGCACATTCTAGCAGTGATACAGATCATAAGGATGAGGACGATTTGCT
CTTTACTAACTATGACAAAAAATTTGATGATCTTTATCCACATCTTGCAAGTG
CAAAGATTCAGGCAGTGTTGTCCGGTATATGGAAAAGCGAAAGTTACTTATT
TAACAAGGATGTTAATCCAATCAACAAGAATAGGACAACGAGTACAAACCA
CAGCGTTGGCCACACTGCTTCACAGAATGCACGTAACTTGCTGAGGGGCCCG
ATGGGTTCCAGCACGACTTTGCACCACCAACGCGTCATTAACTCTCTGCAGCC
GACTACGAGGGCAGTGAATCGCAGGATGGAAAATGTGGGCTACATGCATAC
ACAGCCACAACAAAGGTGAAAACAACCAACACGAAGCACACAGTTTAAAAG
AGTAGCTAATGCTTTCGAGGTAAAACGCGAAGTTCGTAGAGAGCGAATATGT
TTGGACACTTAAGGAACATACTATGTTTATGCACTATAAAAGGACATGTACTT
CTATACGAGCTAACGAGGCGTATTTGTATAACCCGGTTAACGTAATAAATGA
TAAATTATCGAACAAAAAGAAAGAAAAACGTTGAATGCAACCACCGCAAAT
TTAGCGATTTCGCTGGATTCCGTATCTCTTAAAAAATGGCATTAGTAAATATA
CCTTAAACGTGTCTATTCTTTTAGCAATTTTTTGGTAAGTATTCCTCACGGACT
ATAAATACTATCGGTCAGAATCACTACATTCAAATTAATCTTGTTTTACCGAG
TCTCTGACGTTTCATTTACAAGCTTGTCCTTACAAAAAACCTATTTTATTACTT
TAGTCCATTTTCCTTTCAAGTTAGATATTGTTTTCCCTTCACTTA
GPDp-peptide#1-Aga1-Cyc1t
SEQ ID No. 3
ATACTAGCGTTGAATGTTAGCGTCAACAACAAGAAGTTTAATGACGCGGAGG
CCAAGGCAAAAAGATTCCTTGATTACGTAAGGGAGTTAGAATCATTTTGAAT
AAAAAACACGCTTTTTCAGTTCGAGTTTATCATTATCAATACTGCCATTTCAA
AGAATACGTAAATAATTAATAGTAGTGATTTTCCTAACTTTATTTAGTCAAAA
AATTAGCCTTTTAATTCTGCTGTAACCCGTACATGCCCAAAATAGGGGGCGG
GTTACACAGAATATATAACATCGTAGGTGTCTGGGTGAACAGTTTATTCCTGG
CATCCACTAAATATAATGGAGCCCGCTTTTTAAGCTGGCATCCAGAAAAAAA
AAGAATCCCAGCACCAAAATATTGTTTTCTTCACCAACCATCAGTTCATAGGT
CCATTCTCTTAGCGCAACTACAGAGAACAGGGGCACAAACAGGCAAAAAAC
GGGCACAACCTCAATGGAGTGATGCAACCTGCCTGGAGTAAATGATGACACA
AGGCAATTGACCCACGCATGTATCTATCTCATTTTCTTACACCTTCTATTACCT
TCTGCTCTCTCTGATTTGGAAAAAGCTGAAAAAAAAGGTTGAAACCAGTTCC
CTGAAATTATTCCCCTACTTGACTAATAAGTATATAAAGACGGTAGGTATTGA
TTGTAATTCTGTAAATCTATTTCTTAAACTTCTTAAATTCTACTTTTATAGTTA
GTCTTTTTTTTAGTTTTAAAACACCAAGAACTTAGTTTCGAATAAACACACAT
AAACAAACAAAATGGATTCTCAAAAAACTAATCCATCTGATTCTCAAAAAAC
TAATCCATCTGATTCTCAAAAAACTAATCCATCTGATTCTCAAAAAACTAATC
CATCTGATTCTCAAAAAACTAATCCATCTGATTCTCAAAAAACTAATCCATCT
GATTCTCAAAAAACTAATCCATCTGATTCTCAAAAAACTAATCCATCTTAAact
acaatgtatacgacatggtgtccttatagctctgaatctgagactagcacattaaccagtatgcatgaaacggttacaacagacgct
acagtctgcactcacgagtcttgcatgccctcgcagacaacaagtttgattacatcttctataaaaatgtccactaaaaacgtcgca
acttctgtaagcacctcaacggttgaatcctcatatgcatgctccacatgtgctgaaacgtcacactcgtattcttccgtgcaaacag
cttcatcaagttctgtaacacagcagaccacatccacaaagagttgggtaagttcaatgacaacttcggatgaagatttcaataag
cacgctaccggtaagtatcatgtaacatcttcaggtacctcaaccatttcgactagtgtaagtgaagccacgagtacatcaagcatt
gactcagaatctcaagaacaatcatcacacttattatcgacatcggtcctttcatcctcctccttgtctgctacattatcctctgacagt
actattttgctattcagttctgtatcatcactaagtgtcgaacagtcaccagttaccacacttcaaatttcttcaacatcagagattttac
aacccacttcttccacagctattgctacaatatctgcctctacatcatcactttccgcaacatctatctctacaccatctacctctgtgg
aatcgactattgaatcttcatcattgactccgacggtatcttctattttcctctcatcatcatctgctccctcttctctacaaacatctgtta
ccactacagaagtttccactacttcaatctccatacaataccaaacttcatcaatggtaacaattagccaatatatgggcagtggatc
gcaaacgcgtttgccattaggaaagttggtcttcgccatcatggcagttgcttgcaatgtaattttcagttaaACAGGCCCC
TTTTCCTTTGTCGATATCATGTAATTAGTTATGTCACGCTTACATTCACGCCCT
CCTCCCACATCCGCTCTAACCGAAAAGGAAGGAGTTAGACAACCTGAAGTCT
AGGTCCCTATTTATTTTTTTTAATAGTTATGTTAGTATTAAGAACGTTATTTAT
ATTTCAAATTTTTCTTTTTTTTCTGTACAAACGCGTGTACGCATGTAACATTAT
ACTGAAAACCTTGCTTGAGAAGGTTTTGGGACGCTCGAAGGCTTTAATTTGCA
AGCTTCGCAGTTTACACTCTCATC
GPDp-peptide#2-Aga1-Cyc1t
SEQ ID NO. 4
ATACTAGCGTTGAATGTTAGCGTCAACAACAAGAAGTTTAATGACGCGGAGG
CCAAGGCAAAAAGATTCCTTGATTACGTAAGGGAGTTAGAATCATTTTGAAT
AAAAAACACGCTTTTTCAGTTCGAGTTTATCATTATCAATACTGCCATTTCAA
AGAATACGTAAATAATTAATAGTAGTGATTTTCCTAACTTTATTTAGTCAAAA
AATTAGCCTTTTAATTCTGCTGTAACCCGTACATGCCCAAAATAGGGGGCGG
GTTACACAGAATATATAACATCGTAGGTGTCTGGGTGAACAGTTTATTCCTGG
CATCCACTAAATATAATGGAGCCCGCTTTTTAAGCTGGCATCCAGAAAAAAA
AAGAATCCCAGCACCAAAATATTGTTTTCTTCACCAACCATCAGTTCATAGGT
CCATTCTCTTAGCGCAACTACAGAGAACAGGGGCACAAACAGGCAAAAAAC
GGGCACAACCTCAATGGAGTGATGCAACCTGCCTGGAGTAAATGATGACACA
AGGCAATTGACCCACGCATGTATCTATCTCATTTTCTTACACCTTCTATTACCT
TCTGCTCTCTCTGATTTGGAAAAAGCTGAAAAAAAAGGTTGAAACCAGTTCC
CTGAAATTATTCCCCTACTTGACTAATAAGTATATAAAGACGGTAGGTATTGA
TTGTAATTCTGTAAATCTATTTCTTAAACTTCTTAAATTCTACTTTTATAGTTA
GTCTTTTTTTTAGTTTTAAAACACCAAGAACTTAGTTTCGAATAAACACACAT
AAACAAACAAAATGATGCATGGTAAAACTCAAGCTACTTCTGGTACTATTCA
ATCTATGCATGGTAAAACTCAAGCTACTTCTGGTACTATTCAATCTATGCATG
GTAAAACTCAAGCTACTTCTGGTACTATTCAATCTATGCATGGTAAAACTCAA
GCTACTTCTGGTACTATTCAATCTATGCATGGTAAAACTCAAGCTACTTCTGG
TACTATTCAATCTATGCATGGTAAAACTCAAGCTACTTCTGGTACTATTCAAT
CTATGCATGGTAAAACTCAAGCTACTTCTGGTACTATTCAATCTTAAactacaatgt
atacgacatggtgtccttatagctctgaatctgagactagcacattaaccagtatgcatgaaacggttacaacagacgctacagtct
gcactcacgagtcttgcatgccctcgcagacaacaagtttgattacatcttctataaaaatgtccactaaaaacgtcgcaacttctgt
aagcacctcaacggttgaatcctcatatgcatgctccacatgtgctgaaacgtcacactcgtattcttccgtgcaaacagcttcatc
aagttctgtaacacagcagaccacatccacaaagagttgggtaagttcaatgacaacttcggatgaagatttcaataagcacgcta
ccggtaagtatcatgtaacatcttcaggtacctcaaccatttcgactagtgtaagtgaagccacgagtacatcaagcattgactcag
aatctcaagaacaatcatcacacttattatcgacatcggtcctttcatcctcctccttgtctgctacattatcctctgacagtactattttg
ctattcagttctgtatcatcactaagtgtcgaacagtcaccagttaccacacttcaaatttcttcaacatcagagattttacaacccact
tcttccacagctattgctacaatatctgcctctacatcatcactttccgcaacatctatctctacaccatctacctctgtggaatcgact
attgaatcttcatcattgactccgacggtatcttctattttcctctcatcatcatctgctccctcttctctacaaacatctgttaccactaca
gaagtttccactacttcaatctccatacaataccaaacttcatcaatggtaacaattagccaatatatgggcagtggatcgcaaacg
cgtttgccattaggaaagttggtcttcgccatcatggcagttgcttgcaatgtaattttcagttaaACAGGCCCCTTTTC
CTTTGTCGATATCATGTAATTAGTTATGTCACGCTTACATTCACGCCCTCCTCC
CACATCCGCTCTAACCGAAAAGGAAGGAGTTAGACAACCTGAAGTCTAGGTC
CCTATTTATTTTTTTTAATAGTTATGTTAGTATTAAGAACGTTATTTATATTTC
AAATTTTTCTTTTTTTTCTGTACAAACGCGTGTACGCATGTAACATTATACTGA
AAACCTTGCTTGAGAAGGTTTTGGGACGCTCGAAGGCTTTAATTTGCAAGCTT
CGCAGTTTACACTCTCATC
FTH SYNTHESIS Component
SEQ ID No. 5
atgACAACTGCTTCTACATCACAAGTTAGACAAAATTATCATCAAGATTCTGAA
GCTGCAATTAATAGACAAATTAATTTGGAATTATACGCTTCATACGTTTATTT
GTCAATGTCTTATTACTTTGATAGAGATGATGTTGCTTTGAAAAATTTTGCTA
AATACTTTTTGCATCAATCTCATGAAGAAAGAGAACATGCTGAAAAATTGAT
GAAATTACAAAATCAAAGAGGTGGTAGAATTTTCTTGCAAGATATTAAAAAG
CCAGATTGTGATGATTGGGAATCAGGTTTAAATGCAATGGAATGTGCTTTAC
ATTTGGAAAAGAATGTTAATCAATCTTTGTTAGAATTGCATAAATTGGCTACT
GATAAAAATGATCCACATTTGTGTGATTTTATTGAAACTCATTATTTGAATGA
ACAAGTTAAAGCTATTAAAGAATTGGGTGACCATGTTACTAATTTGAGAAAA
ATGGGTGCACCAGAATCAGGTTTGGCTGAATACTTATTTGATAAACATACTTT
GGGTGACTCAGATAATGAATCAtaa
FTL SYNTHESIS Component
SEQ ID No. 6
atgACAACTGCTTCTACATCACAAGTTAGACAAAATTATCATCAAGATTCTGAA
GCTGCAATTAATAGACAAATTAATTTGGAATTATACGCTTCATACGTTTATTT
GTCAATGTCTTATTACTTTGATAGAGATGATGTTGCTTTGAAAAATTTTGCTA
AATACTTTTTGCATCAATCTCATGAAGAAAGAGAACATGCTGAAAAATTGAT
GAAATTACAAAATCAAAGAGGTGGTAGAATTTTCTTGCAAGATATTAAAAAG
CCAGATTGTGATGATTGGGAATCAGGTTTAAATGCAATGGAATGTGCTTTAC
ATTTGGAAAAGAATGTTAATCAATCTTTGTTAGAATTGCATAAATTGGCTACT
GATAAAAATGATCCACATTTGTGTGATTTTATTGAAACTCATTATTTGAATGA
ACAAGTTAAAGCTATTAAAGAATTGGGTGACCATGTTACTAATTTGAGAAAA
ATGGGTGCACCAGAATCAGGTTTGGCTGAATACTTATTTGATAAACATACTTT
GGGTGACTCAGATAATGAATCAtaa
PCBP1 SYNTHESIS Component
SEQ ID No. 7
atgGATGCTGGTGTTACAGAATCTGGTTTGAATGTTACATTGACTATTAGATTG
TTAATGCATGGTAAAGAAGTTGGTTCAATTATTGGTAAAAAGGGTGAATCTG
TTAAAAGAATTAGAGAAGAATCTGGTGCTAGAATTAATATTTCTGAAGGTAA
TTGTCCAGAAAGAATTATTACTTTAACAGGTCCAACTAATGCTATTTTTAAAG
CATTTGCTATGATTATTGATAAATTGGAAGAAGATATTAATTCATCTATGACT
AATTCAACAGCTGCATCAAGACCACCAGTTACTTTGAGATTAGTTGTTCCAGC
AACACAATGTGGTTCTTTGATTGGTAAAGGTGGTTGTAAAATTAAAGAAATT
AGAGAATCTACTGGTGCACAAGTTCAAGTTGCAGGTGACATGTTGCCAAATT
CTACTGAAAGAGCAATTACAATTGCTGGTGTTCCACAATCAGTTACTGAATGT
GTTAAACAAATTTGTTTAGTTATGTTGGAAACATTGTCTCAATCACCACAAGG
TAGAGTTATGACTATTCCATACCAACCAATGCCAGCATCTTCACCAGTTATTT
GTGCTGGTGGTCAAGATAGATGTTCAGATGCTGCAGGTTATCCACATGCAAC
ACATGATTTGGAAGGTCCACCATTGGATGCTTATTCAATTCAAGGTCAACATA
CTATTTCACCATTAGATTTGGCTAAATTGAATCAAGTTGCTAGACAACAATCA
CATTTTGCTATGATGCATGGTGGTACAGGTTTTGCTGGTATTGATTCATCTTCA
CCAGAAGTTAAAGGTTATTGGGCTTCTTTAGATGCTTCTACTCAAACAACTCA
TGAATTGACTATTCCAAATAATTTGATTGGTTGTATTATTGGTAGACAAGGTG
CAAATATTAATGAAATTAGACAAATGTCTGGTGCTCAAATTAAAATTGCAAA
TCCAGTTGAAGGTTCATCTGGTAGACAAGTTACAATTACTGGTTCTGCTGCAT
CTATTTCATTGGCACAATACTTAATTAATGCAAGATTGTCATCTGAAAAAGGT
ATGGGTTGTTCTtaa
Peptide 1 Synthesis
SEQ ID No. 8
ATGGATTCTCAAAAGACAAATCCATCAGATTCTCAAAAGACTAATCCATCAG
ATTCTCAAAAGACAAATCCATCTGATTCTCAAAAGACTAATCCATCTGATTCA
CAAAAGACTAATCCATCAGATTCACAAAAGACAAATCCATCTGATTCACAAA
AGACAAATCCATCAGATTCACAAAAGACTAATCCATCTGGTGGTGGTGGTTC
TGGTGGTGGTGGTTCTGGTGGTGGTGGTTCTGCTTCTGGTGGTGGT
Peptide 2 Synthesis
SEQ ID No. 9
ATGCATGGTAAAACACAAGCTACTTCAGGTACTATTCAATCTATGCATGGTA
AAACACAAGCAACATCTGGTACAATTCAATCTATGCATGGTAAAACTCAAGC
AACTTCTGGTACTATTCAATCTATGCATGGTAAAACTCAAGCTACTTCTGGTA
CAATTCAATCAATGCATGGTAAAACTCAAGCTACATCTGGTACTATTCAATCA
ATGCATGGTAAAACACAAGCTACTTCAGGTACAATTCAATCAATGCATGGTA
AAACACAAGCAACATCAGGTACTATTCAATCTGGTGGTGGTGGTTCTGGTGG
TGGTGGTTCTGGTGGTGGTGGTTCTGCTTCTGGTGGTGGT
pRS316-FTL-FTH-PCBP1
SEQ ID No. 10
tcgcgcgtttcggtgatgacggtgaaaacctctgacacatgcagctcccggagacggtcacagcttgtctgtaagcggatgccg
ggagcagacaagcccgtcagggcgcgtcagcgggtgttggcgggtgtcggggctggcttaactatgcggcatcagagcagat
tgtactgagagtgcaccacgcttttcaattcaattcatcattttttttttattcttttttttgatttcggtttctttgaaatttttttgattcggtaat
ctccgaacagaaggaagaacgaaggaaggagcacagacttagattggtatatatacgcatatgtagtgttgaagaaacatgaaa
ttgcccagtattcttaacccaactgcacagaacaaaaacctgcaggaaacgaagataaatcatgtcgaaagctacatataaggaa
cgtgctgctactcatcctagtcctgttgctgccaagctatttaatatcatgcacgaaaagcaaacaaacttgtgtgcttcattggatgt
tcgtaccaccaaggaattactggagttagttgaagcattaggtcccaaaatttgtttactaaaaacacatgtggatatcttgactgatt
tttccatggagggcacagttaagccgctaaaggcattatccgccaagtacaattttttactcttcgaagacagaaaatttgctgacat
tggtaatacagtcaaattgcagtactctgcgggtgtatacagaatagcagaatgggcagacattacgaatgcacacggtgtggtg
ggcccaggtattgttagcggtttgaagcaggcggcagaagaagtaacaaaggaacctagaggccttttgatgttagcagaattgt
catgcaagggctccctatctactggagaatatactaagggtactgttgacattgcgaagagcgacaaagattttgttatcggctttat
tgctcaaagagacatgggtggaagagatgaaggttacgattggttgattatgacacccggtgtgggtttagatgacaagggaga
cgcattgggtcaacagtatagaaccgtggatgatgtggtctctacaggatctgacattattattgttggaagaggactatttgcaaa
gggaagggatgctaaggtagagggtgaacgttacagaaaagcaggctgggaagcatatttgagaagatgcggccagcaaaa
ctaaaaaactgtattataagtaaatgcatgtatactaaactcacaaattagagcttcaatttaattatatcagttattaccctgcggtgtg
aaataccgcacagatgcgtaaggagaaaataccgcatcaggaaattgtaaacgttaatattttgttaaaattcgcgttaaatttttgtt
aaatcagctcattttttaaccaataggccgaaatcggcaaaatcccttataaatcaaaagaatagaccgagatagggttgagtgttg
ttccagtttggaacaagagtccactattaaagaacgtggactccaacgtcaaagggcgaaaaaccgtctatcagggcgatggcc
cactacgtgaaccatcaccctaatcaagttttttggggtcgaggtgccgtaaagcactaaatcggaaccctaaagggagccccc
gatttagagcttgacggggaaagccggcgaacgtggcgagaaaggaagggaagaaagcgaaaggagcgggcgctagggc
gctggcaagtgtagcggtcacgctgcgcgtaaccaccacacccgccgcgcttaatgcgccgctacagggcgcgtcgcgccat
tcgccattcaggctgcgcaactgttgggaagggcgatcggtgcgggcctcttcgctattacgccagctggcgaaggggggatg
tgctgcaaggcgattaagttgggtaacgccagggttttcccagtcacgacgttgtaaaacgacggccagtgaattgtaatacgac
tcactatagggcgaattggagctccaccgcggtggcggccgcAACCCCTCAGCGTCAGGAAGACGCC
ACGGATCCAACAACATCATCGACGAATGAATTATCTGCCGCTGAGCCAACAA
TGGTCACTTCGACACATGCCACTAAGACAATACAGGCTCAAACACAAGATCC
TCCCACGAAGCACAAGAAGTCCAGTTTCTTCACTAAACTGTTCAAGAAAAAA
AGCAGCCGTTAGCAGTTGTTTACTCGAATTTTGCAATCAACCCTAATTTTTGA
AGCCTGGTTTTAGATTTATTCTCTTCTTTTTCTTCTTGTGAACTTCAATTACTA
ATGTAACTTAATTTTTAATATAACTTTTACAGTTTAATAATATTGATTTTTTTC
GGTCTGGACCAATCGCGCCGCATTTCTCACTAATATTACTAACATACCCTCTT
CTCATTGGCTCGGTACCCCTTTCGTGACCCGCATTTTTTGTTTTCTTTGTTAGC
CCGAATGTCTCACAATGAAGATGTAAAATTAAGATTATATATGAAAAATTGA
TACAAAACAAAATAGTTCAAATAATCAAGTTAAAGCGACATCACTCTCAATT
TTTGCTTTTTTTAGGTTTATAGAATAGAAATATAACAGAACGAAGCCTTTAGA
CTCTTTTTTTTTATTGGTATATTGAACAAAGAAACTTTTTTTTTGTCCTCCCAT
ATCTCGTGCACACAAATATTatgTCATCTCAAATTAGACAAAATTACTCTACTG
ATGTTGAAGCTGCAGTTAATTCTTTGGTTAATTTGTACTTACAAGCATCATAC
ACATATTTGTCTTTAGGTTTTTACTTTGATAGAGATGATGTTGCTTTAGAAGGT
GTTTCTCATTTCTTTAGAGAATTGGCTGAAGAAAAGAGAGAAGGTTACGAAA
GATTGTTAAAAATGCAAAATCAAAGAGGTGGTAGAGCTTTGTTTCAAGATAT
TAAAAAGCCAGCTGAAGATGAATGGGGTAAAACACCAGATGCTATGAAAGC
AGCTATGGCATTGGAAAAGAAATTGAATCAAGCATTGTTAGATTTGCATGCT
TTAGGTTCTGCTAGAACAGATCCACATTTGTGTGATTTCTTGGAAACTCATTT
CTTGGATGAAGAAGTTAAATTGATTAAAAAGATGGGTGACCATTTGACTAAT
TTGCATAGATTGGGTGGTCCAGAAGCTGGTTTGGGTGAATACTTGTTTGAAAG
ATTAACTTTGAAACATGATtaaGTGTAAAGTAACAACACTATACATATTTATTG
TAAAGAAATTTGGGATTGAGAAGCTTTGCTATATACTTGGAACTGGGCTGGA
TTTTCTCGACATACATTTCTTTTACGTTTTAATTTGTTTCTATATTCTCCTCTTT
AAATTTATTTATATTAATGAATTTCAAACTAGTTTCTTTTTCTATTGCCAAGAG
GCCCATCAGGTGATCCATGATAACCTTTATTCGCATTGAATCGTTTTTCATTG
GATCTAATTCGTCATTTGGTCGCCGCCTTTCTTTGCTCCTTCTGCTTTTTTCTTT
TTCTCTTTGACTATCAGGTCATAAGATCTTCTCCTCCATTATGCCCAAGTGTTT
TTCTTTTTTTTGCCTTTATATAAATTACAAAATACATACATATACTTCTCGTGC
AGTATACTGGAATCAGCTTTATACCATATGAAGCACAACTTGGATCAGGACC
GCTCTTCATTAAAAATCGAAGAATTATCTAAATAATTAAtgtttttaatgctgatttcctataat
attaaccggtAACCCCTCAGCGTCAGGAAGACGCCACGGATCCAACAACATCATCG
ACGAATGAATTATCTGCCGCTGAGCCAACAATGGTCACTTCGACACATGCCA
CTAAGACAATACAGGCTCAAACACAAGATCCTCCCACGAAGCACAAGAAGT
CCAGTTTCTTCACTAAACTGTTCAAGAAAAAAAGCAGCCGTTAGCAGTTGTTT
ACTCGAATTTTGCAATCAACCCTAATTTTTGAAGCCTGGTTTTAGATTTATTCT
CTTCTTTTTCTTCTTGTGAACTTCAATTACTAATGTAACTTAATTTTTAATATA
ACTTTTACAGTTTAATAATATTGATTTTTTTCGGTCTGGACCAATCGCGCCGC
ATTTCTCACTAATATTACTAACATACCCTCTTCTCATTGGCTCGGTACCCCTTT
CGTGACCCGCATTTTTTGTTTTCTTTGTTAGCCCGAATGTCTCACAATGAAGAT
GTAAAATTAAGATTATATATGAAAAATTGATACAAAACAAAATAGTTCAAAT
AATCAAGTTAAAGCGACATCACTCTCAATTTTTGCTTTTTTTAGGTTTATAGA
ATAGAAATATAACAGAACGAAGCCTTTAGACTCTTTTTTTTTATTGGTATATT
GAACAAAGAAACTTTTTTTTTGTCCTCCCATATCTCGTGCACACAAATATTatg
ACAACTGCTTCTACATCACAAGTTAGACAAAATTATCATCAAGATTCTGAAG
CTGCAATTAATAGACAAATTAATTTGGAATTATACGCTTCATACGTTTATTTG
TCAATGTCTTATTACTTTGATAGAGATGATGTTGCTTTGAAAAATTTTGCTAA
ATACTTTTTGCATCAATCTCATGAAGAAAGAGAACATGCTGAAAAATTGATG
AAATTACAAAATCAAAGAGGTGGTAGAATTTTCTTGCAAGATATTAAAAAGC
CAGATTGTGATGATTGGGAATCAGGTTTAAATGCAATGGAATGTGCTTTACAT
TTGGAAAAGAATGTTAATCAATCTTTGTTAGAATTGCATAAATTGGCTACTGA
TAAAAATGATCCACATTTGTGTGATTTTATTGAAACTCATTATTTGAATGAAC
AAGTTAAAGCTATTAAAGAATTGGGTGACCATGTTACTAATTTGAGAAAAAT
GGGTGCACCAGAATCAGGTTTGGCTGAATACTTATTTGATAAACATACTTTGG
GTGACTCAGATAATGAATCAtaaGTGTAAAGTAACAACACTATACATATTTATT
GTAAAGAAATTTGGGATTGAGAAGCTTTGCTATATACTTGGAACTGGGCTGG
ATTTTCTCGACATACATTTCTTTTACGTTTTAATTTGTTTCTATATTCTCCTCTT
TAAATTTATTTATATTAATGAATTTCAAACTAGTTTCTTTTTCTATTGCCAAGA
GGCCCATCAGGTGATCCATGATAACCTTTATTCGCATTGAATCGTTTTTCATT
GGATCTAATTCGTCATTTGGTCGCCGCCTTTCTTTGCTCCTTCTGCTTTTTTCTT
TTTCTCTTTGACTATCAGGTCATAAGATCTTCTCCTCCATTATGCCCAAGTGTT
TTTCTTTTTTTTGCCTTTATATAAATTACAAAATACATACATATACTTCTCGTG
CAGTATACTGGAATCAGCTTTATACCATATGAAGCACAACTTGGATCAGGAC
CGCTCTTCATTAAAAATCGAAGAATTATCTAAATAATTAAatccgctctaaccgaaaagg
aaggagttaggaattcAACCCCTCAGCGTCAGGAAGACGCCACGGATCCAACAACATC
ATCGACGAATGAATTATCTGCCGCTGAGCCAACAATGGTCACTTCGACACAT
GCCACTAAGACAATACAGGCTCAAACACAAGATCCTCCCACGAAGCACAAG
AAGTCCAGTTTCTTCACTAAACTGTTCAAGAAAAAAAGCAGCCGTTAGCAGT
TGTTTACTCGAATTTTGCAATCAACCCTAATTTTTGAAGCCTGGTTTTAGATTT
ATTCTCTTCTTTTTCTTCTTGTGAACTTCAATTACTAATGTAACTTAATTTTTAA
TATAACTTTTACAGTTTAATAATATTGATTTTTTTCGGTCTGGACCAATCGCGC
CGCATTTCTCACTAATATTACTAACATACCCTCTTCTCATTGGCTCGGTACCCC
TTTCGTGACCCGCATTTTTTGTTTTCTTTGTTAGCCCGAATGTCTCACAATGAA
GATGTAAAATTAAGATTATATATGAAAAATTGATACAAAACAAAATAGTTCA
AATAATCAAGTTAAAGCGACATCACTCTCAATTTTTGCTTTTTTTAGGTTTATA
GAATAGAAATATAACAGAACGAAGCCTTTAGACTCTTTTTTTTTATTGGTATA
TTGAACAAAGAAACTTTTTTTTTGTCCTCCCATATCTCGTGCACACAAATATTa
tgGATGCTGGTGTTACAGAATCTGGTTTGAATGTTACATTGACTATTAGATTGT
TAATGCATGGTAAAGAAGTTGGTTCAATTATTGGTAAAAAGGGTGAATCTGT
TAAAAGAATTAGAGAAGAATCTGGTGCTAGAATTAATATTTCTGAAGGTAAT
TGTCCAGAAAGAATTATTACTTTAACAGGTCCAACTAATGCTATTTTTAAAGC
ATTTGCTATGATTATTGATAAATTGGAAGAAGATATTAATTCATCTATGACTA
ATTCAACAGCTGCATCAAGACCACCAGTTACTTTGAGATTAGTTGTTCCAGCA
ACACAATGTGGTTCTTTGATTGGTAAAGGTGGTTGTAAAATTAAAGAAATTA
GAGAATCTACTGGTGCACAAGTTCAAGTTGCAGGTGACATGTTGCCAAATTC
TACTGAAAGAGCAATTACAATTGCTGGTGTTCCACAATCAGTTACTGAATGTG
TTAAACAAATTTGTTTAGTTATGTTGGAAACATTGTCTCAATCACCACAAGGT
AGAGTTATGACTATTCCATACCAACCAATGCCAGCATCTTCACCAGTTATTTG
TGCTGGTGGTCAAGATAGATGTTCAGATGCTGCAGGTTATCCACATGCAACA
CATGATTTGGAAGGTCCACCATTGGATGCTTATTCAATTCAAGGTCAACATAC
TATTTCACCATTAGATTTGGCTAAATTGAATCAAGTTGCTAGACAACAATCAC
ATTTTGCTATGATGCATGGTGGTACAGGTTTTGCTGGTATTGATTCATCTTCAC
CAGAAGTTAAAGGTTATTGGGCTTCTTTAGATGCTTCTACTCAAACAACTCAT
GAATTGACTATTCCAAATAATTTGATTGGTTGTATTATTGGTAGACAAGGTGC
AAATATTAATGAAATTAGACAAATGTCTGGTGCTCAAATTAAAATTGCAAAT
CCAGTTGAAGGTTCATCTGGTAGACAAGTTACAATTACTGGTTCTGCTGCATC
TATTTCATTGGCACAATACTTAATTAATGCAAGATTGTCATCTGAAAAAGGTA
TGGGTTGTTCTtaaGTGTAAAGTAACAACACTATACATATTTATTGTAAAGAAA
TTTGGGATTGAGAAGCTTTGCTATATACTTGGAACTGGGCTGGATTTTCTCGA
CATACATTTCTTTTACGTTTTAATTTGTTTCTATATTCTCCTCTTTAAATTTATT
TATATTAATGAATTTCAAACTAGTTTCTTTTTCTATTGCCAAGAGGCCCATCA
GGTGATCCATGATAACCTTTATTCGCATTGAATCGTTTTTCATTGGATCTAATT
CGTCATTTGGTCGCCGCCTTTCTTTGCTCCTTCTGCTTTTTTCTTTTTCTCTTTG
ACTATCAGGTCATAAGATCTTCTCCTCCATTATGCCCAAGTGTTTTTCTTTTTT
TTGCCTTTATATAAATTACAAAATACATACATATACTTCTCGTGCAGTATACT
GGAATCAGCTTTATACCATATGAAGCACAACTTGGATCAGGACCGCTCTTCAT
TAAAAATCGAAGAATTATCTAAATAATTAActcgagggggggcccggtacccagcttttgttccctt
tagtgagggttaattccgagcttggcgtaatcatggtcatagctgtttcctgtgtgaaattgttatccgctcacaattccacacaacat
aggagccggaagcataaagtgtaaagcctggggtgcctaatgagtgaggtaactcacattaattgcgttgcgctcactgcccgct
ttccagtcgggaaacctgtcgtgccagctgcattaatgaatcggccaacgcgcggggagaggcggtttgcgtattgggcgctct
tccgcttcctcgctcactgactcgctgcgctcggtcgttcggctgcggcgagcggtatcagctcactcaaaggcggtaatacggt
tatccacagaatcaggggataacgcaggaaagaacatgtgagcaaaaggccagcaaaaggccaggaaccgtaaaaaggcc
gcgttgctggcgtttttccataggctcggcccccctgacgagcatcacaaaaatcgacgctcaagtcagaggtggcgaaacccg
acaggactataaagataccaggcgttcccccctggaagctccctcgtgcgctctcctgttccgaccctgccgcttaccggatacc
tgtccgcctttctcccttcgggaagcgtggcgctttctcaatgctcacgctgtaggtatctcagttcggtgtaggtcgttcgctccaa
gctgggctgtgtgcacgaaccccccgttcagcccgaccgctgcgccttatccggtaactatcgtcttgagtccaacccggtaaga
cacgacttatcgccactggcagcagccactggtaacaggattagcagagcgaggtatgtaggcggtgctacagagttcttgaag
tggtggcctaactacggctacactagaaggacagtatttggtatctgcgctctgctgaagccagttaccttcggaaaaagagttgg
tagctcttgatccggcaaacaaaccaccgctggtagcggtggtttttttgtttgcaagcagcagattacgcgcagaaaaaaaggat
ctcaagaagatcctttgatcttttctacggggtctgacgctcagtggaacgaaaactcacgttaagggattttggtcatgagattatc
aaaaaggatcttcacctagatccttttaaattaaaaatgaagttttaaatcaatctaaagtatatatgagtaaacttggtctgacagtta
ccaatgcttaatcagtgaggcacctatctcagcgatctgtctatttcgttcatccatagttgcctgactgcccgtcgtgtagataacta
cgatacgggagggcttaccatctggccccagtgctgcaatgataccgcgagacccacgctcaccggctccagatttatcagcaa
taaaccagccagccggaagggccgagcgcagaagtggtcctgcaactttatccgcctccatccagtctattaattgttgccggga
agctagagtaagtagttcgccagttaatagtttgcgcaacgttgttgccattgctacaggcatcgtggtgtcacgctcgtcgtttggt
atggcttcattcagctccggttcccaacgatcaaggcgagttacatgatcccccatgttgtgaaaaaaagcggttagctccttcggt
cctccgatcgttgtcagaagtaagttggccgcagtgttatcactcatggttatggcagcactgcataattctcttactgtcatgccatc
cgtaagatgcttttctgtgactggtgagtactcaaccaagtcattctgagaatagtgtatgcggcgaccgagttgctcttgcccggc
gtcaatacgggataataccgcgccacatagcagaactttaaaagtgctcatcattggaaaacgttcttcggggcgaaaactctca
aggatcttaccgctgttgagatccagttcgatgtaacccactcgtgcacccaactgatcttcagcatcttttactttcaccagcgtttct
gggtgagcaaaaacaggaaggcaaaatgccgcaaaaaagggaataagggcgacacggaaatgttgaatactcatactcttcct
ttttcaatattattgaagcatttatcagggttattgtctcatgagcggatacatatttgaatgtatttagaaaaataaacaaataggggtt
ccgcgcacatttccccgaaaagtgccacctgggtccttttcatcacgtgctataaaaataattataatttaaattttttaatataaatata
taaattaaaaatagaaagtaaaaaaagaaattaaagaaaaaatagtttttgttttccgaagatgtaaaagactctagggggatcgcc
aacaaatactaccttttatcttgctcttcctgctctcaggtattaatgccgaattgtttcatcttgtctgtgtagaagaccacacacgaaa
atcctgtgattttacattttacttatcgttaatcgaatgtatatctatttaatctgatttcttgtctaataaatatatatgtaaagtacgcttttt
gttgaaattttttaaacctttgtttatttttttttcttcattccgtaactcttctaccttctttatttactttctaaaatccaaatacaaaacataaa
aataaataaacacagagtaaattcccaaattattccatcattaaaagatacgaggcgcgtgtaagttacaggcaagcgatccgtcc
taagaaaccattattatcatgacattaacctataaaaataggcgtatcacgaggccctttcgtc
pRS423-TC089
SEQ ID No. 11
TCGCGCGTTTCGGTGATGACGGTGAAAACCTCTGACACATGCAGCTCCCGGA
GACGGTCACAGCTTGTCTGTAAGCGGATGCCGGGAGCAGACAAGCCCGTCAG
GGCGCGTCAGCGGGTGTTGGCGGGTGTCGGGGCTGGCTTAACTATGCGGCAT
CAGAGCAGATTGTACTGAGAGTGCACCATAAATTCCCGTTTTAAGAGCTTGG
TGAGCGCTAGGAGTCACTGCCAGGTATCGTTTGAACACGGCATTAGTCAGGG
AAGTCATAACACAGTCCTTTCCCGCAATTTTCTTTTTCTATTACTCTTGGCCTC
CTCTAGTACACTCTATATTTTTTTATGCCTCGGTAATGATTTTCATTTTTTTTTT
TCCCCTAGCGGATGACTCTTTTTTTTTCTTAGCGATTGGCATTATCACATAATG
AATTATACATTATATAAAGTAATGTGATTTCTTCGAAGAATATACTAAAAAAT
GAGCAGGCAAGATAAACGAAGGCAAAGATGACAGAGCAGAAAGCCCTAGTA
AAGCGTATTACAAATGAAACCAAGATTCAGATTGCGATCTCTTTAAAGGGTG
GTCCCCTAGCGATAGAGCACTCGATCTTCCCAGAAAAAGAGGCAGAAGCAGT
AGCAGAACAGGCCACACAATCGCAAGTGATTAACGTCCACACAGGTATAGG
GTTTCTGGACCATATGATACATGCTCTGGCCAAGCATTCCGGCTGGTCGCTAA
TCGTTGAGTGCATTGGTGACTTACACATAGACGACCATCACACCACTGAAGA
CTGCGGGATTGCTCTCGGTCAAGCTTTTAAAGAGGCCCTACTGGCGCGTGGA
GTAAAAAGGTTTGGATCAGGATTTGCGCCTTTGGATGAGGCACTTTCCAGAG
CGGTGGTAGATCTTTCGAACAGGCCGTACGCAGTTGTCGAACTTGGTTTGCAA
AGGGAGAAAGTAGGAGATCTCTCTTGCGAGATGATCCCGCATTTTCTTGAAA
GCTTTGCAGAGGCTAGCAGAATTACCCTCCACGTTGATTGTCTGCGAGGCAA
GAATGATCATCACCGTAGTGAGAGTGCGTTCAAGGCTCTTGCGGTTGCCATA
AGAGAAGCCACCTCGCCCAATGGTACCAACGATGTTCCCTCCACCAAAGGTG
TTCTTATGTAGTGACACCGATTATTTAAAGCTGCAGCATACGATATATATACA
TGTGTATATATGTATACCTATGAATGTCAGTAAGTATGTATACGAACAGTATG
ATACTGAAGATGACAAGGTAATGCATCATTCTATACGTGTCATTCTGAACGA
GGCGCGCTTTCCTTTTTTCTTTTTGCTTTTTCTTTTTTTTTCTCTTGAACTCGAC
GGATCTATGCGGTGTGAAATACCGCACAGATGCGTAAGGAGAAAATACCGCA
TCAGGAAATTGTAAACGTTAATATTTTGTTAAAATTCGCGTTAAATTTTTGTT
AAATCAGCTCATTTTTTAACCAATAGGCCGAAATCGGCAAAATCCCTTATAA
ATCAAAAGAATAGACCGAGATAGGGTTGAGTGTTGTTCCAGTTTGGAACAAG
AGTCCACTATTAAAGAACGTGGACTCCAACGTCAAAGGGCGAAAAACCGTCT
ATCAGGGCGATGGCCCACTACGTGAACCATCACCCTAATCAAGTTTTTTGGG
GTCGAGGTGCCGTAAAGCACTAAATCGGAACCCTAAAGGGAGCCCCCGATTT
AGAGCTTGACGGGGAAAGCCGGCGAACGTGGCGAGAAAGGAAGGGAAGAA
AGCGAAAGGAGCGGGCGCTAGGGCGCTGGCAAGTGTAGCGGTCACGCTGCG
CGTAACCACCACACCCGCCGCGCTTAATGCGCCGCTACAGGGCGCGTCGCGC
CATTCGCCATTCAGGCTGCGCAACTGTTGGGAAGGGCGATCGGTGCGGGCCT
CTTCGCTATTACGCCAGCTGGCGAAAGGGGGATGTGCTGCAAGGCGATTAAG
TTGGGTAACGCCAGGGTTTTCCCAGTCACGACGTTGTAAAACGACGGCCAGT
GAGCGCGCGTAATACGACTCACTATAGGGCGAATTGGGTACCGGGCCCCCCC
TCGAGGTCGACGGTATCGATAAGCTTGATTAAAAGAGTTTCCAAACCAGCAA
GAAAGGGCAACAGAACTCGTCGAAGCAATACAAGTTCAGACACCAACCAAA
ATAGAAGGAGTGCTGATATAGGTACCGACAAACCAGTAAAGCCCAGATTACC
CCCTCAAAGGACCTCATTAAACGAAATGAGAAGAAGGGTATCCGCTATTTTG
GAGTTCATTTCTAGAACTCAATGGGAATTGAGTGAAGATCAGTCTGATCGAG
AGGAATTTGTACGATTCGTGGAAAACCAGCATTTCGTAGAAAAAGTTGATAC
GATTTACAACGGTTATAATGAAAGTCTATCAATGATGGACGACCTGACTAGA
GAGTTACTACTATGGGAGAAAAAATATTCAAATAACACTAATGCCATTCAAT
AAACGCAAAACACTGCAATATTATTCTCAACCAAAGTATAACTGTAATGAGG
CGAACAAACACATCTATACATATATATACATCTATATGGATATAAAAACGAC
TAATTCAACGTTGTTTTTATCAACCGAGCTTACTCTTGTACGGGTAACCGCAA
GGATAGCTAGTTGCGGATGGTATAGCGATTTGGCTGGCACGATGATTAAGGA
ATCCAAACATCTAATGGACTAGCACATTCTATCGATTTACGGGTCAGGTAAA
CATAGATATTGGGATATATCATATATCCTTACTGAGTAACTATAATTATGGTT
CATCGAGGAAGGACTTTGAAGTCAGACACTGATGTAACATCTCTTAATGCGT
CAACAGTATCACACCAGTCAAAGCCATTTAGACAGTTTTCGACTAGGTCGAG
AGCAAAGAGTAACGCAAGCTTCAAAGGTTTGCGTAGAGTTTTAACACATGAT
GGCACCCTGGATAATGATTATTTTAATAAGCACAACGTTTCTCAGAAATGCA
AGAGTTCTGATGCACTTTTCAGAAAGCGAACGATTAGTGGGTTGAATATGAC
AGCTTTAACAAGAGTAAAGTCCAATCAAGGAAAAAGATCAGCATCCTTTCAT
AGTCCGGTGCATAATACGCTGCTCAGTCCAAAGAACAGCAGTCATTCTAATA
CTGGAACTGCTGGTTTCGGCCTGAAACCACGAAGAAGTAAAAGTACCCAATC
TGTTCTGAGTCTTCGAGATGCGCAAGAATCTAAAAAGAGTGAATCTACTACT
GACGAGGAGGTGGAATGTTTTTCGGAAGACAACATTGAAGATGGAAAGGTG
AATAATGATAAAGTAATAGCCGAGCATGTTATGCCTGAAGAAAAAAAGAAT
GTGCAGCAATTAAATCAGAATGAATTACAATCCCCGGATTCAATAGATGAAC
AAGAAGAAGATAAATCAGGTACTGATGGAAAGGAAAATCATAGAGCTGTAT
CCTTACCATTACCTCATTTATCTTCCAATAACTATTTCGGAGAATCAAGCCAT
TCTATAGAACATCAGAAAGATGGAGAAACATCTCCAAGCTCAATTGAAACAA
AACTGAATGCAACAAGTGTAATCAATGAAGAGGGGCAATCAAAGGTGACGA
AGGAAGCTGATATTGATGACTTGTCCAGCCATTCTCAAAATTTGAGGGCCTCA
TTGGTTAAAGCGGGCGATAATATATCAGAAGCACCATATGATAAAGAAAAAA
AAATTCTTGATGTTGGTAATACCTTAGCTGCACATAAAAGTAATCAAAAACC
AAGTCATTCAGATGAACAGTTTGATCAGGAAGATCACATTGATGCCCCTAGG
AGTAATTCATCAAGAAAAAGCGACTCGAGCTTTATGTCTCTTAGGAGACAAA
GTTCTAAACAACACAAATTATTAAACGAAGAAGAAGATCTAATCAAGCCTGA
TGATATTTCTTCCGCTGGTACCAAGGATATTGAAGGGCATAGCTTACTGGAAA
ATTATGCGCCTAATATGATTCTCTCCCAGTCGACTGGAGTTGAACGTAGATTT
GAAAATTCATCATCCATCCAAAATTCGCTTGGGAATGAAATTCATGACTCGG
GTGAGCATATGGCTTCAGGTGATACTTTTAATGAACTGGATGATGGCAAATT
GCGCAAGAGCAAGAAAAATGGTGGAAGATCTCAACTTGGCCAAAATATACC
GAACTCTCAGTCTACTTTCCCCACCATTGCTAACATCGGTAGTAAAGATAATA
ATGTACCACAGCACAACTTTTCGACCTCCATATCGAGTTTAACCAATAATTTG
AGGAGAGCTGCTCCTGAAAGCTTCCATGGTTCAAGAATGAATAATATTTTTCA
CAAGAAAGGTAATCAGAATCTACTTCTGAGATCCAACGATCTCAACAAAAAT
TCTGCAGCCCCGGCCTCTCCATTGTCCAACGAACATATTACATCTAGTACGAA
CTCCGGTAGCGATGCAAACAGACAATCCAACTCAGGTGCCAAATTTAATAGC
TTCGCCCAGTTCCTTAAATCAGATGGGATTGATGCAGAATCAAGAACACAAA
GAAAATTATGGTTGCAGAGGGAGAATTCTATTATGGACTTAAGTTCACAAAA
TGACGGTAGTGACTCTATCTTTATGGCAGGAAACATTGATGCGAAAAGGGAG
TTTGAGAGAATATCCCATGAATACTCTAATGTAAAAAGATTTTACAACCCATT
AGATGAAGCATTGTTGAGAGTACAACCTATAATAACGGGAAATGCAAATAAT
ATCAGGAAAAAAAGCCATAACGATGCTCAGTCAATCGCACATTCTAGCAGTG
ATACAGATCATAAGGATGAGGACGATTTGCTCTTTACTAACTATGACAAAAA
ATTTGATGATCTTTATCCACATCTTGCAAGTGCAAAGATTCAGGCAGTGTTGT
CCGGTATATGGAAAAGCGAAAGTTACTTATTTAACAAGGATGTTAATCCAAT
CAACAAGAATAGGACAACGAGTACAAACCACAGCGTTGGCCACACTGCTTCA
CAGAATGCACGTAACTTGCTGAGGGGCCCGATGGGTTCCAGCACGACTTTGC
ACCACCAACGCGTCATTAACTCTCTGCAGCCGACTACGAGGGCAGTGAATCG
CAGGATGGAAAATGTGGGCTACATGCATACACAGCCACAACAAAGGTGAAA
ACAACCAACACGAAGCACACAGTTTAAAAGAGTAGCTAATGCTTTCGAGGTA
AAACGCGAAGTTCGTAGAGAGCGAATATGTTTGGACACTTAAGGAACATACT
ATGTTTATGCACTATAAAAGGACATGTACTTCTATACGAGCTAACGAGGCGT
ATTTGTATAACCCGGTTAACGTAATAAATGATAAATTATCGAACAAAAAGAA
AGAAAAACGTTGAATGCAACCACCGCAAATTTAGCGATTTCGCTGGATTCCG
TATCTCTTAAAAAATGGCATTAGTAAATATACCTTAAACGTGTCTATTCTTTT
AGCAATTTTTTGGTAAGTATTCCTCACGGACTATAAATACTATCGGTCAGAAT
CACTACATTCAAATTAATCTTGTTTTACCGAGTCTCTGACGTTTCATTTACAAG
CTTGTCCTTACAAAAAACCTATTTTATTACTTTAGTCCATTTTCCTTTCAAGTT
AGATATTGTTTTCCCTTCACTTAATCGAATTCCTGCAGCCCGGGGGATCCACT
AGTTCTAGAGCGGCCGCCACCGCGGTGGAGCTCCAGCTTTTGTTCCCTTTAGT
GAGGGTTAATTGCGCGCTTGGCGTAATCATGGTCATAGCTGTTTCCTGTGTGA
AATTGTTATCCGCTCACAATTCCACACAACATAGGAGCCGGAAGCATAAAGT
GTAAAGCCTGGGGTGCCTAATGAGTGAGGTAACTCACATTAATTGCGTTGCG
CTCACTGCCCGCTTTCCAGTCGGGAAACCTGTCGTGCCAGCTGCATTAATGAA
TCGGCCAACGCGCGGGGAGAGGCGGTTTGCGTATTGGGCGCTCTTCCGCTTC
CTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGG
AAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCA
TAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAAC
TATCGTCTTGAGTCCAACCCGGTAAGACACGACTTATCGCCACTGGCAGCAG
CCACTGGTAACAGGATTAGCAGAGCGAGGTATGTAGGCGGTGCTACAGAGTT
CTTGAAGTGGTGGCCTAACTACGGCTACACTAGAAGGACAGTATTTGGTATC
TGCGCTCTGCTGAAGCCAGTTACCTTCGGAAAAAGAGTTGGTAGCTCTTGATC
CGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTA
AATCAATCTAAAGTATATATGAGTAAACTTGGTCTGACAGTTACCAATGCTTA
ATCAGTGAGGCACCTATCTCAGCGATCTGTCTATTTCGTTCATCCATAGTTGC
CTGACTCCCCGTCGTGTAGATAACTACGATACGGGAGGGCTTACCATCTGGC
CCCAGTGCTGCAATGATACCGCGAGACCCACGCTCACCGGCTCCAGATTTAT
CAGCAATAAACCAGCCAGCCGGAAGGGCCGAGCGCAGAAGTGGTCCTGCAA
CTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGT
GGTGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTT
TCTGTGACTGGTGAGTACTCAACCAAGTCATTCTGAGAATAGTGTATGCGGC
GACCGAGTTGCTCTTGCCCGGCGTCAATACGGGATAATACCGCGCCACATAG
CAGAACTTTAAAAGTGCTCATCATTGGAAAACGTTCTTCGGGGCGAAAACTC
TCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAA
CAGGAAGGCAAAATGCCGCAAAAAAGGGAATAAGGGCGACACGGAAATGTT
GAATACTCATACTCTTCCTTTTTCAATATTATTGAAGCATTTATCAGGGTTATT
GTCTCATGAGCGGATACATATTTGAATGTATTTAGAAAAATAAACAAATAGG
GGTTCCGCGCACATTTCCCCGAAAAGTGCCACCTGAACGAAGCATCTGTGCT
TCATTTTGTAGAACAAAAATGCAACGCGAGAGCGCTAATTTTTCAAACAAAG
AATCTGAGCTGCATTTTTACAGAACAGAAATGCAACGCGAAAGCGCTATTTT
ACCAACGAAGAATCTGTGCTTCATTTTTGTAAAACAAAAATGCAACGCGAGA
GCGCTAATTTTTCAAACAAAGAATCTGAGCTGCATTTTTACAGAACAGAAAT
GCAACGCGAGAGCGCTATTTTACCAACAAAGAATCTATACTTCTTTTTTGTTC
TACAAAAATGCATCCCGAGAGCGCTATTTTTCTAACAAAGCATCTTAGATTAC
TTTTTTTCTCCTTTGTGCGCTCTATAATGCAGTCTCTTGATAACTTTTTGCACT
GTAGGTCCGTTAAGGTTAGAAGAAGGCTACTTTGGTGTCTATTTTCTCTTCCA
TAAAAAAAGCCTGACTCCACTTCCCGCGTTTACTGATTACTAGCGAAGCTGCG
GGTGCATTTTTTCAAGATAAAGGCATCCCCGATTATATTCTATACCGATGTGG
ATTGCGCATACTTTGTGAACAGAAAGTGATAGCGTTGATGATTCTTCATTGGT
CAGAAAATTATGAACGGTTTCTTCTATTTTGTCTCTATATACTACGTATAGGA
AATGTTTACATTTTCGTATTGTTTTCGATTCACTCTATGAATAGTTCTTACTAC
AATTTTTTTGTCTAAAGAGTAATACTAGAGATAAACATAAAAAATGTAGAGG
TCGAGTTTAGATGCAAGTTCAAGGAGCGAAAGGTGGATGGGTAGGTTATATA
GGGATATAGCACAGAGATATATAGCAAAGAGATACTTTTGAGCAATGTTTGT
GGAAGCGGTATTCGCAATATTTTAGTAGCTCGTTACAGTCCGGTGCGTTTTTG
GTTTTTTGAAAGTGCGTCTTCAGAGCGCTTTTGGTTTTCAAAAGCGCTCTGAA
GTTCCTATACTTTCTAGAGAATAGGAACTTCGGAATAGGAACTTCAAAGCGTT
TCCGAAAACGAGCGCTTCCGAAAATGCAACGCGAGCTGCGCACATACAGCTC
ACTGTTCACGTCGCACCTATATCTGCGTGTTGCCTGTATATATATATACATGA
GAAGAACGGCATAGTGCGTGTTTATGCTTAAATGCGTACTTATATGCGTCTAT
TTATGTAGGATGAAAGGTAGTCTAGTACCTCCTGTGATATTATCCCATTCCAT
GCGGGGTATCGTATGCTTCCTTCAGCACTACCCTTTAGCTGTTCTATATGCTG
CCACTCCTCAATTGGATTAGTCTCATCCTTCAATGCTATCATTTCCTTTGATAT
TGGATCATCTAAGAAACCATTATTATCATGACATTAACCTATAAAAATAGGC
GTATCACGAGGCCCTTTCGTC
pRS425-peptide 1
SEQ ID No. 12
TCGCGCGTTTCGGTGATGACGGTGAAAACCTCTGACACATGCAGCTCCCGGA
GACGGTCACAGCTTGTCTGTAAGCGGAT
GCCGGGAGCAGACAAGCCCGTCAGGGCGCGTCAGCGGGTGTTGGCGGGTGT
CGGGGCTGGCTTAACTATGCGGCATCAGAGCAGATTGTACTGAGAGTGCACC
ATATCGACTACGTCGTAAGGCCGTTTCTGACAGAGTAAAATTCTTGAGGGAA
CTTTCACCATTATGGGAAATGCTTCAAGAAGGTATTGACTTAAACTCCATCAA
ATGGTCAGGTCATTGAGTGTTTTTTATTTGTTGTATTTTTTTTTTTTTAGAGAA
AATCCTCCAATATCAAATTAGGAATCGTAGTTTCATGATTTTCTGTTACACCT
AACTTTTTGTGTGGTGCCCTCCTCCTTGTCAATATTAATGTTAAAGTGCAATTC
TTTTTCCTTATCACGTTGAGCCATTAGTATCAATTTGCTTACCTGTATTCCTTT
ACTATCCTCCTTTTTCTCCTTCTTGATAAATGTATGTAGATTGCGTATATAGTT
TCGTCTACCCTATGAACATATTCCATTTTGTAATTTCGTGTCGTTTCTATTATG
AATTTCATTTATAAAGTTTATGTACAAATATCATAAAAAAAGAGAATCTTTTT
AAGCAAGGATTTTCTTAACTTCTTCGGCGACAGCATCACCGACTTCGGTGGTA
CTGTTGGAACCACCTAAATCACCAGTTCTGATACCTGCATCCAAAACCTTTTT
AACTGCATCTTCAATGGCCTTACCTTCTTCAGGCAAGTTCAATGACAATTTCA
ACATCATTGCAGCAGACAAGATAGTGGCGATAGGGTCAACCTTATTCTTTGG
CAAATCTGGAGCAGAACCGTGGCATGGTTCGTACAAACCAAATGCGGTGTTC
TTGTCTGGCAAAGAGGCCAAGGACGCAGATGGCAACAAACCCAAGGAACCT
GGGATAACGGAGGCTTCATCGGAGATGATATCACCAAACATGTTGCTGGTGA
TTATAATACCATTTAGGTGGGTTGGGTTCTTAACTAGGATCATGGCGGCAGAA
TCAATCAATTGATGTTGAACCTTCAATGTAGGGAATTCGTTCTTGATGGTTTC
CTCCACAGTTTTTCTCCATAATCTTGAAGAGGCCAAAAGATTAGCTTTATCCA
AGGACCAAATAGGCAATGGTGGCTCATGTTGTAGGGCCATGAAAGCGGCCAT
TCTTGTGATTCTTTGCACTTCTGGAACGGTGTATTGTTCACTATCCCAAGCGA
CACCATCACCATCGTCTTCCTTTCTCTTACCAAAGTAAATACCTCCCACTAAT
TCTCTGACAACAACGAAGTCAGTACCTTTAGCAAATTGTGGCTTGATTGGAG
ATAAGTCTAAAAGAGAGTCGGATGCAAAGTTACATGGTCTTAAGTTGGCGTA
CAATTGAAGTTCTTTACGGATTTTTAGTAAACCTTGTTCAGGTCTAACACTAC
CGGTACCCCATTTAGGACCAGCCACAGCACCTAACAAAACGGCATCAACCTT
CTTGGAGGCTTCCAGCGCCTCATCTGGAAGTGGGACACCTGTAGCATCGATA
GCAGCACCACCAATTAAATGATTTTCGAAATCGAACTTGACATTGGAACGAA
CATCAGAAATAGCTTTAAGAACCTTAATGGCTTCGGCTGTGATTTCTTGACCA
ACGTGGTCACCTGGCAAAACGACGATCTTCTTAGGGGCAGACATAGGGGCAG
ACATTAGAATGGTATATCCTTGAAATATATATATATATTGCTGAAATGTAAAA
GGTAAGAAAAGTTAGAAAGTAAGACGATTGCTAACCACCTATTGGAAAAAA
CAATAGGTCCTTAAATAATATTGTCAACTTCAAGTATTGTGATGCAAGCATTT
AGTCATGAACGCTTCTCTATTCTATATGAAAAGCCGGTTCCGGCCTCTCACCT
TTCCTTTTTCTCCCAATTTTTCAGTTGAAAAAGGTATATGCGTCAGGCGACCT
CTGAAATTAACAAAAAATTTCCAGTCATCGAATTTGATTCTGTGCGATAGCGC
CCCTGTGTGTTCTCGTTATGTTGAGGAAAAAAATAATGGTTGCTAAGAGATTC
GAACTCTTGCATCTTACGATACCTGAGTATTCCCACAGTTAACTGCGGTCAAG
ATATTTCTTGAATCAGGCGCCTTAGACCGCTCGGCCAAACAACCAATTACTTG
TTGAGAAATAGAGTATAATTATCCTATAAATATAACGTTTTTGAACACACATG
AACAAGGAAGTACAGGACAATTGATTTTGAAGAGAATGTGGATTTTGATGTA
ATTGTTGGGATTCCATTTTTAATAAGGCAATAATATTAGGTATGTGGATATAC
TAGAAGTTCTCCTCGACCGTCGATATGCGGTGTGAAATACCGCACAGATGCG
TAAGGAGAAAATACCGCATCAGGAAATTGTAAACGTTAATATTTTGTTAAAA
TTCGCGTTAAATTTTTGTTAAATCAGCTCATTTTTTAACCAATAGGCCGAAAT
CGGCAAAATCCCTTATAAATCAAAAGAATAGACCGAGATAGGGTTGAGTGTT
GTTCCAGTTTGGAACAAGAGTCCACTATTAAAGAACGTGGACTCCAACGTCA
AAGGGCGAAAAACCGTCTATCAGGGCGATGGCCCACTACGTGAACCATCACC
CTAATCAAGTTTTTTGGGGTCGAGGTGCCGTAAAGCACTAAATCGGAACCCT
AAAGGGAGCCCCCGATTTAGAGCTTGACGGGGAAAGCCGGCGAACGTGGCG
AGAAAGGAAGGGAAGAAAGCGAAAGGAGCGGGCGCTAGGGCGCTGGCAAG
TGTAGCGGTCACGCTGCGCGTAACCACCACACCCGCCGCGCTTAATGCGCCG
CTACAGGGCGCGTCGCGCCATTCGCCATTCAGGCTGCGCAACTGTTGGGAAG
GGCGATCGGTGCGGGCCTCTTCGCTATTACGCCAGCTGGCGAAAGGGGGATG
TGCTGCAAGGCGATTAAGTTGGGTAACGCCAGGGTTTTCCCAGTCACGACGT
TGTAAAACGACGGCCAGTGAGCGCGCGTAATACGACTCACTATAGGGCGAAT
TGGGTACCGGGCCCCCCCTCGAGATACTAGCGTTGAATGTTAGCGTCAACAA
CAAGAAGTTTAATGACGCGGAGGCCAAGGCAAAAAGATTCCTTGATTACGTA
AGGGAGTTAGAATCATTTTGAATAAAAAACACGCTTTTTCAGTTCGAGTTTAT
CATTATCAATACTGCCATTTCAAAGAATACGTAAATAATTAATAGTAGTGATT
TTCCTAACTTTATTTAGTCAAAAAATTAGCCTTTTAATTCTGCTGTAACCCGTA
CATGCCCAAAATAGGGGGCGGGTTACACAGAATATATAACATCGTAGGTGTC
TGGGTGAACAGTTTATTCCTGGCATCCACTAAATATAATGGAGCCCGCTTTTT
AAGCTGGCATCCAGAAAAAAAAAGAATCCCAGCACCAAAATATTGTTTTCTT
CACCAACCATCAGTTCATAGGTCCATTCTCTTAGCGCAACTACAGAGAACAG
GGGCACAAACAGGCAAAAAACGGGCACAACCTCAATGGAGTGATGCAACCT
GCCTGGAGTAAATGATGACACAAGGCAATTGACCCACGCATGTATCTATCTC
ATTTTCTTACACCTTCTATTACCTTCTGCTCTCTCTGATTTGGAAAAAGCTGAA
AAAAAAGGTTGAAACCAGTTCCCTGAAATTATTCCCCTACTTGACTAATAAGT
ATATAAAGACGGTAGGTATTGATTGTAATTCTGTAAATCTATTTCTTAAACTT
CTTAAATTCTACTTTTATAGTTAGTCTTTTTTTTAGTTTTAAAACACCAAGAAC
TTAGTTTCGAATAAACACACATAAACAAACAAAATGGATTCTCAAAAGACAA
ATCCATCAGATTCTCAAAAGACTAATCCATCAGATTCTCAAAAGACAAATCC
ATCTGATTCTCAAAAGACTAATCCATCTGATTCACAAAAGACTAATCCATCAG
ATTCACAAAAGACAAATCCATCTGATTCACAAAAGACAAATCCATCAGATTC
ACAAAAGACTAATCCATCTGGTGGTGGTGGTTCTGGTGGTGGTGGTTCTGGTG
GTGGTGGTTCTGCTTCTGGTGGTGGTactacaatgtatacgacatggtgtccttatagctctgaatctgaga
ctagcacattaaccagtatgcatgaaacggttacaacagacgctacagtctgcactcacgagtcttgcatgccctcgcagacaac
aagtttgattacatcttctataaaaatgtccactaaaaacgtcgcaacttctgtaagcacctcaacggttgaatcctcatatgcatgct
ccacatgtgctgaaacgtcacactcgtattatccgtgcaaacagcttcatcaagttctgtaacacagcagaccacatccacaaag
agttgggtaagttcaatgacaacttcggatgaagatttcaataagcacgctaccggtaagtatcatgtaacatcttcaggtacctca
accatttcgactagtgtaagtgaagccacgagtacatcaagcattgactcagaatctcaagaacaatcatcacacttattatcgaca
tcggtcctttcatcctcctccttgtctgctacattatcctctgacagtactattttgctattcagttctgtatcatcactaagtgtcgaaca
gtcaccagttaccacacttcaaatttcttcaacatcagagattttacaacccacttcttccacagctattgctacaatatctgcctctac
atcatcactttccgcaacatctatctctacaccatctacctctgtggaatcgactattgaatcttcatcattgactccgacggtatcttct
attttcctctcatcatcatctgctccctcttctctacaaacatctgttaccactacagaagtttccactacttcaatctccatacaatacca
aacttcatcaatggtaacaattagccaatatatgggcagtggatcgcaaacgcgtttgccattaggaaagttggtcttcgccatcat
ggcagttgcttgcaatgtaattttcagttaaACAGGCCCCTTTTCCTTTGTCGATATCATGTAATT
AGTTATGTCACGCTTACATTCACGCCCTCCTCCCACATCCGCTCTAACCGAAA
AGGAAGGAGTTAGACAACCTGAAGTCTAGGTCCCTATTTATTTTTTTTAATAG
TTATGTTAGTATTAAGAACGTTATTTATATTTCAAATTTTTCTTTTTTTTCTGTA
CAAACGCGTGTACGCATGTAACATTATACTGAAAACCTTGCTTGAGAAGGTT
TTGGGACGCTCGAAGGCTTTAATTTGCAAGCTTCGCAGTTTACACTCTCATCG
CGGCCGCCACCGCGGTGGAGCTCCAGCTTTTGTTCCCTTTAGTGAGGGTTAAT
TGCGCGCTTGGCGTAATCATGGTCATAGCTGTTTCCTGTGTGAAATTGTTATC
CGCTCACAATTCCACACAACATAGGAGCCGGAAGCATAAAGTGTAAAGCCTG
GGGTGCCTAATGAGTGAGGTAACTCACATTAATTGCGTTGCGCTCACTGCCCG
CTTTCCAGTCGGGAAACCTGTCGTGCCAGCTGCATTAATGAATCGGCCAACG
CGCGGGGAGAGGCGGTTTGCGTATTGGGCGCTCTTCCGCTTCCTCGCTCACTG
ACTCGCTGCGCTCGGTCGTTCGGCTGCGGCGAGCGGTATCAGCTCACTCAAA
GGCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGCT
GGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTC
CCCCTGGAAGCTCCCTCGTGCGCTCTCCTGTTCCGACCCTGCCGCTTACCGGA
TACCTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTT
GAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTG
GTGGCCTAACTACGGCTACACTAGAAGGACAGTATTTGGTATCTGCGCTCTGC
TGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCA
GAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAA
AGGATCTTCACCTAGATCCTTTTAAATTAAAAATGAAGTTTTAAATCAATCTA
AAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCC
GTCGTGTAGATAACTACGATACGGGAGGGCTTACCATCTGGCCCCAGTGCTG
CAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCCAGT
TAATAGTTTGCGCAACGTTGTTGCCATTGCTACAGGCATCGTGGTGTCACGCT
CGTCGTTTGGTATGGCTTCATTCAGCTCCGGTTCCCAACGATCAAGGCGAGTT
ACATGATCCCCCATGTTGTGCAAAAAAGCGGTTAGCTCCTTCGGTCCTCCGAT
CGTTGTCAGAAGTAAGTTGGCCGCAGTGTTATCACTCATGGTTATGGCAGCAC
TGCATAATTCTCTTACTGTCATGCCATCCGTAAGATGCTTTTCTGTGACTGGTG
AGTACTCAACCAAGTCATTCTGAGAATAGTGTATGCGGCGACCGAGTTGCTC
TTGCCCGGCGTCAATACGGGATAATACCGCGCCACATAGCAGAACTTTAAAA
GTGCTCATCATTGGAAAACGTTCTTCGGGGCGAAAACTCTCAAGGATCTTACC
GCTGTTGAGATCCAGTTCGATGTAACCCACTCGTGCACCCAACTGATCTTCAG
CATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTC
TTCCTTTTTCAATATTATTGAAGCATTTATCAGGGTTATTGTCTCATGAGCGGA
TACATATTTGAATGTATTTAGAAAAATAAACAAATAGGGGTTCCGCGCACAT
TTCCCCGAAAAGTGCCACCTGAACGAAGCATCTGTGCTTCATTTTGTAGAACA
AAAATGCAACGCGAGAGCGCTAATTTTTCAAACAAAGAATCTGAGCTGCATT
TTTACAGAACAGAAATGCAACGCGAAAGCGCTATTTTACCAACGAAGAATCT
GTGCTTCATTTTTGTAAAACAAAAATGCAACGCGAGAGCGCTAATTTTTCAAA
CAAAGAATCTGAGCTGCATTTTTACAGAACAGAAATGCAACGCGAGAGCGCT
ATTTTACCAACAAAGAATCTATACTTCTTTTTTGTTCTACAAAAATGCATCCC
GAGAGCGCTATTTTTCTAACAAAGCATCTTAGATTACTTTTTTTCTCCTTTGTG
CGCTCTATAATGCAGTCTCTTGATAACTTTTTGCACTGTAGGTCCGTTAAGGT
TAGAAGAAGGCTACTTTGGTGTCTATTTTCTCTTCCATAAAAAAAGCCTGACT
CCACTTCCCGCGTTTACTGATTACTAGCGAAGCTGCGGGTGCATTTTTTCAAG
ATAAAGGCATCCCCGATTATATTCTATACCGATGTGGATTGCGCATACTTTGT
GAACAGAAAGTGATAGCGTTGATGATTCTTCATTGGTCAGAAAATTATGAAC
GGTTTCTTCTATTTTGTCTCTATATACTACGTATAGGAAATGTTTACATTTTCG
TATTGTTTTCGATTCACTCTATGAATAGTTCTTACTACAATTTTTTTGTCTAAA
GAGTAATACTAGAGATAAACATAAAAAATGTAGAGGTCGAGTTTAGATGCAA
GTTCAAGGAGCGAAAGGTGGATGGGTAGGTTATATAGGGATATAGCACAGA
GATATATAGCAAAGAGATACTTTTGAGCAATGTTTGTGGAAGCGGTATTCGC
AATATTTTAGTAGCTCGTTACAGTCCGGTGCGTTTTTGGTTTTTTGAAAGTGC
GTCTTCAGAGCGCTTTTGGTTTTCAAAAGCGCTCTGAAGTTCCTATACTTTCTA
GAGAATAGGAACTTCGGAATAGGAACTTCAAAGCGTTTCCGAAAACGAGCGC
TTCCGAAAATGCAACGCGAGCTGCGCACATACAGCTCACTGTTCACGTCGCA
CCTATATCTGCGTGTTGCCTGTATATATATATACATGAGAAGAACGGCATAGT
GCGTGTTTATGCTTAAATGCGTACTTATATGCGTCTATTTATGTAGGATGAAA
GGTAGTCTAGTACCTCCTGTGATATTATCCCATTCCATGCGGGGTATCGTATG
CTTCCTTCAGCACTACCCTTTAGCTGTTCTATATGCTGCCACTCCTCAATTGGA
TTAGTCTCATCCTTCAATGCTATCATTTCCTTTGATATTGGATCATACTAAGAA
ACCATTATTATCATGACATTAACCTATAAAAATAGGCGTATCACGAGGCCCTT
TCGTC
PRS425-peptide 2
SEQ ID No. 13
TCGCGCGTTTCGGTGATGACGGTGAAAACCTCTGACACATGCAGCTCCCGGA
GACGGTCACAGCTTGTCTGTAAGCGGATGCCGGGAGCAGACAAGCCCGTCAG
GGCGCGTCAGCGGGTGTTGGCGGGTGTCGGGGCTGGCTTAACTATGCGGCAT
CAGAGCAGATTGTACTGAGAGTGCACCATATCGACTACGTCGTAAGGCCGTT
TCTGACAGAGTAAAATTCTTGAGGGAACTTTCACCATTATGGGAAATGCTTCA
AGAAGGTATTGACTTAAACTCCATCAAATGGTCAGGTCATTGAGTGTTTTTTA
TTTGTTGTATTTTTTTTTTTTTAGAGAAAATCCTCCAATATCAAATTAGGAATC
GTAGTTTCATGATTTTCTGTTACACCTAACTTTTTGTGTGGTGCCCTCCTCCTT
GTCAATATTAATGTTAAAGTGCAATTCTTTTTCCTTATCACGTTGAGCCATTA
GTATCAATTTGCTTACCTGTATTCCTTTACTATCCTCCTTTTTCTCCTTCTTGAT
AAATGTATGTAGATTGCGTATATAGTTTCGTCTACCCTATGAACATATTCCAT
TTTGTAATTTCGTGTCGTTTCTATTATGAATTTCATTTATAAAGTTTATGTACA
AATATCATAAAAAAAGAGAATCTTTTTAAGCAAGGATTTTCTTAACTTCTTCG
GCGACAGCATCACCGACTTCGGTGGTACTGTTGGAACCACCTAAATCACCAG
TTCTGATACCTGCATCCAAAACCTTTTTAACTGCATCTTCAATGGCCTTACCTT
CTTCAGGCAAGTTCAATGACAATTTCAACATCATTGCAGCAGACAAGATAGT
GGCGATAGGGTCAACCTTATTCTTTGGCAAATCTGGAGCAGAACCGTGGCAT
GGTTCGTACAAACCAAATGCGGTGTTCTTGTCTGGCAAAGAGGCCAAGGACG
CAGATGGCAACAAACCCAAGGAACCTGGGATAACGGAGGCTTCATCGGAGA
TGATATCACCAAACATGTTGCTGGTGATTATAATACCATTTAGGTGGGTTGGG
TTCTTAACTAGGATCATGGCGGCAGAATCAATCAATTGATGTTGAACCTTCAA
TGTAGGGAATTCGTTCTTGATGGTTTCCTCCACAGTTTTTCTCCATAATCTTGA
AGAGGCCAAAAGATTAGCTTTATCCAAGGACCAAATAGGCAATGGTGGCTCA
TGTTGTAGGGCCATGAAAGCGGCCATTCTTGTGATTCTTTGCACTTCTGGAAC
GGTGTATTGTTCACTATCCCAAGCGACACCATCACCATCGTCTTCCTTTCTCTT
ACCAAAGTAAATACCTCCCACTAATTCTCTGACAACAACGAAGTCAGTACCT
TTAGCAAATTGTGGCTTGATTGGAGATAAGTCTAAAAGAGAGTCGGATGCAA
AGTTACATGGTCTTAAGTTGGCGTACAATTGAAGTTCTTTACGGATTTTTAGT
AAACCTTGTTCAGGTCTAACACTACCGGTACCCCATTTAGGACCAGCCACAG
CACCTAACAAAACGGCATCAACCTTCTTGGAGGCTTCCAGCGCCTCATCTGG
AAGTGGGACACCTGTAGCATCGATAGCAGCACCACCAATTAAATGATTTTCG
AAATCGAACTTGACATTGGAACGAACATCAGAAATAGCTTTAAGAACCTTAA
TGGCTTCGGCTGTGATTTCTTGACCAACGTGGTCACCTGGCAAAACGACGATC
TTCTTAGGGGCAGACATAGGGGCAGACATTAGAATGGTATATCCTTGAAATA
TATATATATATTGCTGAAATGTAAAAGGTAAGAAAAGTTAGAAAGTAAGACG
ATTGCTAACCACCTATTGGAAAAAACAATAGGTCCTTAAATAATATTGTCAA
CTTCAAGTATTGTGATGCAAGCATTTAGTCATGAACGCTTCTCTATTCTATAT
GAAAAGCCGGTTCCGGCCTCTCACCTTTCCTTTTTCTCCCAATTTTTCAGTTGA
AAAAGGTATATGCGTCAGGCGACCTCTGAAATTAACAAAAAATTTCCAGTCA
TCGAATTTGATTCTGTGCGATAGCGCCCCTGTGTGTTCTCGTTATGTTGAGGA
AAAAAATAATGGTTGCTAAGAGATTCGAACTCTTGCATCTTACGATACCTGA
GTATTCCCACAGTTAACTGCGGTCAAGATATTTCTTGAATCAGGCGCCTTAGA
CCGCTCGGCCAAACAACCAATTACTTGTTGAGAAATAGAGTATAATTATCCT
ATAAATATAACGTTTTTGAACACACATGAACAAGGAAGTACAGGACAATTGA
TTTTGAAGAGAATGTGGATTTTGATGTAATTGTTGGGATTCCATTTTTAATAA
GGCAATAATATTAGGTATGTGGATATACTAGAAGTTCTCCTCGACCGTCGATA
TGCGGTGTGAAATACCGCACAGATGCGTAAGGAGAAAATACCGCATCAGGA
AATTGTAAACGTTAATATTTTGTTAAAATTCGCGTTAAATTTTTGTTAAATCA
GCTCATTTTTTAACCAATAGGCCGAAATCGGCAAAATCCCTTATAAATCAAA
AGAATAGACCGAGATAGGGTTGAGTGTTGTTCCAGTTTGGAACAAGAGTCCA
CTATTAAAGAACGTGGACTCCAACGTCAAAGGGCGAAAAACCGTCTATCAGG
GCGATGGCCCACTACGTGAACCATCACCCTAATCAAGTTTTTTGGGGTCGAG
GTGCCGTAAAGCACTAAATCGGAACCCTAAAGGGAGCCCCCGATTTAGAGCT
TGACGGGGAAAGCCGGCGAACGTGGCGAGAAAGGAAGGGAAGAAAGCGAA
AGGAGCGGGCGCTAGGGCGCTGGCAAGTGTAGCGGTCACGCTGCGCGTAAC
CACCACACCCGCCGCGCTTAATGCGCCGCTACAGGGCGCGTCGCGCCATTCG
CCATTCAGGCTGCGCAACTGTTGGGAAGGGCGATCGGTGCGGGCCTCTTCGC
TATTACGCCAGCTGGCGAAAGGGGGATGTGCTGCAAGGCGATTAAGTTGGGT
AACGCCAGGGTTTTCCCAGTCACGACGTTGTAAAACGACGGCCAGTGAGCGC
GCGTAATACGACTCACTATAGGGCGAATTGGGTACCGGGCCCCCCCTCGAGA
TACTAGCGTTGAATGTTAGCGTCAACAACAAGAAGTTTAATGACGCGGAGGC
CAAGGCAAAAAGATTCCTTGATTACGTAAGGGAGTTAGAATCATTTTGAATA
AAAAACACGCTTTTTCAGTTCGAGTTTATCATTATCAATACTGCCATTTCAAA
GAATACGTAAATAATTAATAGTAGTGATTTTCCTAACTTTATTTAGTCAAAAA
ATTAGCCTTTTAATTCTGCTGTAACCCGTACATGCCCAAAATAGGGGGCGGGT
TACACAGAATATATAACATCGTAGGTGTCTGGGTGAACAGTTTATTCCTGGCA
TCCACTAAATATAATGGAGCCCGCTTTTTAAGCTGGCATCCAGAAAAAAAAA
GAATCCCAGCACCAAAATATTGTTTTCTTCACCAACCATCAGTTCATAGGTCC
ATTCTCTTAGCGCAACTACAGAGAACAGGGGCACAAACAGGCAAAAAACGG
GCACAACCTCAATGGAGTGATGCAACCTGCCTGGAGTAAATGATGACACAAG
GCAATTGACCCACGCATGTATCTATCTCATTTTCTTACACCTTCTATTACCTTC
TGCTCTCTCTGATTTGGAAAAAGCTGAAAAAAAAGGTTGAAACCAGTTCCCT
GAAATTATTCCCCTACTTGACTAATAAGTATATAAAGACGGTAGGTATTGATT
GTAATTCTGTAAATCTATTTCTTAAACTTCTTAAATTCTACTTTTATAGTTAGT
CTTTTTTTTAGTTTTAAAACACCAAGAACTTAGTTTCGAATAAACACACATAA
ACAAACAAAATGCATGGTAAAACACAAGCTACTTCAGGTACTATTCAATCTA
TGCATGGTAAAACACAAGCAACATCTGGTACAATTCAATCTATGCATGGTAA
AACTCAAGCAACTTCTGGTACTATTCAATCTATGCATGGTAAAACTCAAGCTA
CTTCTGGTACAATTCAATCAATGCATGGTAAAACTCAAGCTACATCTGGTACT
ATTCAATCAATGCATGGTAAAACACAAGCTACTTCAGGTACAATTCAATCAA
TGCATGGTAAAACACAAGCAACATCAGGTACTATTCAATCTGGTGGTGGTGG
TTCTGGTGGTGGTGGTTCTGGTGGTGGTGGTTCTGCTTCTGGTGGTGGTactacaat
gtatacgacatggtgtccttatagctctgaatctgagactagcacattaaccagtatgcatgaaacggttacaacagacgctacagt
ctgcactcacgagtcttgcatgccctcgcagacaacaagtttgattacatcttctataaaaatgtccactaaaaacgtcgcaacttct
gtaagcacctcaacggttgaatcctcatatgcatgctccacatgtgctgaaacgtcacactcgtattcttccgtgcaaacagcttcat
caagttctgtaacacagcagaccacatccacaaagagttgggtaagttcaatgacaacttcggatgaagatttcaataagcacgct
accggtaagtatcatgtaacatcttcaggtacctcaaccatttcgactagtgtaagtgaagccacgagtacatcaagcattgactca
gaatctcaagaacaatcatcacacttattatcgacatcggtcctttcatcctcctccttgtctgctacattatcctctgacagtactatttt
gctattcagttctgtatcatcactaagtgtcgaacagtcaccagttaccacacttcaaatttatcaacatcagagattttacaaccca
cttcttccacagctattgctacaatatctgcctctacatcatcactttccgcaacatctatctctacaccatctacctagtggaatcga
ctattgaatatcatcattgactccgacggtatcttctattttcctctcatcatcatctgaccctatactacaaacatctgttaccacta
cagaagtttccactacttcaatctccatacaataccaaacttcatcaatggtaacaattagccaatatatgggcagtggatcgcaaa
cgcgtttgccattaggaaagttggtcttcgccatcatggcagttgcttgcaatgtaattttcagttaaACAGGCCCCTTTT
CCTTTGTCGATATCATGTAATTAGTTATGTCACGCTTACATTCACGCCCTCCTC
CCACATCCGCTCTAACCGAAAAGGAAGGAGTTAGACAACCTGAAGTCTAGGT
CCCTATTTATTTTTTTTAATAGTTATGTTAGTATTAAGAACGTTATTTATATTT
CAAATTTTTCTTTTTTTTCTGTACAAACGCGTGTACGCATGTAACATTATACTG
AAAACCTTGCTTGAGAAGGTTTTGGGACGCTCGAAGGCTTTAATTTGCAAGCT
TCGCAGTTTACACTCTCATCGCGGCCGCCACCGCGGTGGAGCTCCAGCTTTTG
TTCCCTTTAGTGAGGGTTAATTGCGCGCTTGGCGTAATCATGGTCATAGCTGT
TTCCTGTGTGAAATTGTTATCCGCTCACAATTCCACACAACATAGGAGCCGGA
AGCATAAAGTGTAAAGCCTGGGGTGCCTAATGAGTGAGGTAACTCACATTAA
TTGCGTTGCGCTCACTGCCCGCTTTCCAGTCGGGAAACCTGTCGTGCCAGCTG
CATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGA
GCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACT
ATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTG
GCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCC
TTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGT
GCTACAGAGTTCTTGAAGTGGTGGCCTAACTACGGCTACACTAGAAGGACAG
TATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTT
GGTCATGAGATTATCAAAAAGGATCTTCACCTAGATCCTTTTAAATTAAAAAT
GAAGTTTTAAATCAATCTAAAGTATATATGAGTAAACTTGGTCTGACAGTTAC
CAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTA
CCATCTGGCCCCAGTGCTGCAATGATACCGCGAGACCCACGCTCACCGGCTC
CAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCT
AGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTAT
CACTCATGGTTATGGCAGCACTGCATAATTCTCTTACTGTCATGCCATCCGTA
AGATGCTTTTCTGTGACTGGTGAGTACTCAACCAAGTCATTCTGAGAATAGTG
TATGCGGCGACCGAGTTGCTCTTGCCCGGCGTCAATACGGGATAATACCGCG
CCACATAGCAGAACTTTAAAAGTGCTCATCATTGGAAAACGTTCTTCGGGGC
GAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTG
AGCAAAAACAGGAAGGCAAAATGCCGCAAAAAAGGGAATAAGGGCGACAC
GGAAATGTTGAATACTCATACTCTTCCTTTTTCAATATTATTGAAGCATTTATC
AGGGTTATTGTCTCATGAGCGGATACATATTTGAATGTATTTAGAAAAATAA
ACAAATAGGGGTTCCGCGCACATTTCCCCGAAAAGTGCCACCTGAACGAAGC
ATCTGTGCTTCATTTTGTAGAACAAAAATGCAACGCGAGAGCGCTAATTTTTC
AAACAAAGAATCTGAGCTGCATTTTTACAGAACAGAAATGCAACGCGAAAGC
GCTATTTTACCAACGAAGAATCTGTGCTTCATTTTTGTAAAACAAAAATGCAA
CGCGAGAGCGCTAATTTTTCAAACAAAGAATCTGAGCTGCATTTTTACAGAA
CAGAAATGCAACGCGAGAGCGCTATTTTACCAACAAAGAATCTATACTTCTT
TTTTGTTCTACAAAAATGCATCCCGAGAGCGCTATTTTTCTAACAAAGCATCT
TAGATTACTTTTTTTCTCCTTTGTGCGCTCTATAATGCAGTCTCTTGATAACTT
TTTGCACTGTAGGTCCGTTAAGGTTAGAAGAAGGCTACTTTGGTGTCTATTTT
CTCTTCCATAAAAAAAGCCTGACTCCACTTCCCGCGTTTACTGATTACTAGCG
AAGCTGCGGGTGCATTTTTTCAAGATAAAGGCATCCCCGATTATATTCTATAC
CGATGTGGATTGCGCATACTTTGTGAACAGAAAGTGATAGCGTTGATGATTCT
TCATTGGTCAGAAAATTATGAACGGTTTCTTCTATTTTGTCTCTATATACTACG
TATAGGAAATGTTTACATTTTCGTATTGTTTTCGATTCACTCTATGAATAGTTC
TTACTACAATTTTTTTGTCTAAAGAGTAATACTAGAGATAAACATAAAAAATG
TAGAGGTCGAGTTTAGATGCAAGTTCAAGGAGCGAAAGGTGGATGGGTAGGT
TATATAGGGATATAGCACAGAGATATATAGCAAAGAGATACTTTTGAGCAAT
GTTTGTGGAAGCGGTATTCGCAATATTTTAGTAGCTCGTTACAGTCCGGTGCG
TTTTTGGTTTTTTGAAAGTGCGTCTTCAGAGCGCTTTTGGTTTTCAAAAGCGCT
CTGAAGTTCCTATACTTTCTAGAGAATAGGAACTTCGGAATAGGAACTTCAA
AGCGTTTCCGAAAACGAGCGCTTCCGAAAATGCAACGCGAGCTGCGCACATA
CAGCTCACTGTTCACGTCGCACCTATATCTGCGTGTTGCCTGTATATATATAT
ACATGAGAAGAACGGCATAGTGCGTGTTTATGCTTAAATGCGTACTTATATG
CGTCTATTTATGTAGGATGAAAGGTAGTCTAGTACCTCCTGTGATATTATCCC
ATTCCATGCGGGGTATCGTATGCTTCCTTCAGCACTACCCTTTAGCTGTTCTAT
ATGCTGCCACTCCTCAATTGGATTAGTCTCATCCTTCAATGCTATCATTTCCTT
TGATATTGGATCATACTAAGAAACCATTATTATCATGACATTAACCTATAAAA
ATAGGCGTATCACGAGGCCCTTTCGTC

The present disclosure will be more readily understood by referring to the following examples which are given to illustrate the disclosure rather than to limit its scope.

### Example 1

### Engineered Yeast for Nonmagnetic Fines Recovery

### Engineer Yeast Strain BY4741

Yeast strain BY4741 was created to deposit iron oxide nanocrystals within its cellular envelope by virtue of knockout of the ccc1 gene (via plasmid 2, knock in of the ferritin complex genes FTL, FTH1, and Pcbp1 as delivered by the Plasmid of FIG. 1 (SEQ ID No. 10) and of overexpression of gene TCO89 as delivered by Plasmid SEQ ID No. 11 in FIG. 3.

FIG. 1 is a plasmid map corresponding to U1260DF290-17 bearing human ferritin gene complex FTh-FTL-PCBP1 on a PRS316 plasmid (SEQ ID NO. 10);

FIG. 2 is a plasmid map corresponding to U1260DF290-5 bearing KanMX4 gene and the ccc1 gene knocked out of a BY4742 plasmid;

FIG. 3 is a plasmid map corresponding to U1260DF290-4 bearing additional copy or copies of the TCO89 gene on a PRS423 plasmid (SEQ ID NO. 11);

FIG. 4 is a plasmid map corresponding to U1260DF290-12 bearing a metal binding peptide (peptide No. 1) on a PRS425 plasmid; and FIG. 5 is a plasmid map corresponding to U1260DF290-12 bearing a metal binding peptide (peptide No. 2) on a PRS425 plasmid.

The modifications perpetrated on S. cerevisiae by these plasmids collectively and synergistically increased the magnetic susceptibility of the created yeast in comparison to wild type yeast. The details of the sequences used are as follows:

SEQ ID No. 1 is a non-optimized sequence of the ferritin complex coding region to be integrated into the genome of S. cerevisiae as CCC1p-FTL-CCC1t-CCC1p-FTH-CCC1t-CCC1p-Pcbp1-Adh3t. SEQ ID No. 5 represents the optimized nucleic acid expression sequence for FTH synthesis, SEQ ID No. 6 represents the optimized nucleic acid expression sequence for FTL synthesis, and SEQ ID No. 7 represents the optimized nucleic acid expression sequence for PCBP1 synthesis. All together, these three expression sequences contribute to the plasmid pRS316-FTL-FTH-PCBP1 of SEQ ID No. 10 preferably used to manufacture Yeast according to embodiments of the disclosure.

SEQ ID No. 2 is a non-optimized sequence of the TCO89 gene expression cassette, while SEQ ID No. 11 is the optimized plasmid pRS423 bearing TCO89.

SEQ ID No. 3 is a non-optimized expressing portion of the GPDp-peptide #1-Aga1-Cyc1t plasmid, wherein the C-terminal 320 amino acid domain of Aga1 is designed for use as a protein anchor. In detail, SEQ ID No. 8 expresses Peptide 1, a metal binding protein of the disclosure. SEQ ID No. 12 is the plasmid pRS425 bearing the Peptide #1 expressing Gene.

SEQ ID No. 4 illustrates a non-optimized expressing portion of the GPDp-peptide #2-Aga1-Cyc1t plasmid, and SEQ ID No. 9 codes for Peptide 2 itself. SEQ ID No. 13 is the plasmid pRS425 bearing the Peptide #2 expressing Gene.

All genetic products were custom manufactured by Genscript, NJ using CloneEZ™.

The created Yeast called Strain #0 was produced as follows: Yeast strain BY4741 (MATα his3Δ1 leu2Δ0 lys2Δ0 ura3Δ0), was transformed into BY4741 ccc1Δ (MATα his3Δ1 leu2 Δ0 lys2Δ0 ura3Δ0 ccc1::kanMX4) by knockout of the ccc1 gene using the kanMX4 gene knockout method (NISHIDA K, 2012) to form BY4742 ccc1Δ. Briefly, a PCR-generated (BAUDIN; OZIER-KALOGEROPOULOS; DENOUEL; LACROUTE et al., 1993) (WACH; BRACHAT; PÖHLMANN; PHILIPPSEN, 1994) deletion strategy was used to systematically replace each yeast open reading frame from its start- to stop-codon with KanMX and two unique 20 mer molecular bar codes. The presence of the tags is detected by hybridization to a high-density oligonucleotide array, allowing growth phenotypes of individual strains to be analyzed in parallel. The correct replacement of the gene with KanMX was verified in mutated yeast by the detection of PCR products of the expected size using primers that span the left and right junctions of the deletion module within the genome.

The CCC1 gene is responsible for iron transport in and out of the yeast's vacuole, where excess iron is typically stored, and its deletion causes the yeast to store iron.

The human ferritin gene complex was added to the yeast to enable the yeast to tolerate the uptake of high concentrations of iron citrate. This enhanced tolerance occurs because of the gelatinous iron-bearing protein matrix generated by the human ferritin gene complex. Strain BY4741 ccc1Δ was transformed into magnetic Strain #0 by the addition of the ferritin genes FTL, FTH1, and Pcbp1 incorporated into the created yeast by single-copy plasmid pRS316 (FIG. 1, SEQ ID No. 10), and by the addition of the TCO89 gene on multi-copy plasmid pRS423 (plasmid map shown in FIG. 3, and SEQ ID No. 11 is the sequence). The TCO89 gene expression acts to adjusts the redox state within the yeast cell to the point that the iron ions held in the ferritin protein matrix will react with oxygen and crystallize into clusters of ferromagnetic iron oxide crystals. The overall effect is the increase of the magnetic susceptibility of the yeast cells from the diamagnetic range into the paramagnetic range (M=about 4.5-5.5 emu/g), at which level they can be concentrated in an HGMS unit.

In a final step, a mineral-binding peptide was attached to the alpha-agglutinin anchor protein, which caused the now paramagnetic yeast to selectively bind to whichever mineral phase the peptide targets. The plasmid of FIG. 4 (SEQ ID No. 3) or FIG. 5, (SEQ ID No. 4), is added to the Yeast). This characteristic of the yeast increases the magnetic susceptibility of the targeted mineral phase to the point at which it could be concentrated in an HGMS unit.

Strain #1 was created according to Strain #0 techniques but the addition of genes for the yeast alpha-agglutinin Aga2p subunit and the copper-binding repeating polypeptides DSQKTNPS×8 (DUNBAR; CURTIS, 2008) SEQ ID NO. 193, on yeast multi-copy plasmid pRS425 (Genscript, custom preparation, SEQ ID No. 3).

Strain #2 was generated from base Strain #0 by the addition of genes for the yeast alpha-agglutinin Aga2p subunit and the gold-binding repeating polypeptides MHGKTQATSGTIQS (SEQ ID No. 27)×7 (BROWN, 1997) on yeast multi-copy plasmid pRS425 (Genscript, custom preparation, SEQ ID No. 4).

Culture: Strains #0, #1 and #2 were precultured in yeast nitrogen base classification media containing glucose and lysine. Leucine was added to the Strain #0 media only.

Fleischmann's Baker's Yeast was used as a control in the following experiments, was also precultured in potato starch extract (VWR).

At mid-log phase of the cultures, as determined by OD600 measurements, aliquots of cells of each strain were transferred to new cultures containing the same media described above, but with the addition of up to 20 mM ferric citrate (Sigma Aldrich) as well as PIPES (piperazine-N, N′-bis(2-ethanesulfonic acid)) buffer (Sigma Aldrich) to maintain near neutral pH. Cells were once again grown to mid-log phase and then subjected to the analyses described below.

### Example 2

### Attraction Test

Following the procedures described above, cells of the control strain (Fleischmann's Baker's Yeast) were grown in 20 mM ferric citrate, cells of Strain #1 were grown in 5 mM ferric citrate, and cells of Strain #1 and Strain #2 were grown in 20 mM ferric citrate. Each test group of cells were then subjected to centrifugation at 4000 rpm for 2 minutes and re-suspended in Millipore filter sterilized water to dilute the solution to an 0.5 OD⁶⁰⁰. A 5 mL aliquot of each Strain was layered onto 1 mL of Optiprep™ density gradient medium (Sigma Aldrich) in one quadrant of a 4-compartment Petri dish. The dish was then placed onto a 4×4 grid of axial pole ring magnets “product number R848” (K&J Magnetics, Inc. PA) covered with a circle of black construction paper, and the attraction of the cells by the underlying magnets was observed. The results are not shown due to the faintness of the colonies. After a 20-minute incubation period, the cells of Strain #1 and Strain #2 grown in 20 mM ferric citrate settled out of solution directly onto the magnets, producing a visible outline of the underlying 4×4 grid. In comparison, the cells of Strain #1 grown in 5 mM ferric citrate produced only a faint and diffuse outline of the underlying magnetic grid while cells of the control strain grown in 30 mM ferric citrate showed no outline of the magnetic grid.

### Example 3

### Transmission Electron Microscopy

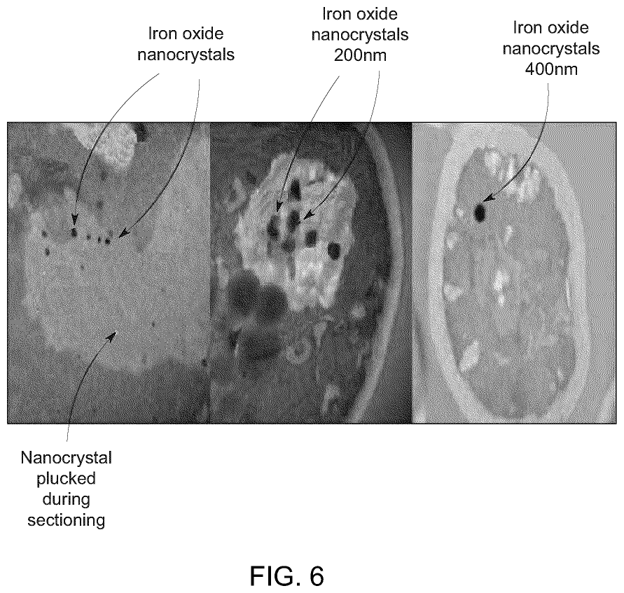

Cells of the three engineered strains (#0, #1, and #2) were chemically fixed and embedded in epoxy. Sections 70 nm thick were then cut on a microtome and placed on copper grids. One section of each strain was stained with 0.2% lead citrate, while a second section was left unstained. Electron micrographs were captured with a Hitachi H7600 transmission electron microscope. Crystals of iron oxide of up to 300 nm in diameter were seen inside Strains #0, #1, and #2 in electron micrographs in FIG. 6, which is three electron microscope images of modified Strain #0, #1 and #2 of S. cerevisiae respectively, according to the disclosure, taken at exposure=800 ms, gain=1, bin=1, gamma=1, no sharpening, normal contrast, HV=80.0 kV. Direct magnifications, from left to right, were: 80,000× for Strain #0, 60,000× for Strain #1, and 50,000× for Strain #2. Nanocrystals are indicated by arrows;

software developed at NIH by Wayne Rasband. (RASBAND, 1997)

### Example 4

### Fines Recovery

A known mass of dry gold or chalcopyrite fines (<45-micron diameter) were mixed for 1 hour with 1.5 OD⁶⁰⁰ of cells of each strain (control, #0, #1, and #2) in weighed in 2 mL microcentrifuge tubes. The mixtures were then magnetically decanted for 30 minutes, after which, any materials not attracted by the magnet were poured off into separate weighed microcentrifuge tubes. The samples were then dried at 60° C. for 96 h to remove all moisture. Each tube was then weighed a second time, and the masses of mineral fines affixed to the magnets were compared to the masses of the mineral fines transferred into the supernatant.

### Cell Coverage Assay

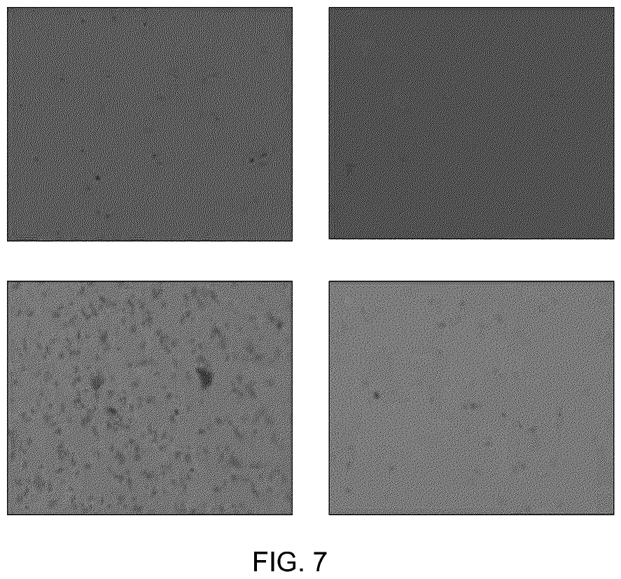

The percent surface area coverage of the gold-coated microscope slides with Strains #0, #1 and #2 show that cells displaying no mineral binding peptides as in FIG. 7, and cells displaying peptides with an affinity for chalcopyrite cover approximately 1% of the gold surface, while cells displaying peptides with an affinity for gold cover over 12% of the gold surface. Baker's yeast was not used as a control in this case, as they display filamentous surface proteins that would have interfered in the study. Instead, strain #0 acts as a control in this case.

Cell Coverage Assay. Using a modified version of the procedures described by others (PEELLE; KRAULAND; WITTRUP; BELCHER, 2005), gold-covered glass coverslips (Platypus Technologies, WI) were contacted with 5 mL of 1 OD⁶⁰⁰ samples of each of the three cell samples in phosphate buffered saline containing 0.1% Tween-20 (PBST, Sigma Aldrich) in 15 mL Falcon tubes, and rocked for one hour to ensure thorough contact of the cells with the gold surface. The coverslips were then washed in fresh PBST for 30 minutes.

Digital images of the coverslips were then taken with a Zeiss AxioCam™ IC 1 camera on a Zeiss Axio™ Imager A2m optical microscope, at 20× magnification. FIG. 7 is a series of photographs at 20× magnification in reflected, plane polarized light of (clockwise from top left): Control, Strain #0 on gold (18% coverage), Strain #1 on gold (0.85% coverage), and Strain #2 on gold (12.12% coverage) coated cover slips imaged immediately upon removal from phosphate buffered saline containing 0.1% Tween-20 (PBST) solution.

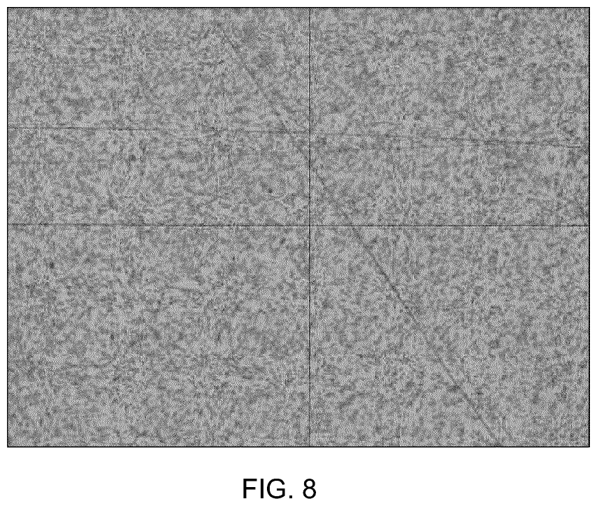

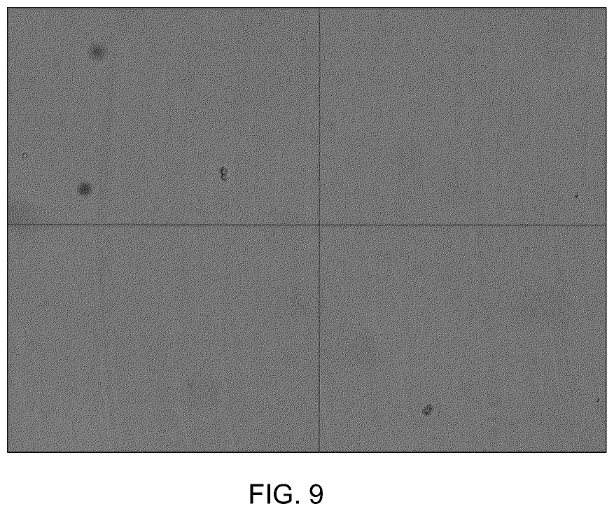

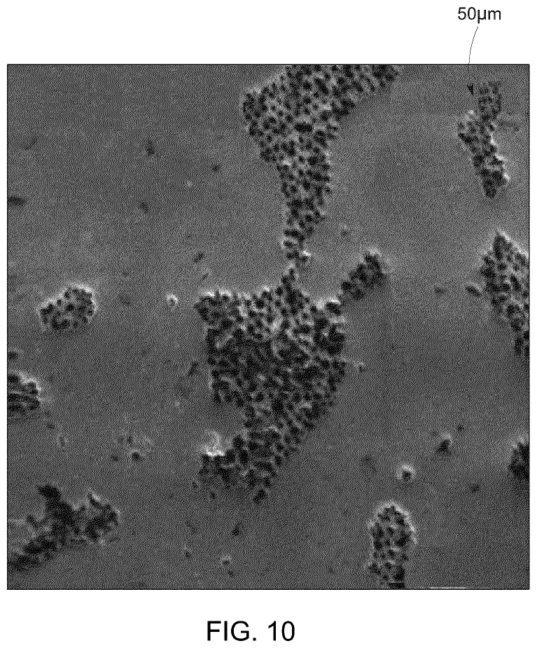

FIG. 8 shows Yeast+ expressing a gold-binding peptide bound to the surface of a gold-coated microscope slide at 5× magnification in reflected, plane polarized light. The Yeast+ surface coverage is 36.96%. The image was captured on a Zeiss optical microscope at 5× magnification in reflected, plane polarized light. Surface coverage was calculated with Image J. FIG. 9 shows a single cell of Yeast+ expressing a gold-binding peptide [MHGKTQATSGTIQS×7 (SEQ ID No. 27) (BROWN, 1997)] bound to a quartz control slide. The image showing 0% surface coverage was captured on a Zeiss optical microscope at 5× magnification in transmitted, plane polarized light. Finally, FIG. 10. shows the intact Yeast+ biofilm from the top left quadrant of FIG. 10 after 3 cycles of dehydration and rehydration with PB ST. While the cells have clustered together during the drying process, intact Yeast+ cells remain present at high concentration, 9.09% surface coverage. The image was captured at 300× magnification with a Bauch & Lombe scanning electron microscope in backscatter mode. The percent area covered was quantified using ImageJ

The relatively high coverage of the gold surface with cells displaying gold-binding peptides in comparison to the other strains indicates selective binding of Strain #2 to gold.

### Magnetization of Mineral Fines

In the gold fines experiments, Strain #2 displaying gold-binding peptide concentrated 91% of the gold. In comparison, control cells and cells of Strains #0 and #1 concentrated 61%, 62%, and 63% of the gold respectively.

In the chalcopyrite fines experiments, Strain #1 displaying chalcopyrite-binding peptide concentrated 75% of the chalcopyrite. In comparison, control cells concentrated no chalcopyrite and cells of Strains #0 and #2 both concentrated 50% of the chalcopyrite.

### Example 5

### Augmenting Process of Metal Binding Yeast in Situ

In an effort to increase the magnetic susceptibility of chalcopyrite to the point where very fine grains can be economically concentrated from a froth flotation waste stream using a standard wet carousel HGMS unit (Metseo Corporation), Strain #1 is continuously mixed in a tank with the tailings stream from a froth flotation operation where the yeast selectively coat the chalcopyrite grains. The contents of the tank are continuously passed through a wet carousel HGMS unit, where ˜15-20% of very fine chalcopyrite grains missed by the froth flotation process are captured by the magnetic filaments within the unit and separated from the nonmagnetic contents of the slurry which are relegated to a tailings pond.

### Example 6

### Creating Magnetic Characteristics: Gold

For the gold-binding magnetic yeast Strain #2, the yeast will be added to the gold processing circuit after the gravity separation unit operation. Any fines missed by the gravity concentrator will be selectively coated with yeast in a stirred tank, magnetized, and separated in a wet carousel HGMS unit.

While preferred embodiments have been described above and illustrated in the accompanying drawings, it will be evident to those skilled in the art that modifications may be made without departing from this disclosure. Such modifications are considered as possible variants comprised in the scope of the disclosure.

### Example 7

### Measuring Binding Quality: Gold Binding Peptide Yeast

To form the biofilm, Yeast+ cells were first concentrated to 1 OD₆₀₀ in phosphate buffered saline (0.1 mM, pH 7.4) with 0.1% Tween-20 (PBST). The cells were mixed with the gold target on a slide for 2 hours, then rinsed with fresh PBST for 30 minutes. The gold-coated slide was imaged immediately after removal from the rinse solution. The image was captured on a Zeiss optical microscope at 5× magnification in reflected, plane polarized light. Surface coverage was calculated with ImageJ. (RASBAND, 1997) Yeast+ expressing a gold-binding peptide bound to the surface of a gold-coated microscope slide is shown in FIG. 8. Magnification is 5×, in reflected, plane polarized light. The Yeast+ surface coverage is 35.96%.

As a control, a quartz slide with no gold target was submitted to the same process. The same imaging specifications were used. As shown in FIG. 9, no Yeast+ adhere to that slide.

### Example 8

### The Effects of Dehydration Cycles on Yeast+ Performance

The yeast bound slide of FIG. 8 underwent three cycles of dehydration and rehydration with PB ST. FIG. 10 shows the results, and while the cells have clustered together during the drying process, several intact Yeast+ cells remain present at 9.09% surface coverage. The image was captured at 300× magnification with a Bauch & Lombe scanning electron microscope in backscatter mode. Surface coverage was calculated with Image J.

### REFERENCES CITED

BAUDIN, A.; OZIER-KALOGEROPOULOS, O.; DENOUEL, A.; LACROUTE, F. et al. A simple and efficient method for direct gene deletion in Saccharomyces cerevisiae. Nucleic Acids Res, 21, n. 14, p. 3329-3330, July 1993.
BROWN, S. Metal-Recognition by Repeating Polypeptides. Nature Biotechnology, 15, p. 269-272, 1997.
CURTIS, S. B.; LEDERER, F. L.; DUNBAR, W. S.; MACGILLIVRAY, R. T. Identification of mineral-binding peptides that discriminate between chalcopyrite and enargite. Biotechnol Bioeng, 114, n. 5, p. 998-1005, May 2017.
DUNBAR, W.; CURTIS, S. A. M., R. Can Bacteriophage be Used to Separate Minerals?. In: International Future Mining Conference and Exhibition 2008, Sydney, NSW. AUSIMM.
GREENE, R. C. The Separation of Copper Sulfide Ore Minerals From Gangue using Magnetic Nanoparticles Functionalized with Peptides Selected via Phage Display. 2017. 63 f. (Master of Applied Science)—The Faculty of Graduate and Postdoctoral Studies (Mining Engineering), The University of British Columbia, Vancouver, Canada.
HWANG, J.-Y.; UNIVERSITY, M. T. Reagents for magnetizing nonmagnetic materials. 1989.
METSEO. Basics in Mineral Processing. METSEO CORPORATION. 2012: 348 p. 2012.
NISHIDA K, S. P. Induction of Biogenic Magnetization and Redox Control by a Component of the Target of Rapamycin Complex 1 Signaling Pathway. PLoS Biol, 10, n. 2, p. e1001269, 2012.
PEELLE, B. R.; KRAULAND, E. M.; WITTRUP, K. D.; BELCHER, A. M. Design criteria for engineering inorganic material-specific peptides. Langmuir, 21, n. 15, p. 6929-6933, July 2005.
RASBAND, W. ImageJ. Versão 2020. Bethesda, Md., USA.: National Institute of Mental Health, 1997.
THOTA, V.; PERRY, C. C. A Review on Recent Patents and Applications of Inorganic Material Binding Peptides. Recent Pat Nanotechnol, 11, n. 3, p. 168-180, 2017.
WACH, A.; BRACHAT, A.; PÖHLMANN, R.; PHILIPPSEN, P. New heterologous modules for classical or PCR-based gene disruptions in Saccharomyces cerevisiae. Yeast, 10, n. 13, p. 1793-1808, December 1994.

1. A magnetic reagent comprised of a recombinant yeast cell having a CCC1 gene including the following genetic modifications:
a) impairment of the CCC1 gene;
b) addition of at least one copy of a human ferritin gene complex;
c) addition of at least one copy of a TCO89 gene; and
d) addition of at least one copy of a mineral- or metal ion-adsorbing target peptide, and
wherein the magnetic susceptibility or mass magnetization of said recombinant yeast cell is increased.

2. The magnetic reagent of claim 1 wherein the increase is to greater than 4.5-5.5 emu/g.

3. The magnetic reagent of claim 1 wherein the mineral- or metal ion-adsorbing target peptide is operably associated with an α-agglutinin anchor domain.

4. The magnetic reagent of claim 1 wherein the human ferritin gene complex is expressed on a plasmid.

5. The magnetic reagent of claim 1 wherein the TCO89 gene is expressed on a plasmid.

6. The magnetic reagent of claim 3 wherein the expression of a mineral- or metal ion-adsorbing target peptide attached to the α-agglutinin anchor domain is mediated by a plasmid.

7. The magnetic reagent of any one of claims 4-6, wherein the plasmid is selected from a group consisting of pRS316, pRS423 and pRS425.

8. The magnetic reagent of any one of claims 1-7 wherein the yeast cell is derived from Saccharomyces cerevisiae.

9. The magnetic reagent of claim 8 wherein the yeast is Saccharomyces cerevisiae knockout strain BY4742.

10. The magnetic reagent of claim 1, wherein the mineral- or metal ion-adsorbing target peptide comprises an amino acid sequence at least 99% identical to any one of SEQ ID NOs: 14 to 934.

11. The magnetic reagent of claim 1, wherein the mineral- or metal ion-adsorbing target peptide comprises an amino acid sequence at least 95% identical to any one of SEQ ID NOs: 14 to 934.

12. The magnetic reagent of claim 1, wherein the mineral- or metal ion-adsorbing target peptide comprises an amino acid sequence identical to any one of SEQ ID NOs: 14 to 934.

13. The magnetic reagent of claim 1, wherein the mineral- or metal ion-adsorbing target peptide comprises the amino acid sequence DSQKTNPS.

14. The magnetic reagent of claim 1, wherein the mineral- or metal ion-adsorbing target peptide comprises the amino acid sequence MHGKTQATSGTIQS.

15. A method of extracting metals from ore slurries using the magnetic reagent of any one of claims 1-14.

16. The method of claim 15 wherein the metal is copper.

17. The method of claim 15 wherein the metal is gold.

18. The method of claim 15 wherein the metal is silver.

19. A mining aid comprising the recombinant yeast cell of any one of claims 1-14.
